(19) United States
(12) Patent Application Publication
(10) Pub. No.: US 2022/0289727 A1
(43) Pub. Date: Sep. 15, 2022

(54) ENZYME INHIBITORS

(71) Applicant: KALVISTA PHARMACEUTICALS LIMITED, Salisbury (GB)

(72) Inventors: Mitchell Lewis CHILDS, Salisbury (GB); Rebecca Louise DAVIE, Salisbury (GB); Hannah Joy EDWARDS, Salisbury (GB); David Michael EVANS, Salisbury (GB); Simon Teanby HODGSON, Ampthill (GB); Alessandro MAZZACANI, Salisbury (GB); David Edward CLARK, Harlow Essex (GB); Paul Stuart HINCHLIFFE, Saffron Walden Essex (GB); Thomas Matthew BAKER, Nottingham (GB); Colin Peter SAMBROOK SMITH, Nottingham (GB); Alun John SMITH, Nottingham (GB); Joseph William WRIGGLESWORTH, Nottingham (GB); Xuezheng YANG, Nottingham (GB)

(21) Appl. No.: 17/634,593

(22) PCT Filed: Feb. 13, 2020
(86) PCT No.: PCT/GB2020/050334

§ 371 Date: Feb. 11, 2022

(30) Foreign Application Priority Data
Aug. 21, 2019 (GB) PCT/GB2019/052358

(51) Int. Cl. C07D 409/12; C07D 409/14; C07D 413/14; C07D 417/14; C07D 471/04; C07D 491/113; C07D 495/04

(52) U.S. Cl. CPC C07D 409/12; C07D 409/14; C07D 413/14; C07D 417/14; C07D 471/04; C07D 491/113; C07D 495/04

(57) ABSTRACT

The present invention provides compounds of formula (I) or (Ia) compositions comprising such compounds; the use of such compounds in therapy; and methods of treating patients with such compounds; wherein A, B, n, R2, R3, R4, R5, and R6 are as defined herein.

This invention relates to enzyme inhibitors that are inhibitors of Factor XIIa (FXIIa), and to the pharmaceutical compositions, and uses of, such inhibitors.

### BACKGROUND TO THE INVENTION

The compounds of the present invention are inhibitors of factor XIIa (FXIIa) and thus have a number of possible therapeutic applications, particularly in the treatment of diseases or conditions in which factor XIIa inhibition is implicated.

FXIIa is a serine protease (EC 3.4.21.38) derived from its zymogen precursor, factor XII (FXII), which is expressed by the F12 gene. Single chain FXII has a low level of amidolytic activity that is increased upon interaction with negatively charged surfaces and has been implicated in its activation (see Invanov et al., Blood. 2017 Mar. 16; 129(11):1527-1537. doi: 10.1182/blood-2016-10-744110). Proteolytic cleavage of FXII to heavy and light chains of FXIIa dramatically increases catalytic activity. FXIIa that retains its full heavy chain is αFXIIa. FXIIa that retains a small fragment of its heavy chain is βFXIIa. The separate catalytic activities of αFXIIa and βFXIIa contribute to the activation and biochemical functions of FXIIa. Mutations and polymorphisms in the F12 gene can alter the cleavage of FXII and FXIIa.

FXIIa has a unique and specific structure that is different from many other serine proteases. For instance, the Tyr99 in FXIIa points towards the active site, partially blocking the S2 pocket and giving it a closed characteristic. Other serine proteases containing a Tyr99 residue (e.g. FXa, tPA and FIXa) have a more open S2 pocket. Moreover, in several trypsin-like serine proteases the P4 pocket is lined by an “aromatic box” which is responsible for the P4-driven activity and selectivity of the corresponding inhibitors. However, FXIIa has an incomplete “aromatic box” resulting in more open P4 pocket. See e.g. “Crystal structures of the recombinant β-factor XIIa protease with bound Thr-Arg and Pro-Arg substrate mimetics” M. Pathak et al., Acta. Cryst. 2019, D75, 1-14; “Structures of human plasma V-factor XIIa cocrystallized with potent inhibitors” A Dementiev et al., Blood Advances 2018, 2(5), 549-558; “Design of Small-Molecule Active-Site Inhibitors of the S1A Family Proteases as Procoagulant and Anticoagulant Drugs” P. M. Fischer, J. Med. Chem., 2018, 61(9), 3799-3822; “Assessment of the protein interaction between coagulationfactor XII and corn trypsin inhibitor by molecular docking and biochemical validation” B. K. Hamad et al. Journal of Thrombosis and Haemostasis, 15: 1818-1828.

FXIIa converts plasma prekallikrein (PK) to plasma kallikrein (PKa), which provides positive feedback activation of FXII to FXIIa. FXII, PK, and high molecular weight kininogen (HK) together represent the contact system. The contact system is activated via a number of mechanisms, including interactions with negatively charged surfaces, negatively charged molecules, unfolded proteins, artificial surfaces, foreign tissue (e.g. biological transplants, that include bio-prosthetic heart valves, and organ/tissue transplants), bacteria, and biological surfaces (including endothelium and extracellular matrix) that mediate assembly of contact system components. In addition, the contact system is activated by plasmin, and cleavage of FXII by other enzymes can facilitate its activation.

Activation of the contact system leads to activation of the kallikrein kinin system (KKS), complement system, and intrinsic coagulation pathway (see https://www.genome.jp/kegg-bin/show_pathway?map04610). In addition, FXIIa has additional substrates both directly, and indirectly via PKa, including Proteinase-activated receptors (PARs), plasminogen, and neuropeptide Y (NPY) which can contribute to the biological activity of FXIIa. Inhibition of FXIIa could provide clinical benefits by treating diseases and conditions associated with these systems, pathways, receptors, and hormones.

PKa activation of PAR2 mediates neuroinflammation and may contribute to neuroinflammatory disorders including multiple sclerosis (see Göbel et al., Proc Natl Acad Sci USA. 2019 Jan. 2; 116(1):271-276. doi: 10.1073/pnas.1810020116). PKa activation of PAR1 and PAR2 on vascular smooth muscle cells has been implicated in vascular hypertrophy and atherosclerosis (see Abdallah et al., J Biol Chem. 2010 Nov. 5; 285(45):35206-15. doi: 10.1074/jbc.M110.171769). FXIIa activation of plasminogen to plasmin contributes to fibrinolysis (see Konings et al., Thromb Res. 2015 August; 136(2):474-80. doi: 10.1016/j.thromres.2015.06.028). PKa proteolytically cleaves NPY and thereby alters its binding to NPY receptors (Abid et al., J Biol Chem. 2009 Sep. 11; 284(37):24715-24. doi: 10.1074/jbc.M109.035253). Inhibition of FXIIa could provide clinical benefits by treating diseases and conditions caused by PAR signaling, NPY metabolism, and plasminogen activation.

FXIIa-mediated activation of the KKS results in the production of bradykinin (BK), which can mediate, for example, angioedema, pain, inflammation, vascular hyperpermeability, and vasodilatation (see Kaplan et al., Adv Immunol. 2014; 121:41-89. doi: 10.1016/B978-0-12-800100-4.00002-7; and Hopp et al., J Neuroinflammation. 2017 Feb. 20; 14(1):39. doi: 10.1186/s12974-017-0815-8). CSL-312, an antibody inhibitory against FXIIa, is currently in clinical trials for the prophylactic prevention and treatment of both C1 inhibitor deficient and normal C1 inhibitor hereditary angioedema (HAE), which results in intermittent swelling of face, hands, throat, gastro-intestinal tract and genitals (see https://www.clinicaltrials.gov/ct2/show/NCT03712228). Mutations in FXII that facilitate its activation to FXIIa have been identified as a cause of HAE (see Björkqvist et al., J Clin Invest. 2015 Aug. 3; 125(8):3132-46. doi: 10.1172/JC177139; and de Maat et al., J Allergy Clin Immunol. 2016 November; 138(5):1414-1423.e9. doi: 10.1016/j.jaci.2016.02.021). Since FXIIa mediates the generation of PK to PKa, inhibitors of FXIIa could provide protective effects of all form of BK-mediated angioedema, including HAE and non-hereditary bradykinin-mediated angioedema (BK-AEnH).

“Hereditary angioedema” can be defined as any disorder characterised by recurrent episodes of bradykinin-mediated angioedema (e.g. severe swelling) caused by an inherited genetic dysfunction/fault/mutation. There are currently three known categories of HAE: (i) HAE type 1, (ii) HAE type 2, and (iii) normal C1 inhibitor HAE (normal C1-Inh HAE). However, work on characterizing the etiologies of HAE is ongoing so it is expected that further types of HAE might be defined in the future.

Without wishing to be bound by theory, it is thought that HAE type 1 is caused by mutations in the SERPING1 gene that lead to reduced levels of C1 inhibitor in the blood. Without wishing to be bound by theory, it is thought that HAE type 2 is caused by mutations in the SERPING1 gene that lead to dysfunction of the C1 inhibitor in the blood. Without wishing to be bound by theory, the cause of normal C1-Inh HAE is less well defined and the underlying genetic dysfunction/fault/mutation can sometimes remain unknown. What is known is that the cause of normal C1-Inh HAE is not related to reduced levels or dysfunction of the C1 inhibitor (in contrast to HAE types 1 and 2). Normal C1-Inh HAE can be diagnosed by reviewing the family history and noting that angioedema has been inherited from a previous generation (and thus it is hereditary angioedema). Normal C1-Inh HAE can also be diagnosed by determining that there is a dysfunction/fault/mutation in a gene other than those related to C1 inhibitor.

For example, it has been reported that dysfunction/fault/mutation with plasminogen can cause normal C1-Inh HAE (see e.g. Veronez et al., Front Med (Lausanne). 2019 Feb. 21; 6:28. doi: 10.3389/fmed.2019.00028; or Recke et al., Clin Transl Allergy. 2019 Feb. 14; 9:9. doi: 10.1186/s13601-019-0247-x.). It has also been reported that dysfunction/fault/mutation with Factor XII can cause normal C1-Inh HAE (see e.g. Mansi et al. 2014 The Association for the Publication of the Journal of Internal Medicine Journal of Internal Medicine, 2015, 277; 585-593; or Maat et al. J Thromb Haemost. 2019 January; 17(1):183-194. doi: 10.1111/jth.14325).

However, angioedemas are not necessarily inherited. Indeed, another class of angioedema is bradykinin mediated angioedema non-hereditary (BK-AEnH), which is not caused by an inherited genetic dysfunction/fault/mutation. Often the underlying cause of BK-AEnH is unknown and/or undefined. However, the signs and symptoms of BK-AEnH are similar to those of HAE, which, without being bound by theory, is thought to be on account of the shared bradykinin mediated pathway between HAE and BK-AEnH. Specifically, BK-AEnH is characterised by recurrent acute attacks where fluids accumulate outside of the blood vessels, blocking the normal flow of blood or lymphatic fluid and causing rapid swelling of tissues such as in the hands, feet, limbs, face, intestinal tract, airway or genitals.

Specific types of BK-AEnH include: non hereditary angioedema with normal C1 Inhibitor (AE-nC1 Inh), which can be environmental, hormonal, or drug induced; acquired angioedema; anaphylaxis associated angioedema; angiotensin converting enzyme (ACE) inhibitor induced angioedema; dipeptidyl peptidase 4 inhibitor induced angioedema; and tPA induced angioedema (tissue plasminogen activator induced angioedema). However, reasons why these factors and conditions cause angioedema in only a relatively small proportion of individuals are unknown.

Environmental factors that can induce AE-nC1 Inh include air pollution (Kedarisetty et al, Otolaryngol Head Neck Surg. 2019 Apr. 30:194599819846446. doi: 10.1177/0194599819846446) and silver nanoparticles such as those used as antibacterial components in healthcare, biomedical and consumer products (Long et al., Nanotoxicology. 2016; 10(4):501-11. doi: 10.3109/17435390.2015.1088589).

Various publications suggest a link between the bradykinin and contact system pathways and BK-AEnHs, and also the potential efficacy of treatments, see e.g.: Bas et al. (N Engl J Med 2015; Leibfried and Kovary. J Pharm Pract 2017); van den Elzen et al. (Clinic Rev Allerg Immunol 2018); Han et al (JCI 2002).

For instance, BK-medicated AE can be caused by thrombolytic therapy. For example, tPA induced angioedema is discussed in various publications as being a potentially life threatening complication following thrombolytic therapy in acute stroke victims (see e.g. Simlo et al., Blood. 2017 Apr. 20; 129(16):2280-2290. doi: 10.1182/blood-2016-09-740670; Fröhlich et al., Stroke. 2019 Jun. 11:STROKEAHA119025260. doi: 10.1161/STROKEAHA.119.025260; Rathbun, Oxf Med Case Reports. 2019 Jan. 24; 2019(1):omy112. doi: 10.1093/omcr/omy112; Lekoubou et al., Neurol Res. 2014 July; 36(7):687-94. doi: 10.1179/1743132813Y.0000000302; Hill et al., Neurology. 2003 May 13; 60(9):1525-7).

Stone et al. (Immunol Allergy Clin North Am. 2017 August; 37(3):483-495.) reports that certain drugs can cause angioedema.

Scott et al. (Curr Diabetes Rev. 2018; 14(4):327-333. doi: 10.2174/1573399813666170214113856) reports cases of dipeptidyl Peptidase-4 Inhibitor induced angioedema.

Hermanrud et al., (BMJ Case Rep. 2017 Jan. 10; 2017. pii: bcr2016217802) reports recurrent angioedema associated with pharmacological inhibition of dipeptidyl peptidase IV and also discusses acquired angioedema related to angiotensin-converting enzyme inhibitors (ACEI-AAE). Kim et al. (Basic Clin Pharmacol Toxicol. 2019 January; 124(1):115-122. doi: 10.1111/bcpt.13097) reports angiotensin II receptor blocker (ARB)-related angioedema. Reichman et al., (Pharmacoepidemiol Drug Saf. 2017 October; 26(10):1190-1196. doi: 10.1002/pds.4260) also reports angioedema risk for patients taking ACE inhibitors, ARB inhibitors and beta blockers. Diestro et al. (J Stroke Cerebrovasc Dis. 2019 May; 28(5):e44-e45. doi: 10.1016/j.jstrokecerebrovasdis.2019.01.030) also reports a possible association between certain angioedemas and ARBs.

Giard et al. (Dermatology. 2012; 225(1):62-9. doi: 10.1159/000340029) reports that bradykinin mediated angioedema can be precipitated by estrogen contraception, so called “oestrogen associated angioedema”.

Contact system mediated activation of the KKS has also been implicated in retinal edema and diabetic retinopathy (see Liu et al., Biol Chem. 2013 March; 394(3):319-28. doi: 10.1515/hsz-2012-0316). FXIIa concentrations are increased in the vitreous fluid from patients with advance diabetic retinopathy and in Diabetic Macular Edema (DME) (see Gao et al., Nat Med. 2007 February; 13(2):181-8. Epub 2007 Jan. 28 and Gao et al., J Proteome Res. 2008 June; 7(6):2516-25. doi: 10.1021/pr800112g). FXIIa has been implicated in mediating both vascular endothelial growth factor (VEGF) independent DME (see Kita et al., Diabetes. 2015 October; 64(10):3588-99. doi: 10.2337/db15-0317) and VEGF mediated DME (see Clermont et al., Invest Ophthalmol Vis Sci. 2016 May 1; 57(6):2390-9. doi: 10.1167/iovs.15-18272). FXII deficiency is protective against VEGF induced retinal edema in mice (Clermont et al., ARVO talk 2019). Therefore it has been proposed that FXIIa inhibition will provide therapeutic effects for diabetic retinopathy and retinal edema caused by retinal vascular hyperpermeability, including DME, retinal vein occlusion, age-related macular degeneration (AMD).

As noted above, the contact system can be activated by interaction with bacteria, and therefore FXIIa has been implicated in the treatment of sepsis and bacterial sepsis (see Morrison et al., J Exp Med. 1974 September 1; 140(3):797-811). Therefore, FXIIa inhibitors could provide therapeutic benefits in treating sepsis, bacterial sepsis and disseminated intravascular coagulation (DIC).

FXIIa mediated activation of the KKS and production of BK have been implicated in neurodegenerative diseases including Alzheimer's disease, multiple sclerosis, epilepsy and migraine (see Zamolodchikov et al., Proc Natl Acad Sci USA. 2015 March 31; 112(13):4068-73. doi: 10.1073/pnas.1423764112; Simões et al., J Neurochem. 2019 August; 150(3):296-311. doi: 10.1111/jnc.14793; Göbel et al., Nat Commun. 2016 May 18; 7:11626. doi: 10.1038/ncomms11626; and https://clinicaltrials.gov/ct2/show/NCT03108469). Therefore, FXIIa inhibitors could provide therapeutic benefits in reducing the progression and clinical symptoms of these neurodegenerative diseases.

FXIIa has also been implicated in anaphylaxis (see Bender et al., Front Immunol. 2017 September 15; 8:1115. doi: 10.3389/fimmu.2017.01115; and Sala-Cunill et al., J Allergy Clin Immunol. 2015 April; 135(4):1031-43.e6. doi: 10.1016/j.jaci.2014.07.057). Therefore, FXIIa inhibitors could provide therapeutic benefits in reducing the clinical severity and incidence of anaphylactic reactions.

The role of FXIIa in coagulation was identified over 50 years ago, and has been extensively documented in publications using biochemical, pharmacological, genetic and molecular studies (see Davie et al., Science. 1964 September 18; 145(3638):1310-2). FXIIa mediated activation of factor XI (FXI) triggers the intrinsic coagulation pathway. In addition, FXIIa can increase coagulation in a FXI independent manner (see Radcliffe et al., Blood. 1977 October; 50(4):611-7; and Puy et al., J Thromb Haemost. 2013 July; 11(7):1341-52. doi: 10.1111/jth.12295). Studies on both humans and experimental animal models have demonstrated that FXII deficiency prolongs activated partial prothrombin time (APTT) without adversely affecting hemostasis (see Renne et al., J Exp Med. 2005 Jul. 18; 202(2):271-81; and Simão et al., Front Med (Lausanne). 2017 July 31; 4:121. doi: 10.3389/fmed.2017.00121). Pharmacological inhibition of FXIIa also prolongs APTT without increasing bleeding (see Worm et al., Ann Transl Med. 2015 October; 3(17):247. doi: 10.3978/j.issn.2305-5839.2015.09.07). These data suggest that inhibition of FXIIa could provide therapeutic effects against thrombosis without inhibiting bleeding. Therefore, FXIIa inhibitors could be used to treat a spectrum of prothrombotic conditions including venous thromboembolism (VTE); cancer associated thrombosis; complications caused by mechanical and bioprosthetic heart valves, catheters, extracorporeal membrane oxygenation (ECMO), left ventricular assisted devices (LVAD), dialysis, cardiopulmonary bypass (CPB); sickle cell disease, joint arthroplasty, thrombosis induced by tPA, Paget-Schroetter syndrome and Budd-Chari syndrome. FXIIa inhibitor could be used for the treatment and/or prevention of thrombosis, edema, and inflammation associated with these conditions.

Surfaces of medical devices that come into contact with blood can cause thrombosis. FXIIa inhibitors may also be useful for treating or preventing thromboembolism by lowering the propensity of devices that come into contact with blood to clot blood. Examples of devices that come into contact with blood include vascular grafts, stents, in-dwelling catheters, external catheters, orthopedic prosthesis, cardiac prosthesis, and extracorporeal circulation systems.

Preclinical studies have shown that FXIIa has been shown to contribute to stroke and its complications following both ischemic stroke, and hemorrhagic accidents (see Barbieri et al., J Pharmacol Exp Ther. 2017 March; 360(3):466-475. doi: 10.1124/jpet.116.238493; Krupka et al., PLoS One. 2016 Jan. 27; 11(1):e0146783. doi: 10.1371/journal.pone.0146783; Leung et al., Transl Stroke Res. 2012 September; 3(3):381-9. doi: 10.1007/s12975-012-0186-5; Simão et al., Blood. 2017 Apr. 20; 129(16):2280-2290. doi: 10.1182/blood-2016-09-740670; and Liu et al., Nat Med. 2011 February; 17(2):206-10. doi: 10.1038/nm.2295). Therefore, FXIIa inhibition may improve clinical neurological outcomes in the treatment of patients with stroke.

FXII deficiency has been shown to reduce the formation of atherosclerotic lesions in Apoe^{-/-} mice (Didiasova et al., Cell Signal. 2018 November; 51:257-265. doi: 10.1016/j.cellsig.2018.08.006). Therefore, FXIIa inhibitors could be used in the treatment of atherosclerosis.

FXIIa, either directly, or indirectly via PKa, has been shown to activate the complement system (Ghebrehiwet et al., Immunol Rev. 2016 November; 274(1):281-289. doi: 10.1111/imr.12469). BK increases complement C3 in the retina, and an in vitreous increase in complement C3 is associated with DME (Murugesan et al., Exp Eye Res. 2019 Jul. 24; 186:107744. doi: 10.1016/j.exer.2019.107744). Both FXIIa and PKa activate the complement system (see Irmscher et al., J Innate Immun. 2018; 10(2):94-105. doi: 10.1159/000484257; and Ghebrehiwet et al., J Exp Med. 1981 Mar. 1; 153(3):665-76).

Compounds that are said to be FXIIa inhibitors have been described by Rao et al. (“Factor XIIa Inhibitors” WO2018/093695), Hicks et al. (“Factor XIIa Inhibitors” WO2018/093716), Breslow et al. (“Aminotriazole immunomodulators for treating autoimmune diseases” WO2017/123518) and Ponda et al. (“Aminacylindazole immunomodulators for treatment of autoimmune diseases” WO2017/205296 and “Pyranopyrazole and pyrazolopyridine immunomodulators for treatment of autoimmune diseases” WO2019/108565). FXII/FXIIa inhibitors are said to have been described by Nolte et al. (“Factor XII inhibitors for the administration with medical procedures comprising contact with artificial surfaces” WO2012/120128).

However, there remains a need to develop new FXIIa inhibitors that will have utility to treat a wide range of disorders, in particular angioedema; HAE, including: (i) HAE type 1, (ii) HAE type 2, and (iii) normal C1 inhibitor HAE (normal C1-Inh HAE); BK-AEnH, including AE-nC1 Inh, ACE and tPA induced angioedema; vascular hyperpermeability; stroke including ischemic stroke and haemorrhagic accidents; retinal edema; diabetic retinopathy; DME; retinal vein occlusion; AMD; neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; anaphylaxis; thrombosis; thromboembolism caused by increased propensity of medical devices that come into contact with blood to clot blood; prothrombotic conditions including disseminated intravascular coagulation (DIC), venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; and atherosclerosis. In particular, there remains a need to develop new FXIIa inhibitors.

### DESCRIPTION OF THE INVENTION

The present invention relates to a series of heterocyclic derivatives that are inhibitors of Factor XIIa (FXIIa). The compounds of the invention are potentially useful in the treatment of diseases or conditions in which factor XIIa inhibition is implicated. The invention further relates to pharmaceutical compositions of the inhibitors, to the use of the compositions as therapeutic agents, and to methods of treatment using these composition.

In a first aspect, the present invention provides a compound of formula (I) or (Ia),

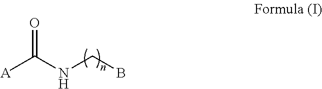

wherein:
n is 0, 1, or 2;
A is (i) a 5-membered heteroaryl of formula (II),

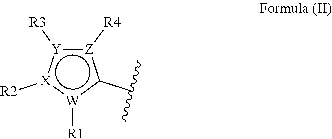

wherein W is S;
Z is C or N;
X and Y are C;
R1 is absent;
R4 is absent or H;
R2 are R3 are independently selected from H, halo, alkyl, —SO₂NR13R14, —(CH₂)₀₋₃heterocyclyl, —(CH₂)₀₋₃NR12(CH₂)₀₋₃(heterocyclyl), and —(CH₂)₀₋₃aryl; and
wherein one of R2 or R3 is not H; or
wherein W is S;
X, Y and Z are C;
R1 is absent;
R3 is halo or alkyl;
R4 is H, halo, or alkyl; and
R2 is selected from —(CH₂)₀₋₃NR13R14, —(CH₂)₀₋₃NR12(CH₂)₀₋₃(aryl), —(CH₂)₀₋₃NR12(CH₂)₀₋₃(heterocyclyl), —(CH₂)₀₋₃—O—(CH₂)₀₋₃(aryl), —(CH₂)₀₋₃—O—(CH₂)₀₋₃(heterocyclyl), —(CH₂)₀₋₃—O—(CH₂)₀₋₃(heteroaryl), —(CH₂)₀₋₃—O—(CH₂)₁₋₄NR13R14, and —(CH₂)₀₋₃heterocyclyl; or
wherein X, Y and Z are independently N, C or S;
wherein at least one of X, Y and Z is N or S;
W is C;
R3 and R4 are independently absent or independently selected from H, alkyl and halo;
R2 is selected from H, halo, alkyl, and cycloalkyl; and
R1 is selected from —(CH₂)₀₋₃NR12(CH₂)₀₋₃(heterocyclyl), —(CH₂)₀₋₃NR12CO(CH₂)₀₋₃(heterocyclyl), —(CH₂)₀₋₃—O—(CH₂)₀₋₃(heterocyclyl), and —(CH₂)₀₋₃heterocycyl; or
wherein Y and Z are N;
W and X are C;
R1 and R2 are selected from H, halo, alkyl, cycloalkyl, and —(CH₂)₀₋₃aryl;
R3 and R4 are independently absent or independently selected from —(CH₂)₀₋₃heterocyclyl, and —(CH₂)₀₋₃aryl; and
wherein at least one of R3 or R4 is selected from —(CH₂)₀₋₃heterocyclyl, and —(CH₂)₀₋₃aryl; or
wherein Y or Z are independently C, N or S;
wherein at least one of Y and Z is N or S;
W and X are C;
R1 is H;
R2 is selected from H, alkyl, aryl, and halo;
R4 is absent, or selected from H and alkyl; and
R3 is (CH₂)₀₋₃(heterocyclyl); or
wherein Y and X are independently C or N;
wherein at least one of Y or X is N;
W and Z are C;
R1 and R4 are independently selected from H, alkyl, and halo; and
one of R2 and R3 is absent and the other of R2 and R3 is

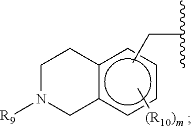

m is 0, 1, 2, or 3;
R9 is selected from H and alkyl;
Each R10 is independently selected from alkyl and halo;
A is (ii) a 9-membered heteroaromatic bicycle of formula (III)

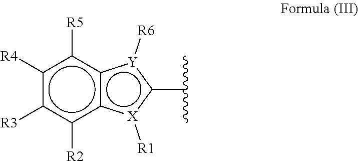

wherein X and Y are independently selected from C, N or S;
wherein at least one of X and Y is N or S;
wherein R1 and R6 are independently absent or independently selected from H and —(CH₂)₀₋₃heterocyclyl;
wherein R2 is selected from H, halo, —(CH₂)₀₋₃NR12(CH₂)₀₋₃(heterocyclyl), and —(CH₂)₀₋₃heterocyclyl;
R3, R4, and R5 are independently selected from H, alkyl and halo; and wherein at least one of R2, R3, R4, R5 is not absent or H;
or,

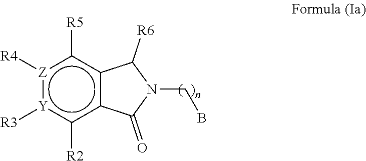

wherein n is 0, 1, or 2;
wherein Z and Y and independently selected from C and N;
wherein R6 is selected from H and alkyl;
wherein R4 and R5 are independently absent, or independently selected from H, alkyl, and halo; and
wherein one of R2 and R5 is —(CH₂)₀₋₃NR12(CH₂)₀₋₃(heterocyclyl), and the other of R2 and
R5 is selected from H, alkyl, and halo;
B is:
   (i) a fused 6,5- or 6,6-heteroaromatic bicyclic ring, containing N and, optionally, one or two additional heteroatoms independently selected from N, O and S;
   wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF₃ and —NR13R14;
   wherein the 6,5-heteroaromatic bicyclic ring may be attached via the 6- or 5-membered ring; or
   (ii) phenyl substituted with —(CH₂)₁₋₃NH₂ and two groups selected from methyl, ethyl and propyl; or
   (iii) pyridine substituted with NH₂ and two groups selected from methyl, ethyl and propyl;
   (iv) a fused 6,5- or 6,6-bicyclic ring containing N and containing an aromatic ring fused to a non-aromatic ring and, optionally, one or two additional heteroatoms independently selected from N, O and S;
   wherein the fused 6,5- or 6,6-bicyclic ring may be optionally substituted with 1, 2, or 3 substituents selected from alkyl, alkoxy, OH, halo, CN, —COOR13, —CONR13R14, CF₃ and —NR13R14;
   wherein the 6,5-bicyclic ring may be attached via the 6- or 5-membered ring;

alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, —N(R12)₂ and fluoro;

alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₁-C₆)alkoxy, OH, —NR13R14, —NHCOCH₃, —CO(heterocyclyl^{b}), —COOR13, —CONR13R14, CN, CF₃, halo, oxo, and heterocyclyl^{b};

alkyl^{b} is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₁-C₆)alkoxy, OH, —N(R12)₂, —NHCOCH₃, CF₃, halo, oxo, cyclopropane, —O(aryl^{b}), aryl^{b}, and heterocyclyl^{b};

alkylene is a bivalent linear saturated hydrocarbon having 1 to 5 carbon atoms (C₁-C₅); alkylene may optionally be substituted with 1 or 2 substituents independently selected from alkyl, (C₁-C₆)alkoxy, OH, CN, CF₃, and halo;

aryl is phenyl, biphenyl or naphthyl; aryl may be optionally substituted with 1, 2 or 3 substituents independently selected from alkyl, alkoxy, OH, —SO₂CH₃, halo, CN, —(CH₂)₀₋₃—O-heteroaryl^{b}, aryl^{b}, —O-aryl^{b}, —(CH₂)₀₋₃-heterocyclyl^{b}, —(CH₂)₁₋₃-aryl^{b}, —(CH₂)₀₋₃-heteroaryl^{b}, —COOR13, —CONR13R14, —(CH₂)₀₋₃—NR13R14, OCF₃ and CF₃; or two adjacent carbon ring atoms on the aryl may be optionally linked by a heteroalkylene to form a non-aromatic ring containing 5, 6, or 7 ring members; or optionally wherein two adjacent ring atoms on aryl are linked to form a 5- or 6-membered aromatic ring containing 1 or 2 heteroatoms that are selected from N, NR8, S, and O;

aryl^{b} is phenyl, biphenyl or naphthyl, which may be optionally substituted with 1, 2 or 3 substituents independently selected from methyl, ethyl, propyl, isopropyl, alkoxy, OH, —SO₂CH₃, N(R12)₂, halo, CN, and CF₃; or two adjacent carbon ring atoms on the aryl may be optionally linked by a heteroalkylene to form a non-aromatic ring containing 5, 6, or 7 ring members;

cycloalkyl is a monocyclic saturated hydrocarbon ring of between 3 and 6 carbon atoms (C₃-C₆); cycloalkyl may optionally be substituted with 1 or 2 substituents independently selected from alkyl^{b}, (C₁-C₆)alkoxy, OH, CN, CF₃, and halo;

halo is F, Cl, Br, or I;

heteroalkylene is a bivalent linear saturated hydrocarbon having 2 to 5 carbon atoms (C₂-C₅), wherein 1 or 2 of the 2 to 5 carbon atoms are replaced with NR8, S, or O; heteroalkylene may optionally be substituted with 1 or 2 substituents independently selected from alkyl (C₁-C₆)alkoxy, OH, CN, CF₃, and halo;

heteroaryl is a 5- or 6-membered carbon-containing aromatic ring containing 1, 2, 3, or 4 ring members that are selected from N, NR8, S, and O; heteroaryl may be optionally substituted with 1, 2 or 3 substituents independently selected from alkyl, alkoxy, aryl^{b}, OH, OCF₃, halo, heterocyclyl^{b}, CN, and CF₃; heteroaryl^{b} is a 5- or 6-membered carbon-containing aromatic ring containing one, two or three ring members that are selected from N, NR8, S, and O; heteroaryl^{b} may be optionally substituted with 1, 2 or 3 substituents independently selected from methyl, ethyl, propyl, isopropyl, alkoxy, OH, OCF₃, halo, CN, and CF₃;

heterocyclyl is a 4-, 5-, 6-, or 7-membered carbon-containing non-aromatic ring containing one or two ring members that are selected from N, NR8, S, SO, SO₂ and O; heterocyclyl may be optionally substituted with 1, 2, 3, or 4 substituents independently selected from alkyl^{b}, alkoxy, OH, OCF₃, halo, oxo, CN, —NR13R14, —O(aryl^{b}), —O(heteroaryl^{b}) and CF₃; or optionally wherein two ring atoms on heterocyclyl are linked with an alkylene to form a non-aromatic ring containing 5, 6, or 7 ring members; or optionally wherein two adjacent ring atoms on heterocyclyl are linked to form a 5- or 6-membered aromatic ring containing 1 or 2 heteroatoms that are selected from N, NR8, S, and O; or optionally wherein a carbon ring atom on heterocyclyl is substituted with a heteroalkylene such that the carbon ring atom on heterocyclyl together with the heteroalkylene forms a heterocyclyl^{b} that is spiro to ring heterocyclyl;

heterocyclyl^{b} is a 4-, 5-, 6-, or 7-membered carbon-containing non-aromatic ring containing one or two ring members that are selected from N, NR12, S, SO, SO₂ and O; heterocyclyl^{b} may be optionally substituted with 1, 2, 3, or 4 substituents independently selected from methyl, ethyl, propyl, isopropyl, alkoxy, OH, OCF₃, halo, oxo, CN, and CF₃;

R13 and R14 are independently selected from H, —SO₂CH₃, alkyl^{b}, heteroaryl^{b}, and cycloalkyl; or R13 and R14 together with the nitrogen atom to which they are attached form a carbon-containing 4-, 5-, 6- or 7-membered heterocylic ring, optionally containing an additional heteroatom selected from N, NR8, S, SO, SO₂, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃; or R13 and R14 together with the nitrogen atom to which they are attached form a carbon-containing 5- or 6-membered heterocylic ring, which is fused to an aryl^{b} or a heteroaryl^{b};

R8 is independently selected from H, —SO₂CH₃, alkyl^{b}, —(CH₂)₀₋₃aryl^{b}, —(CH₂)₀₋₃heteroaryl^{b}, —(CH₂)₀₋₃cycloalkyl, and —(CH₂)₀₋₃heterocyclyl^{b}; or R8 is a carbon-containing 4-, 5-, 6- or 7-membered heterocylic ring containing 1, 2 or 3 heteroatoms independently selected from N, N12, S, SO, SO₂, and O, which may be saturated or unsaturated with 1 or 2 double bonds and which may be optionally mono- or di-substituted with substituents independently selected from oxo, alkyl, alkoxy, OH, halo, —SO₂CH₃, and CF₃;

R12 is independently selected from H, —SO₂CH₃, —COCH₃, methyl, ethyl, propyl, isopropyl, and cycloalkyl;

and tautomers, isomers, stereoisomers (including enantiomers, diastereoisomers and racemic and scalemic mixtures thereof), deuterated isotopes, and pharmaceutically acceptable salts and/or solvates thereof.

The invention is also described by the appended numbered embodiments.

The compounds of the present invention have been developed to be inhibitors of FXIIa. As noted above, FXIIa has a unique and specific binding site and there is a need for small molecule FXIIa inhibitors.

The present invention also provides a prodrug of a compound as herein defined, or a pharmaceutically acceptable salt and/or solvate thereof.

The present invention also provides an N-oxide of a compound as herein defined, or a prodrug or pharmaceutically acceptable salt and/or solvate thereof.

It will be understood that certain compounds of the present invention may exist in solvated, for example hydrated, as well as unsolvated forms. It is to be understood that the present invention encompasses all such solvated forms.

It will be understood that “pharmaceutically acceptable salts and/or solvates thereof” means “pharmaceutically acceptable salts thereof”, “pharmaceutically acceptable solvates thereof”, and “pharmaceutically acceptable solvates of salts thereof”.

It will be understood that substituents may be named as its free unbonded structure (e.g. piperidine) or by its bonded structure (e.g. piperidinyl). No difference is intended.

It will be understood that the compounds of the invention comprise several substituents. When any of these substituents is defined more specifically herein, the substituents/optional substituents to these groups described above also apply, unless stated otherwise. For example, R2 can be —(CH₂)₀₋₃heterocyclyl, which more specifically can be piperidinyl. In this case, piperidinyl can be optionally substituted in the same manner as “heterocyclyl”.

It will be understood that “alkylene” has two free valencies i.e. it is bivalent, meaning that it is capable of being bonded to twice. For example, when two adjacent ring atoms on A″ are linked by an alkylene to form a cyclopentane, the alkylene will be —CH₂CH₂CH₂—.

It will be understood that when any variable (e.g. alkyl) occurs more than once, its definition on each occurrence is independent of every other occurrence.

It will be understood that combinations of substituents and variables are permissible only if such combinations result in stable compounds.

As is clear from the definitions above, and for the avoidance of any doubt, it will be understood that “B” and “Y” define closed groups as defined above, and do not encompass boron and yttrium, respectively.

As noted above, “heteroalkylene” is a bivalent linear saturated hydrocarbon having 2 to 5 carbon atoms (C₂-C₅), wherein at least one of the 2 to 5 carbon atoms is replaced with NR8, S, or O. For example, —CH₂O is a “heteroalkylene” having 2 carbon atoms wherein one of the 2 carbon atoms has been replaced with O.

As used herein the term “bradykinin-mediated angioedema” means hereditary angioedema, and any non-hereditary bradykinin-mediated angioedema. For example, “bradykinin-mediated angioedema” encompasses hereditary angioedema and acute bradykinin-mediated angioedema of unknown origin.

As used herein, the term “hereditary angioedema” means any bradykinin-mediated angioedema caused by an inherited genetic dysfunction, fault, or mutation. As a result, the term “HAE” includes at least HAE type 1, HAE type 2, and normal C1 inhibitor HAE (normal C1-Inh HAE).

As noted above, A can be a 5-membered heteroaryl of formula (II),

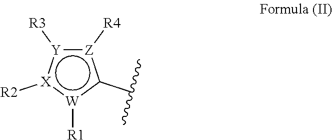

wherein W is S;
Z is C or N;
X and Y are C;
R1 is absent;
R4 is absent or H;
R2 are R3 are independently selected from H, halo, alkyl, —SO₂NR13R14, —(CH₂)₀₋₃heterocyclyl, —(CH₂)₀₋₃NR12(CH₂)₀₋₃(heterocyclyl), and —(CH₂)₀₋₃aryl; and
wherein one of R2 or R3 is not H.

Z can be C. Z can be N.

When Z is N, R4 is absent. When Z is C, R4 is H.

At least one of R2 and R3 can be either (i) halo, or (ii) selected from —(CH₂)₀₋₃heterocyclyl, —(CH₂)₀₋₃NR12(CH₂)₀₋₃(heterocyclyl), and —(CH₂)₀₋₃aryl. One of R2 and R3 can be either (i) halo, or (ii) selected from —(CH₂)₀₋₃heterocyclyl, —(CH₂)₀₋₃NR12(CH₂)₀₋₃(heterocyclyl), and —(CH₂)₀₋₃aryl. R2 and R3 can be either (i) halo, or (ii) selected from —(CH₂)₀₋₃heterocyclyl, —(CH₂)₀₋₃NR12(CH₂)₀₋₃(heterocyclyl), and —(CH₂)₀₋₃aryl. For example, one of R2 and R3 can be halo, and the other of R2 and R3 can be selected from —(CH₂)₀₋₃heterocyclyl, —(CH₂)₀₋₃NR12(CH₂)₀₋₃(heterocyclyl), and —(CH₂)₀₋₃aryl

More specifically, R2 are R3 can be independently selected from H, halo, alkyl, —SO₂NR13R14, —(CH₂)₀₋₃heterocyclyl, and —(CH₂)₀₋₃aryl.

One of R2 and R3 can be alkyl (e.g. methyl) and the other of R2 and R3 can be halo (e.g. chloro). More specifically, R2 can be alkyl and R3 can be halo. R2 can be methyl and R3 can be chloro. Alternatively, R2 can methyl and R3 can be bromo.

One of R2 and R3 can be H and the other of R2 and R3 can be —(CH₂)₀₋₃heterocyclyl. More specifically, R2 can be H and R3 can be —(CH₂)₀₋₃heterocyclyl. R2 can be H and R3 can be —(CH₂)₀₋₃(piperidinyl) e.g. —(CH₂)₂(piperidinyl).

Z can be C and R3 can be halo. Z can be C, R3 can be halo, and R2 can be alkyl. Z can be C, R3 can be chloro, and R2 can be methyl.

Z can be N and R3 can be —(CH₂)₀₋₃heterocyclyl. Z can be N and R3 can be —(CH₂)₀₋₃(piperidinyl). Z can be N and R3 can be —(CH₂)₂(piperidinyl). Z can be N, R3 can be —(CH₂)₂(piperidinyl) and R2 can be H.

“Heterocyclyl” is preferably piperidinyl, which as noted above, may be optionally substituted in the same manner as “heterocyclyl”. When the “heterocyclyl” has an NR8 group, R8 can be H or alkyl. More specifically, R8 can be H or methyl.

“Halo” can be chloro or bromo. Preferably, halo can be chloro.

“Aryl” is preferably phenyl, which as noted above, may be optionally substituted in the same manner as “aryl”. “Aryl” can be substituted with —OH and/or alkoxy (e.g. methoxy).

B is preferably a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF₃ and —NR13R14; wherein the 6,5-heteroaromatic bicyclic ring may be attached via the 6- or 5-membered ring.

B can be a fused 6,5-heteroaromatic bicyclic ring. The fused 6,5-heteroaromatic bicyclic ring can be attached via the 6-membered ring. The fused 6,5-heteroaromatic bicyclic ring can be attached via the 5-membered ring. Exemplary fused 6,5-heteroaromatic bicyclic rings can be selected from: 5-azathianaphthene, indolizine, indole, isoindole, indazole, benzimidazole, and benzothiazole, which can all be optionally substituted in the same manner as “a fused 6,5-heteroaromatic bicyclic ring”.

More specifically, when present, the fused 6,5-heteroaromatic bicyclic ring can be indole. The indole can be substituted with halo (e.g. chloro). Additionally, or in the alternative, the indole can be substituted once with alkyl (e.g. methyl) or twice with alkyl (e.g. twice with methyl).

B can be a fused 6,6-heteroaromatic bicyclic ring. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present, the fused 6,6-heteroaromatic bicyclic ring can be isoquinoline. The isoquinoline can be substituted with —NR13R14, preferably —NH₂. Additionally, or in the alternative, the isoquinoline can also be substituted with halo (e.g. fluoro).

B can also be phenyl substituted with —(CH₂)₁₋₃NH₂ and two groups selected from methyl, ethyl and propyl.

More specifically, B can be phenyl substituted with —(CH₂)₁₋₃NH₂ and two methyl groups.

Alternatively, A can be a 5-membered heteroaryl of formula (II),

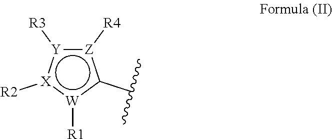

wherein W is S;
X, Y and Z are C;
R1 is absent;
R3 is halo or alkyl;
R4 is H, halo, or alkyl; and
R2 is selected from —(CH₂)₀₋₃NR13R14, —(CH₂)₀₋₃NR12(CH₂)₀₋₃(aryl), —(CH₂)₀₋₃NR12(CH₂)₀₋₃(heterocyclyl), —(CH₂)₀₋₃—O—(CH₂)₀₋₃(aryl), —(CH₂)₀₋₃—O—(CH₂)₀₋₃(heterocyclyl), —(CH₂)₀₋₃—O—(CH₂)₀₋₃(heteroaryl), —(CH₂)₀₋₃—O—(CH₂)₁₋₄NR13R14, and —(CH₂)₀₋₃heterocyclyl.

R3 can preferably be halo. When R3 is halo, it is preferably chloro.

R3 can be alkyl. When R3 is alkyl, it is preferably methyl.

R4 can be H. R4 can be halo (e.g. chloro). R4 can be alkyl (e.g. methyl).

R2 can be —(CH₂)₀₋₃NR13R14. More specifically, —NR13R14 can be —CH₂NR13R14. For example, —NR13R14 can be —N(alkyl^{b})₂, e.g. —N(CH₃)₂. —NR13R14 can also be —NH(alkyl^{b}), e.g. —NHCH₂CH₂N(R12)₂, wherein R12 can be methyl.

R2 can be —(CH₂)₀₋₃—O—(CH₂)₁₋₄NR13R14. More specifically, R2 can be —CH₂—O—(CH₂)₁₋₄NR13R14. For example, —NR13R14 can be —N(alkyl^{b})₂, e.g. —N(CH₃)₂. —NR13R14 can also be —NH(alkyl^{b}), e.g. —NHCH₂CH₂N(R12)₂, wherein R12 can be methyl.

Alternatively, R13 and R14 together with the nitrogen atom to which they are attached form a carbon-containing 4-, 5-, 6- or 7-membered heterocylic ring, optionally containing an additional heteroatom selected from N, NR8, S, SO, SO₂, and O, which may be saturated or unsaturated with 1 or 2 double bonds and which may be optionally mono- or di-substituted with substituents independently selected from oxo, alkyl, alkoxy, OH, halo, —SO₂CH₃. For example, R13 and R14, together with the N to which they are attached can form morpholine, piperazine, azepane, pyrrolidine, azetidine, pyrazolidine, imidazolidine, and piperidine, which can be optionally substituted as for R13 and R14.

R3 can be halo, R4 can be H, and R2 can be —(CH₂)₀₋₃NR13R14. More specifically, R3 can be halo, R4 can be H, and R2 can be —CH₂NR13R14. More specifically, R3 can be halo, R4 can be H, and R2 can be —CH₂NR13R14, wherein R13 and R14 together with the nitrogen atom to which they are attached form a carbon-containing 6-membered heterocylic ring, containing an additional heteroatom, which is NR8. More specifically, R3 can be halo, R4 can be H, and R2 can be —CH₂NR13R14, wherein R13 and R14 together with the nitrogen atom to which they are attached form a carbon-containing 6-membered heterocylic ring, containing an additional heteroatom, which is NR8, wherein R8 is heteroaryl^{b}. More specifically, R3 can be chloro, R4 can be H, and R2 can be —CH₂NR13R14, wherein R13 and R14 together with the nitrogen atom to which they are attached form a carbon-containing 6-membered heterocylic ring, containing an additional heteroatom, which is NR8, wherein R8 is pyridine.

R3 can be alkyl, R4 can be H, and R2 can be —(CH₂)₀₋₃NR13R14. More specifically, R3 can be alkyl, R4 can be H, and R2 can be —CH₂NR13R14. More specifically, R3 can be halo, R4 can be H, and R2 can be —CH₂NR13R14, wherein R13 and R14 together with the nitrogen atom to which they are attached form a carbon-containing 6-membered heterocylic ring, containing an additional heteroatom, which is NR8. More specifically, R3 can be alkyl, R4 can be H, and R2 can be —CH₂NR13R14, wherein R13 and R14 together with the nitrogen atom to which they are attached form a carbon-containing 6-membered heterocylic ring, containing an additional heteroatom, which is NR8, wherein R8 is heteroaryl^{b}. More specifically, R3 can be methyl, R4 can be H, and R2 can be —CH₂NR13R14, wherein R13 and R14 together with the nitrogen atom to which they are attached form a carbon-containing 6-membered heterocylic ring, containing an additional heteroatom, which is NR8, wherein R8 is pyridine.

R3 can be alkyl, R4 can be H, and R2 can be —(CH₂)₀₋₃NR13R14. More specifically, R3 can be alkyl, R4 can be H, and R2 can be —CH₂NR13R14. More specifically, R3 can be halo, R4 can be H, and R2 can be —CH₂NR13R14, wherein R13 and R14 together with the nitrogen atom to which they are attached form a carbon-containing 6-membered heterocylic ring, containing an additional heteroatom, which is NR8. More specifically, R3 can be alkyl, R4 can be H, and R2 can be —CH₂NR13R14, wherein R13 and R14 together with the nitrogen atom to which they are attached form a carbon-containing 6-membered heterocylic ring, containing an additional heteroatom, which is NR8, wherein R8 is heteroaryl^{b}. More specifically, R3 can be methyl, R4 can be H, and R2 can be —CH₂NR13R14, wherein R13 and R14 together with the nitrogen atom to which they are attached form a carbon-containing 6-membered heterocylic ring, containing an additional heteroatom, which is NR8, wherein R8 is pyrimidine.

Alternatively, R13 and R14 together with the nitrogen atom to which they are attached form a carbon-containing 5- or 6-membered heterocylic ring, which is fused to an aryl^{b} or a heteroaryl^{b}. For example, the aryl^{b} can be phenyl. For example, the heteroaryl^{b} can be pyridine.

R2 can be —(CH₂)₀₋₃NR12(CH₂)₀₋₃(aryl). More specifically, R2 can be —CH₂NR12(CH₂)₀₋₃(aryl), e.g. —CH₂NH(CH₂)₀₋₃(aryl).

R2 can be —(CH₂)₀₋₃O—(CH₂)₀₋₃(aryl). More specifically, R2 can be —CH₂O—(CH₂)₀₋₃(aryl).

“Aryl” is preferably phenyl, which as noted above, can be optionally substituted in the same manner as “aryl”.

R2 can be —(CH₂)₀₋₃NR12(CH₂)₀₋₃(heterocyclyl). More specifically, R2 can be —CH₂NR12(CH₂)₀₋₃(heterocyclyl), e.g. —CH₂NH(CH₂)₀₋₃(heterocyclyl).

R2 can be —(CH₂)₀₋₃—O—(CH₂)₀₋₃(heterocyclyl). More specifically, R2 can be —CH₂O—(CH₂)₀₋₃(heterocyclyl).

R2 can be —(CH₂)₀₋₃heterocyclyl.

“Heterocyclyl” can be selected from piperidine, pyrrolidine, piperazine, tetrahydropyran, azepane, morpholine, and azetidine, which can be optionally substituted in the same manner as “heterocyclyl”.

B can be a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF₃ and —NR13R14; wherein the 6,5-heteroaromatic bicyclic ring may be attached via the 6- or 5-membered ring.

B can preferably be a fused 6,6-heteroaromatic bicyclic ring. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present the fused 6,6-heteroaromatic bicyclic ring can preferably be isoquinoline. The isoquinoline can be substituted with —NR13R14, preferably —NH₂. Additionally, or in the alternative, the isoquinoline can also be substituted with halo (e.g. fluoro).

Alternatively, B can be a fused 6,5- or 6,6-bicyclic ring containing N and containing an aromatic ring fused to a non-aromatic ring and, optionally, one or two additional heteroatoms independently selected from N, O and S; wherein the fused 6,5- or 6,6-bicyclic ring may be optionally substituted with 1, 2, or 3 substituents selected from alkyl, alkoxy, OH, halo, CN, —COOR13, —CONR13R14, CF₃ and —NR13R14; wherein the 6,5-bicyclic ring may be attached via the 6- or 5-membered ring.

More specifically, B can be a fused 6,5-bicyclic ring containing N, and containing an aromatic ring fused to a non-aromatic ring. More specifically, the 6,5-bicyclic ring can be attached via the 5-membered ring.

Specifically, the 5-membered ring can be cyclopentane, and the 6-membered ring can be pyridine. More specifically, the 5-membered ring can be cyclopentane, and the 6-membered ring can be pyridine substituted with —NR13R14, e.g. —NH₂.

Alternatively, A can be a 5-membered heteroaryl of formula (II),

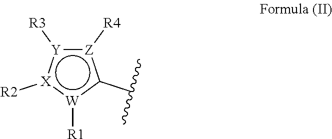

wherein X, Y and Z are independently N, C or S;
wherein at least one of X, Y and Z is N or S;
W is C;
R3 and R4 are independently absent or independently selected from H, alkyl and halo;
R2 is selected from H, halo, alkyl, and cycloalkyl; and
R1 is selected from —(CH₂)₀₋₃NR12(CH₂)₀₋₃(heterocyclyl), —(CH₂)₀₋₃NR12CO(CH₂)₀₋₃(heterocyclyl), —(CH₂)₀₋₃—O—(CH₂)₀₋₃(heterocyclyl), and —(CH₂)₀₋₃heterocyclyl.

Y and Z can be N, X can be C.

X can be S and Z can be N.

Z can be S and X and Y can be C.

X and Z can be N and Y can be C.

R3 can be H. R3 can be alkyl e.g. methyl. R3 can be halo, e.g. chloro.

R4 can be H. R4 can be alkyl e.g. methyl. R4 can be halo, e.g. chloro.

R2 can be H. R2 can be halo, e.g. chloro. R2 can be alkyl, e.g. methyl. R2 can be cycloalkyl e.g. cyclopropane.

R1 can be —(CH₂)₀₋₃NR12(CH₂)₀₋₃(heterocyclyl). Specifically, R1 can be —NR12(CH₂)₀₋₃(heterocyclyl), e.g. —NH(CH₂)₀₋₃(heterocyclyl) or —N(COCH₃)(CH₂)₀₋₃(heterocyclyl). More specifically, R1 can be —NR12CH₂(heterocyclyl), e.g. —NHCH₂(heterocyclyl) or —N(COCH₃)CH₂(heterocyclyl).

R1 can be —(CH₂)₀₋₃NR12CO(CH₂)₀₋₃(heterocyclyl). Specifically, R1 can be —NHCO(CH₂)₀₋₃(heterocyclyl). More specifically, R1 can be —NHCO(heterocyclyl).

R1 can be —(CH₂)₀₋₃—O—(CH₂)₀₋₃(heterocyclyl). Specifically, R1 can be —O—(CH₂)₀₋₃(heterocyclyl). More specifically, R1 can be —O—CH₂(heterocyclyl).

R1 can be —(CH₂)₀₋₃heterocyclyl. More specifically, R1 can be —(CH₂)₂(heterocyclyl).

Z can be S, Y and X can be C, R3 can be alkyl, R2 can be H, and R1 can be —(CH₂)₀₋₃NR12(CH₂)₀₋₃(heterocyclyl). More specifically, Z can be S, Y and X can be C, R3 can be alkyl, R2 can be H, and R1 can be —NR12(CH₂)₀₋₃(heterocyclyl). More specifically, Z can be S, Y and X can be C, R3 can be alkyl, R2 can be H, and R1 can be —NHCH₂(heterocyclyl). More specifically, Z can be S, Y and X can be C, R3 can be methyl, R2 can be H, and R1 can be —NHCH₂(heterocyclyl).

Z can be S, Y can be C, X can be N, R3 can be H and R1 can be —(CH₂)₀₋₃NR12(CH₂)₀₋₃(heterocyclyl). More specifically, Z can be S, Y can be C, X can be N, R3 can be alkyl, R2 can be H, and R1 can be —NR12(CH₂)₀₋₃(heterocyclyl). More specifically, Z can be S, Y can be C, X can be N, R3 can be alkyl, R2 can be H, and R1 can be —NHCH₂(heterocyclyl). More specifically, Z can be S, Y can be C, X can be N, R3 can be methyl, R2 can be H, and R1 can be —NHCH₂(heterocyclyl).

“Heterocyclyl” can preferably be piperidinyl. When present, NR8 is preferably NCH₃.

B can be a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF₃ and —NR13R14; wherein the 6,5-heteroaromatic bicyclic ring may be attached via the 6- or 5-membered ring.

B can preferably be a fused 6,6-heteroaromatic bicyclic ring. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present the fused 6,6-heteroaromatic bicyclic ring can preferably be isoquinoline. The isoquinoline can be substituted with —NR13R14, preferably —NH₂. Additionally, or in the alternative, the isoquinoline can also be substituted with halo (e.g. fluoro).

Alternatively, A can be a 5-membered heteroaryl of formula (II),

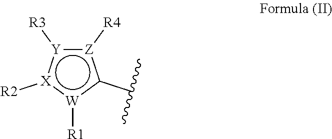

wherein Y and Z are N;
W and X are C;
R1 and R2 are selected from H, halo, alkyl, cycloalkyl, and —(CH₂)₀₋₃aryl;
R3 and R4 are independently absent or independently selected from —(CH₂)₀₋₃heterocyclyl, and —(CH₂)₀₋₃aryl; and
wherein at least one of R3 or R4 is selected from —(CH₂)₀₋₃heterocyclyl, and —(CH₂)₀₋₃aryl.

R1 and R2 can be selected from H, halo, alkyl, and cycloalkyl.

R1 can be H. R1 can be alkyl (e.g. methyl). R1 can be halo (e.g. chloro). R1 can be cycloalkyl (e.g. cyclopropane).

R2 can be H. R2 can be alkyl (e.g. methyl). R2 can be halo (e.g. chloro). R2 can be cycloalkyl (e.g. cyclopropane).

R3 can be selected from —(CH₂)₀₋₃heterocyclyl, and —(CH₂)₀₋₃aryl.

R3 can be —(CH₂)₀₋₃heterocyclyl. Specifically, R3 can be heterocyclyl. Alternatively, R3 can be —CH₂(heterocyclyl). Alternatively, R3 can be —(CH₂)₂heterocyclyl. Alternatively, R3 can be —(CH₂)₃heterocyclyl.

R3 can be —(CH₂)₀₋₃aryl. Specifically, R3 can be aryl. Alternatively, R3 can be —CH₂(aryl). Alternatively, R3 can be —(CH₂)₂aryl. Alternatively, R3 can be —(CH₂)₃(aryl).

R4 can be selected from —(CH₂)₀₋₃heterocyclyl, and —(CH₂)₀₋₃aryl.

R4 can be —(CH₂)₀₋₃heterocyclyl. Specifically, R4 can be heterocyclyl. Alternatively, R3 can be —CH₂(heterocyclyl). Alternatively, R4 can be —(CH₂)₂heterocyclyl. Alternatively, R4 can be —(CH₂)₃heterocyclyl.

R4 can be —(CH₂)₀₋₃aryl. Specifically, R4 can be aryl. Alternatively, R4 can be —CH₂(aryl). Alternatively, R4 can be —(CH₂)₂aryl. Alternatively, R4 can be —(CH₂)₃(aryl).

Specifically, R1 can be H, R2 can be halo, R3 can be absent, and R4 can be —(CH₂)₀₋₃heterocyclyl. More specifically, R1 can be H, R2 can be halo, R3 can be absent, and R4 can be —(CH₂)₂heterocyclyl. More specifically, R1 can be H, R2 can be chloro, R3 can be absent, and R4 can be —(CH₂)₂heterocyclyl.

Specifically, R1 can be H, R2 can be H, R3 can be absent, and R4 can be —(CH₂)₀₋₃heterocyclyl. More specifically, R1 can be H, R2 can be H, R3 can be absent, and R4 can be —(CH₂)₂heterocyclyl.

“Heterocyclyl” can preferably be piperidinyl. When present, piperidine preferably has an NR8, which is preferably NCH₃.

B is preferably a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF₃ and —NR13R14; wherein the 6,5-heteroaromatic bicyclic ring may be attached via the 6- or 5-membered ring.

B can be a fused 6,5-heteroaromatic bicyclic ring. The fused 6,5-heteroaromatic bicyclic ring can be attached via the 6-membered ring. The fused 6,5-heteroaromatic bicyclic ring can be attached via the 5-membered ring. Exemplary fused 6,5-heteroaromatic bicyclic rings can be selected from: 5-azathianaphthene, indolizine, indole, isoindole, indazole, benzimidazole, and benzothiazole, which can all be optionally substituted in the same manner as “a fused 6,5-heteroaromatic bicyclic ring”.

More specifically, when present, the fused 6,5-heteroaromatic bicyclic ring can be 5-azathianaphthenyl. The 5-azathianaphthenyl can be substituted with —NR13R14 (e.g. —NH₂).

B can be a fused 6,6-heteroaromatic bicyclic ring. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present the fused 6,6-heteroaromatic bicyclic ring can be isoquinoline. The isoquinoline can be substituted with —NR13R14, preferably —NH₂. Additionally, or in the alternative, the isoquinoline can also be substituted with halo (e.g. fluoro).

Alternatively, A can be a 5-membered heteroaryl of formula (II),

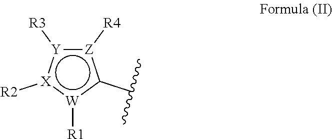

wherein Y or Z are independently C, N or S;
wherein at least one of Y and Z is N or S;
W and X are C;
R1 is H;
R2 is selected from H, alkyl, aryl, and halo;
R4 is absent, or selected from H and alkyl; and
R3 is —(CH₂)₀₋₃(heterocyclyl);

Y can be N and Z can be C.

Z can be N and Y can be C.

Z can be S and Y can be C.

Y and Z can be N.

R2 can be H. R2 can be alkyl (e.g. methyl or ethyl). R2 can be aryl (e.g. phenyl). R2 can be halo (e.g. chloro).

Z can be N and R4 can be absent.

Z can be S and R4 can be absent.

R4 can be H. R4 can be alkyl (e.g. methyl or ethyl).

R3 can be —CH₂(heterocyclyl). R3 can be —(CH₂)₂(heterocyclyl). R3 can be —(CH₂)₃(heterocyclyl). “Heterocyclyl” can be selected from morpholinyl, piperazinyl and piperidinyl. When present, NR8 can be NCH₃, NCOCH₃ or N(heteroaryl^{b}) (e.g. N(pyridinyl)).

Y and Z can be N, R2 can be H, R4 can be absent and R3 can be —(CH₂)₀₋₃(heterocyclyl). More specifically, Y and Z can be N, R2 can be H, R4 can be absent and R3 can be —(CH₂)₂(heterocyclyl). More specifically, Y and Z can be N, R2 can be H, R4 can be absent and R3 can be —(CH₂)₀₋₃(piperidine). More specifically, Y and Z can be N, R2 can be H, R4 can be absent and R3 can be —(CH₂)₀₋₃(piperidine), NR8 is present and is NCH₃.

Y can be C and Z can be N, R2 can be alkyl, R1 can be H, R4 can be alkyl, and R3 can be —(CH₂)₀₋₃(heterocyclyl). More specifically, Y can be C and Z can be N, R2 can be alkyl (e.g. methyl or ethyl), R1 can be H, R4 can be alkyl (e.g. methyl or ethyl), and R3 can be —CH₂(heterocyclyl). More specifically, Y can be C and Z can be N, R2 can be alkyl (e.g. methyl or ethyl), R1 can be H, R4 can be alkyl (e.g. methyl or ethyl), and R3 can be —CH₂(heterocyclyl), wherein heterocyclyl is piperazine. More specifically, Y can be C and Z can be N, R2 can be alkyl (e.g. methyl or ethyl), R1 can be H, R4 can be ethyl, and R3 can be —CH₂(heterocyclyl), wherein heterocyclyl is piperazine, wherein the piperazine contains an NR8 wherein R8 is heteroaryl^{b}. More specifically, Y can be C and Z can be N, R2 can be alkyl (e.g. methyl or ethyl), R1 can be H, and R3 can be —CH₂(heterocyclyl), wherein heterocyclyl is piperazine, wherein the piperazine contains an NR8 wherein R8 is pyridine.

Y can be C, Z can be S, R4 can be absent, R2 can be alkyl (e.g. methyl or ethyl), R1 can be H, and R3 can be —CH₂(heterocyclyl). More specifically, Y can be C, Z can be S, R4 can be absent, R2 can be alkyl (e.g. methyl or ethyl), R1 can be H, and R3 can be —CH₂(heterocyclyl), wherein heterocyclyl is piperazine. More specifically, Y can be C, Z can be S, R4 can be absent, R2 can be alkyl (e.g. methyl or ethyl), R1 can be H, and R3 can be —CH₂(heterocyclyl), wherein heterocyclyl is piperazine, wherein the piperazine contains an NR8 wherein R8 is heteroaryl^{b}. More specifically, Y can be C, Z can be S, R4 can be absent, R2 can be alkyl (e.g. methyl or ethyl), R1 can be H, and R3 can be —CH₂(heterocyclyl), wherein heterocyclyl is piperazine, wherein the piperazine contains an NR8 wherein R8 is pyridine.

B can be a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF₃ and —NR13R14; wherein the 6,5-heteroaromatic bicyclic ring may be attached via the 6- or 5-membered ring.

B can preferably be a fused 6,6-heteroaromatic bicyclic ring. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present, the fused 6,6-heteroaromatic bicyclic ring can preferably be isoquinoline. The isoquinoline can be substituted with —NR13R14, preferably —NH₂. Additionally, or in the alternative, the isoquinoline can also be substituted with halo (e.g. fluoro).

Alternatively, A can be a 5-membered heteroaryl of formula (II),

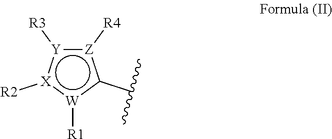

wherein Y and X are independently C or N;
wherein at least one of Y or X is N;
W and Z are C;
R1 and R4 are independently selected from H, alkyl, and halo; and
one of R2 and R3 is absent and the other of R2 and R3 is

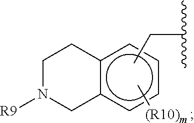

m is 0, 1, 2, or 3;
R9 is selected from H and alkyl;
Each R10 is independently selected from alkyl and halo.

Y can be N and X can be C. X can be N and Y can be C Y and X can both be N.

R1 can be H. R1 can be alkyl (e.g. methyl, ethyl, or CH₂OCH₃). R1 can be halo (e.g. chloro).

R4 can be H. R4 can be alkyl (e.g. methyl, ethyl, or CH₂OCH₃). R4 can be halo (e.g. chloro).

When Y is N, R3 can be absent and R4 can be

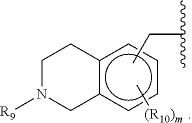

When X is N, R2 can be absent and R4 can be

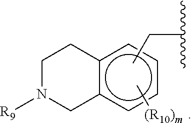

A preferred R2 or R3 group is

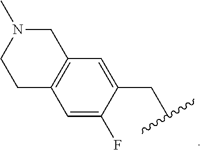 [SMILES: *Cc1cc2c(cc1F)CCN(C)C2]

m can be 0. m can be 1. m can be 2. m can be 3.

R9 can be H. R9 can be alkyl (e.g. methyl).

Each R10 can independently be alkyl (e.g. methyl). Each R10 can independently be halo. More specifically, each R10 can independently be F. More specifically, each R10 can independently be Cl.

B can be a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF₃ and —NR13R14; wherein the 6,5-heteroaromatic bicyclic ring may be attached via the 6- or 5-membered ring.

B can preferably be a fused 6,6-heteroaromatic bicyclic ring. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present the fused 6,6-heteroaromatic bicyclic ring can preferably be isoquinoline. The isoquinoline can be substituted with —NR13R14, preferably —NH₂. Additionally, or in the alternative, the isoquinoline can also be substituted with halo (e.g. fluoro).

Alternatively, A can be a 9-membered heteroaromatic bicycle of formula (III)

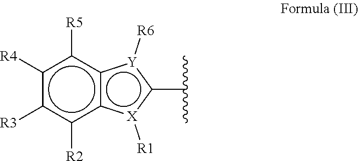

wherein X and Y are independently selected from C, N or S;
wherein at least one of X and Y is N or S;
wherein R1 and R6 are independently absent or independently selected from H and —(CH₂)₀₋₃heterocyclyl;
wherein R2 is selected from H, halo, —(CH₂)₀₋₃NR12(CH₂)₀₋₃(heterocyclyl), and —(CH₂)₀₋₃heterocycyl;
R3, R4, and R5 are independently selected from H, alkyl and halo; and
wherein at least one of R1, R2, R3, R4, R5 and R6 is not H;

X can be N. X can be N and Y can be C.

Y can be N. Y can be N and C can be C.

X and Y can both be N.

Y can be S. Y can be S and X can be C.

As noted above, at least one of R1, R2, R3, R4, R5 and R6 is not H. More specifically, either (i) at least one of R2, R3, R4 or R5 can be halo, or (ii) at least one of R1 or R2 is —(CH₂)₀₋₃NR12(CH₂)₀₋₃(heterocyclyl), or —(CH₂)₀₋₃heterocyclyl.

R1 can be —(CH₂)₀₋₃heterocyclyl. X can be N and R1 can be —(CH₂)₀₋₃heterocyclyl.

R2 can be —(CH₂)₀₋₃NR12(CH₂)₀₋₃(heterocyclyl). R2 can be —NR12(heterocyclyl). R2 can be —NR12(CH₂)(heterocyclyl).

More specifically, Y can be S, X can be C and R2 can be —(CH₂)₀₋₃NR12(CH₂)₀₋₃(heterocyclyl). More specifically, Y can be S, X can be C and R2 can be —NR12(heterocyclyl). Alternatively, Y can be S, X can be C and R2 can be —NR12(CH₂)(heterocyclyl).

Alternatively, X and Y can be N, R6 can be absent, and R2 can be —(CH₂)₀₋₃heterocyclyl. More specifically, X and Y can be N, R6 can be absent, and R2 can be —CH₂(heterocyclyl).

Alternatively, X can be N, Y can be C, R6 can be H, and R2 can be —(CH₂)₀₋₃heterocyclyl. More specifically, X and Y can be N, R6 can be absent, and R2 can be —CH₂(heterocyclyl).

“Heterocyclyl” can preferably be piperidine. “Heterocyclyl” can preferably contain an NR8 group, and in particular, N(alkyl^{b}), e.g. NCH₃ or NCH₂CH₃.

R2 can be H.

R2 can be halo. R2 can be fluoro. R2 can be chloro.

More specifically, Y can be S, X can be C, and R2 can be halo. More specifically, Y can be S, X can be C, and R2 can be chloro. More specifically, Y can be S, X can be C, and R2 can be fluoro. Additionally, R1, R3, R4, and R5 can be H.

More specifically, Y can be N, X can be C, R6 can be H, and R2 can be halo. More specifically, Y can be N, X can be C, R6 can be H, and R2 can be chloro. More specifically, Y can be N, X can be C, R6 can be H, and R2 can be fluoro. Additionally, R1, R3, R4, and R5 can be H.

R3 can be alkyl (e.g. methyl). R3 can be halo. R3 can be fluoro. R3 can be chloro.

More specifically, Y can be 5, X can be C, and R3 can be halo. More specifically, Y can be 5, X can be C, and R3 can be chloro. More specifically, Y can be 5, X can be C, and R3 can be fluoro.

More specifically, Y can be N, X can be C, R6 can be H, and R3 can be halo. More specifically, Y can be N, X can be C, R6 can be H, and R3 can be chloro. More specifically, Y can be N, X can be C, R6 can be H, and R3 can be fluoro.

R4 can be alkyl (e.g. methyl). R4 can be halo. R4 can be fluoro. R4 can be chloro.

More specifically, Y can be 5, X can be C, and R4 can be halo. More specifically, Y can be 5, X can be C, and R4 can be chloro. More specifically, Y can be 5, X can be C, and R4 can be fluoro.

More specifically, Y can be N, X can be C, R6 can be H, and R4 can be halo. More specifically, Y can be N, X can be C, R6 can be H, and R4 can be chloro. More specifically, Y can be N, X can be C, R6 can be H, and R4 can be fluoro.

R5 can be alkyl (e.g. methyl). R5 can be halo. R5 can be fluoro. R5 can be chloro.

More specifically, Y can be 5, X can be C, and R5 can be halo. More specifically, Y can be 5, X can be C, and R5 can be chloro. More specifically, Y can be 5, X can be C, and R5 can be fluoro.

More specifically, Y can be N, X can be C, R6 can be H, and R5 can be halo. More specifically, Y can be N, X can be C, R6 can be H, and R5 can be chloro. More specifically, Y can be N, X can be C, R6 can be H, and R5 can be fluoro.

B can be a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF₃ and —NR13R14; wherein the 6,5-heteroaromatic bicyclic ring may be attached via the 6- or 5-membered ring.

B can preferably be a fused 6,6-heteroaromatic bicyclic ring. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present the fused 6,6-heteroaromatic bicyclic ring can preferably be isoquinoline. The isoquinoline can be substituted with —NR13R14, preferably —NH₂. Additionally, or in the alternative, the isoquinoline can also be substituted with halo (e.g. fluoro).

Alternatively, the invention provides a compound of formula (Ia),

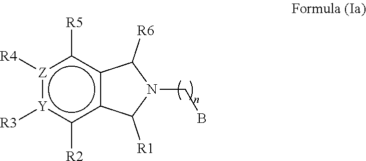

wherein n is 0, 1, or 2;
wherein Z and Y and independently selected from C and N;
wherein R6 is selected from H and alkyl;
wherein R4 and R5 are independently absent, or independently selected from H, alkyl, and halo; and
wherein one of R2 and R5 is —(CH₂)₀₋₃NR12(CH₂)₀₋₃(heterocyclyl), and the other of R2 and R5 is selected from H, alkyl, and halo.

Z can be N. Z can be N and Y can be C.

Y can be N. Y can be N and Z can be C.

Both Z and Y can be N.

Both Z and Y can be C.

When Z is N, R4 is absent.

When Y is N, R3 is absent.

R6 can be H. R6 can be alkyl, e.g. methyl.

R2 can be —(CH₂)₀₋₃NR12(CH₂)₀₋₃(heterocyclyl). More specifically, R2 can be —NR12(CH₂)₀₋₃(heterocyclyl). More specifically, R2 can be —NR12CH₂(heterocyclyl). “Heterocyclyl” can preferably be piperidine. “Heterocyclyl” can preferably contain an NR8 group, and in particular, N(alkyl^{b}), e.g. NCH₃ or NCH₂CH₃.

B can be a fused 6,5- or 6,6-heteroaromatic bicyclic ring, containing N and, optionally, one or two additional heteroatoms independently selected from N, O and S; wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF₃ and —NR13R14; wherein the 6,5-heteroaromatic bicyclic ring may be attached via the 6- or 5-membered ring.

B can preferably be a fused 6,6-heteroaromatic bicyclic ring. Exemplary fused 6,6-heteroaromatic bicyclic rings can be selected from: quinolone, isoquinoline, cinnoline, quinazoline, quinoxaline, 1,8-napthyridine, and phthalazine, which can all be optionally substituted in the same manner as “a fused 6,6-heteroaromatic bicyclic ring”.

More specifically, when present the fused 6,6-heteroaromatic bicyclic ring can preferably be isoquinoline. The isoquinoline can be substituted with —NR13R14, preferably —NH₂. Additionally, or in the alternative, the isoquinoline can also be substituted with halo (e.g. fluoro).

The present invention also encompasses, but is not limited to, the compounds below in Tables 1 to 12, and pharmaceutically acceptable salts and/or solvates thereof.

TABLE 1

| Structure | Example |
|---|---|
| Molecular formula | No. |
| 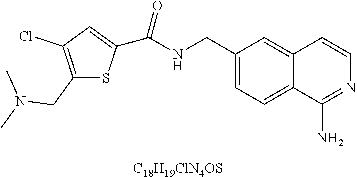 [SMILES: CN(C)Cc1sc(C(=O)NCc2ccc3c(N)nccc3c2)cc1Cl ; "C18H19ClN4OS"] | 2.01 |
| 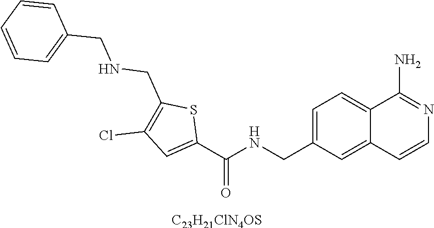 [SMILES: Nc1nccc2cc(CNC(=O)c3cc(Cl)c(CNCc4ccccc4)s3)ccc12 ; "C23H21ClN4OS"] | 2.02 |
| 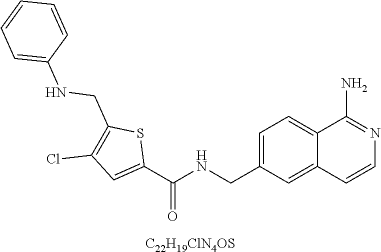 [SMILES: Nc1nccc2cc(CNC(=O)c3cc(Cl)c(CNc4ccccc4)s3)ccc12 ; "C22H19ClN4OS"] | 2.03 |
| 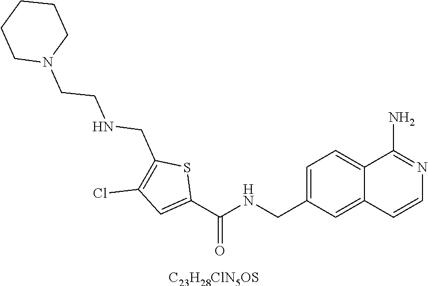 [SMILES: Nc1nccc2cc(CNC(=O)c3cc(Cl)c(CNCCN4CCCCC4)s3)ccc12 ; "C23H28ClN5OS"] | 2.04 |
| 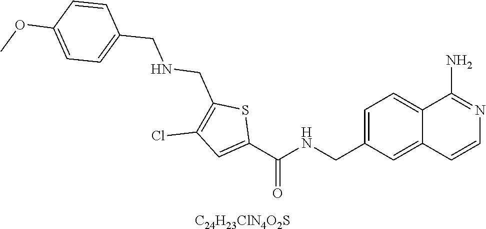 [SMILES: COc1ccc(CNCc2sc(C(=O)NCc3ccc4c(N)nccc4c3)cc2Cl)cc1 ; "C24H23ClN4O2S"] | 2.05 |
| 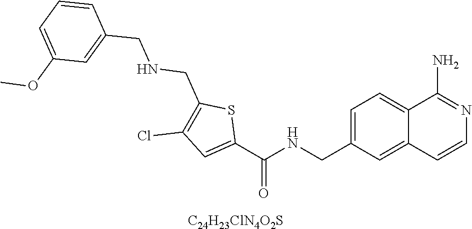 [SMILES: COc1cccc(CNCc2sc(C(=O)NCc3ccc4c(N)nccc4c3)cc2Cl)c1 ; "C24H23ClN4O2S"] | 2.06 |
| 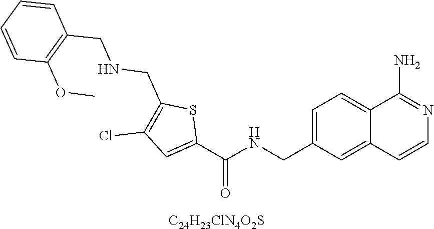 [SMILES: COc1ccccc1CNCc1sc(C(=O)NCc2ccc3c(N)nccc3c2)cc1Cl ; "C24H23ClN4O2S"] | 2.07 |
| 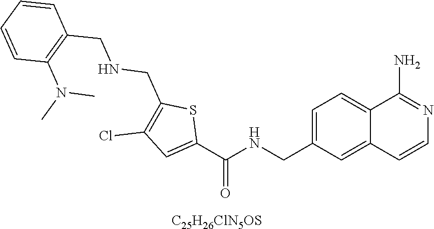 [SMILES: CN(C)c1ccccc1CNCc1sc(C(=O)NCc2ccc3c(N)nccc3c2)cc1Cl ; "C25H26ClN5OS"] | 2.08 |
| 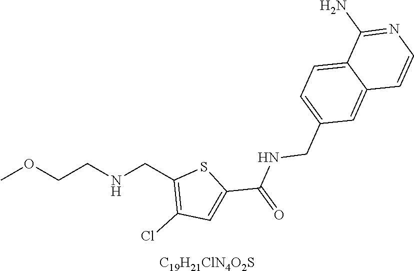 [SMILES: COCCNCc1sc(C(=O)NCc2ccc3c(N)nccc3c2)cc1Cl ; "C19H21ClN4O2S"] | 2.09 |
| 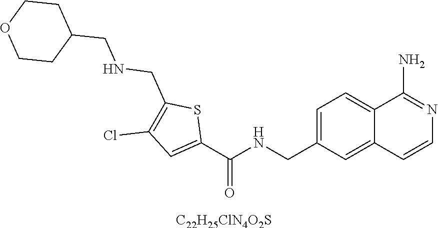 [SMILES: Nc1nccc2cc(CNC(=O)c3cc(Cl)c(CNCC4CCOCC4)s3)ccc12 ; "C22H25ClN4O2S"] | 2.10 |
| 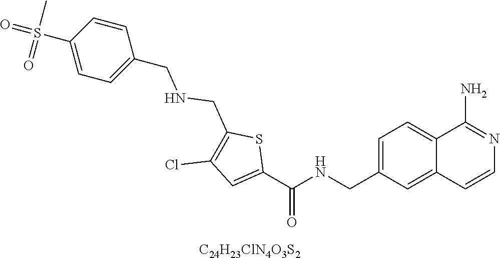 [SMILES: CS(=O)(=O)c1ccc(CNCc2sc(C(=O)NCc3ccc4c(N)nccc4c3)cc2Cl)cc1 ; "C24H23ClN4O3S2"] | 2.11 |
| 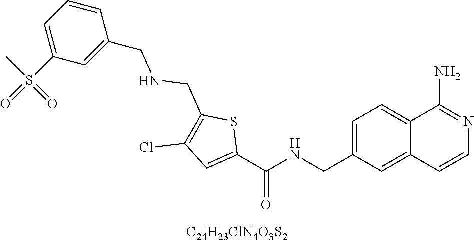 [SMILES: CS(=O)(=O)c1cccc(CNCc2sc(C(=O)NCc3ccc4c(N)nccc4c3)cc2Cl)c1 ; "C24H23ClN4O3S2"] | 2.12 |
| 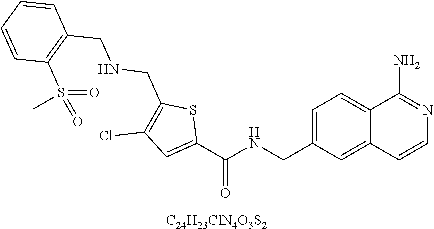 [SMILES: CS(=O)(=O)c1ccccc1CNCc1sc(C(=O)NCc2ccc3c(N)nccc3c2)cc1Cl ; "C24H23ClN4O3S2"] | 2.13 |
| 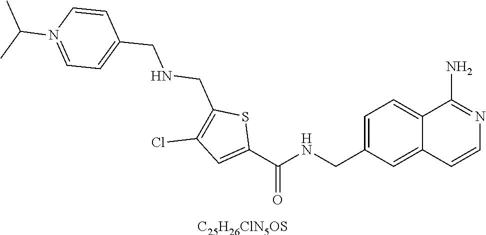 | 2.14 |
| 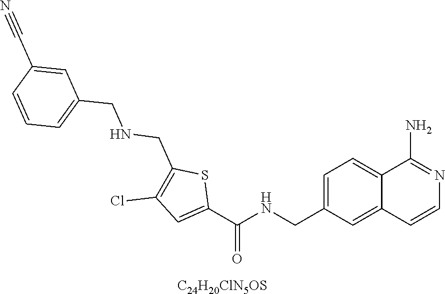 [SMILES: N#Cc1cccc(CNCc2sc(C(=O)NCc3ccc4c(N)nccc4c3)cc2Cl)c1 ; "C24H20ClN5OS"] | 2.15 |
| 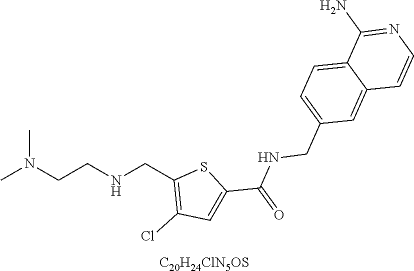 | 2.16 |
| 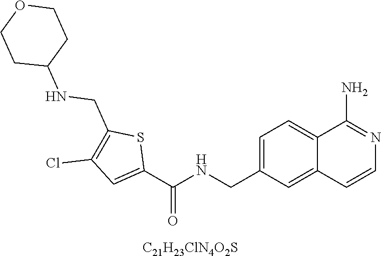 [SMILES: Nc1nccc2cc(CNC(=O)c3cc(Cl)c(CNC4CCOCC4)s3)ccc12 ; "C21H23ClN4O2S"] | 2.17 |
| 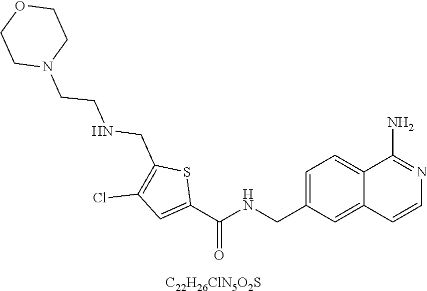 [SMILES: Nc1nccc2cc(CNC(=O)c3cc(Cl)c(CNCCN4CCOCC4)s3)ccc12 ; "C22H26ClN5O2S"] | 2.18 |
| 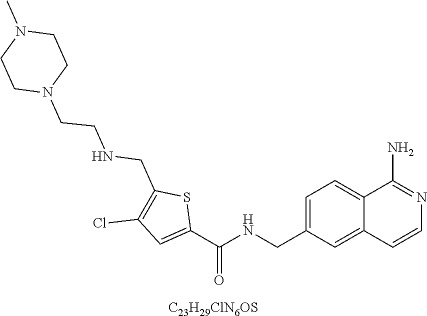 [SMILES: CN1CCN(CCNCc2sc(C(=O)NCc3ccc4c(N)nccc4c3)cc2Cl)CC1 ; "C23H29ClN6OS"] | 2.19 |
| 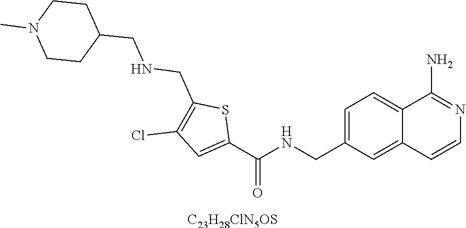 [SMILES: CN1CCC(CNCc2sc(C(=O)NCc3ccc4c(N)nccc4c3)cc2Cl)CC1 ; "C23H28ClN5OS"] | 2.20 |
| 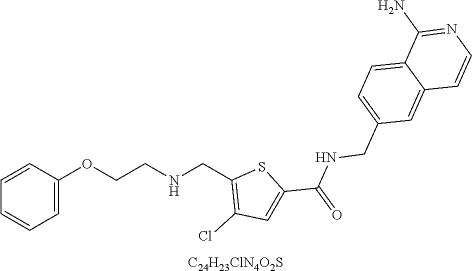 [SMILES: Nc1nccc2cc(CNC(=O)c3cc(Cl)c(CNCCOc4ccccc4)s3)ccc12 ; "C24H23ClN4O2S"] | 2.21 |
| 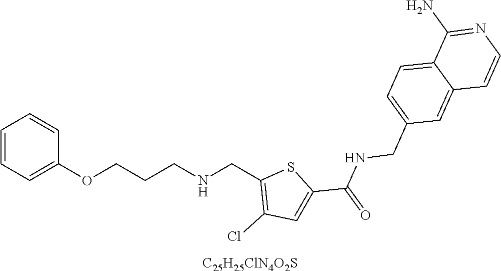 [SMILES: Nc1nccc2cc(CNC(=O)c3cc(Cl)c(CNCCCOc4ccccc4)s3)ccc12 ; "C25H25ClN4O2S"] | 2.22 |
| 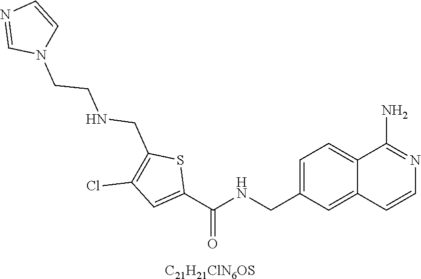 [SMILES: Nc1nccc2cc(CNC(=O)c3cc(Cl)c(CNCCn4ccnc4)s3)ccc12 ; "C21H21ClN6OS"] | 2.23 |
| 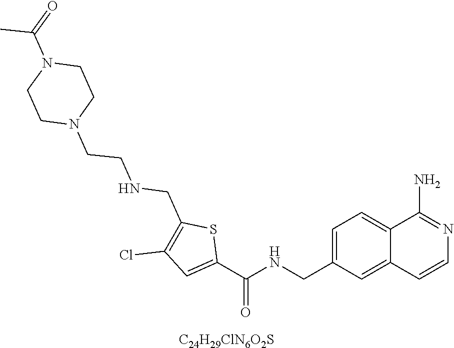 [SMILES: CC(=O)N1CCN(CCNCc2sc(C(=O)NCc3ccc4c(N)nccc4c3)cc2Cl)CC1 ; "C24H29ClN6O2S"] | 2.24 |
| 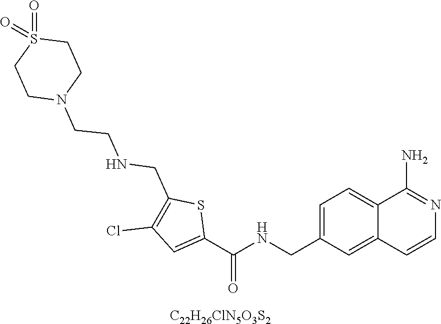 [SMILES: Nc1nccc2cc(CNC(=O)c3cc(Cl)c(CNCCN4CCS(=O)(=O)CC4)s3)ccc12 ; "C22H26ClN5O3S2"] | 2.25 |
| 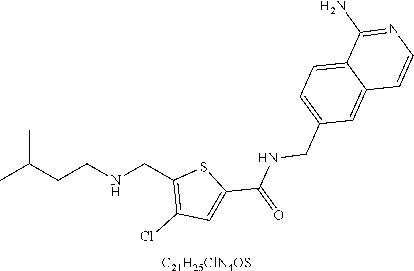 [SMILES: CC(C)CCNCc1sc(C(=O)NCc2ccc3c(N)nccc3c2)cc1Cl ; "C21H25ClN4OS"] | 2.26 |
| 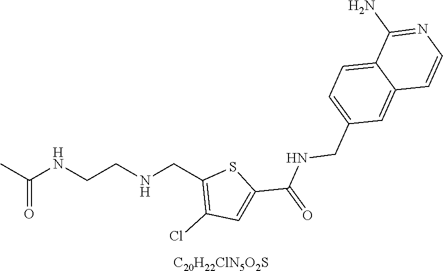 | 2.27 |
| 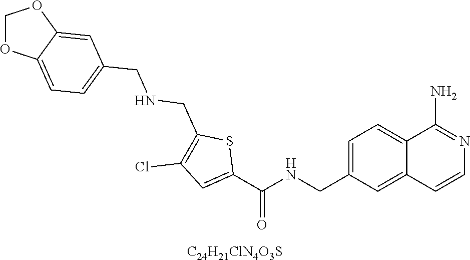 [SMILES: Nc1nccc2cc(CNC(=O)c3cc(Cl)c(CNCc4ccc5c(c4)OCO5)s3)ccc12 ; "C24H21ClN4O3S"] | 2.28 |
| 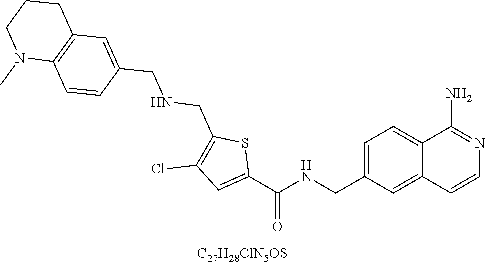 [SMILES: CN1CCCc2cc(CNCc3sc(C(=O)NCc4ccc5c(N)nccc5c4)cc3Cl)ccc21 ; "C27H28ClN5OS"] | 2.29 |
| 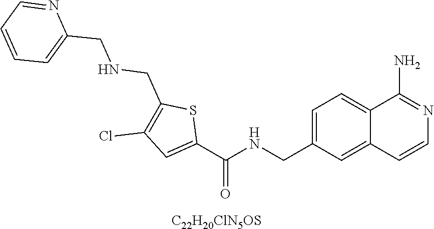 [SMILES: Nc1nccc2cc(CNC(=O)c3cc(Cl)c(CNCc4ccccn4)s3)ccc12 ; "C22H20ClN5OS"] | 2.30 |
| 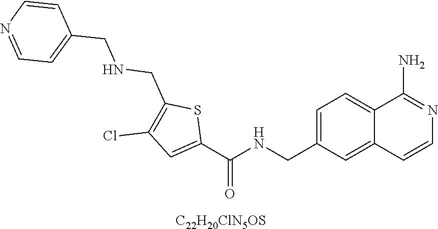 [SMILES: Nc1nccc2cc(CNC(=O)c3cc(Cl)c(CNCc4ccncc4)s3)ccc12 ; "C22H20ClN5OS"] | 2.31 |
| 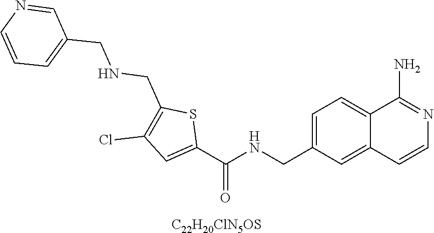 [SMILES: Nc1nccc2cc(CNC(=O)c3cc(Cl)c(CNCc4cccnc4)s3)ccc12 ; "C22H20ClN5OS"] | 2.32 |
| 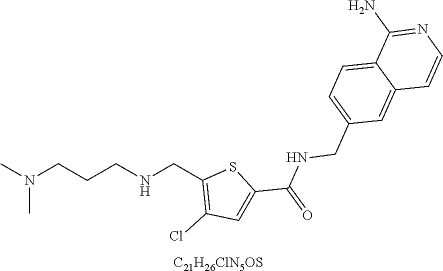 | 2.33 |
| 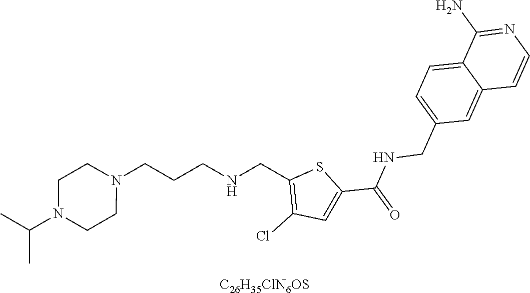 [SMILES: CC(C)N1CCN(CCCNCc2sc(C(=O)NCc3ccc4c(N)nccc4c3)cc2Cl)CC1 ; "C26H35ClN6OS"] | 2.34 |
| 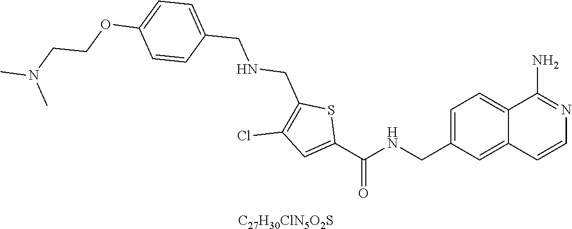 [SMILES: CN(C)CCOc1ccc(CNCc2sc(C(=O)NCc3ccc4c(N)nccc4c3)cc2Cl)cc1 ; "C27H30ClN5O2S"] | 2.35 |
| 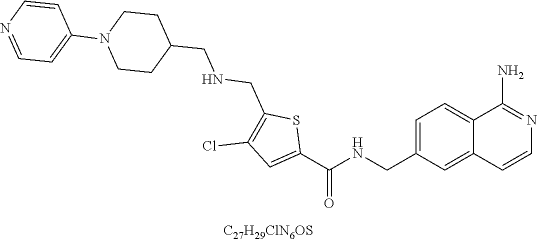 [SMILES: Nc1nccc2cc(CNC(=O)c3cc(Cl)c(CNCC4CCN(c5ccncc5)CC4)s3)ccc12 ; "C27H29ClN6OS"] | 2.36 |
| 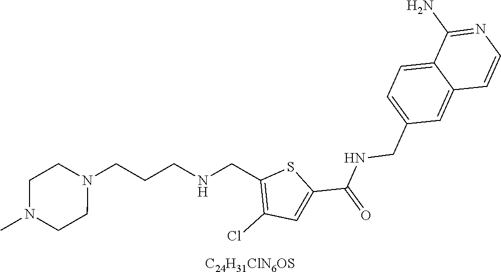 [SMILES: CN1CCN(CCCNCc2sc(C(=O)NCc3ccc4c(N)nccc4c3)cc2Cl)CC1 ; "C24H31ClN6OS"] | 2.37 |
| 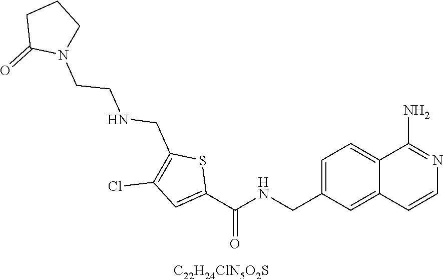 [SMILES: Nc1nccc2cc(CNC(=O)c3cc(Cl)c(CNCCN4CCCC4=O)s3)ccc12 ; "C22H24ClN5O2S"] | 2.38 |
| 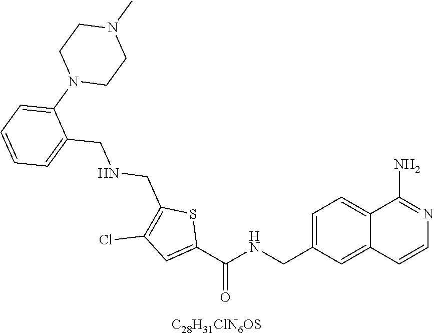 [SMILES: CN1CCN(c2ccccc2CNCc2sc(C(=O)NCc3ccc4c(N)nccc4c3)cc2Cl)CC1 ; "C28H31ClN6OS"] | 2.39 |
| 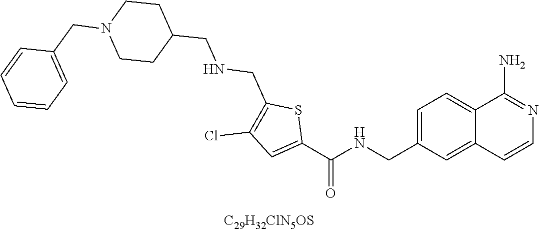 [SMILES: Nc1nccc2cc(CNC(=O)c3cc(Cl)c(CNCC4CCN(Cc5ccccc5)CC4)s3)ccc12 ; "C29H32ClN5OS"] | 2.40 |
| 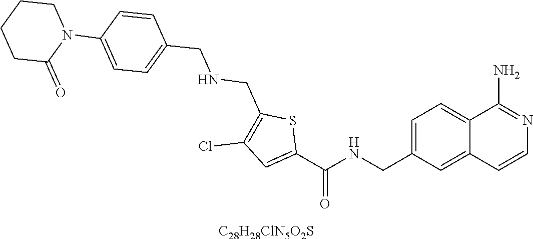 [SMILES: Nc1nccc2cc(CNC(=O)c3cc(Cl)c(CNCc4ccc(N5CCCCC5=O)cc4)s3)ccc12 ; "C28H28ClN5O2S"] | 2.41 |
| 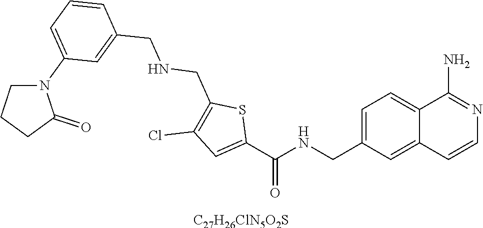 [SMILES: Nc1nccc2cc(CNC(=O)c3cc(Cl)c(CNCc4cccc(N5CCCC5=O)c4)s3)ccc12 ; "C27H26ClN5O2S"] | 2.42 |
| 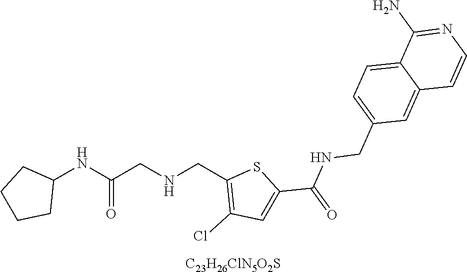 | 2.43 |
| 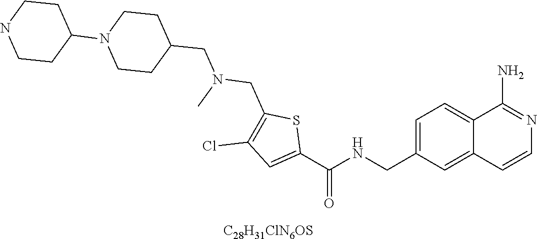 [SMILES: CN(Cc1sc(C(=O)NCc2ccc3c(N)nccc3c2)cc1Cl)CC1CCN(C2CCNCC2)CC1 ; "C28H31ClN6OS"] | 2.44 |
| 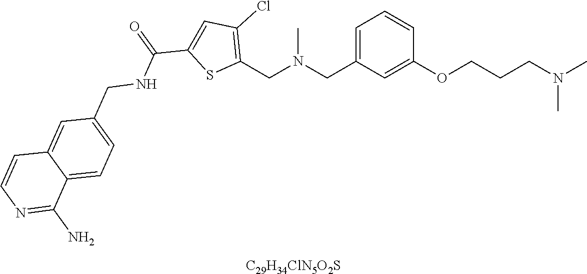 [SMILES: CN(C)CCCOc1cccc(CN(C)Cc2sc(C(=O)NCc3ccc4c(N)nccc4c3)cc2Cl)c1 ; "C29H34ClN5O2S"] | 2.45 |
| 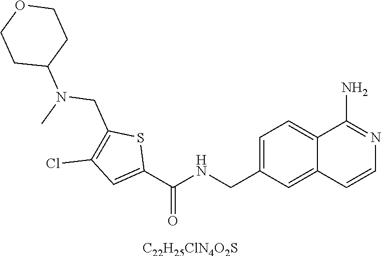 [SMILES: CN(Cc1sc(C(=O)NCc2ccc3c(N)nccc3c2)cc1Cl)C1CCOCC1 ; "C22H25ClN4O2S"] | 2.46 |
| 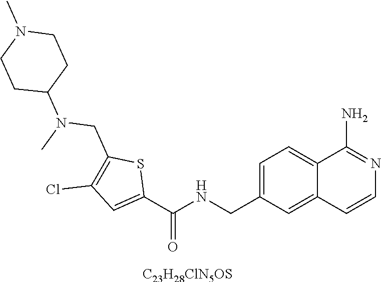 | 2.47 |
| 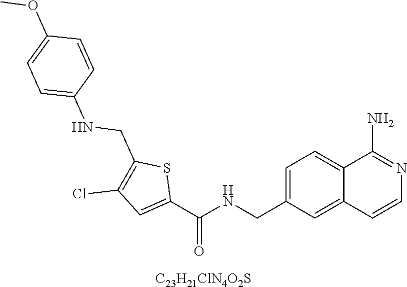 [SMILES: COc1ccc(NCc2sc(C(=O)NCc3ccc4c(N)nccc4c3)cc2Cl)cc1 ; "C23H21ClN4O2S"] | 2.48 |
| 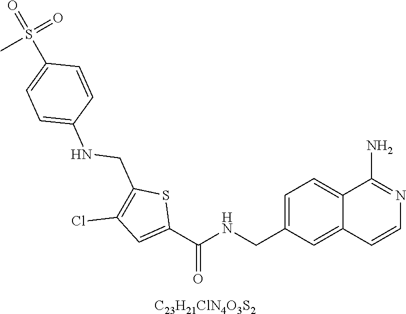 [SMILES: CS(=O)(=O)c1ccc(NCc2sc(C(=O)NCc3ccc4c(N)nccc4c3)cc2Cl)cc1 ; "C23H21ClN4O3S2"] | 2.49 |
| 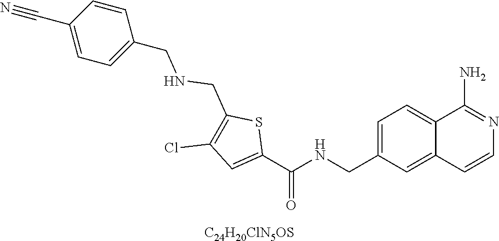 [SMILES: N#Cc1ccc(CNCc2sc(C(=O)NCc3ccc4c(N)nccc4c3)cc2Cl)cc1 ; "C24H20ClN5OS"] | 2.50 |
| 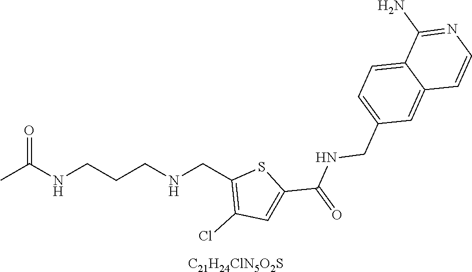 | 2.51 |
| 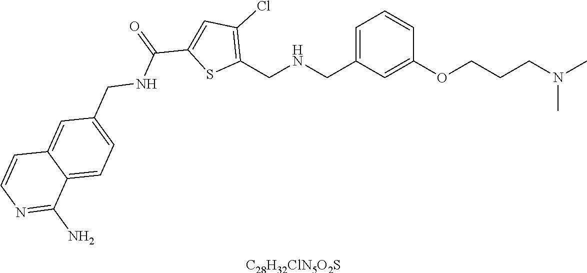 [SMILES: CN(C)CCCOc1cccc(CNCc2sc(C(=O)NCc3ccc4c(N)nccc4c3)cc2Cl)c1 ; "C28H32ClN5O2S"] | 2.52 |
| 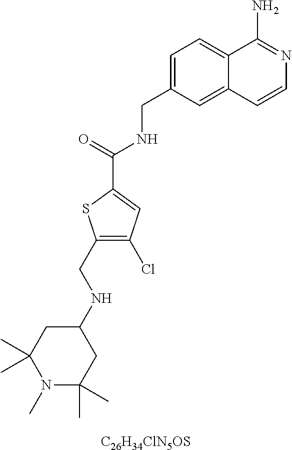 | 2.53 |
| 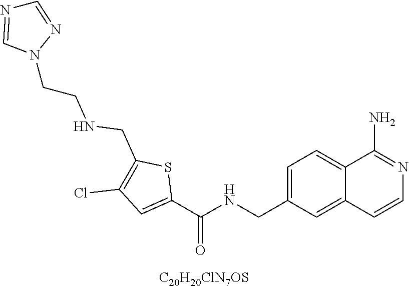 | 2.54 |
| 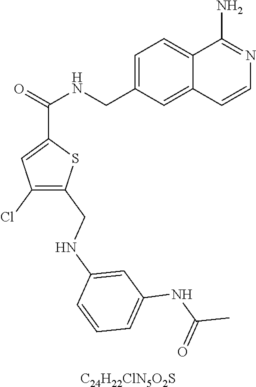 [SMILES: CC(=O)Nc1cccc(NCc2sc(C(=O)NCc3ccc4c(N)nccc4c3)cc2Cl)c1 ; "C24H22ClN5O2S"] | 2.55 |
| 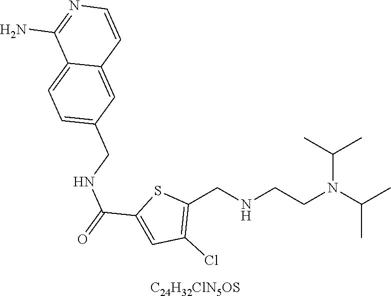 | 2.56 |
| 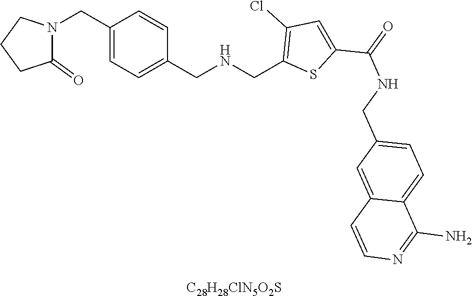 [SMILES: Nc1nccc2cc(CNC(=O)c3cc(Cl)c(CNCc4ccc(CN5CCCC5=O)cc4)s3)ccc12 ; "C28H28ClN5O2S"] | 2.57 |
| 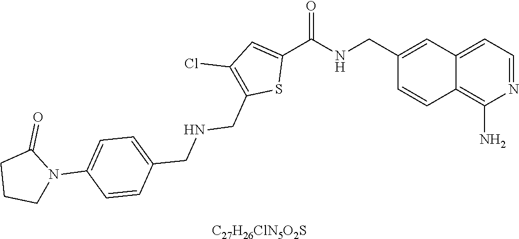 [SMILES: Nc1nccc2cc(CNC(=O)c3cc(Cl)c(CNCc4ccc(N5CCCC5=O)cc4)s3)ccc12 ; "C27H26ClN5O2S"] | 2.58 |

TABLE 2

| Structure | Example |
|---|---|
| Molecular formula | No. |
| 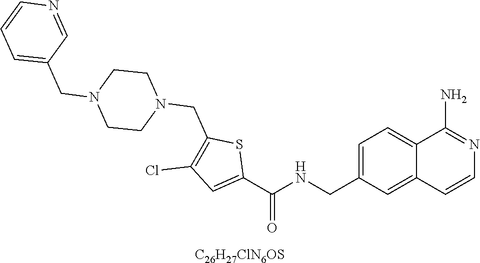 [SMILES: Nc1nccc2cc(CNC(=O)c3cc(Cl)c(CN4CCN(Cc5cccnc5)CC4)s3)ccc12 ; "C26H27ClN6OS"] | 5.01 |
| 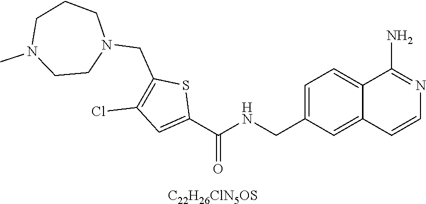 [SMILES: CN1CCCN(Cc2sc(C(=O)NCc3ccc4c(N)nccc4c3)cc2Cl)CC1 ; "C22H26ClN5OS"] | 5.02 |
| 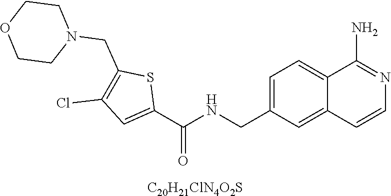 [SMILES: Nc1nccc2cc(CNC(=O)c3cc(Cl)c(CN4CCOCC4)s3)ccc12 ; "C20H21ClN4O2S"] | 5.03 |
| 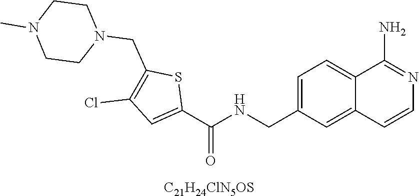 [SMILES: CN1CCN(Cc2sc(C(=O)NCc3ccc4c(N)nccc4c3)cc2Cl)CC1 ; "C21H24ClN5OS"] | 5.04 |
| 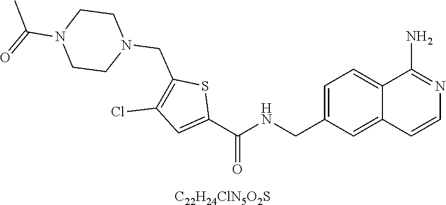 [SMILES: CC(=O)N1CCN(Cc2sc(C(=O)NCc3ccc4c(N)nccc4c3)cc2Cl)CC1 ; "C22H24ClN5O2S"] | 5.05 |
| 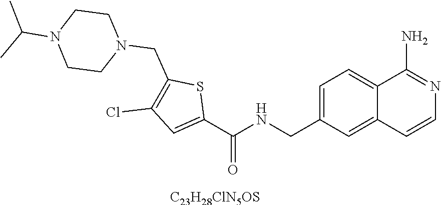 [SMILES: CC(C)N1CCN(Cc2sc(C(=O)NCc3ccc4c(N)nccc4c3)cc2Cl)CC1 ; "C23H28ClN5OS"] | 5.06 |
| 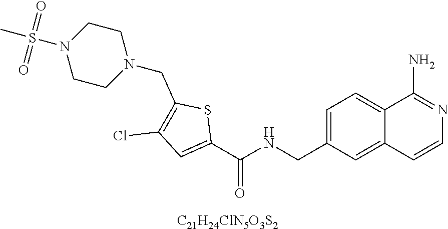 [SMILES: CS(=O)(=O)N1CCN(Cc2sc(C(=O)NCc3ccc4c(N)nccc4c3)cc2Cl)CC1 ; "C21H24ClN5O3S2"] | 5.07 |
| 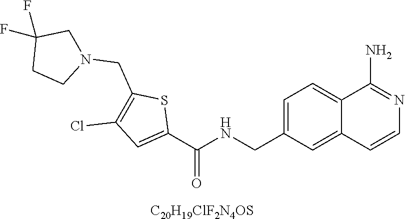 [SMILES: Nc1nccc2cc(CNC(=O)c3cc(Cl)c(CN4CCC(F)(F)C4)s3)ccc12 ; "C20H19ClF2N4OS"] | 5.08 |
| 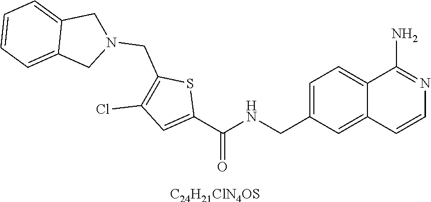 [SMILES: Nc1nccc2cc(CNC(=O)c3cc(Cl)c(CN4Cc5ccccc5C4)s3)ccc12 ; "C24H21ClN4OS"] | 5.09 |
| 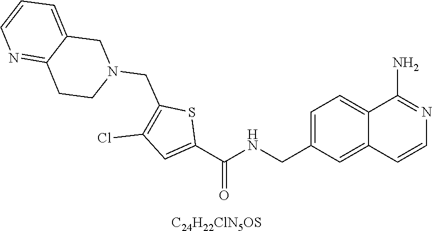 [SMILES: Nc1nccc2cc(CNC(=O)c3cc(Cl)c(CN4CCc5ncccc5C4)s3)ccc12 ; "C24H22ClN5OS"] | 5.10 |
| 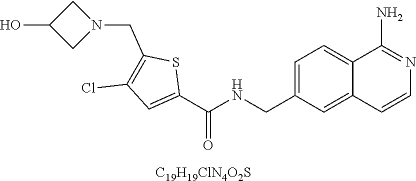 [SMILES: Nc1nccc2cc(CNC(=O)c3cc(Cl)c(CN4CC(O)C4)s3)ccc12 ; "C19H19ClN4O2S"] | 5.11 |
| 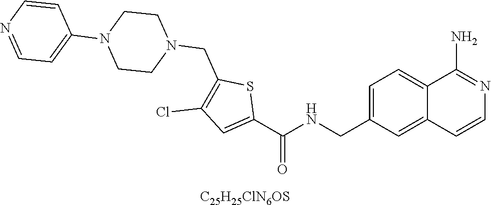 [SMILES: Nc1nccc2cc(CNC(=O)c3cc(Cl)c(CN4CCN(c5ccncc5)CC4)s3)ccc12 ; "C25H25ClN6OS"] | 5.12 |
| 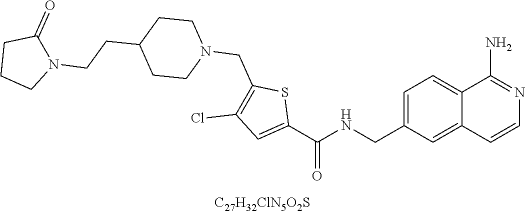 [SMILES: Nc1nccc2cc(CNC(=O)c3cc(Cl)c(CN4CCC(CCN5CCCC5=O)CC4)s3)ccc12 ; "C27H32ClN5O2S"] | 5.13 |
| 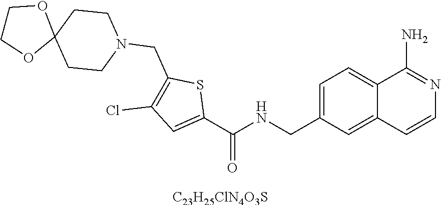 [SMILES: Nc1nccc2cc(CNC(=O)c3cc(Cl)c(CN4CCC5(CC4)OCCO5)s3)ccc12 ; "C23H25ClN4O3S"] | 5.14 |
| 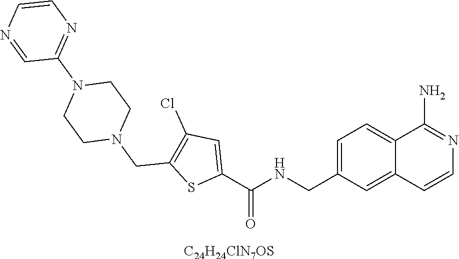 [SMILES: Nc1nccc2cc(CNC(=O)c3cc(Cl)c(CN4CCN(c5cnccn5)CC4)s3)ccc12 ; "C24H24ClN7OS"] | 5.15 |
| 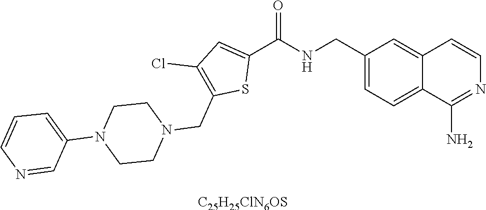 [SMILES: Nc1nccc2cc(CNC(=O)c3cc(Cl)c(CN4CCN(c5cccnc5)CC4)s3)ccc12 ; "C25H25ClN6OS"] | 5.16 |
| 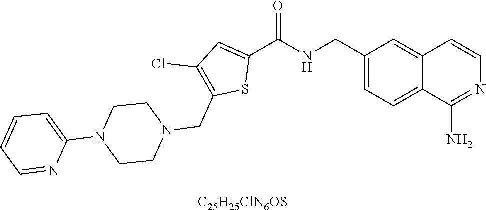 [SMILES: Nc1nccc2cc(CNC(=O)c3cc(Cl)c(CN4CCN(c5ccccn5)CC4)s3)ccc12 ; "C25H25ClN6OS"] | 5.17 |
| 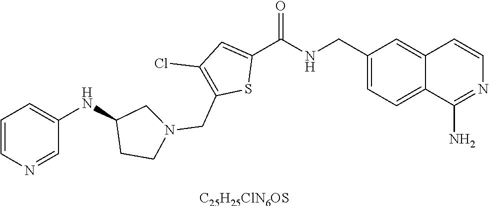 [SMILES: Nc1nccc2cc(CNC(=O)c3cc(Cl)c(CN4CC[C@@H](Nc5cccnc5)C4)s3)ccc12 ; "C25H25ClN6OS"] | 5.18 |
| 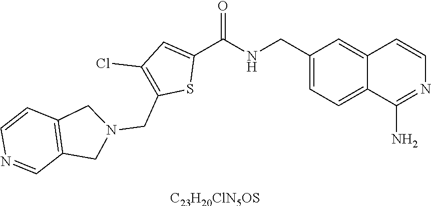 [SMILES: Nc1nccc2cc(CNC(=O)c3cc(Cl)c(CN4Cc5ccncc5C4)s3)ccc12 ; "C23H20ClN5OS"] | 5.19 |
| 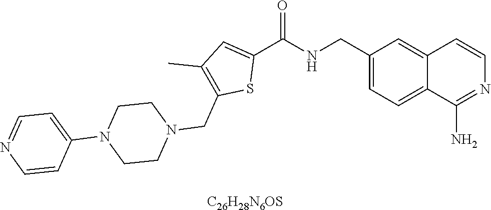 [SMILES: Cc1cc(C(=O)NCc2ccc3c(N)nccc3c2)sc1CN1CCN(c2ccncc2)CC1 ; "C26H28N6OS"] | 5.20 |
| 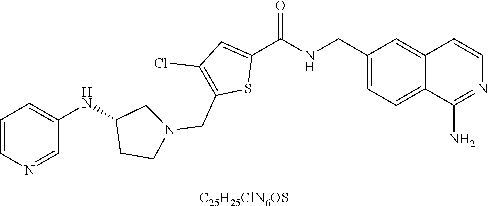 [SMILES: Nc1nccc2cc(CNC(=O)c3cc(Cl)c(CN4CC[C@H](Nc5cccnc5)C4)s3)ccc12 ; "C25H25ClN6OS"] | 5.21 |
| 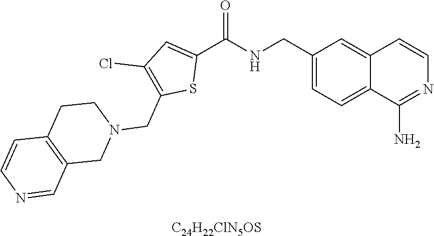 [SMILES: Nc1nccc2cc(CNC(=O)c3cc(Cl)c(CN4CCc5ccncc5C4)s3)ccc12 ; "C24H22ClN5OS"] | 5.22 |
| 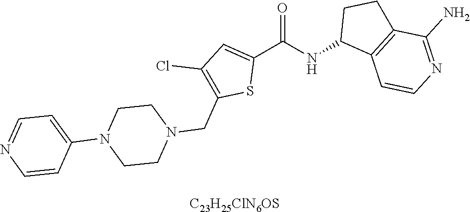 | 5.23 |
| 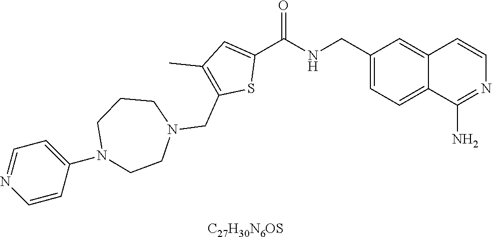 [SMILES: Cc1cc(C(=O)NCc2ccc3c(N)nccc3c2)sc1CN1CCCN(c2ccncc2)CC1 ; "C27H30N6OS"] | 5.24 |
| 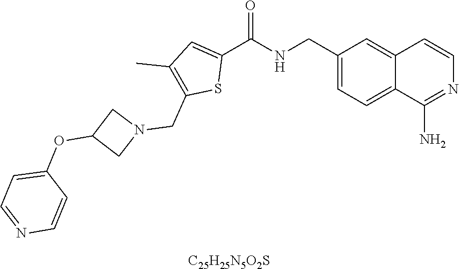 [SMILES: Cc1cc(C(=O)NCc2ccc3c(N)nccc3c2)sc1CN1CC(Oc2ccncc2)C1 ; "C25H25N5O2S"] | 5.25 |
| 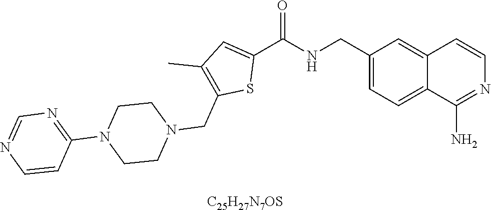 [SMILES: Cc1cc(C(=O)NCc2ccc3c(N)nccc3c2)sc1CN1CCN(c2ccncn2)CC1 ; "C25H27N7OS"] | 5.26 |
| 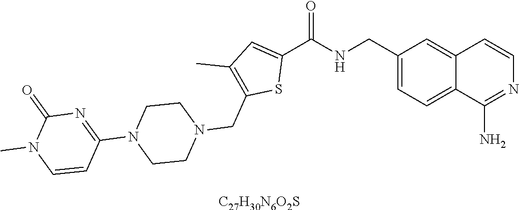 [SMILES: Cc1cc(C(=O)NCc2ccc3c(N)nccc3c2)sc1CN1CCN(c2ccn(C)c(=O)n2)CC1 ; "C27H30N6O2S"] | 5.27 |
| 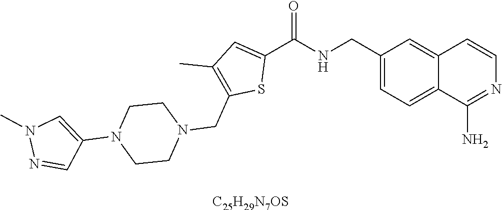 [SMILES: Cc1cc(C(=O)NCc2ccc3c(N)nccc3c2)sc1CN1CCN(c2cnn(C)c2)CC1 ; "C25H29N7OS"] | 5.28 |
| 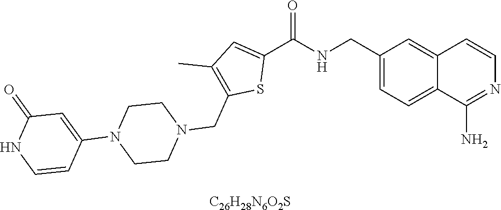 [SMILES: Cc1cc(C(=O)NCc2ccc3c(N)nccc3c2)sc1CN1CCN(c2cc[nH]c(=O)c2)CC1 ; "C26H28N6O2S"] | 5.29 |

TABLE 3

| Structure | Example |
|---|---|
| Molecular formula | No. |
| 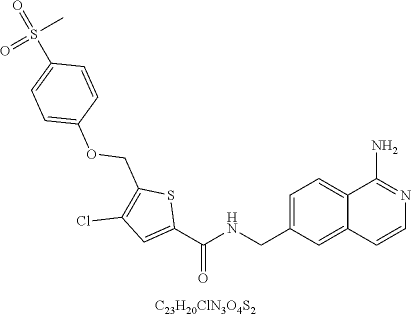 [SMILES: CS(=O)(=O)c1ccc(OCc2sc(C(=O)NCc3ccc4c(N)nccc4c3)cc2Cl)cc1 ; "C23H20ClN3O4S2"] | 7.01 |
| 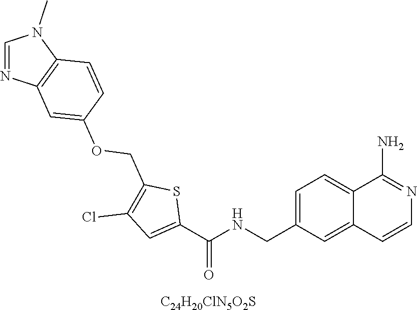 [SMILES: Cn1cnc2cc(OCc3sc(C(=O)NCc4ccc5c(N)nccc5c4)cc3Cl)ccc21 ; "C24H20ClN5O2S"] | 7.02 |
| 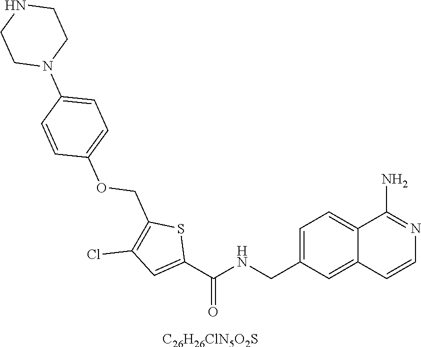 [SMILES: Nc1nccc2cc(CNC(=O)c3cc(Cl)c(COc4ccc(N5CCNCC5)cc4)s3)ccc12 ; "C26H26ClN5O2S"] | 7.03 |
| 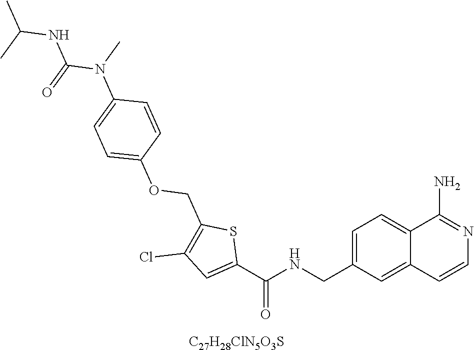 [SMILES: CC(C)NC(=O)N(C)c1ccc(OCc2sc(C(=O)NCc3ccc4c(N)nccc4c3)cc2Cl)cc1 ; "C27H28ClN5O3S"] | 7.04 |
| 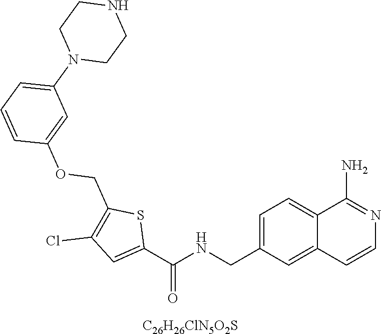 | 7.05 |
| 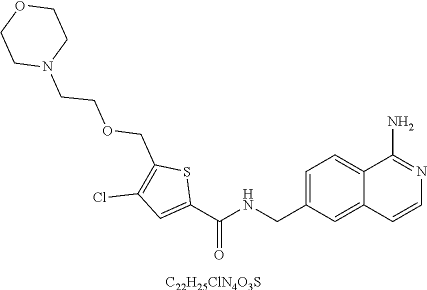 [SMILES: Nc1nccc2cc(CNC(=O)c3cc(Cl)c(COCCN4CCOCC4)s3)ccc12 ; "C22H25ClN4O3S"] | 7.06 |
| 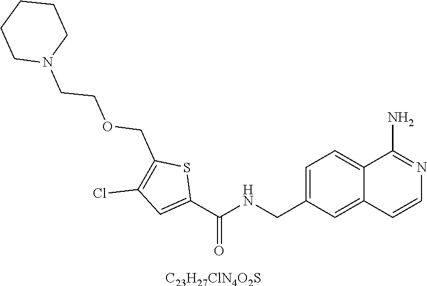 [SMILES: Nc1nccc2cc(CNC(=O)c3cc(Cl)c(COCCN4CCCCC4)s3)ccc12 ; "C23H27ClN4O2S"] | 7.07 |
| 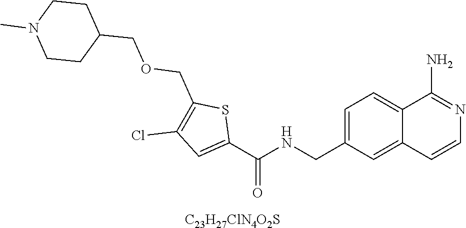 [SMILES: CN1CCC(COCc2sc(C(=O)NCc3ccc4c(N)nccc4c3)cc2Cl)CC1 ; "C23H27ClN4O2S"] | 7.08 |
| 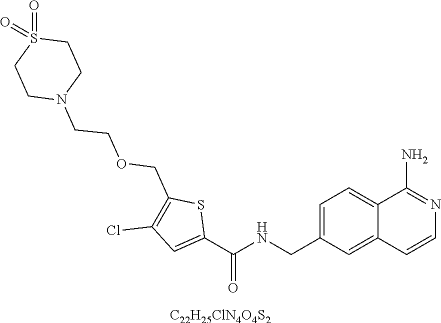 [SMILES: Nc1nccc2cc(CNC(=O)c3cc(Cl)c(COCCN4CCS(=O)(=O)CC4)s3)ccc12 ; "C22H25ClN4O4S2"] | 7.09 |
| 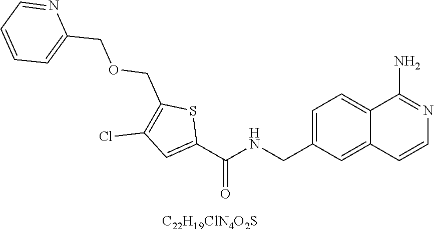 [SMILES: Nc1nccc2cc(CNC(=O)c3cc(Cl)c(COCc4ccccn4)s3)ccc12 ; "C22H19ClN4O2S"] | 7.10 |
| 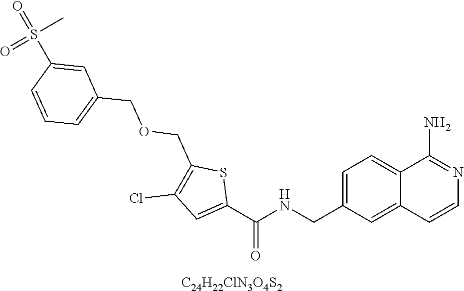 [SMILES: CS(=O)(=O)c1cccc(COCc2sc(C(=O)NCc3ccc4c(N)nccc4c3)cc2Cl)c1 ; "C24H22ClN3O4S2"] | 7.11 |
| 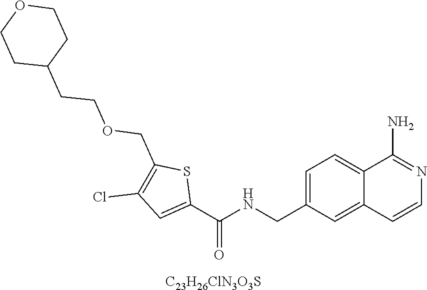 [SMILES: Nc1nccc2cc(CNC(=O)c3cc(Cl)c(COCCC4CCOCC4)s3)ccc12 ; "C23H26ClN3O3S"] | 7.12 |
| 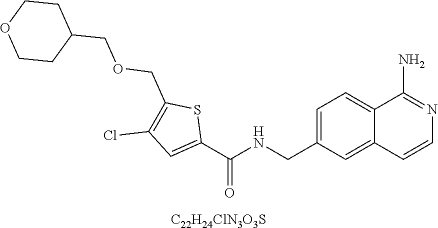 [SMILES: Nc1nccc2cc(CNC(=O)c3cc(Cl)c(COCC4CCOCC4)s3)ccc12 ; "C22H24ClN3O3S"] | 7.13 |
| 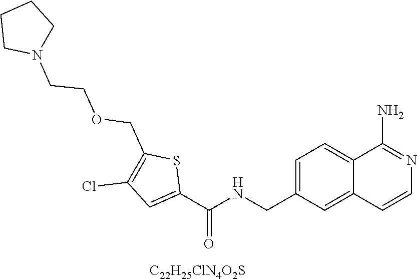 [SMILES: Nc1nccc2cc(CNC(=O)c3cc(Cl)c(COCCN4CCCC4)s3)ccc12 ; "C22H25ClN4O2S"] | 7.14 |
| 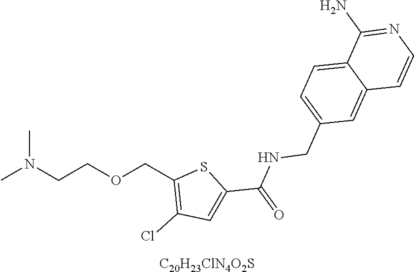 [SMILES: CN(C)CCOCc1sc(C(=O)NCc2ccc3c(N)nccc3c2)cc1Cl ; "C20H23ClN4O2S"] | 7.15 |
| 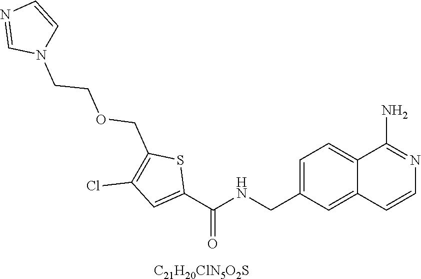 [SMILES: Nc1nccc2cc(CNC(=O)c3cc(Cl)c(COCCn4ccnc4)s3)ccc12 ; "C21H20ClN5O2S"] | 7.16 |
| 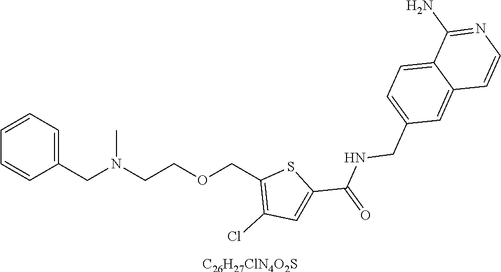 [SMILES: CN(CCOCc1sc(C(=O)NCc2ccc3c(N)nccc3c2)cc1Cl)Cc1ccccc1 ; "C26H27ClN4O2S"] | 7.17 |
| 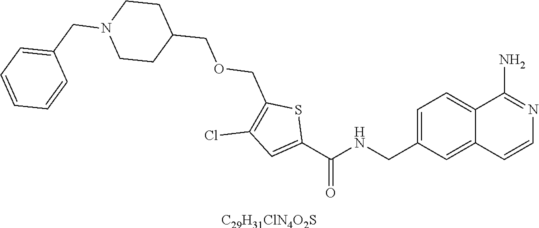 [SMILES: Nc1nccc2cc(CNC(=O)c3cc(Cl)c(COCC4CCN(Cc5ccccc5)CC4)s3)ccc12 ; "C29H31ClN4O2S"] | 7.18 |
| 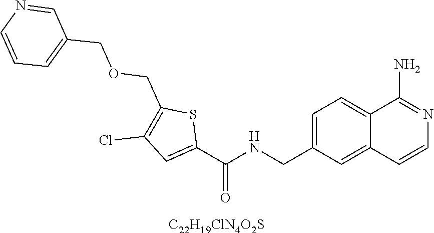 [SMILES: Nc1nccc2cc(CNC(=O)c3cc(Cl)c(COCc4cccnc4)s3)ccc12 ; "C22H19ClN4O2S"] | 7.19 |
| 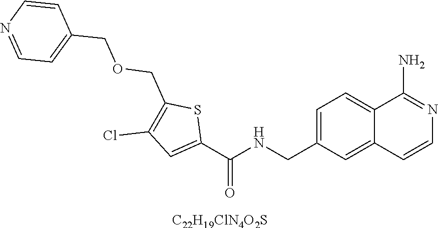 [SMILES: Nc1nccc2cc(CNC(=O)c3cc(Cl)c(COCc4ccncc4)s3)ccc12 ; "C22H19ClN4O2S"] | 7.20 |
| 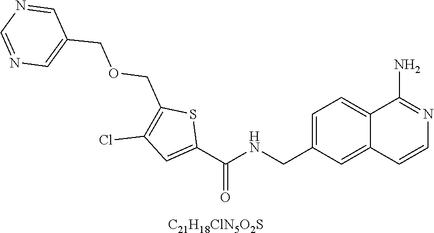 [SMILES: Nc1nccc2cc(CNC(=O)c3cc(Cl)c(COCc4cncnc4)s3)ccc12 ; "C21H18ClN5O2S"] | 7.21 |
| 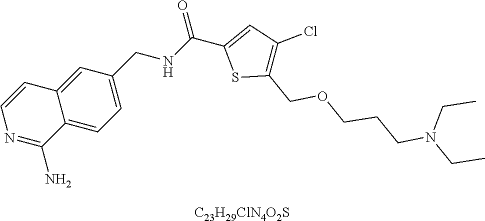 [SMILES: CCN(CC)CCCOCc1sc(C(=O)NCc2ccc3c(N)nccc3c2)cc1Cl ; "C23H29ClN4O2S"] | 7.22 |
| 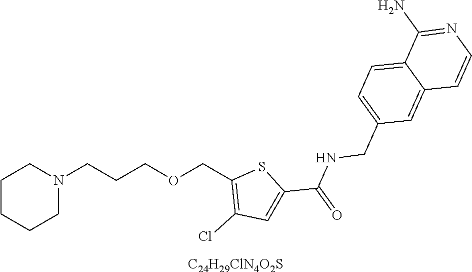 [SMILES: Nc1nccc2cc(CNC(=O)c3cc(Cl)c(COCCCN4CCCCC4)s3)ccc12 ; "C24H29ClN4O2S"] | 7.23 |
| 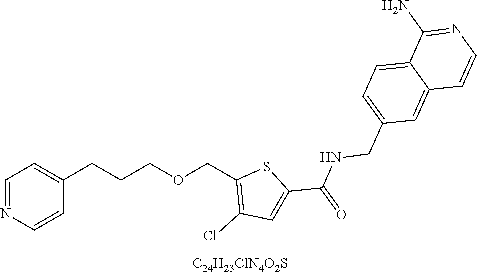 [SMILES: Nc1nccc2cc(CNC(=O)c3cc(Cl)c(COCCCc4ccncc4)s3)ccc12 ; "C24H23ClN4O2S"] | 7.24 |
| 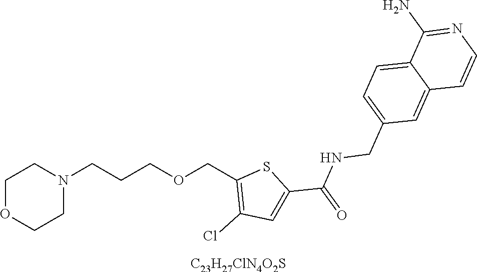 [SMILES: Nc1nccc2cc(CNC(=O)c3cc(Cl)c(COCCCN4CCOCC4)s3)ccc12 ; "C23H27ClN4O2S"] | 7.25 |
| 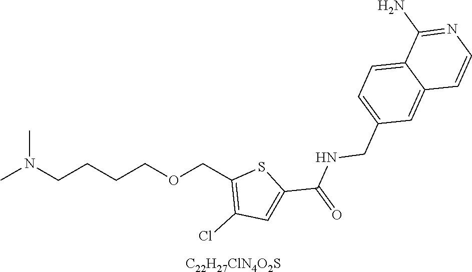 [SMILES: CN(C)CCCCOCc1sc(C(=O)NCc2ccc3c(N)nccc3c2)cc1Cl ; "C22H27ClN4O2S"] | 7.26 |
| 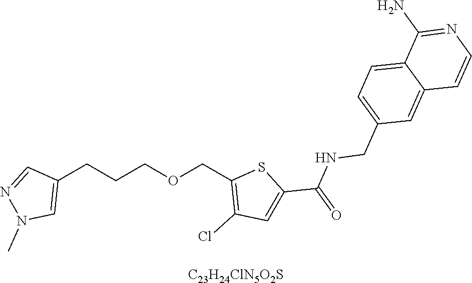 | 7.27 |
| 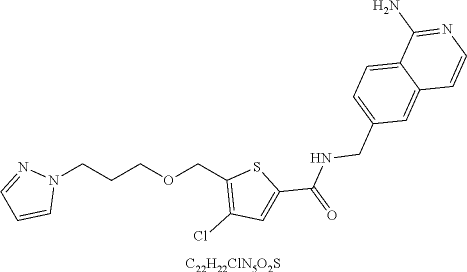 | 7.28 |
| 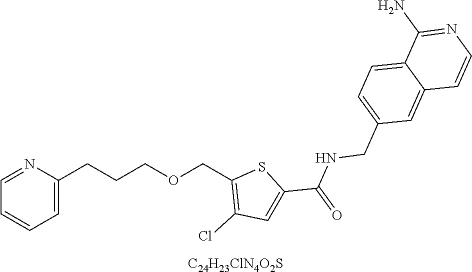 [SMILES: Nc1nccc2cc(CNC(=O)c3cc(Cl)c(COCCCc4ccccn4)s3)ccc12 ; "C24H23ClN4O2S"] | 7.29 |
| 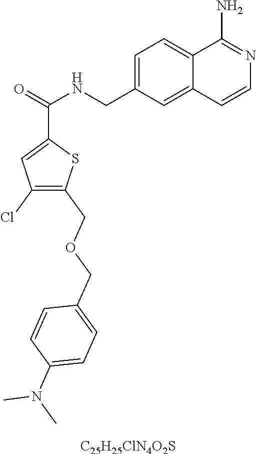 [SMILES: CN(C)c1ccc(COCc2sc(C(=O)NCc3ccc4c(N)nccc4c3)cc2Cl)cc1 ; "C25H25ClN4O2S"] | 7.30 |
| 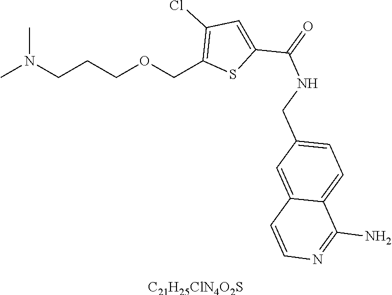 [SMILES: CN(C)CCCOCc1sc(C(=O)NCc2ccc3c(N)nccc3c2)cc1Cl ; "C21H25ClN4O2S"] | 7.31 |

TABLE 4

| | Exam- |
|---|---|
| Structure | ple |
| Molecular formula | No. |
| 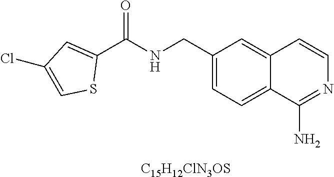 [SMILES: Nc1nccc2cc(CNC(=O)c3cc(Cl)cs3)ccc12 ; "C15H12ClN3OS"] | 25.01 |
| 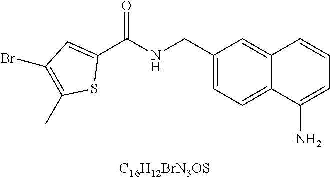 [SMILES: "C16H12BrN3OS" ; Cc1sc(C(=O)NCc2ccc3c(N)cccc3c2)cc1Br] | 25.02 |
| 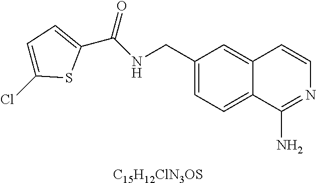 [SMILES: Nc1nccc2cc(CNC(=O)c3ccc(Cl)s3)ccc12 ; "C15H12ClN3OS"] | 25.03 |
| 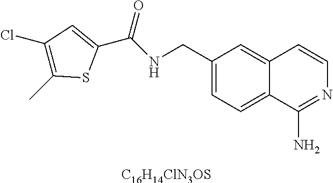 [SMILES: Cc1sc(C(=O)NCc2ccc3c(N)nccc3c2)cc1Cl ; "C16H14ClN3OS"] | 25.04 |
| 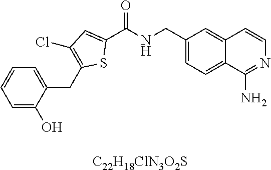 [SMILES: Nc1nccc2cc(CNC(=O)c3cc(Cl)c(Cc4ccccc4O)s3)ccc12 ; "C22H18ClN3O2S"] | 25.05 |
| 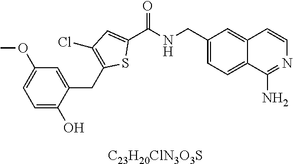 [SMILES: COc1ccc(O)c(Cc2sc(C(=O)NCc3ccc4c(N)nccc4c3)cc2Cl)c1 ; "C23H20ClN3O3S"] | 25.06 |
| 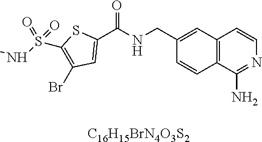 [SMILES: CNS(=O)(=O)c1sc(C(=O)NCc2ccc3c(N)nccc3c2)cc1Br ; "C16H15BrN4O3S2"] | 25.07 |
| 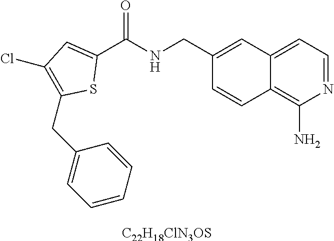 [SMILES: Nc1nccc2cc(CNC(=O)c3cc(Cl)c(Cc4ccccc4)s3)ccc12 ; "C22H18ClN3OS"] | 25.08 |
| 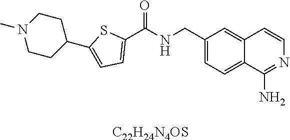 [SMILES: CN1CCC(c2ccc(C(=O)NCc3ccc4c(N)nccc4c3)s2)CC1 ; "C22H24N4OS"] | 25.09 |
| 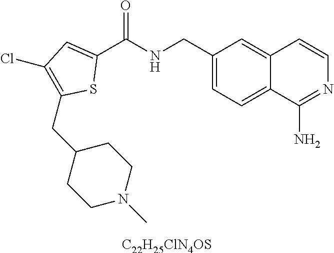 [SMILES: CN1CCC(Cc2sc(C(=O)NCc3ccc4c(N)nccc4c3)cc2Cl)CC1 ; "C22H25ClN4OS"] | 25.10 |
| 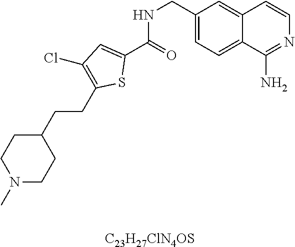 [SMILES: CN1CCC(CCc2sc(C(=O)NCc3ccc4c(N)nccc4c3)cc2Cl)CC1 ; "C23H27ClN4OS"] | 25.11 |
| 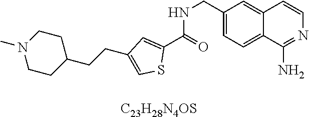 [SMILES: CN1CCC(CCc2csc(C(=O)NCc3ccc4c(N)nccc4c3)c2)CC1 ; "C23H28N4OS"] | 25.12 |
| 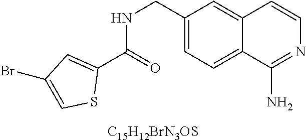 [SMILES: Nc1nccc2cc(CNC(=O)c3cc(Br)cs3)ccc12 ; "C15H12BrN3OS"] | 25.13 |
| 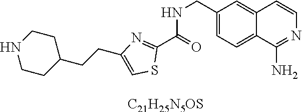 [SMILES: Nc1nccc2cc(CNC(=O)c3nc(CCC4CCNCC4)cs3)ccc12 ; "C21H25N5OS"] | 25.14 |
| 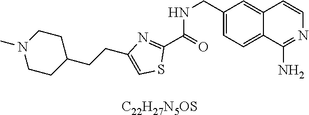 [SMILES: CN1CCC(CCc2csc(C(=O)NCc3ccc4c(N)nccc4c3)n2)CC1 ; "C22H27N5OS"] | 25.15 |
| 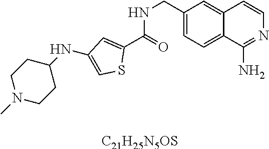 [SMILES: CN1CCC(Nc2csc(C(=O)NCc3ccc4c(N)nccc4c3)c2)CC1 ; "C21H25N5OS"] | 25.16 |
| 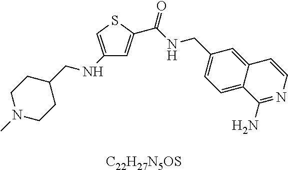 [SMILES: CN1CCC(CNc2csc(C(=O)NCc3ccc4c(N)nccc4c3)c2)CC1 ; "C22H27N5OS"] | 25.17 |

TABLE 5

| Structure | Example |
|---|---|
| Molecular formula | No. |
| 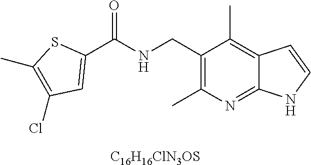 [SMILES: Cc1nc2[nH]ccc2c(C)c1CNC(=O)c1cc(Cl)c(C)s1 ; "C16H16ClN3OS"] | 25.101 |
| 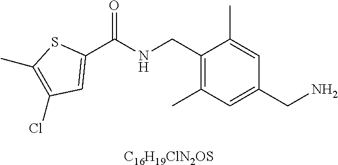 [SMILES: Cc1cc(CN)cc(C)c1CNC(=O)c1cc(Cl)c(C)s1 ; "C16H19ClN2OS"] | 25.102 |
| 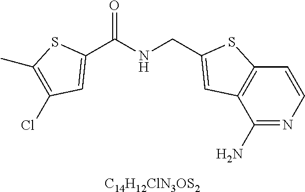 [SMILES: Cc1sc(C(=O)NCc2cc3c(N)nccc3s2)cc1Cl ; "C14H12ClN3OS2"] | 25.103 |
| 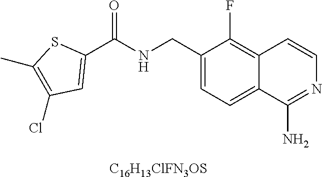 [SMILES: "C16H13ClFN3OS" ; Cc1sc(C(=O)NCc2ccc3c(N)nccc3c2F)cc1Cl] | 25.104 |
| 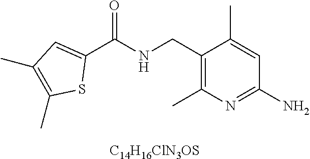 [SMILES: Cc1cc(C(=O)NCc2c(C)cc(N)nc2C)sc1C ; "C14H16ClN3OS"] | 25.105 |

TABLE 6

| Structure | Example |
|---|---|
| Molecular formula | No. |
| 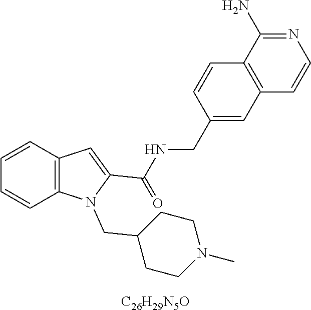 [SMILES: CN1CCC(Cn2c(C(=O)NCc3ccc4c(N)nccc4c3)cc3ccccc32)CC1 ; "C26H29N5O"] | 25.201 |
| 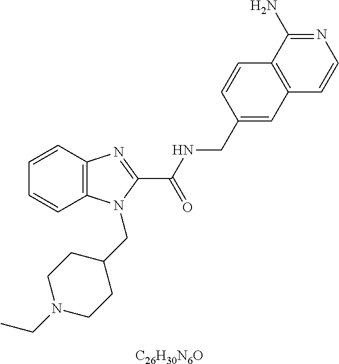 [SMILES: CCN1CCC(Cn2c(C(=O)NCc3ccc4c(N)nccc4c3)nc3ccccc32)CC1 ; "C26H30N6O"] | 25.202 |
| 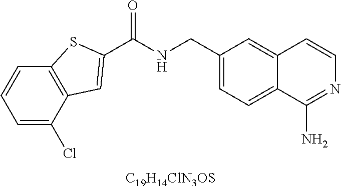 [SMILES: "C19H14ClN3OS" ; Nc1nccc2cc(CNC(=O)c3cc4c(Cl)cccc4s3)ccc12] | 25.203 |
| 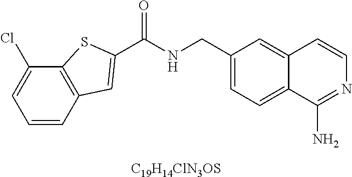 [SMILES: "C19H14ClN3OS" ; Nc1nccc2cc(CNC(=O)c3cc4cccc(Cl)c4s3)ccc12] | 25.204 |
| 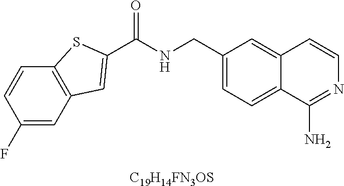 [SMILES: Nc1nccc2cc(CNC(=O)c3cc4cc(F)ccc4s3)ccc12 ; "C19H14FN3OS"] | 25.205 |
| 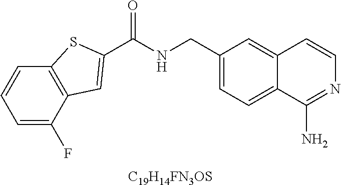 [SMILES: Nc1nccc2cc(CNC(=O)c3cc4c(F)cccc4s3)ccc12 ; "C19H14FN3OS"] | 25.206 |
| 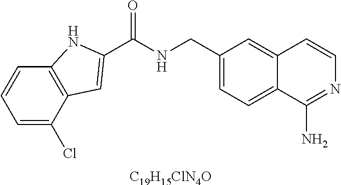 [SMILES: Nc1nccc2cc(CNC(=O)c3cc4c(Cl)cccc4[nH]3)ccc12 ; "C19H15ClN4O"] | 25.207 |
| 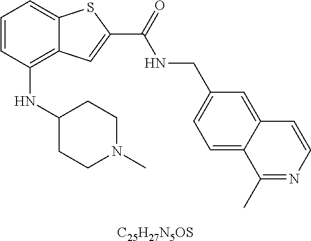 [SMILES: Cc1nccc2cc(CNC(=O)c3cc4c(NC5CCN(C)CC5)cccc4s3)ccc12 ; "C25H27N5OS"] | 25.208 |
| 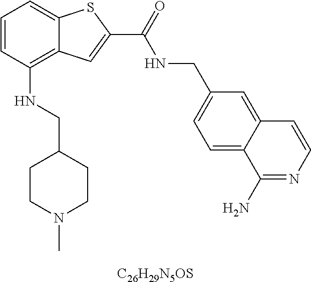 [SMILES: CN1CCC(CNc2cccc3sc(C(=O)NCc4ccc5c(N)nccc5c4)cc23)CC1 ; "C26H29N5OS"] | 25.209 |
| 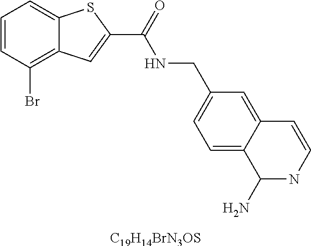 [SMILES: "C19H14BrN3OS" ; NC1NC=Cc2cc(CNC(=O)c3cc4c(Br)cccc4s3)ccc21] | 25.210 |

TABLE 7

| Structure | Example |
|---|---|
| Molecular formula | No. |
| 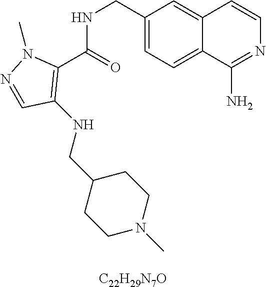 [SMILES: CN1CCC(CNc2cnn(C)c2C(=O)NCc2ccc3c(N)nccc3c2)CC1 ; "C22H29N7O"] | 26.01 |
| 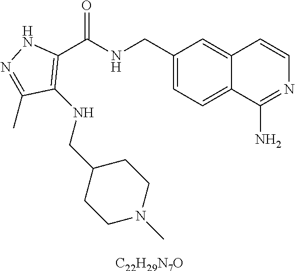 [SMILES: Cc1n[nH]c(C(=O)NCc2ccc3c(N)nccc3c2)c1NCC1CCN(C)CC1 ; "C22H29N7O"] | 26.02 |
| 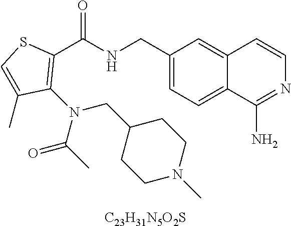 [SMILES: CC(=O)N(CC1CCN(C)CC1)c1c(C)csc1C(=O)NCc1ccc2c(N)nccc2c1 ; "C23H31N5O2S"] | 26.03 |
| 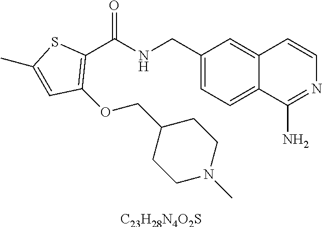 [SMILES: Cc1cc(OCC2CCN(C)CC2)c(C(=O)NCc2ccc3c(N)nccc3c2)s1 ; "C23H28N4O2S"] | 26.04 |
| 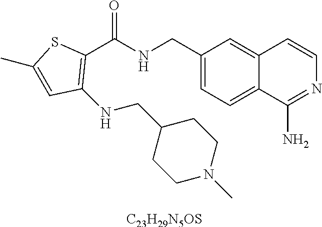 [SMILES: Cc1cc(NCC2CCN(C)CC2)c(C(=O)NCc2ccc3c(N)nccc3c2)s1 ; "C23H29N5OS"] | 26.05 |
| 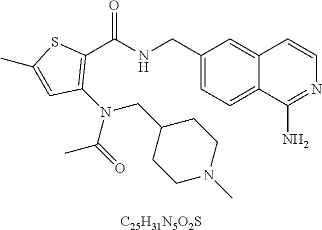 [SMILES: CC(=O)N(CC1CCN(C)CC1)c1cc(C)sc1C(=O)NCc1ccc2c(N)nccc2c1 ; "C25H31N5O2S"] | 26.06 |
| 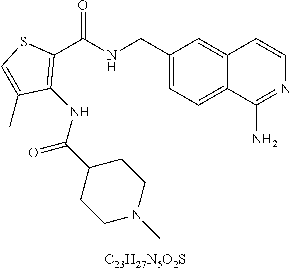 [SMILES: Cc1csc(C(=O)NCc2ccc3c(N)nccc3c2)c1NC(=O)C1CCN(C)CC1 ; "C23H27N5O2S"] | 26.07 |
| 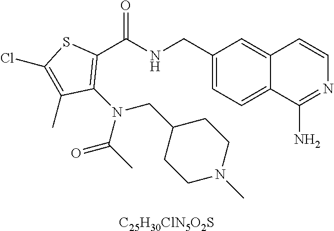 [SMILES: CC(=O)N(CC1CCN(C)CC1)c1c(C(=O)NCc2ccc3c(N)nccc3c2)sc(Cl)c1C ; "C25H30ClN5O2S"] | 26.08 |
| 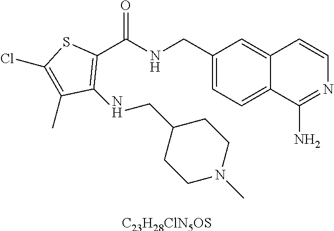 [SMILES: Cc1c(Cl)sc(C(=O)NCc2ccc3c(N)nccc3c2)c1NCC1CCN(C)CC1 ; "C23H28ClN5OS"] | 26.09 |
| 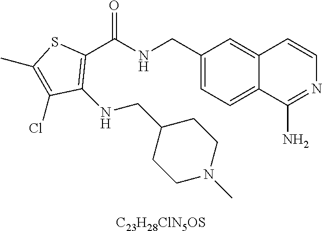 [SMILES: Cc1sc(C(=O)NCc2ccc3c(N)nccc3c2)c(NCC2CCN(C)CC2)c1Cl ; "C23H28ClN5OS"] | 26.10 |
| 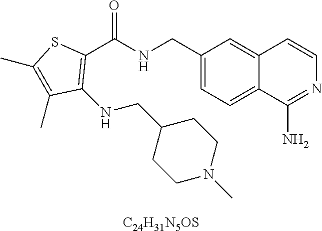 [SMILES: Cc1sc(C(=O)NCc2ccc3c(N)nccc3c2)c(NCC2CCN(C)CC2)c1C ; "C24H31N5OS"] | 26.11 |
| 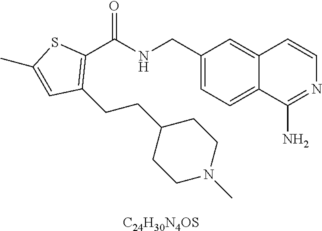 [SMILES: Cc1cc(CCC2CCN(C)CC2)c(C(=O)NCc2ccc3c(N)nccc3c2)s1 ; "C24H30N4OS"] | 26.12 |
| 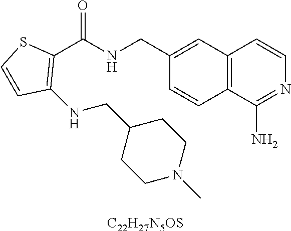 [SMILES: CN1CCC(CNc2ccsc2C(=O)NCc2ccc3c(N)nccc3c2)CC1 ; "C22H27N5OS"] | 26.13 |
| 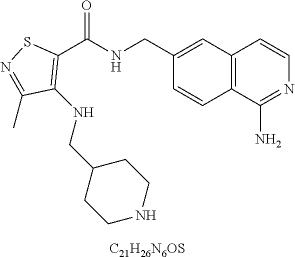 | 26.14 |
| 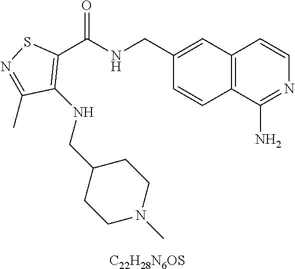 | 26.15 |
| 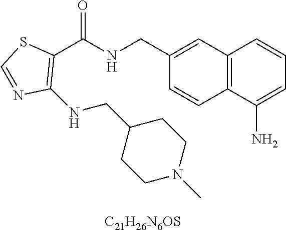 [SMILES: CN1CCC(CNc2ncsc2C(=O)NCc2ccc3c(N)cccc3c2)CC1 ; "C21H26N6OS"] | 26.16 |

TABLE 8

| Structure | Example |
|---|---|
| Molecular formula | No. |
| 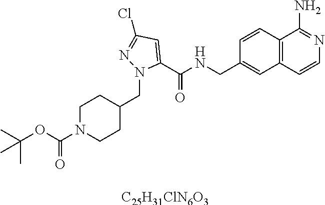 [SMILES: CC(C)(C)OC(=O)N1CCC(Cn2nc(Cl)cc2C(=O)NCc2ccc3c(N)nccc3c2)CC1 ; "C25H31ClN6O3"] | 35.01 |
| 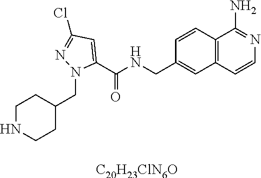 [SMILES: Nc1nccc2cc(CNC(=O)c3cc(Cl)nn3CC3CCNCC3)ccc12 ; "C20H23ClN6O"] | 35.02 |
| 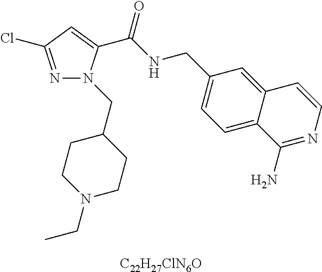 [SMILES: CCN1CCC(Cn2nc(Cl)cc2C(=O)NCc2ccc3c(N)nccc3c2)CC1 ; "C22H27ClN6O"] | 35.03 |
| 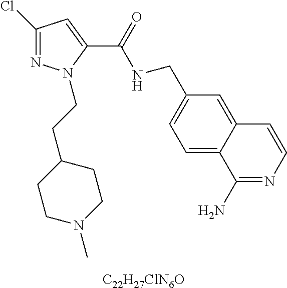 [SMILES: CN1CCC(CCn2nc(Cl)cc2C(=O)NCc2ccc3c(N)nccc3c2)CC1 ; "C22H27ClN6O"] | 35.04 |
| 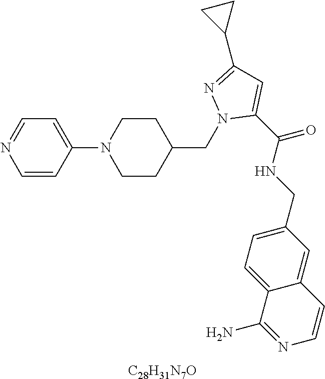 [SMILES: Nc1nccc2cc(CNC(=O)c3cc(C4CC4)nn3CC3CCN(c4ccncc4)CC3)ccc12 ; "C28H31N7O"] | 35.05 |
| 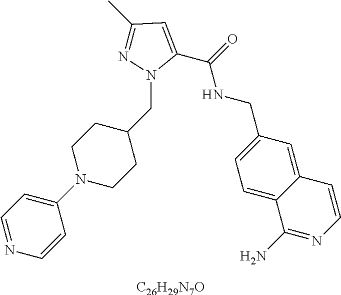 [SMILES: Cc1cc(C(=O)NCc2ccc3c(N)nccc3c2)n(CC2CCN(c3ccncc3)CC2)n1 ; "C26H29N7O"] | 35.06 |
| 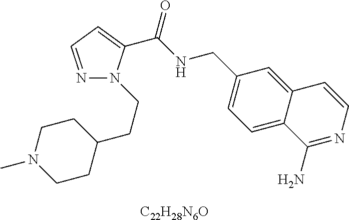 [SMILES: CN1CCC(CCn2nccc2C(=O)NCc2ccc3c(N)nccc3c2)CC1 ; "C22H28N6O"] | 35.07 |
| 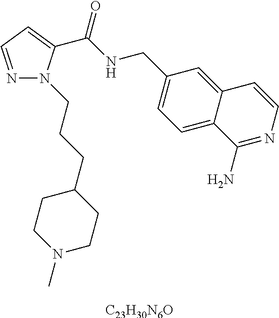 [SMILES: CN1CCC(CCCn2nccc2C(=O)NCc2ccc3c(N)nccc3c2)CC1 ; "C23H30N6O"] | 35.08 |
| 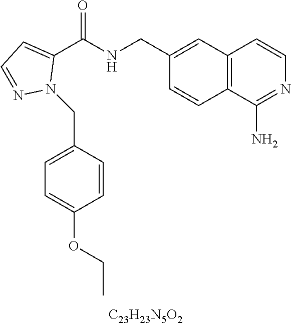 [SMILES: CCOc1ccc(Cn2nccc2C(=O)NCc2ccc3c(N)nccc3c2)cc1 ; "C23H23N5O2"] | 35.09 |

TABLE 9

| Structure | Example |
|---|---|
| Molecular formula | No. |
| 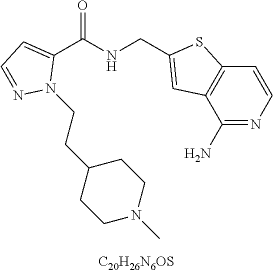 | 46.01 |

TABLE 10

| | Ex- |
|---|---|
| | am- |
| Structure | ple |
| Molecular formula | No. |
| 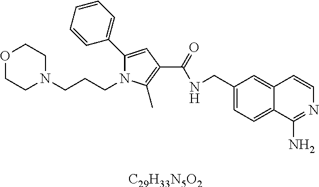 [SMILES: Cc1c(C(=O)NCc2ccc3c(N)nccc3c2)cc(-c2ccccc2)n1CCCN1CCOCC1 ; "C29H33N5O2"] | 51.01 |
| 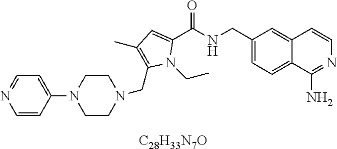 [SMILES: CCn1c(C(=O)NCc2ccc3c(N)nccc3c2)cc(C)c1CN1CCN(c2ccncc2)CC1 ; "C28H33N7O"] | 51.02 |
| 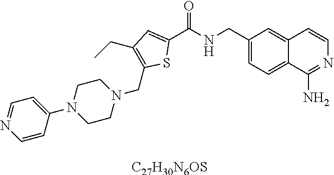 [SMILES: CCc1cc(C(=O)NCc2ccc3c(N)nccc3c2)sc1CN1CCN(c2ccncc2)CC1 ; "C27H30N6OS"] | 51.03 |
| 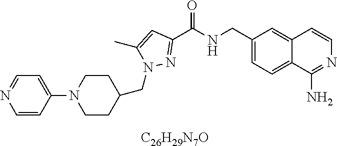 [SMILES: Cc1cc(C(=O)NCc2ccc3c(N)nccc3c2)nn1CC1CCN(c2ccncc2)CC1 ; "C26H29N7O"] | 51.04 |
| 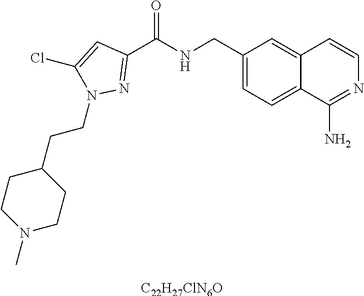 [SMILES: CN1CCC(CCn2nc(C(=O)NCc3ccc4c(N)nccc4c3)cc2Cl)CC1 ; "C22H27ClN6O"] | 51.05 |
| 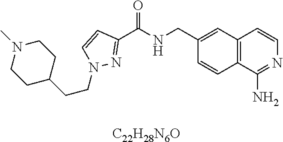 [SMILES: CN1CCC(CCn2ccc(C(=O)NCc3ccc4c(N)nccc4c3)n2)CC1 ; "C22H28N6O"] | 51.06 |
| 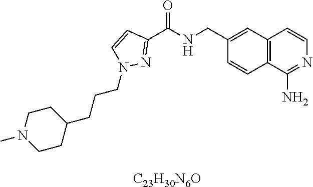 [SMILES: CN1CCC(CCCn2ccc(C(=O)NCc3ccc4c(N)nccc4c3)n2)CC1 ; "C23H30N6O"] | 51.07 |
| 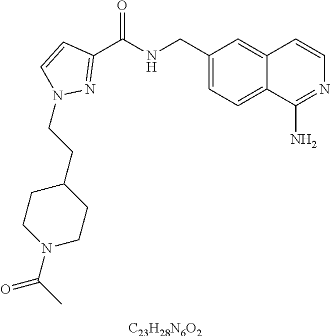 [SMILES: CC(=O)N1CCC(CCn2ccc(C(=O)NCc3ccc4c(N)nccc4c3)n2)CC1 ; "C23H28N6O2"] | 51.08 |

TABLE 11

| Structure | Example |
|---|---|
| Molecular formula | No. |
| 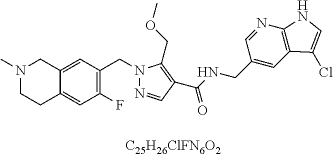 [SMILES: COCc1c(C(=O)NCc2cnc3[nH]cc(Cl)c3c2)cnn1Cc1cc2c(cc1F)CCN(C)C2 ; "C25H26ClFN6O2"] | 69.01 |

TABLE 12

| Structure | Example |
|---|---|
| Molecular formula | No. |
| 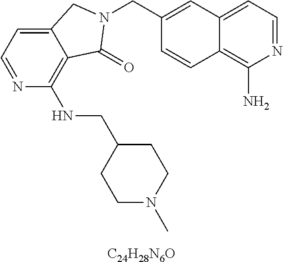 [SMILES: CN1CCC(CNc2nccc3c2C(=O)N(Cc2ccc4c(N)nccc4c2)C3)CC1 ; "C24H28N6O"] | 82.01 |

The compounds of the invention can be preferably selected from examples: 25.15, 25.21, 35.04, 51.05, 2.36, 7.03, 7.05, 7.08, 7.22, 7.23, 7.26, 7.31, 25.07, 25.11, 25.14, 25.202, 25.203, 25.207, 26.05, 26.09, 26.1, 26.16, 35.07, 35.08, 51.06, 51.07, 69.01; and pharmaceutically acceptable salts and/or solvates thereof. In particular, the compounds of the invention can be selected from examples: 25.15, 25.21, 35.04, 51.05; and pharmaceutically acceptable salts and/or solvates thereof.

The compounds of the invention can be selected from Table 1, and pharmaceutically acceptable salts and/or solvates thereof.

The compounds of the invention can be selected from Table 2, and pharmaceutically acceptable salts and/or solvates thereof.

The compounds of the invention can be selected from Table 3, and pharmaceutically acceptable salts and/or solvates thereof.

The compounds of the invention can be selected from Table 4, and pharmaceutically acceptable salts and/or solvates thereof.

The compounds of the invention can be selected from Table 5, and pharmaceutically acceptable salts and/or solvates thereof.

The compounds of the invention can be selected from Table 6, and pharmaceutically acceptable salts and/or solvates thereof.

The compounds of the invention can be selected from Table 7, and pharmaceutically acceptable salts and/or solvates thereof.

The compounds of the invention can be selected from Table 8, and pharmaceutically acceptable salts and/or solvates thereof.

The compounds of the invention can be selected from Table 9, and pharmaceutically acceptable salts and/or solvates thereof.

The compounds of the invention can be selected from Table 10, and pharmaceutically acceptable salts and/or solvates thereof.

The compounds of the invention can be selected from Table 11, and pharmaceutically acceptable salts and/or solvates thereof.

The compounds of the invention can be selected from Table 12, and pharmaceutically acceptable salts and/or solvates thereof.

Therapeutic Applications

As noted above, the compounds (or pharmaceutically acceptable salts and/or solvates thereof), and pharmaceutical compositions comprising the compounds (or pharmaceutically acceptable salts and/or solvates thereof) of the present invention are inhibitors of FXIIa. They are therefore useful in the treatment of disease conditions for which FXIIa is a causative factor.

Accordingly, the present invention provides a compound of the invention (or a pharmaceutically acceptable salt and/or solvate thereof), or a pharmaceutical composition comprising a compound of the invention (or a pharmaceutically acceptable salt and/or solvate thereof), for use in medicine.

The present invention also provides for the use of a compound of the invention (or a pharmaceutically acceptable salt and/or solvate thereof), or a pharmaceutical composition comprising the compound of the invention (or a pharmaceutically acceptable salt and/or solvate thereof), in the manufacture of a medicament for the treatment or prevention of a disease or condition in which FXIIa activity is implicated.

The present invention also provides a method of treatment of a disease or condition in which FXIIa activity is implicated comprising administration to a subject in need thereof a therapeutically effective amount of a compound of the invention (or a pharmaceutically acceptable salt and/or solvate thereof), or a pharmaceutical composition comprising the compound of the invention (or a pharmaceutically acceptable salt and/or solvate thereof).

As discussed above, FXIIa can mediate the conversion of plasma kallikrein from plasma prekallikrein. Plasma kallikrein can then cause the cleavage of high molecular weight kininogen to generate bradykinin, which is a potent inflammatory hormone. Inhibiting FXIIa has the potential to inhibit (or even prevent) plasma kallikrein production. Thus, the disease or condition in which FXIIa activity is implicated can be a bradykinin-mediated angioedema.

The bradykinin-mediated angioedema can be non-hereditary. For example, the non-hereditary bradykinin-mediated angioedema can be selected from non-hereditary angioedema with normal C1 Inhibitor (AE-nC1 Inh), which can be environmental, hormonal, or drug-induced; acquired angioedema; anaphylaxis associated angioedema; angiotensin converting enzyme (ACE or ace) inhibitor-induced angioedema; dipeptidyl peptidase-4 inhibitor-induced angioedema; and tPA-induced angioedema (tissue plasminogen activator-induced angioedema).

Alternatively, and preferably, the bradykinin-mediated angioedema can be hereditary angioedema (HAE), which is angioedema caused by an inherited dysfunction/fault/mutation. Types of HAE that can be treated with compounds according to the invention include HAE type 1, HAE type 2, and normal C1 inhibitor HAE (normal C1 Inh HAE).

The disease or condition in which FXIIa activity is implicated can be selected from vascular hyperpermeability, stroke including ischemic stroke and haemorrhagic accidents; retinal edema; diabetic retinopathy; DME; retinal vein occlusion; and AMD. These conditions can also be bradykinin-mediated.

As discussed above, FXIIa can activate FXIa to cause a coagulation cascade. Thrombotic disorders are linked to this cascade. Thus, the disease or condition in which FXIIa activity is implicated can be a thrombotic disorder. More specifically, the thrombotic disorder can be thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; and atherosclerosis.

Surfaces of medical devices that come into contact with blood can cause thrombosis. The compounds (or pharmaceutically acceptable salts and/or solvates thereof) and pharmaceutical compositions of the present invention can be coated on the surfaces of devices that come into contact with blood to mitigate the risk of the device causing thrombosis. For instance, they can lower the propensity these devices to clot blood and therefore cause thrombosis. Examples of devices that come into contact with blood include vascular grafts, stents, in dwelling catheters, external catheters, orthopedic prosthesis, cardiac prosthesis, and extracorporeal circulation systems.

Other disease conditions for which FXIIa is a causative factor include: neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

Combination Therapy

The compounds of the present invention (or pharmaceutically acceptable salts and/or solvates thereof) may be administered in combination with other therapeutic agents. Suitable combination therapies include any compound of the present invention (or a pharmaceutically acceptable salt and/or solvate thereof) combined with one or more agents selected from agents that inhibit platelet-derived growth factor (PDGF), endothelial growth factor (VEGF), integrin alpha5beta1, steroids, other agents that inhibit FXIIa and other inhibitors of inflammation.

Some specific examples of therapeutic agents that may be combined with the compounds of the present invention include those disclosed in EP2281885A and by S. Patel in Retina, 2009 June; 29(6 Suppl):545-8.

Other suitable combination therapies include a compound of the invention (or a pharmaceutically acceptable salt and/or solvate thereof) combined with one or more agents selected from agents that treat HAE (as defined generally herein), for example bradykinin B2 antagonists such icatibant (Firazyr®); plasma kallikrein inhibitors such as ecallantide (Kalbitor®) and lanadelumab (Takhzyro®); or C1 esterase inhibitor such as Cinryze® and Haegarda® and Berinert® and Ruconest®.

Other suitable combination therapies include a compound of the invention (or a pharmaceutically acceptable salt and/or solvate thereof) combined with one or more agents selected from agents that are antithrombotics (as outlined above), for example other Factor XIIa inhibitors, thrombin receptor antagonists, thrombin inhibitors, factor Vila inhibitors, factor Xa inhibitors, factor XIa inhibitors, factor IXa inhibitors, adenosine diphosphate antiplatelet agents (e.g., P2Y12 antagonists), fibrinogen receptor antagonists (e.g. to treat or prevent unstable angina or to prevent reocclusion after angioplasty and restenosis) and aspirin) and platelet aggregation inhibitors.

When combination therapy is employed, the compounds of the present invention and said combination agents may exist in the same or different pharmaceutical compositions, and may be administered separately, sequentially or simultaneously.

The compounds of the present invention can be administered in combination with laser treatment of the retina. The combination of laser therapy with intravitreal injection of an inhibitor of VEGF for the treatment of diabetic macular edema is known (Elman M, Aiello L, Beck R, et al. “Randomized trial evaluating ranibizumab plus prompt or deferred laser or triamcinolone plus prompt laser for diabetic macular edema” Ophthalmology. 27 Apr. 2010).

### Definitions

As noted above, n can be 0, 1, or 2. n is preferable 1.

As noted above, “alkoxy” is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, —N(R12)₂ and fluoro. Examples of such alkoxy groups include, but are not limited to, C₁-methoxy, C₂-ethoxy, C₃-n-propoxy 10 and C₄-n-butoxy for linear alkoxy, and C₃-iso-propoxy, and C₄-sec-butoxy and tert-butoxy for branched alkoxy, optionally substituted as noted aboves. More specifically, alkoxy can be linear groups of between 1 and 4 carbon atoms (C₁-C₄), more specifically, between 1 and 3 carbon atoms (C₁-C₃). More specifically, alkoxy can be branched groups of between 3 and 4 carbon atoms (C₃-C₄), optionally substituted as noted above.

As noted above, “alkyl” is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₁-C₆)alkoxy, OH, —NR13R14, —NHCOCH₃, —CO(heterocyclyl^{b}), —COOR13, —CONR13R14, CN, CF₃, halo, oxo, and heterocyclyl^{b}. As noted above, “alkyl^{b}” is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₁-C₆)alkoxy, OH, —N(R12)₂, —NHCOCH₃, CF₃, halo, oxo, cyclopropane, —O(aryl^{b}), aryl^{b}, and heterocyclyl^{b}. Examples of such alkyl or alkyl^{b} groups include, but are not limited, to C₁-methyl, C₂-ethyl, C₃-propyl and C₄-n-butyl, C₃-iso-propyl, C₄-sec-butyl, C₄-iso-butyl, C₄-tert-butyl and C₅-neo-pentyl), optionally substituted as noted above. More specifically, “alkyl” or “alkyl^{b}” can be a linear saturated hydrocarbon having up to 6 carbon atoms (C₁-C₆) or a branched saturated hydrocarbon of between 3 and 6 carbon atoms (C₃-C₆), optionally substituted as noted above. Even more specifically, “alkyl” or “alkyl^{b}” can be a linear saturated hydrocarbon having up to 4 carbon atoms (C₁-C₄) or a branched saturated hydrocarbon of between 3 and 4 carbon atoms (C₃-C₄), optionally substituted as noted above, which is herein called “small alkyl” or “small alkyl^{b}”, respectively. Preferably, “alkyl” or “alkyl^{b}” can be defined as a “small alkyl” or “small alkyl^{b}”.

As noted above, “alkylene” is a bivalent linear saturated hydrocarbon having 1 to 5 carbon atoms (C₁-C₅); alkylene may optionally be substituted with 1 or 2 substituents independently selected from alkyl, (C₁-C₆)alkoxy, OH, CN, CF₃, and halo. More specifically, alkylene can be a bivalent linear saturated hydrocarbon having 2 to 4 carbon atoms (C₂-C₄), more specifically having 2 to 3 carbon atoms (C₂-C₃), optionally substituted as noted above.

“Aryl” and “aryl^{b}” are defined above. Typically, aryl or aryl^{b} will be optionally substituted with 1, 2 or 3 substituents. Optional substituents are selected from those stated above. Examples of suitable aryl or aryl groups include phenyl and naphthyl (each optionally substituted as stated above). Preferably aryl is selected from phenyl and substituted phenyl (wherein said substituents are selected from those stated above).

As noted above, “cycloalkyl” is a monocyclic saturated hydrocarbon ring of between 3 and 6 carbon atoms (C₃-C₆); cycloalkyl may optionally be substituted with 1 or 2 substituents independently selected from alkyl^{b}, (C₁-C₆)alkoxy, OH, CN, CF₃, and halo. Examples of suitable monocyclic cycloalkyl groups include cyclopropyl, cyclobutyl, cyclopentyl, and cyclohexyl), optionally substituted as noted above. More specifically, cycloalkyl can be a monocyclic saturated hydrocarbon ring of between 3 and 5 carbon atoms, more specifically, between 3 and 4 carbon atoms), optionally substituted as noted above.

Halo can be selected from Cl, F, Br and I. More specifically, halo can be selected from Cl and F. Preferably, halo is Cl.

As noted above, the term “heteroalkylene” is a bivalent linear saturated hydrocarbon having 2 to 5 carbon atoms (C₂-C₅), wherein 1 or 2 of the 2 to 5 carbon atoms are replaced with NR8, S, or O; heteroalkylene may optionally be substituted with 1 or 2 substituents independently selected from alkyl (C₁-C₆)alkoxy, OH, CN, CF₃, and halo. More specifically, heteroalkylene can be a valent linear saturated hydrocarbon having 2 to 4 carbon atoms (C₂-C₄), wherein at least one of the 2 to 4 carbon atoms is replaced with NR8, S, or O, or having 2 to 3 carbon atoms (C₂-C₃), wherein at least one of the 2 to 3 carbon atoms is replaced with NR8, S, or O, each optionally substituted as noted above.

“Heteroaryl” and “heteroaryl^{b}” are as defined above. Typically, “heteroaryl” or “heteroaryl^{b}” will be optionally substituted with 1, 2 or 3 substituents. Optional substituents are selected from those stated above. Examples of suitable heteroaryl or heteroaryl^{b} groups include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, indolyl, benzimidazolyl, benzotriazolyl, quinolinyl and isoquinolinyl (optionally substituted as stated above).

As noted above, “heterocyclyl” is a 4-, 5-, 6-, or 7-membered carbon-containing non-aromatic ring containing one or two ring members that are selected from N, NR8, S, SO, SO₂ and O; heterocyclyl may be optionally substituted with 1, 2, 3, or 4 substituents independently selected from alkyl, alkoxy, OH, OCF₃, halo, oxo, CN, —NR13R14, —O(aryl^{b}), —O(heteroaryl^{b}) and CF₃; or optionally wherein two ring atoms on heterocyclyl are linked with an alkylene to form a non-aromatic ring containing 5, 6, or 7 ring members; or optionally wherein two adjacent ring atoms on heterocyclyl are linked to form a 5- or 6-membered aromatic ring containing 1 or 2 heteroatoms that are selected from N, NR8, S, and O; or optionally wherein a carbon ring atom on heterocyclyl is substituted with a heteroalkylene such that the carbon ring atom on heterocyclyl together with the heteroalkylene forms a heterocyclyl^{b} that is spiro to ring heterocyclyl.

More specifically, “heterocyclyl” can be a 4-, 5-, 6-, or 7-membered carbon-containing non-aromatic ring containing one or two ring members that are selected from N, NR8, and O (optionally substituted in the same manner as “heterocyclyl”).

As noted above, “heterocyclyl” is a 4-, 5-, 6-, or 7-membered carbon-containing non-aromatic ring containing one or two ring members that are selected from N, NR12, S, SO, SO₂ and O; heterocyclyl^{b} may be optionally substituted with 1, 2, 3, or 4 substituents independently selected from methyl, ethyl, propyl, isopropyl, alkoxy, OH, OCF₃, halo, oxo, CN, and CF₃. More specifically, “heterocyclyl” is a 4-, 5-, 6-, or 7-membered carbon-containing non-aromatic ring containing one or two ring members that are selected from N, NR12, and O (optionally substituted in the same manner as “heterocyclyl^{b}”.

The term “O-linked”, such as in “O-linked hydrocarbon residue”, means that the hydrocarbon residue is joined to the remainder of the molecule via an oxygen atom.

The term “N-linked”, such as in “N-linked pyrrolidinyl”, means that the heterocycloalkyl group is joined to the remainder of the molecule via a ring nitrogen atom.

In groups such as —(CH₂)₁₋₃-aryl, “-” denotes the point of attachment of the substituent group to the remainder of the molecule.

“Pharmaceutically acceptable salt” means a physiologically or toxicologically tolerable salt and includes, when appropriate, pharmaceutically acceptable base addition salts and pharmaceutically acceptable acid addition salts. For example (i) where a compound of the invention contains one or more acidic groups, for example carboxy groups, pharmaceutically acceptable base addition salts that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002).

“Prodrug” refers to a compound which is convertible in vivo by metabolic means (e.g. by hydrolysis, reduction or oxidation) to a compound of the invention. Suitable groups for forming prodrugs are described in ‘The Practice of Medicinal Chemistry, 2^{nd} Ed. pp 561-585 (2003) and in F. J. Leinweber, Drug Metab. Res., 1987, 18, 379.

The compounds of the invention can exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when the solvent is water.

Where compounds of the invention exist in one or more geometrical, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis).

Unless otherwise stated, the compounds of the invention include compounds that differ only in the presence of one or more isotopically enriched atoms. For example, compounds wherein hydrogen is replaced by deuterium or tritium, or wherein carbon is replaced by ¹³C or ¹⁴C, are within the scope of the present invention. Such compounds are useful, for example, as analytical tools or probes in biological assays.

In the context of the present invention, references herein to “treatment” include references to curative, palliative and prophylactic treatment.

General Methods

The compounds of the invention may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention which may impart either a functional (i.e., drug release rate controlling) and/or a non-functional (i.e., processing aid or diluent) characteristic to the formulations. The choice of excipient will to a large extent depend on factors such as the particular mode of administration, the effect of the excipient on solubility and stability, and the nature of the dosage form.

Compounds of the invention intended for pharmaceutical use may be administered as a solid or liquid, such as a tablet, capsule or solution. Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Accordingly, the present invention provides a pharmaceutical composition comprising a compound of the invention and a pharmaceutically acceptable carrier, diluent or excipient.

For the treatment of conditions such as retinal vascular permeability associated with diabetic retinopathy and diabetic macular edema, the compounds of the invention may be administered in a form suitable for injection into the ocular region of a patient, in particular, in a form suitable for intra-vitreal injection. It is envisaged that formulations suitable for such use will take the form of sterile solutions of a compound of the invention in a suitable aqueous vehicle. The compositions may be administered to the patient under the supervision of the attending physician.

The compounds of the invention may also be administered directly into the blood stream, into subcutaneous tissue, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous or oily solutions. Where the solution is aqueous, excipients such as sugars (including but not restricted to glucose, manitol, sorbitol, etc.), salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

Parenteral formulations may include implants derived from degradable polymers such as polyesters (i.e., polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of co-solvents and/or solubility-enhancing agents such as surfactants, micelle structures and cyclodextrins.

The compounds of the invention can be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solids and liquids (including multiple phases or dispersed systems). Exemplary formulations suitable for oral administration include tablets; soft or hard capsules containing multi- or nano-particulates, liquids, emulsions or powders; lozenges (including liquid-filled); chews; gels; fast dispersing dosage forms; films; ovules; sprays; and buccal/mucoadhesive patches.

Liquid (including multiple phases and dispersed systems) formulations include emulsions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents, 2001, 11 (6), 981-986.

The formulation of tablets is discussed in Pharmaceutical Dosage Forms: Tablets, Vol. 1, by H. Lieberman and L Lachman (Marcel Dekker, New York, 1980).

For administration to human patients, the total daily dose of the compounds of the invention is typically in the range 0.1 mg and 10,000 mg, or between 1 mg and 5000 mg, or between 10 mg and 1000 mg depending, of course, on the mode of administration.

The total dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

Synthetic Methods

The compounds of the present invention can be prepared according to the procedures of the following schemes and examples, using appropriate materials, and are further exemplified by the specific examples provided herein below. Moreover, by utilising the procedures described herein, one of ordinary skill in the art can readily prepare additional compounds that fall within the scope of the present invention claimed herein. The compounds illustrated in the examples are not, however, to be construed as forming the only genus that is considered as the invention. The examples further illustrate details for the preparation of the compounds of the present invention. Those skilled in the art will readily understand that known variations of the conditions, processes and order in which the synthetic steps are performed in the following preparative procedures can be used to prepare these compounds.

The compounds and intermediates of the invention may be isolated in the form of their pharmaceutically acceptable salts, such as those described previously herein above. The interconversion between free form and salt form would be readily known to those skilled in the art.

It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by T. W. Greene and P. G. M. Wuts in “Protective groups in organic chemistry” John Wiley and Sons, 4^{th} Edition, 2006, may be used. For example, a common amino protecting group suitable for use herein is tert-butoxy carbonyl (Boc), which is readily removed by treatment with an acid such as trifluoroacetic acid or hydrogen chloride in an organic solvent such as dichloromethane. Alternatively the amino protecting group may be a benzyloxycarbonyl (Z) group which can be removed by hydrogenation with a palladium catalyst under a hydrogen atmosphere or 9-fluorenylmethyloxycarbonyl (Fmoc) group which can be removed by solutions of secondary organic amines such as diethylamine or piperidine in an organic solvent. Carboxyl groups are typically protected as esters such as methyl, ethyl, benzyl or tert-butyl which can all be removed by hydrolysis in the presence of bases such as lithium or sodium hydroxide. Benzyl protecting groups can also be removed by hydrogenation with a palladium catalyst under a hydrogen atmosphere whilst tert-butyl groups can also be removed by trifluoroacetic acid. Alternatively a trichloroethyl ester protecting group is removed with zinc in acetic acid. A common hydroxy protecting group suitable for use herein is a methyl ether, deprotection conditions comprise refluxing in 48% aqueous HBr, or by stirring with borane tribromide in an organic solvent such as DCM. Alternatively where a hydroxy group is protected as a benzyl ether, deprotection conditions comprise hydrogenation with a palladium catalyst under a hydrogen atmosphere.

The compounds according to general formula I can be prepared using conventional synthetic methods for example, but not limited to, the route outlined in Schemes 1-8.

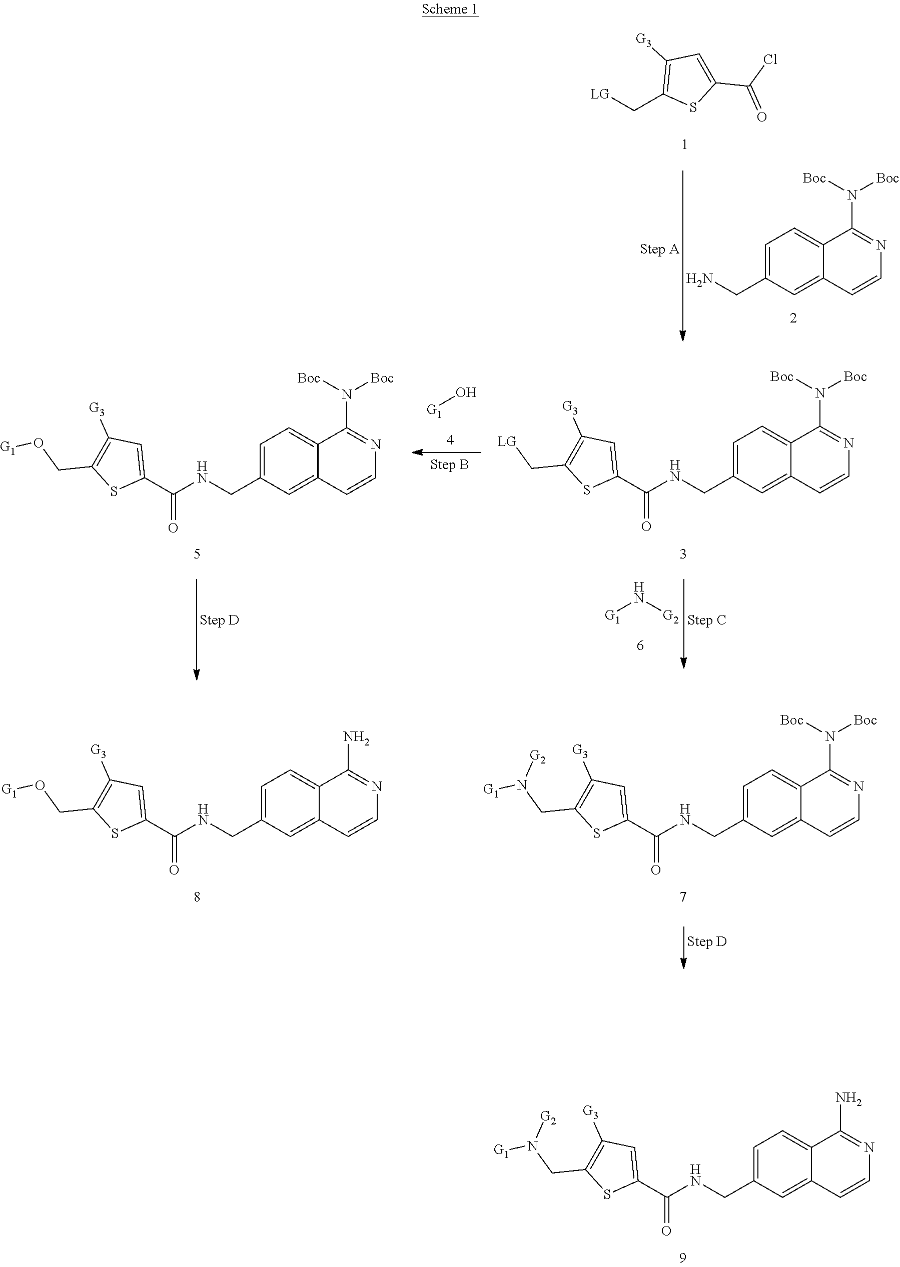

Where LG is Cl or Br the acid chloride 1 is coupled to amine 2 using standard coupling conditions, for example in the presence of pyridine (Step A). The alkyl halide 3 can be reacted with, for example, phenols such as 4 using catalyst 2-tert-butylimino-2-diethylamino-1,3-dimethylperhyro-1,3,2-diazaphosphorine (BEMP) in the presence of a solvent such as DMF, or, for example, with alcohols such as 4 using potassium tert-butoxide in a solvent such as NMP (Step B). Alternatively, the alkyl halide 3 can be reacted with amines such as 6 using standard alkylation conditions (Step C), for example in the presence of a base such as N,N-diisopropylethylamine in a solvent such as DMF. Both ether 5 and amine 7 are deprotected (Step D) using acidic conditions such as trifluoroacetic acid or HCl to give amines 8 and 9 respectively. These products can either be isolated in the form of the acid salt, for example the trifluoroacetate or HCl, or as the free base.

Alternatively, compounds can also be assembled in a different order, as shown in Scheme 2.

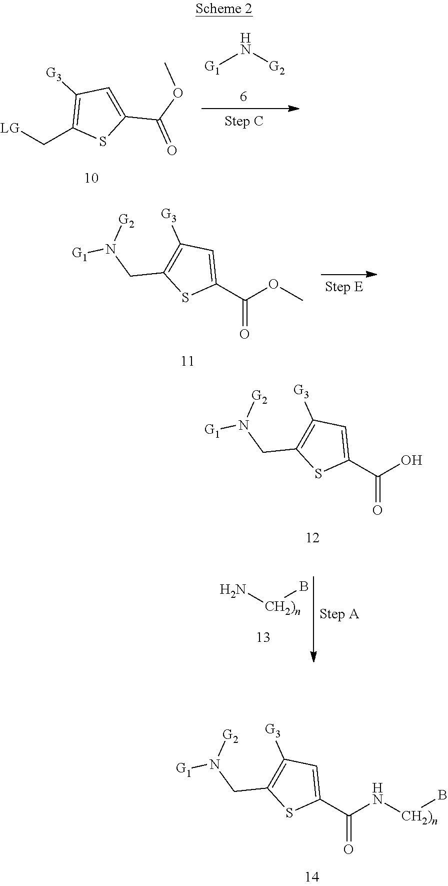

The halide 10 can be reacted with primary and secondary amines (such as 6) using standard alkylation conditions (Step C) for example, in the presence of a base such as N,N-diisopropylethylamine, potassium carbonate or caesium carbonate in a solvent such as DMF, dioxane or acetonitrile. The ester 11 is hydrolysed (Step E) using standard literature conditions such as NaOH, KOH, LiOH, or TMSOK. The acid (or salt) 12 is coupled to amine (or salt) 13 (Step A) to give compound 14. This coupling is typically carried out using standard coupling conditions such as hydroxybenzotriazole (HOBt) and carbodiimide such as water soluble carbodiimide in the presence of an organic base. Other standard coupling methods include the reaction of acids with amines in the presence of 2-(1H-benzotriazole-1-yl)-1,1,3,3-tetramethylaminium hexafluorophosphate (HBTU) orbenzotriazole-1-yl-oxy-tris-pyrrolidino-phosphoium hexafluorophosphate (PyBOP) or bromo-trispyrolidino-phosphonium hexafluorophosphate (PyBroP) or 2-(3H-[1,2,3]triazolo[4,5-b]pyridin-3-yl)-1,1,3,3-tetramethylisouronium hexafluorophosphate(V) (HATU), or 1-ethyl-3-(3-dimethylaminopropyl)carbodiimide (EDC) in the presence of organic bases such as triethylamine, diisopropylethylamine or N-methylmorpholine. Alternatively, the amide formation can take place via an acid chloride in the presence of an organic base. Such acid chlorides can be formed by methods well known in the literature, for example reaction of the acid with oxalyl chloride or thionyl chloride. Alternatively, the carboxylic acid can be activated using 1,1′-carbonyldiimidazole (CDI) and then amine added. The amine 13 may be commercially available or prepared from readily available starting materials using methods known in the art, or as detailed in specific examples herein. Depending on B the final compound may require removal of protecting groups using methods known in the art.

Furthermore, addition of the amine 6 can also be completed via reductive alkylation as in shown in Scheme 3 and may be carried out using standard conditions for such a transformation (Step F).

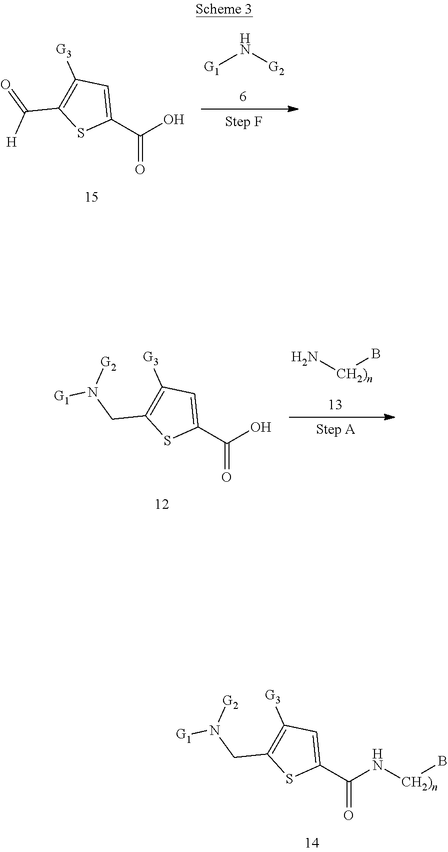

In Step F, aldehyde 15 is treated with amine 6 followed by the addition of a reducing agent such as sodium triacetoxyborohydride to give compound 12. Alternative reducing agents include sodium borohydride and sodium cyanoborohydride.

When the substituent is attached via a heteroatom to the central heteroaryl ring, examples can be prepared using conventional synthetic methods for example, but not limited to, the routes outlined in Schemes 4-6

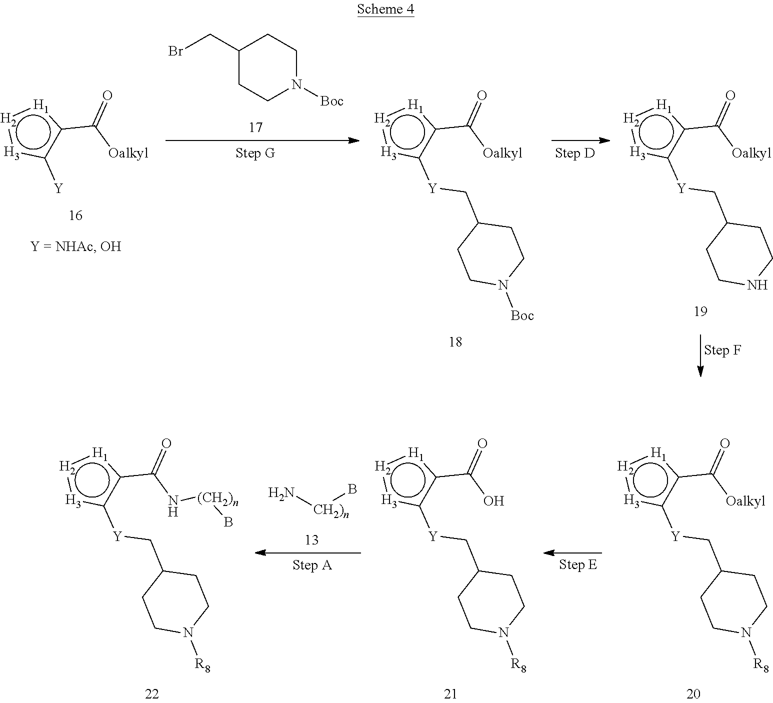

The alcohol or protected amine 16 (exemplified in Scheme 4 with acetyl as the amine protecting group) is reacted with an alkyl bromide such as compound 17 under standard alkylation conditions via a formal deprotonation. Methods for such transformations are known in the art, typically in the presence of sodium hydride in a solvent such as dimethylformamide (Step G). The Boc protecting group is removed (Step D) using standard acidic conditions such as trifluoroacetic acid to give amine 19. Typically, this intermediate would be isolated in the form of the acid salt, for example the trifluoroacetate. Methylation of the amine (Step F) may be carried out using standard conditions for such a transformation. For example, amine 19 is treated with formaldehyde (37% in water) followed by the addition of a reducing agent such as sodium triacetoxyborohydride to give compound 20. Alternative alkylations may be carried out by use of the appropriate alkanone, for example amine 19 is treated with the alkanone, for example acetone, in an organic solvent such as DCM followed by the addition of a reducing agent such as sodium triacetoxyborohydride to give compound 20. Alternative reducing agents include sodium borohydride and sodium cyanoborohydride. The ester is hydrolysed (Step E) using standard literature conditions such as NaOH, KOH, or LiOH. The acid (or salt) 21 is coupled to amine (or salt) 13 (Step A) to give compound 22. This coupling is typically carried out using standard coupling conditions as previously described.

When Y is protected with the use of a protecting group such as acetyl, the protecting group is removed during the synthetic sequence a shown in Scheme 5. Protected amine 20-a is deprotected using methane sulfonic acid and heating at 100° C. to give compound 20-b (Step H).

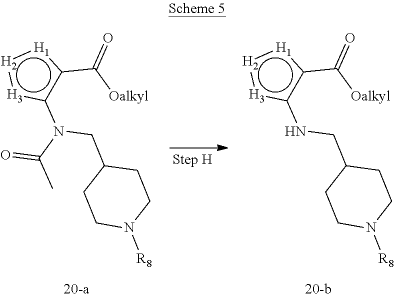

In a variation to Scheme 4, When Y is NH₂, intermediate 18 can also be prepared via reductive alkylation carried out by use of the appropriate alkanone (Step F) as shown in Scheme 6.

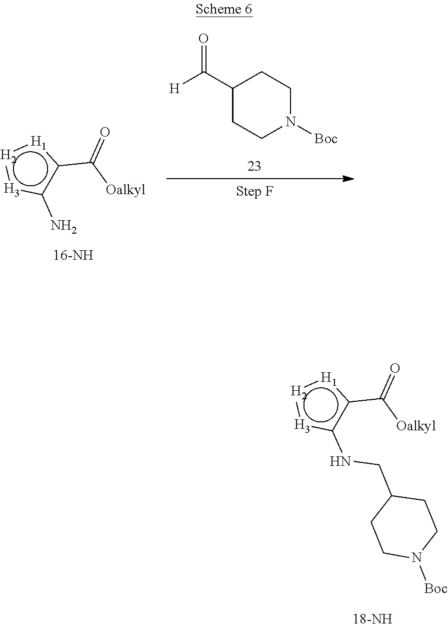

Amine 16-NH is treated with the alkanone 23, in an organic solvent such as DCM followed by the addition of a reducing agent such as sodium triacetoxyborohydride to give compound 18-NH. As previously described, alternative reducing agents and procedures can be used and are known in the art.

In a further variation to Scheme 4, a substituted heteroaromatic ring can be alkylated using conventional synthetic methods for example, but not limited to, the routes outlined in Schemes 7 and 8.

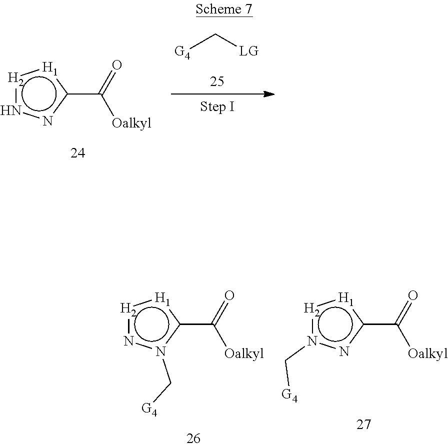

A heteroaromatic ring, such as 24, may be alkylated using alcohol 25 (where LG is hydroxy) as shown in Scheme 7 (Step 1) under Mitsunobu conditions in the presence of triphenylphosphine. In this case there are two possible nitrogens for the alkylation to occur at therefore there is the possibility of two regioisomers being formed. Regioisomers may be separated at this stage or at a subsequent stage in the synthesis using separation methods well known to those skilled in the art, for example by chromatography or by fractional crystallisation, confirming their identity by ¹H NMR analysis. Alternatively, where LG is a halide or sulfonate, the alkylation may be carried out in the presence of a base such as potassium carbonate, caesium carbonate, sodium carbonate or sodium hydride.

Alternatively, the alkylation can be carried out via in situ sulfonyl transfer (see Jane Panteleev et al., “Alkylation of Nitrogen-Containing Heterocycles via In Situ Sulfonyl Transfer”, Synlett 26(08)), as shown in Scheme 8 (Step J).

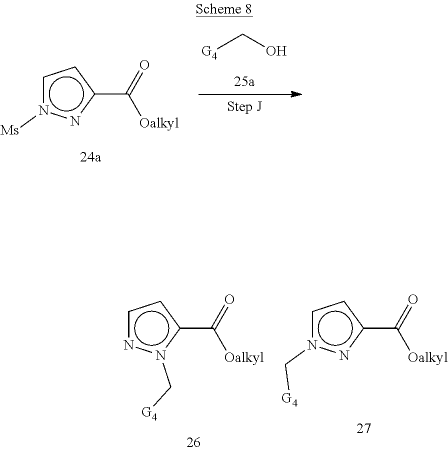

The pyrazole mesylate 24a is prepared by treating pyrazole 24 with methanesulfanyl chloride (MsCl) with a base such as triethylamine in a solvent such as dichloromethane. Alternatively other sulfonyl groups can be used such as toluenesulfonyl (Ts) or benzenesulfonyl. The pyrazole mesylate 24a may be coupled to the alcohol 25a in the presence of a base such as caesium carbonate in a solvent such as acetonitrile. Regioisomers 26 and 27 may be separated at this stage or at a subsequent stage in the synthesis using separation methods well known to those skilled in the art, for example by chromatography or by fractional crystallisation, confirming their identity by ¹H NMR analysis.

### EXAMPLES

The invention is illustrated by the following non-limiting examples in which the following abbreviations and definitions are used:
Aq Aqueous solution
AIBN Azobisisobutyronitrile
BEMP 2-tert-Butylimino-2-diethylamino-1,3-dimethykperhyro-1,3,2-diazaphosphorine
tBu Tert-Butyl
CDI 1,1′-Carbonyldiimidazole
COMU [[(E)-(1-Cyano-2-ethoxy-2-oxo-ethylidene)amino]oxy-morpholino-methylene]-dimethyl-ammonium hexa-fluorophosphate
DCM Dichloromethane
DIPEA N,N-Diisopropylethylamine
DMF N,N-Dimethylformamide
DMSO Dimethyl sulfoxide
Eq Equivalent
Et₂O Diethyl ether
Et Ethyl
EtOH Ethanol
EtOAc Ethyl Acetate
HATU 2-(3H-[1,2,3]triazolo[4,5-b]pyridin-3-yl)-1,1,3,3-tetramethylisouronium hexafluorophosphate(V)
hrs Hours
HOBt Hydroxybenzotriazole
LCMS Liquid chromatography mass spectrometry
Me Methyl
MeCN Acetonitrile
MsCl Methanesulfonyl chloride
MeOH Methanol
Min Minutes
MS Mass spectrum
Ms Methanesulfonyl
NMR Nuclear magnetic resonance spectrum
NMP N-Methyl-2-pyrrolidone
Pet. Ether Petroleum ether fraction boiling at 60-80° C.
Ph Phenyl
iPr Iso-propyl
nPr n-Propyl
SWFI Sterile water for injection
rt room temperature
T3P Propylphosphonic anhydride
TBDMS tert-Butyldimethylsilyl
TBME tert-Butyl methyl ether
THF Tetrahydrofuran
TEA Triethylamine
TFA Trifluoroacetic acid

All reactions were carried out under an atmosphere of nitrogen unless specified otherwise.

¹H NMR spectra were recorded on a Bruker (500 MHz or 400 MHz) spectrometer and reported as chemical shift (ppm).

Molecular ions were obtained using LCMS with appropriate conditions selected from
Chromolith Speedrod RP-18e column, 50×4.6 mm, with a linear gradient 10% to 90% 0.1% HCO₂H/MeCN into 0.1% HCO₂H/H₂O over 13 min, flow rate 1.5 mL/min;
Agilent, X-Select, acidic, 5-95% MeCN/water over 4 min. Data was collected using a Thermofinnigan Surveyor MSQmass spectrometer with electospray ionisation in conjunction with a Thermofinnigan Surveyor LC system;
LCMS (Waters Acquity UPLC, C18, Waters X-Bridge UPLC C18, 1.7 μm, 2.1×30 mm, Basic (0.1% Ammonium Bicarbonate) 3 min method;
LCMS (Agilent, X-Select, Waters X-Select C18, 2.5 μm, 4.6×30 mm, Acidic 4 min method, 95-5 MeCN/water);
LCMS (Agilent, Basic, Waters X-Bridge C18, 2.5 μm, 4.6×30 mm, Basic 4 min method, 5-95 MeCN/water;
Acquity UPLC BEH C18 1.7 μM column, 50×2.1 mm, with a linear gradient 10% to 90% 0.1% HCO₂H/MeCN into 0.1% HCO₂H/H₂O over 3 minutes, flow rate 1 mL/min. Data was collected using a Waters Acquity UPLC mass spectrometer with quadropole dalton, photodiode array and electrospray ionisation detectors.

Flash chromatography was typically carried out over ‘silica’ (silica gel for chromatography, 0.035 to 0.070 mm (220 to 440 mesh) (e.g. Merck silica gel 60)), and an applied pressure of nitrogen up to 10 p.s.i accelerated column elution. Alternatively, pre-prepared cartridges of silica gel were used. Reverse phase preparative HPLC purifications were carried out using a Waters 2525 binary gradient pumping system at flow rates of typically 20 mL/min using a Waters 2996 photodiode array detector.

All solvents and commercial reagents were used as received.

Chemical names were generated using automated software such as ChemDraw (PerkinElmer) or the Autonom software provided as part of the ISIS Draw package from MDL Information Systems or the Chemaxon software provided as a component of MarvinSketch or as a component of the IDBS E-WorkBook.

### Synthesis of Intermediates

General Method A: Amide Formation

(i) Coupling Reagent, Eg HATU

### Example 5.23 N-[(5R)-1-Amino-6,7-dihydro-5H-cyclopenta[c]pyridin-5-yl]-4-chloro-5-[[4-(4-pyridyl)piperazin-1-yl]methyl]thiophene-2-carboxamide

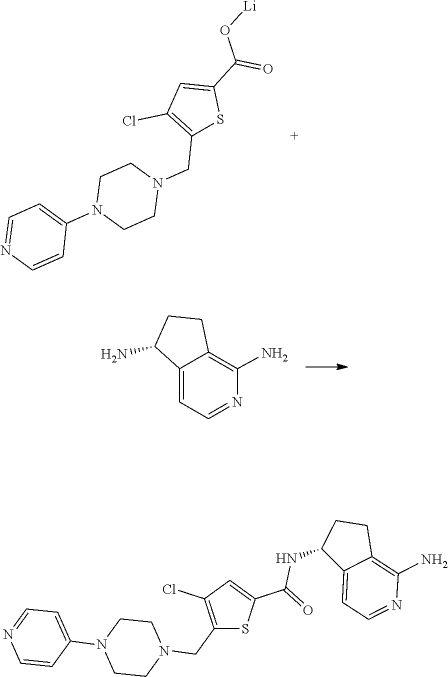

N,N-diisopropylethylamine (0.15 mL, 0.86 mmol) was added to a solution of [4-chloro-5-[[4-(4-pyridyl)piperazin-1-yl]methyl]thiophene-2-carbonyl]oxylithium (60 mg, 0.18 mmol), (5R)-6,7-dihydro-5Hcyclopenta[c]pyridine-1,5-diamine dihydrochloride (43 mg, 0.19 mmol) and HATU (80 mg, 0.21 mmol) in NMP (1 mL) and stirred for 3 hrs. The reaction was diluted with MeOH (10 mL), absorbed onto SOC, washed with MeOH (30 mL) and the product eluted with 0.7M NH₃/MeOH. The MeOH was evaporated in vacuo and the residual gum treated with Et₂O and the resulting solid filtered off and dried to afford the title compound (73 mg, 88% yield), as a beige solid.

[M+H]⁺=469.2/471.2

¹H NMR (DMSO-d6, 500 MHz) δ 1.86-1.92 (1H, m), 2.41-2.47 (1H, m), 2.55-2.61 (5H, m), 2.76-2.82 (1H, m), 3.32-3.43 (4H, m), 3.76 (2H, s), 5.35-5.41 (1H, m), 5.82 (2H, d, J=5.9 Hz), 6.45 (1H, d, J=5.1 Hz), 6.80-6.85 (2H, m), 7.74-7.80 (2H, m), 8.14-8.19 (2H, m), 8.81 (1H, d, J=8.4 Hz).

General Method A: Amide Formation

(ii) Acid Chloride

### tert-Butyl (tert-butoxycarbonyl) (6-((4-chloro-5-(chloromethyl)thiophene-2-carboxamido)methyl) isoquinolin-1-yl)carbamate

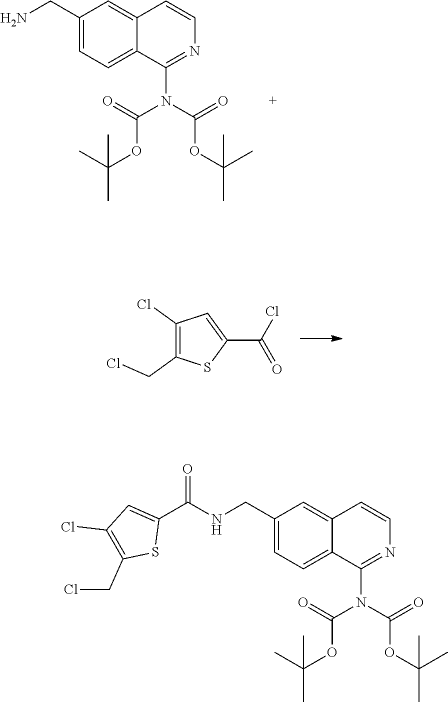 [SMILES: CC(C)(C)OC(=O)N(C(=O)OC(C)(C)C)c1nccc2cc(CN)ccc12 ; O=C(Cl)c1cc(Cl)c(CCl)s1 ; CC(C)(C)OC(=O)N(C(=O)OC(C)(C)C)c1nccc2cc(CNC(=O)c3cc(Cl)c(CCl)s3)ccc12]

A solution of 4-chloro-5-(chloromethyl)thiophene-2-carbonyl chloride (6.57 g, 28.6 mmol) in anhydrous DCM (80 mL) at 0° C. was treated dropwise with a solution of tert-butyl N-[6-(aminomethyl)-1-isoquinolyl]-N-tert-butoxycarbonyl-carbamate (11.9 g, 28.6 mmol) and pyridine (2.78 mL, 34.4 mmol) in anhydrous DCM (50 mL). The mixture was stirred at rt for 2 hrs then the solvents removed under vacuum. The residue was purified by flash chromatography (0-50 EtOAc/Isohexane) to afford the title compound (10.6 g, 63% yield) as a white solid.

[M+H]⁺=566.2

General Method B (i): Phenol Alkylation

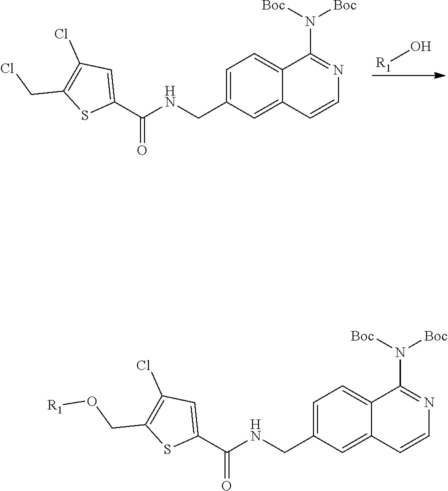

Stock solutions of the electrophile, tert-butyl (tert-butoxycarbonyl)(6-((4-chloro-5-(chloromethyl)thiophene-2-carboxamido)methyl)isoquinolin-1-yl)carbamate (395.5 mg made up to 2.8 mL in anhydrous DMF) and BEMP (406 μL made up to 2.8 mL in anhydrous DMF) were prepared. Phenolic reagents (0.2 mmol) were added to a 96 well plate. 0.4 mL of the electrophile solution was added to each well, followed by 0.4 mL of the BEMP solution. The mixtures were shaken (Thermo Scientific, 880 rpm). The crude products were filtered and purified by prep HPLC.

General Method B (ii): Alcohol Alkylation

### Example 7.26

### N-((1-Aminoisoquinolin-6-yl)methyl)-4-chloro-5-((4-(dimethylamino)butoxy)methyl)thiophene-2-carboxamide

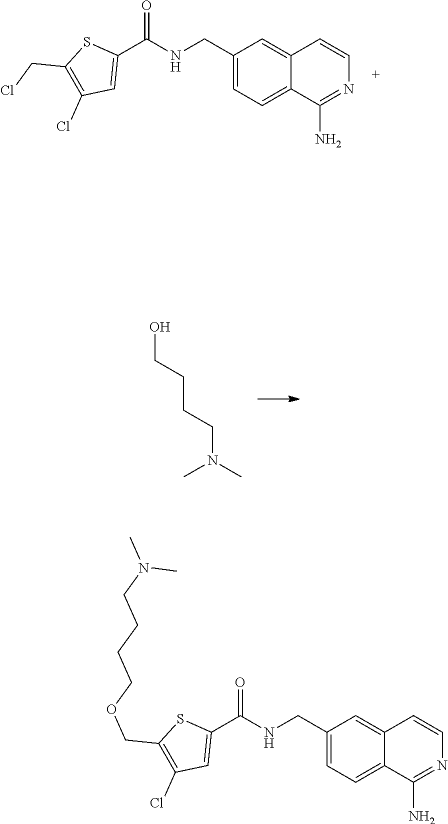

A solution of the alcohol 4-(dimethylamino)butan-1-ol (0.3 mmol, 35.16 mmol) was treated with a 1M THF solution of potassium tert-butoxide (0.5 mL, 0.5 mmol) and the mixture was inverted and then shaken on a plate shaker for 30 min. A solution of N-((1-aminoisoquinolin-6-yl)methyl)-4-chloro-5-(chloromethyl)thiophene-2-carboxamide hydrochloride (40.3 mg, 0.1 mmol) in anhydrous NMP (0.5 mL) was then added and the mixture was inverted and shaken vigorously for 2 hrs on a plate shaker. Then the mixture was quenched with acetic acid (0.03 mL) and water (0.2 mL). The product was purified preparative HPLC to afford title compound (7.4 mg, 17% yield).

[M+H]⁺=447.5

General Method C: N-Alkylation

(i) DIPEA

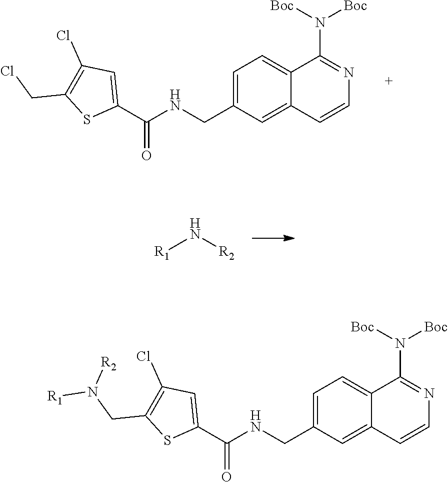

To a stirred solution of tert-butyl N-tert-butoxycarbonyl-N-[6-[[[4-chloro-5-(chloromethyl)thiophene-2-carbonyl]amino]methyl]-1-isoquinolyl]carbamate (120 mg, 0.21 mmol) in DMF (2 mL) at rt was added N,N diisopropylethylamine (150 μL, 0.86 mmol) and the required amine (0.42 mmol). The resulting mixture was stirred at rt overnight. The reaction was diluted with EtOAc (20 mL) then washed with water (5×10 mL) and brine (10 mL), dried (MgSO₄), filtered and evaporated in vacuo. The crude product was purified by flash chromatography (0-10% MeOH in EtOAc) to afford the desired products.

### tert-Butyl (R)-(tert-butoxycarbonyl)(6-((4-chloro-5-((3-(pyridin-3-ylmethyl)pyrrolidin-1-yl)methyl) thiophene-2-carboxamido)methyl)isoquinolin-1-yl)carbamate

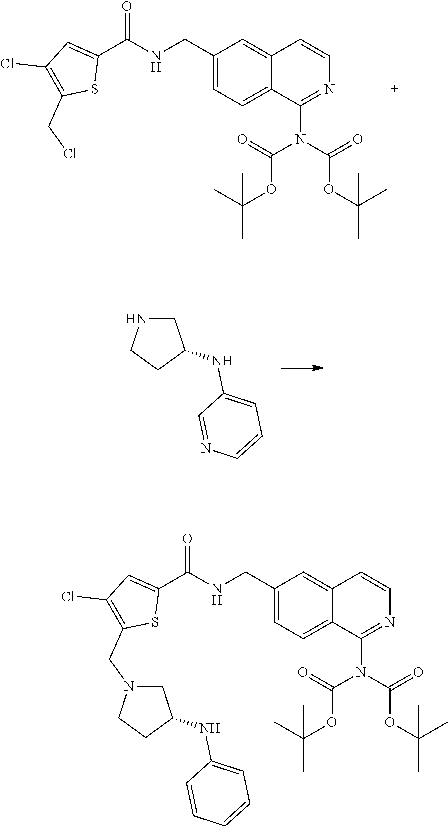 [SMILES: CC(C)(C)OC(=O)N(C(=O)OC(C)(C)C)c1nccc2cc(CNC(=O)c3cc(Cl)c(CCl)s3)ccc12 ; c1cncc(N[C@@H]2CCNC2)c1 ; CC(C)(C)OC(=O)N(C(=O)OC(C)(C)C)c1nccc2cc(CNC(=O)c3cc(Cl)c(CN4CC[C@@H](Nc5ccccc5)C4)s3)ccc12]

N-[(3R)-pyrrolidin-3-yl]pyridin-3-amine dihydrochloride (100 mg, 0.42 mmol) was reacted under the general conditions above. The title compound was isolated (62 mg, 42% yield) as a colourless oil.

[M+H]⁺=693.2

General Method C: N-Alkylation

(ii) K₂CO₃

### Ethyl 4-chloro-5-[[4-(4-pyridyl)piperazin-1-yl]methyl]thiophene-2-carboxylate

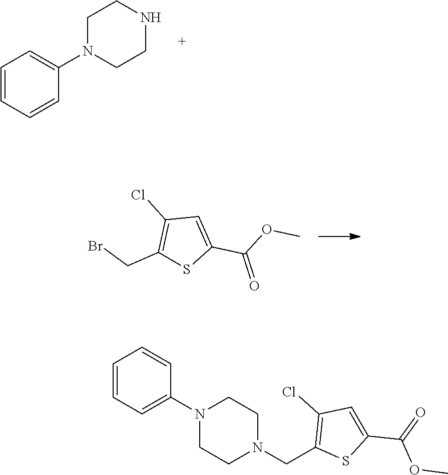 [SMILES: c1ccc(N2CCNCC2)cc1 ; COC(=O)c1cc(Cl)c(CBr)s1 ; COC(=O)c1cc(Cl)c(CN2CCN(c3ccccc3)CC2)s1]

1-(4-pyridyl)piperazine (968 mg, 5.93 mmol), ethyl 5-(bromomethyl)-4-chloro-thiophene-2-carboxylate (840 mg, 2.96 mmol) and K₂CO₃ (1.23 g, 8.9 mmol) were stirred in DMF (10 mL) for 20 hrs. The reaction was diluted with EtOAc (100 mL), washed with water (3×30 mL) and brine (20 mL), dried (MgSO₄), filtered and evaporated in vacuo. The residue was purified by flash chromatography (0 to 10% MeOH (1% NH₃)/DCM) to afford the title compound (800 mg, 69% yield) as a pale yellow oil.

[M+H]⁺=366.2

¹H NMR (DMSO-d6, 500 MHz) d 1.29 (3H, t, J=7.1 Hz), 2.57-2.63 (4H, m), 3.30-3.36 (4H, m), 3.80 (2H, s), 4.29 (2H, q, J=7.1 Hz), 6.79-6.84 (2H, m), 7.71 (1H, s), 8.14-8.19 (2H, m).

General Method D: Boc Deprotection

(i) TFA

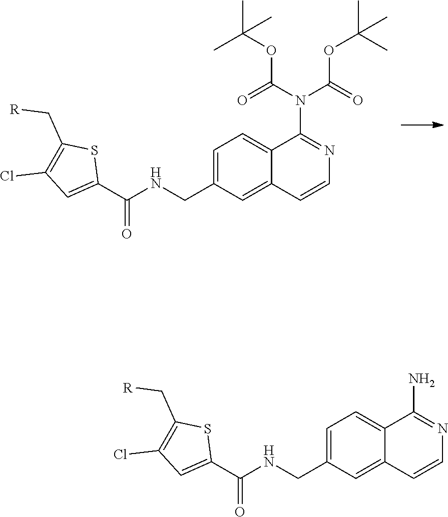

The Boc protected reagents (0.1 mmol) were dissolved in anhydrous DCM (1 mL) and treated with TFA (1 mL), then allowed to shake for 5 hrs and then concentrated. The crude products were purified by prep HPLC.

General Method D: Boc Deprotection

(ii) HCl

### Example 5.24 N-((1-Aminoisoquinolin-6-yl) methyl)-4-methyl-5-((4-(pyridin-4-yl)-1,4-diazepan-1-yl) methyl) thiophene-2-carboxamide

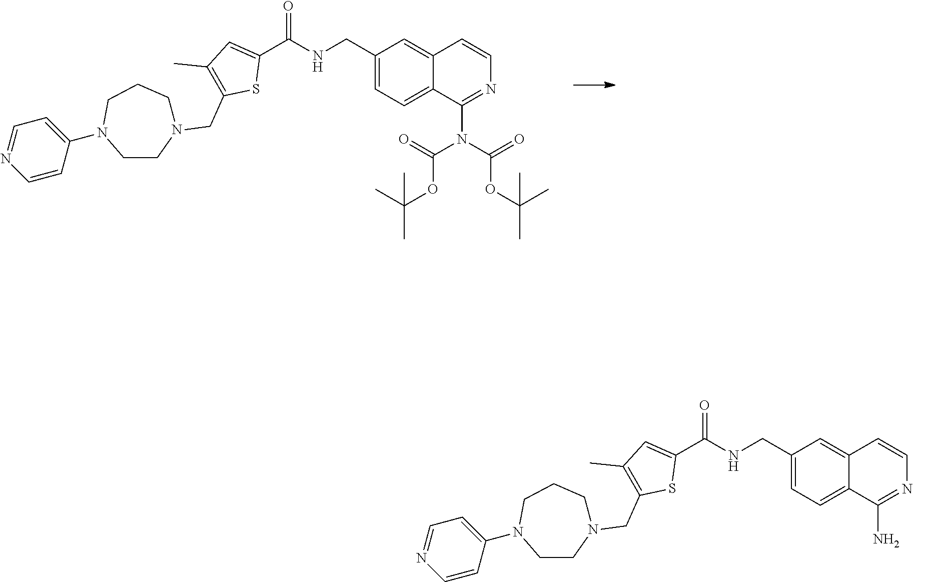

4M HCl in dioxane (3 mL, 12 mmol) was added to a solution of tert-butyl N-tert-butoxycarbonyl-N-[6-[[[4-methyl-5-[[4-(4-pyridyl)-1,4-diazepan-1-yl]methyl]thiophene-2-carbonyl]amino]methyl]-1-isoquinolyl]carbamate (58 mg, 0.084 mmol) in MeOH (1 mL) and stirred for 5 hrs. The solvent was evaporated in vacuo and the residual solid treated with ether, filtered off and dried (MgSO₄) to afford the title compound (49 mg, 94% yield) as a cream solid.

[M+H]⁺=485.3

General Method E: Ester Hydrolysis

### [4-Chloro-5-[[4-(4-pyridyl)piperazin-1-yl]methyl]thiophene-2-carbonyl]oxylithium

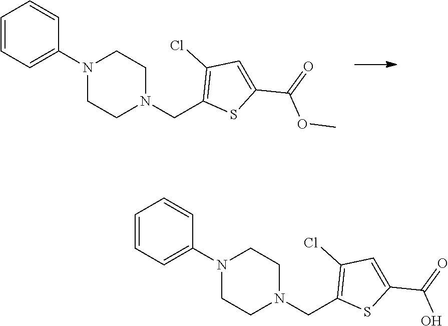 [SMILES: COC(=O)c1cc(Cl)c(CN2CCN(c3ccccc3)CC2)s1 ; O=C(O)c1cc(Cl)c(CN2CCN(c3ccccc3)CC2)s1]

A solution of lithium hydroxide (63 mg, 2.63 mmol) in water (6 mL) was added to a solution of ethyl 4-chloro-5-[[4-(4-pyridyl)piperazin-1-yl]methyl]thiophene-2-carboxylate (800 mg, 2.19 mmol) in THF (6 mL)/MeOH (12 mL) and stirred at 40° C. for 20 hrs. The solvents were evaporated in vacuo and the residue treated with 1,4-dioxane (10 mL). The resulting solid was filtered off, washed with 1,4-dioxane (10 mL) and Et₂O (10 mL) to afford the title compound (753 mg, quantitative yield) as an off-white solid.

¹H NMR (DMSO-d6, 500 MHz) d 2.51-2.56 (4H, m), 3.29-3.35 (4H, m), 3.65 (2H, s), 6.78-6.83 (2H, m), 7.00 (1H, s), 8.12-8.17 (2H, m).

[M+H]+=338.1

General Method F: Reductive Amination

(i) Using Formaldehyde

### Methyl 5-methyl-3-((1-methylpiperidin-4-yl)methoxy)thiophene-2-carboxylate

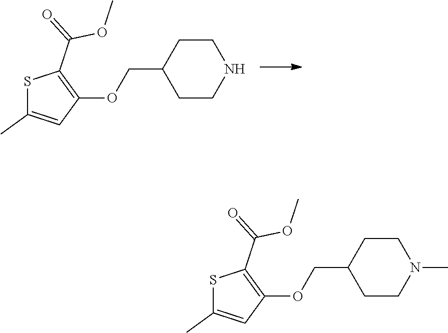

Sodium triacetoxyborohydride (4.5 g, 21.23 mmol) was added to a solution of methyl 5-methyl-3-(piperidin-4-ylmethoxy)thiophene-2-carboxylate (715 mg, 2.65 mmol), paraformaldehyde (638 mg, 21.23 mmol) and acetic acid (0.15 mL, 2.62 mmol) in DCM (12.5 mL) and DMF (2.5 mL). After stirring at rt for 5 min, the solution was heated to 40° C. and left stirring for 2 hrs. The solution was cooled to rt, quenched with H₂O (30 mL) and diluted with EtOAc (50 mL) before being washed with HCl (1M, aq., 30 mL). The aqueous layer was then basified to pH 10 with Na₂CO₃ before being extracted with DCM (3×30 mL), passing through a phase separator and concentrating in vacuo. The crude product was dissolved in DCM (10 mL) and adsorbed on SCX (6 g), before being washed with MeOH (80 mL). The product was released from SOC by washing with 7M ammonia in MeOH (100 mL). The filtrate was concentrated in vacuo to afford the title compound (0.64 g, 77% yield) as yellow viscous oil.

[M+H]⁺=284.2

¹H NMR (500 MHz, DMSO-d6) δ 1.23-1.35 (m, 2H), 1.60-1.69 (m, 1H), 1.68-1.77 (m, 2H), 1.79-1.90 (m, 2H), 2.15 (s, 3H), 2.42 (d, J=1.0 Hz, 3H), 2.72-2.81 (m, 2H), 3.68 (s, 3H), 3.94 (d, J=6.5 Hz, 2H), 6.90 (d, J=1.1 Hz, 1H).

### Methyl 3-chloro-1-(2-(1-methylpiperidin-4-yl)ethyl)-1H-pyrazole-5-carboxylate

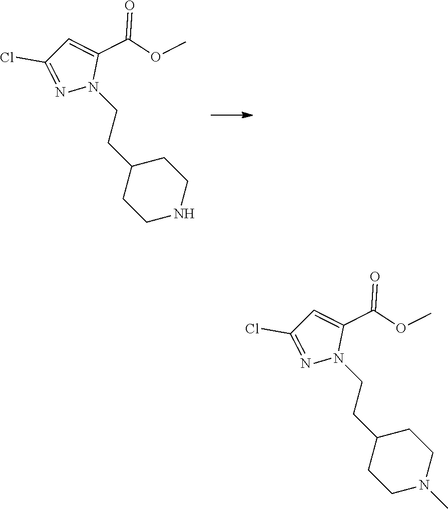 [SMILES: COC(=O)c1cc(Cl)nn1CCC1CCNCC1 ; COC(=O)c1cc(Cl)nn1CCC1CCN(C)CC1]

Polymer supported cyanoborohydride 2 mmol/g (3.32 g, 6.64 mmol) was added to a solution of methyl 3-chloro-1-(2-(piperidin-4-yl)ethyl)-1H-pyrazole-5-carboxylate (451 mg, 1.66 mmol), formaldehyde (aq. 37%) and acetic acid (47 μL, 0.83 mmol) in MeOH (2 ml). The mixture was stirred for 4 hrs then filtered and the filtrate concentrated in vacuo. The residue was purified by flash chromatography (0-100% (10% NH₃ in MeOH) in DCM) to afford title compound (390 mg, 82% yield) as colourless oil.

[M+H]⁺=286.4/288.0

General Method F: Reductive Amination

(ii) Sodium Triacetoxyborohydride and Core Aldehydes

### Ethyl 1-ethyl-4-methyl-5-((4-(pyridin-4-yl)piperazin-1-yl)methyl)-1H-pyrrole-2-carboxylate

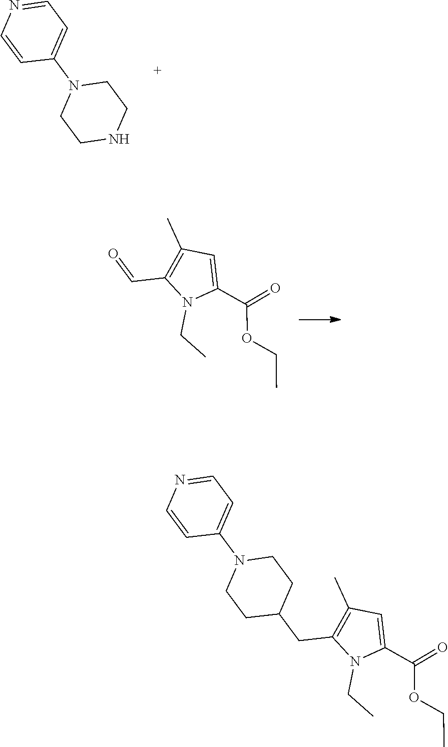 [SMILES: c1cc(N2CCNCC2)ccn1 ; CCOC(=O)c1cc(C)c(C=O)n1CC ; CCOC(=O)c1cc(C)c(CC2CCN(c3ccncc3)CC2)n1CC]

A solution of ethyl 1-ethyl-5-formyl-4-methyl-pyrrole-2-carboxylate (110 mg, 0.526 mmol) and Et₃N (0.183 mL, 1.31 mmol) in anhydrous DCM (6 mL) was treated with 1-(4-pyridyl)piperazine (103 mg, 0.631 mmol). The reaction mixture was stirred for 30 min at rt before the addition of sodium triacetoxyborohydride (245 mg, 1.16 mmol) and then stirred for 18 hrs. The reaction mixture was partitioned between NaHCO₃ (15 mL) solution and DCM (15 mL). The aqueous phase was extracted with further DCM (2×8 mL) and the combined organic layers washed with NaHCO₃ solution (15 mL) and brine (10 mL) then dried (Na₂SO₄), filtered and concentrated in vacuo. The residue was purified by flash chromatography (0-6% (1% NH₃ in MeOH) in DCM) to afford title compound (49 mg, 24% yield) as a colourless gum.

[M+H]⁺=357.3

¹H NMR (DMSO-d6) δ: 1.18-1.31 (6H, m), 2.00 (3H, s), 2.41-2.49 (4H, m), 3.22-3.30 (4H, m), 3.46 (2H, s), 4.18 (2H, q, J=7.1 Hz), 4.34 (2H, q, J=6.9 Hz), 6.68 (1H, s), 6.79 (2H, d, J=6.7 Hz), 8.14 (2H, d, J=6.6 Hz)

General Method G: Alkylations Using NaH

(i) N-Alkylation Using NaH

### tert-Butyl 4-((N-(2-(methoxycarbonyl)-4-methylthiophen-3-yl)acetamido)methyl)piperidine-1-carboxylate

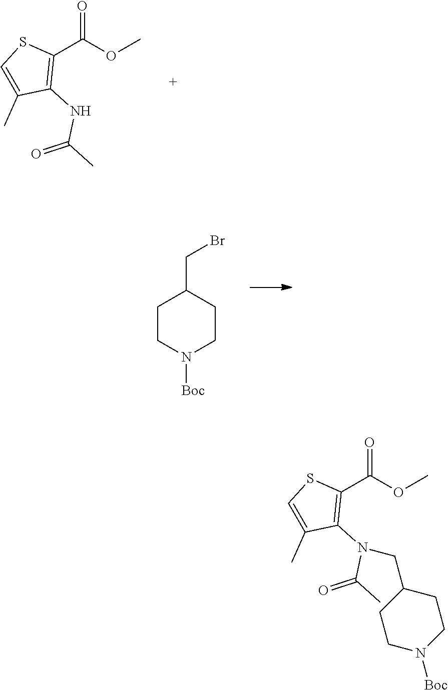 [SMILES: COC(=O)c1scc(C)c1NC(C)=O ; CC(C)(C)OC(=O)N1CCC(CBr)CC1 ; COC(=O)c1scc(C)c1N(CC1CCN(C(=O)OC(C)(C)C)CC1)C(C)=O]

Sodium hydride (60 wt % in mineral oil) (94 mg, 2.35 mmol) was added to a solution of methyl 3-acetamido-4-methylthiophene-2-carboxylate (0.500 g, 2.35 mmol) in DMF (1.5 mL) at 0° C., which was stirred for 10 min. The solution was allowed to warm to rt and stirred for 30 min, before adding a solution of tert-butyl 4-(bromomethyl)piperidine-1-carboxylate (0.652 g, 2.35 mmol) in DMF (1.5 ml. The solution was heated to 60° C. and stirred for 4 hrs. The reaction mixture was cooled and quenched with water (30 mL), NaHCO₃ (sat. aq., 10 mL) was added and the combined aqueous extracted with DCM (3×25 mL). The combined organic extracts were washed with water (2×20 mL) and brine (30 mL) before drying over MgSO₄ and concentrating in vacuo. The crude product was purified by flash chromatography (0-100% EtOAc in iso-hexane) to afford the title compound (600 mg, 62% yield) as a white solid.

[M+Na]+=433.1

¹H NMR (500 MHz, DMSO-d6) δ 0.93-1.06 (m, 2H), 1.38 (s, 9H), 1.53-1.59 (m, 1H), 1.60-1.64 (m, 1H), 1.65 (s, 3H), 2.08-2.11 (m, 3H), 3.32 (s, 3H), 3.39-3.43 (m, 2H), 3.79 (s, 3H), 3.83-3.89 (m, 2H), 7.70-7.76 (m, 1H).

General Method G: Alkylations Using NaH

(ii) O-Alkylation Using NaH

### tert-Butyl 4-(((2-(methoxycarbonyl)-5-methylthiophen-3-yl)oxy)methyl)piperidine-1-carboxylate

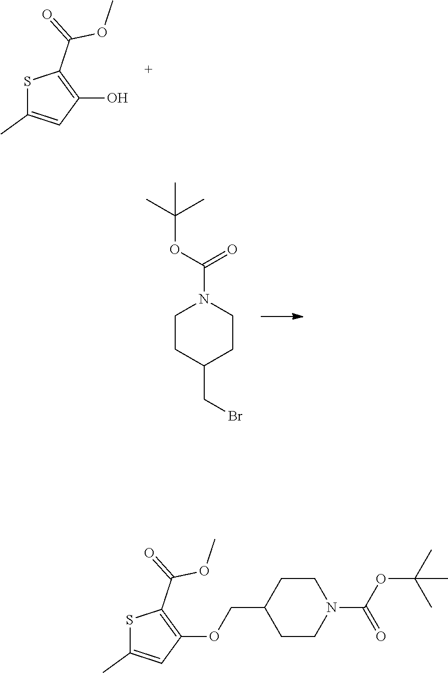

Methyl 3-hydroxy-5-methylthiophene-2-carboxylate (1 g, 5.81 mmol) was dissolved in DMF (20 mL) under nitrogen atmosphere. Sodium hydride, 60% in mineral oil (0.24 g, 6.10 mmol) was added, followed by tert-butyl 4-(bromomethyl)piperidine-1-carboxylate (1.8 g, 6.47 mmol). The reaction mixture was heated to 60° C. overnight. The reaction mixture was cooled to rt and quenched with NH₄Cl (sat., aq., 40 mL). EtOAc (100 mL) was added and the aqueous layer was separated. The organic layer was washed with brine (5×10 mL) and dried over MgSO₄ before being filtered and concentrated in vacuo. The crude product was purified by flash chromatography (0-50% EtOAc in isohexane) to afford the title compound (1.13 g, 49% yield) as a pale white powder.

[M+H]+=270.2

General Method H: N-Acyl Deprotection of Aminothiazoles and Amionothiophenes

### Methyl 4,5-dimethyl-3-(((1-methylpiperidin-4-yl)methyl)aminothiophene-2-carboxylate

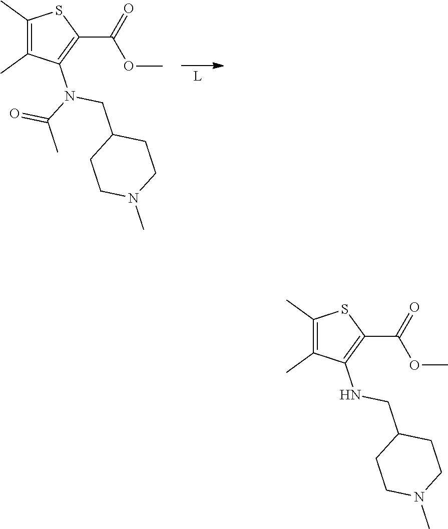

A solution of methyl 4,5-dimethyl-3-(N-((1-methylpiperidin-4-yl)methyl)acetamido)thiophene-2-carboxylate (100 mg, 0.295 mmol) and methanesulfonic acid (181 μL, 2.79 mmol) was heated at 100° C. for 12 hrs. The reaction was basified to pH 10 with Na₂CO₃ (sat. aq., 30 mL) and then extracted with EtOAc (3×30 mL). The combined organic layers were dried (MgSO₄), filtered and concentrated. The residue was purified by flash chromatography (0-15% (1% NH₃ in MeOH) in DCM) to afford the title compound (20 mg, 17% yield) as a yellow oil.

[M+H]⁺=297.1

General Method I: Core Alkylations

(i) Mitsunobu Conditions

### Methyl 1-(2-(1-methylpiperidin-4-yl)ethyl)-1H-pyrazole-3-carboxylate and Methyl 1-(2-(1-methylpiperidin-4-yl)ethyl)-1H-pyrazole-5-carboxylate

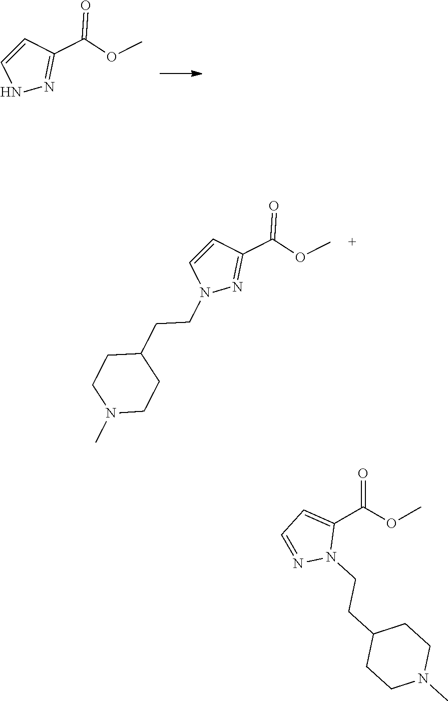

DIAD (1.1 mL, 5.66 mmol) was added dropwise over a period of 5 min to a solution of 2-(1-methylpiperidin-4-yl)ethan-1-ol (500 mg, 3.49 mmol), methyl 1H-pyrazole-3-carboxylate (294 mg, 2.327 mmol) and triphenylphosphine (1.6 g, 6.10 mmol) in THF (10 mL) at 0° C. The solution was warmed to rt and heated to 40° C. for 16 hrs. The reaction was cooled and added directly through SOC and washed with MeOH (20 mL). The required compound was eluted with 7M NH₃ in MeOH (50 mL) and concentrated in vacuo. The residue was purified by flash chromatography (0 to 10% (0.7M NH₃ in MeOH) in DCM) to afford a mixture of regioisomers as a colourless glass. This was further purified by reverse phase flash chromatography (5 to 50% MeCN in 10 mM ammonium bicarbonate) to afford the pyrazole-3-carboxylate analogue (77 mg, 13% yield) and the pyrazole-5-carboxylate analogue (352 mg, 54% yield) both as colourless gums. Structures were confirmed by ¹H NMR analysis.

### Methyl 1-(2-(1-methylpiperidin-4-yl)ethyl)-1H-pyrazole-3-carboxylate

[M+H]⁺=252.1

¹H NMR (500 MHz, DMSO-d6) δ 1.07-1.23 (m, 3H), 1.58-1.67 (m, 2H), 1.68-1.79 (m, 4H), 2.11 (s, 3H), 2.66-2.75 (m, 2H), 3.78 (s, 3H), 4.17-4.25 (m, 2H), 6.73 (d, J=2.3 Hz, 1H), 7.89 (d, J=2.4 Hz, 1H).

### Methyl 1-(2-(1-methylpiperidin-4-yl)ethyl)-1H-pyrazole-5-carboxylate

[M+H]⁺=252.1

¹H NMR (500 MHz, DMSO-d6) δ 0.97-1.28 (m, 3H), 1.53-1.71 (m, 2H), 1.71-1.83 (m, 4H), 2.12 (s, 3H), 2.65-2.75 (m, 2H), 3.83 (s, 3H), 4.46-4.54 (m, 2H), 6.88 (d, J=2.0 Hz, 1H), 7.56 (d, J=2.1 Hz, 1H).

General Method I: Core Alkylations

(ii) K₂CO₃

### tert-Butyl 4-(2-(5-chloro-3-(methoxycarbonyl)-1H-pyrazol-1-yl)ethyl)piperidine-1-carboxylate and tert-Butyl 4-(2-(3-chloro-5-(methoxycarbonyl)-1H-pyrazol-1-yl)ethyl)piperidine-1-carboxylate

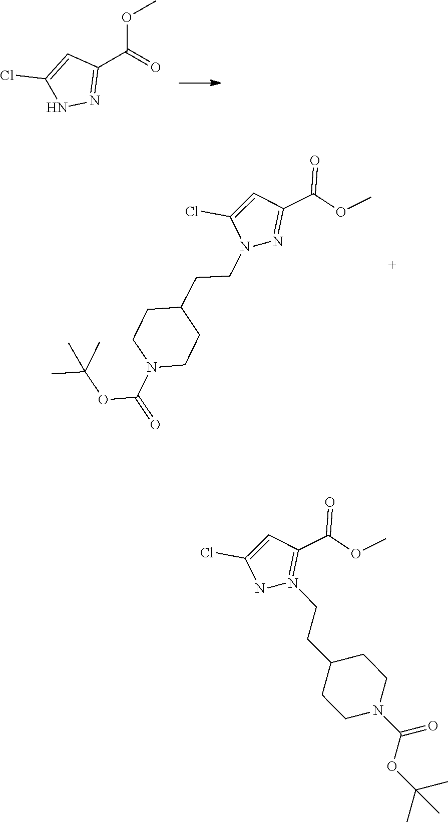

To a solution of methyl 5-chloro-1H-pyrazole-3-carboxylate (450 mg, 2.80 mmol) in MeCN (30 mL) was added potassium carbonate (368 mg, 2.66 mmol) and the mixture heated for 2 hrs at 80° C. The mixture was cooled to rt, concentrated in vacuo to half of the initial volume and quenched with water (5 ml). The reaction mixture was extracted with EtOAc (2×30 mL), combined organic layers were dried over MgSO₄, filtered and concentrated in vacuo. The residue was purified by flash chromatography (0 to 50% EtOAc in cyclohexane) to afford tert-butyl 4-(2-(5-chloro-3-(methoxycarbonyl)-1H-pyrazol-1-yl)ethyl)piperidine-1-carboxylate (296 mg, 28% yield) and tert-butyl 4-(2-(3-chloro-5-(methoxycarbonyl)-1H-pyrazol-1-yl)ethyl)piperidine-1-carboxylate (675 mg, 65% yield) both as colourless gums.

[M-Boc+H]⁺=272.1

General Method J: Pyrazole Alkylation Via Sulfonyl Transfer

### tert-Butyl 4-(2-(3-(methoxycarbonyl)-1H-pyrazol-1-yl)ethyl)piperidine-1-carboxylate

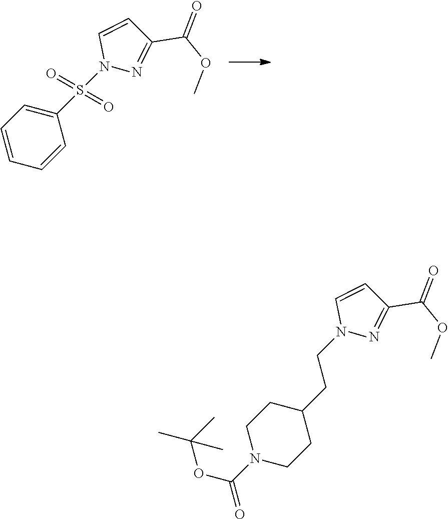 [SMILES: COC(=O)c1ccn(S(=O)(=O)c2ccccc2)n1 ; COC(=O)c1ccn(CCC2CCN(C(=O)OC(C)(C)C)CC2)n1]

Methyl-1-(phenylsulfonyl)-1H-pyrazole-3-carboxylate (200 mg, 0.75 mmol), N-boc-4-(2-hydroxyethyl)piperidine (172 mg, 0.75 mmol) and caesium carbonate (520 mg, 1.596 mmol) were dissolved in MeCN (8 mL) and stirred at rt for 18 hrs. The mixture was taken up in water (30 mL) and extracted with DCM (2×50 mL). Organic layers were combined and dried over Na₂SO₄, then filtered and concentrated in vacuo. The residue was purified by flash chromatography (0-100% EtOAc in Pet. Ether). Two regioisomers were obtained and their identity was confirmed by ¹H NMR analysis. The title compound (120 mg, 47% yield) was obtained as colourless oil.

[M-Boc+H]⁺=238.1

¹H NMR (Chloroform-d, 400 MHz) δ 1.10-1.20 (2H, m), 1.33-1.43 (1H, m), 1.45 (9H, s), 1.65 (2H, d, J=15.0 Hz), 1.86 (2H, q, J=7.1 Hz), 2.66 (2H, t, J=13.4 Hz), 3.93 (3H, s), 4.02-4.15 (2H, m), 4.19-4.29 (2H, m), 6.82 (1H, d, J=2.4 Hz), 7.40 (1H, d, J=2.3 Hz)

### INTERMEDIATES

### tert-Butyl N-[[4-(2-oxopyrrolidin-1-yl)phenyl]methyl]carbamate

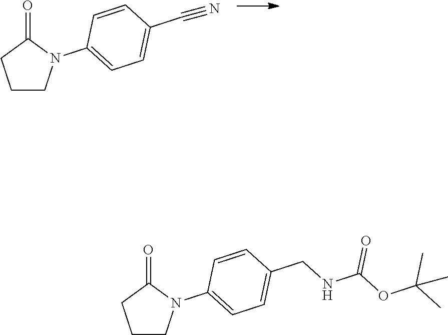 [SMILES: N#Cc1ccc(N2CCCC2=O)cc1 ; CC(C)(C)OC(=O)NCc1ccc(N2CCCC2=O)cc1]

4-(2-Oxopyrrolidin-1-yl)benzonitrile (CAS 167833-93-4, 260 mg, 1.4 mmol) was dissolved in anhydrous MeOH (15 mL) to which dichloronickel (36.2 mg, 0.28 mmol) was added followed by di-tert-butyl dicarbonate (609 mg, 2.79 mmol). This was cooled in an ice-salt bath to −5° C., then sodium borohydride (370 mg, 9.77 mmol) was added portionwise maintaining the temperature below 0° C. On completion, the ice-bath was removed and the mixture warmed to rt for 60 min. The reaction mixture was concentrated in vacuo and the residue partitioned between DCM (20 mL) and sat. aq. NaHCO₃ solution (20 mL). The aqueous layer was extracted with further DCM (2×20 mL) and the combined organics washed with water (20 mL) and brine (20 mL), dried (MgSO₄), filtered and concentrated. The residue was purified by flash chromatography (0-100% EtOAc in Isohexanes) to afford the title compound (222 mg, 53% yield) as a white solid.

[M+H]⁺=313.3

NMR (DMSO) δ: 1.39 (9H, s), 2.05 (2H, p, J=7.6 Hz), 2.44-2.49 (2H, m), 3.81 (2H, t, J=7.0 Hz), 4.08 (2H, d, J=6.2 Hz), 7.22 (2H, d, J=8.7 Hz), 7.35 (1H, t, J=6.2 Hz), 7.57 (2H, d, J=8.6 Hz)

### 1-[4-(Aminomethyl)phenyl]pyrrolidin-2-one

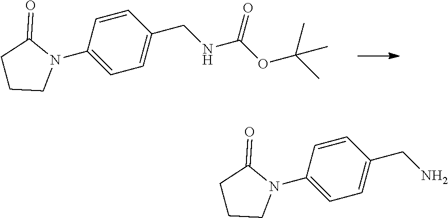 [SMILES: CC(C)(C)OC(=O)NCc1ccc(N2CCCC2=O)cc1 ; NCc1ccc(N2CCCC2=O)cc1]

Following general procedure D, tert-butyl N-[[4-(2-oxopyrrolidin-1-yl)phenyl]methyl]carbamate (218 mg, 0.75 mmol) was deprotected to afford the title compound (143 mg, 100% yield) as a waxy white solid.

[M+H]⁺=191.3

NMR (DMSO) δ: 1.99-2.10 (2H, m), 2.47 (2H, t, J=7.3 Hz), 3.70 (2H, s), 3.77-3.85 (2H, m), 7.31 (2H, d, J=8.6 Hz), 7.57 (2H, d, J=8.6 Hz)

### tert-Butyl (3S)-3-(3-pyridylamino)pyrrolidine-1-carboxylate

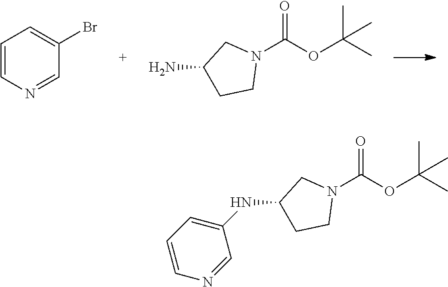 [SMILES: Brc1cccnc1 ; CC(C)(C)OC(=O)N1CC[C@H](N)C1 ; CC(C)(C)OC(=O)N1CC[C@H](Nc2cccnc2)C1]

3-Bromopyridine (120 μL, 1.25 mmol), tert-butyl (35)-3-aminopyrrolidine-1-carboxylate (300 μL, 1.77 mmol), caesium acetate (480 mg, 2.5 mmol) and copper powder (8 mg, 0.13 mmol) in DMSO (1.5 mL) was heated to 100° C. for 18 hrs. The reaction was cooled to rt, diluted with EtOAc (20 mL) and filtered through a silica gel plug, washing with EtOAc. The filtrate was washed with water (30 mL) and brine (20 mL) before drying via hydrophobic frit and concentrated in vacuo. The residue was purified by flash chromatography (50-100% EtOAc in hexane) to afford the title compound (100 mg, 30% yield) as a pale brown oil.

[M+H]⁺=264.1

### (S)—N-(Pyrrolidin-3-yl)pyridin-3-amine

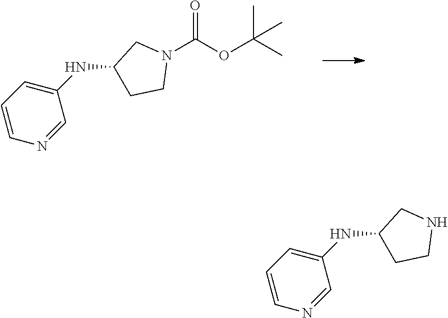 [SMILES: CC(C)(C)OC(=O)N1CC[C@H](Nc2cccnc2)C1 ; c1cncc(N[C@H]2CCNC2)c1]

Following general method D, tert-butyl (35)-3-(3-pyridylamino)pyrrolidine-1-carboxylate (100 mg, 0.38 mmol) was deprotected to afford the hydrochloride salt of title compound (quantitative yield) as a brown gum.

[M+H]⁺=164.1

### Ethyl 5-methylthiophene-2-carboxylate

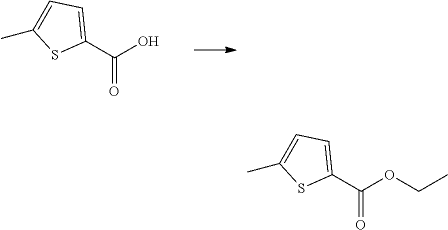

5-Methylthiophene-2-carboxylic acid (58 g, 408 mmol) was dissolved in ethanol (1.5 L) and treated with sulfuric acid (13 mL, 245 mmol). The mixture was heated at reflux 135° C. for 72 hrs. The solution was concentrated in vacuo and the residue was dissolved in EtOAc (500 mL) and washed with water (2×200 mL), 2M NaOH (2×100 mL), water (1×200 mL) and brine (1×200 ml). The organic layer was dried (Na₂SO₄), filtered and concentrated to afford the title compound (69.3 g, 91% yield) as a yellow oil.

[M+H]⁺=171.2

### Ethyl 4-chloro-5-methyl-thiophene-2-carboxylate

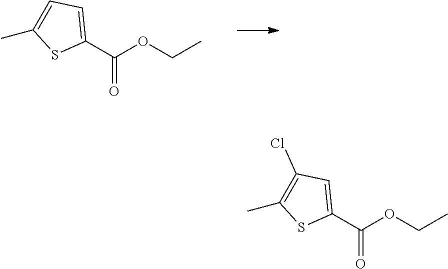

An ice-cooled solution of ethyl 5-methylthiophene-2-carboxylate (40 g, 235 mmol) in anhydrous MeCN (400 mL) under N₂ was treated with a 1M DCM solution of sulfuryl chloride (799 mL, 799 mmol) via cannula. The ice-bath was removed, and the mixture stirred at rt for 2 hrs. Water (100 mL) was added and the reaction concentrated in vacuo to remove MeCN. The remaining oil was partitioned between DCM (500 mL) and sat. aq. NaHCO₃ solution (500 ml). The organic layer was washed with further sat. aq. NaHCO₃ (200 mL) and brine (100 ml), dried (Na₂SO₄), filtered and concentrated in vacuo. The residue was purified by flash chromatography (0-30% DCM in Isohexanes) to afford the title compound (16.2 g, 34% yield) as a yellow oil.

### Ethyl 5-(bromomethyl)-4-chloro-thiophene-2-carboxylate

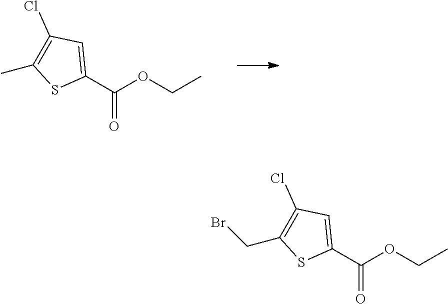

A neat mixture of ethyl 4-chloro-5-methyl-thiophene-2-carboxylate (795 mg, 3.88 mmol) and NBS (691 mg, 3.88 mmol) were combined and stirred at rt for 1 week. The mixture was diluted with DCM and the reaction was filtered and then concentrated under vacuum. The crude mixture was purified by flash chromatography, (0-25% DCM in Isohexane) to afford the title compound (655 mg, 57% yield) as a colourless oil which crystallized on standing.

¹H NMR (CDCl₃) δ: 1.37 (3H, t, J=7.1 Hz), 4.35 (2H, q, J=7.1 Hz), 4.63 (2H, s), 7.56 (1H, s).

### Ethyl 5-(acetoxymethyl)-4-chloro-thiophene-2-carboxylate

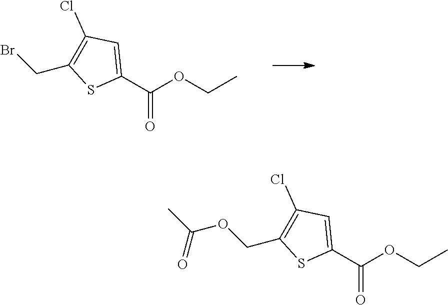 [SMILES: CCOC(=O)c1cc(Cl)c(CBr)s1 ; CCOC(=O)c1cc(Cl)c(COC(C)=O)s1]

A mixture of ethyl 5-(bromomethyl)-4-chloro-thiophene-2-carboxylate (13.2 g, 46.6 mmol) and sodium acetate (7.64 g, 93.2 mmol) in glacial acetic acid (115 mL) was heated at 120° C. for 12 hrs, then at rt for 4 hrs. The mixture was poured carefully into a mixture of sat. aq. NaHCO₃ solution (500 mL) and NaHCO₃ powder until pH 8, then extracted with EtOAc (3×250 mL). The combined organics were dried (MgSO₄), filtered and concentrated in vacuo to afford the title compound (11.52 g, 91% yield) as a brown oil.

¹H NMR (CDCl₃) δ: 1.36 (3H, t, J=7.1 Hz), 2.12 (3H, s), 4.34 (2H, q, J=7.1 Hz), 5.24 (2H, s), 7.58 (1H, s)

### 4-Chloro-5-(hydroxymethyl)thiophene-2-carboxylic Acid

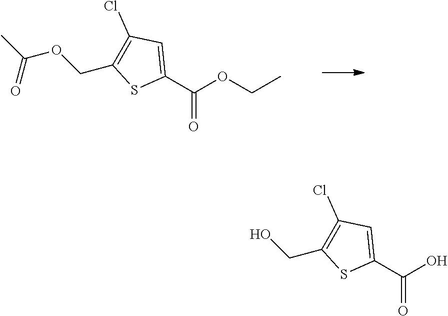 [SMILES: CCOC(=O)c1cc(Cl)c(COC(C)=O)s1 ; O=C(O)c1cc(Cl)c(CO)s1]

Following general procedure E, ethyl 5-(acetoxymethyl)-4-chloro-thiophene-2-carboxylate (11.5 g, 43.9 mmol) was hydrolysed to afford the title compound (7.57 g, 85% yield) as a brown solid.

[M−H]⁻=191

¹HNMR (DMSO) δ: 4.55-4.69 (2H, brs), 5.92 (1H, brs), 7.60 (1H, s), 13.38 (1H, s)

### 4-Chloro-5-(chloromethyl)thiophene-2-carbonyl chloride

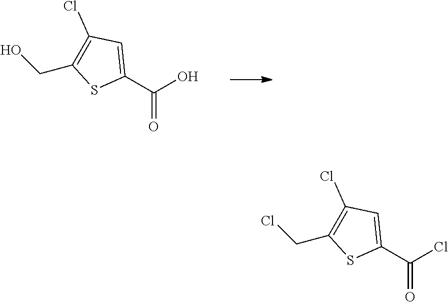 [SMILES: O=C(O)c1cc(Cl)c(CO)s1 ; O=C(Cl)c1cc(Cl)c(CCl)s1]

A suspension of 4-chloro-5-(hydroxymethyl)thiophene-2-carboxylic acid (5.58 g, 29 mmol) in thionylchloride (52.9 mL, 724 mmol) was heated to 80° C. for 10 hrs, then cooled to rt and stirred for 18 hrs. The reaction was concentrated under vacuum, followed by concentration from 1,2-dichloroethane (3×50 ml) in vacuo to afford the title compound (6.57 g, 99% yield) as a brown oil.

### N-((1-Aminoisoquinolin-6-yl)methyl)-4-chloro-5-(chloromethyl)thiophene-2-carboxamide

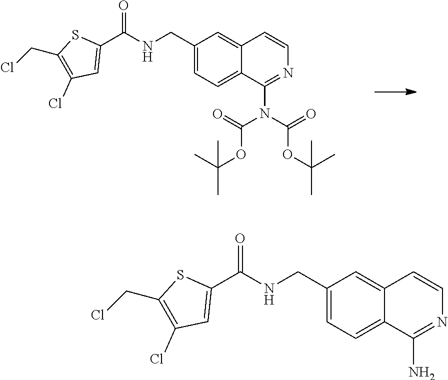 [SMILES: CC(C)(C)OC(=O)N(C(=O)OC(C)(C)C)c1nccc2cc(CNC(=O)c3cc(Cl)c(CCl)s3)ccc12 ; Nc1nccc2cc(CNC(=O)c3cc(Cl)c(CCl)s3)ccc12]

Following general method D (ii), tert-butyl (tert-butoxycarbonyl)(6-((4-chloro-5-(chloromethyl) thiophene-2-carboxamido)methyl)isoquinolin-1-yl)carbamate (5.1 g, 9.0 mmol) was treated with 4M HCl in Dioxane (22.5 mL, 90 mmol) to afford the title compound (3.44 g, 93%) as a white powder.

[M+H]⁺=364.1/366.1

¹H NMR (DMSO) δ: 4.66 (2H, d, J=5.9 Hz), 5.00 (2H, s), 7.23 (1H, d, J=7.0 Hz), 7.68 (1H, d, J=7.0 Hz), 7.72 (1H, dd, J=8.7, 1.7 Hz), 7.83 (1H, s), 7.93 (1H, s), 8.56 (1H, d, J=8.6 Hz), 9.12 (2H, s), 9.57 (1H, t, J=6.0 Hz), 13.28 (1H, s)

### Ethyl 4-chloro-5-[[4-(4-pyridyl)piperazin-1-yl]methyl]thiophene-2-carboxylate

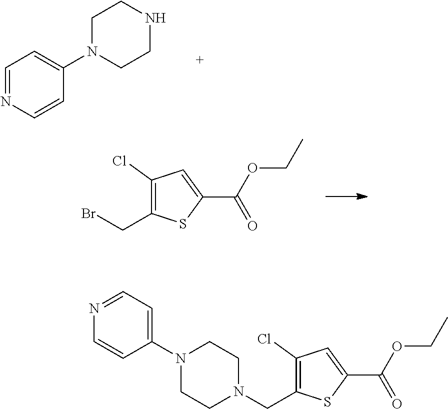 [SMILES: c1cc(N2CCNCC2)ccn1 ; CCOC(=O)c1cc(Cl)c(CBr)s1 ; CCOC(=O)c1cc(Cl)c(CN2CCN(c3ccncc3)CC2)s1]

Following general procedure C (ii), 1-(4-pyridyl)piperazine (968 mg, 5.93 mmol) was reacted with ethyl 5-(bromomethyl)-4-chloro-thiophene-2-carboxylate (840 mg, 2.96 mmol) to afford the title compound (800 mg, 69% yield) as a yellow oil.

[M+H]⁺=366.2

¹H NMR (DMSO-d6, 500 MHz) δ 1.29 (3H, t, J=7.1 Hz), 2.57-2.63 (4H, m), 3.30-3.36 (4H, m), 3.80 (2H, s), 4.29 (2H, q, J=7.1 Hz), 6.79-6.84 (2H, m), 7.71 (1H, s), 8.14-8.19 (2H, m)

### [4-Chloro-5-[[4-(4-pyridyl)piperazin-1-yl]methyl]thiophene-2-carbonyl]oxylithium

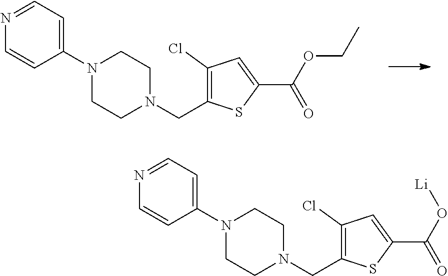

Following general conditions E, ethyl 4-chloro-5-[[4-(4-pyridyl)piperazin-1-yl]methyl]thiophene-2-carboxylate (800 mg, 2.19 mmol) was hydrolysed to afford the title compound (790 mg, quantitative yield) as an off-white solid.

[M+H]⁺=338.1

¹H NMR (DMSO-d6, 500 MHz) δ 2.51-2.56 (4H, m), 3.29-3.35 (4H, m), 3.65 (2H, s), 6.78-6.83 (2H, m), 7.00 (1H, s), 8.12-8.17 (2H, m)

### 2-(3-Ethyithiophen-2-yl)-1,3-dimethylimidazolidine

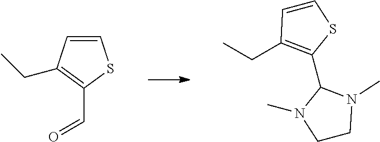

3-Ethylthiophene-2-carbaldehyde (1.35 g, 9.63 mmol) and N,N′-dimethylethane-1,2-diamine (1.1 mL, 10.2 mmol)) were dissolved in toluene (30 mL), a Dean-Stark attached and the reaction was heated at reflux for 4 hrs. The solvent was evaporated in vacuo to afford title product (1.85 g, 82% yield) as pale yellow oil.

[M+H]⁺=211.2

¹H NMR (DMSO-d6, 500 MHz) δ 1.10-1.21 (3H, m), 2.12 (6H, s), 2.44-2.56 (2H, m), 2.61 (2H, q, J=7.5 Hz), 3.14-3.23 (2H, m), 3.73 (1H, s), 6.86 (1H, d, J=5.1 Hz), 7.38 (1H, d, J=5.2 Hz)

### 4-Ethyl-5-formylthiophene-2-carboxylic Acid

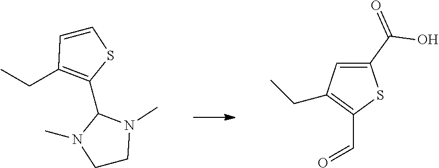

2-(3-Ethylthiophen-2-yl)-1,3-dimethylimidazolidine (1.8 g, 8.56 mmol) and N,N,N′,N′-tetramethylethane-1,2-diamine (1.4 mL, 9.34 mmol) were stirred in THF (60 mL) and cooled to −78° C. under a nitrogen atmosphere. n-butyllithium (3.8 mL, 9.5 mmol) was added dropwise over 5 min and the reaction stirred at −78° C. for 2 hrs. The reaction was then poured onto crushed dry-ice and allowed to warm to rt whilst being stirred for 2 hrs. The THF was evaporated in vacuo, the residue partitioned between saturated NaHCO₃aq (200 mL) and EtOAc (200 mL) and the aqueous layer acidified with conc. HCL. The aqueous layer was then extracted with EtOAc (2×100 mL), washed with brine (50 mL), dried over Na₂SO₄, filtered and evaporated in vacuo to afford title compound (1.33 g, 82% yield), as a cream solid.

¹H NMR (DMSO-d6, 500 MHz) δ 1.24 (3H, t, J=7.6 Hz), 3.00 (2H, q, J=7.6 Hz), 7.74 (1H, s), 10.11 (1H, s), 13.69 (1H, s).

[M+H]⁺=185.0

### 4-Ethyl-5-((4-(pyridin-4-yl)piperazin-1-yl)methyl)thiophene-2-carboxylic Acid

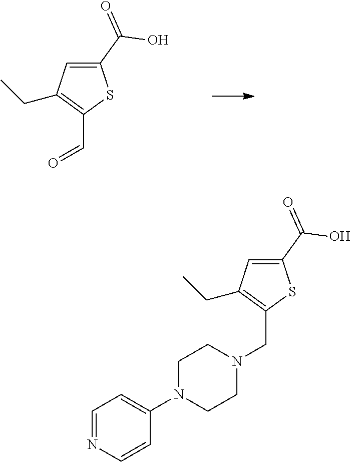 [SMILES: CCc1cc(C(=O)O)sc1C=O ; CCc1cc(C(=O)O)sc1CN1CCN(c2ccncc2)CC1]

4-Ethyl-5-formylthiophene-2-carboxylic acid (240 mg, 1.30 mmol), 1-(4-pyridyl)piperazine (224 mg, 1.37 mmol) and acetic acid (0.23 mL, 4.02 mmol) were partially dissolved in THF (10 mL) and stirred for 15 min. Sodium triacetoxyborohydride (690 mg, 3.26 mmol) was then added and the reaction stirred for further 20 hrs. The reaction was diluted with MeOH (20 mL), absorbed onto SCX, washed with MeOH (50 mL) and the product was eluted with 1M NH₃ in MeOH (50 mL). The solvent was evaporated in vacuo and the solid residue treated with TBME (20 mL), filtered off and dried. The solid was then stirred with EtOAc (10 mL) for 20 hrs, filtered and dried to afford title compound (340 mg, 76% yield), as a white solid.

¹H NMR (DMSO-d6, 500 MHz) δ 1.13 (3H, t, J=7.5 Hz), 2.52-2.60 (6H, m), 3.35 (4H, t, J=5.0 Hz), 3.68 (2H, s), 6.81-6.87 (2H, m), 7.48 (1H, s), 8.14-8.21 (2H, m), acidic proton not visible.

[M+H]⁺=332.2

### 1,3-Dimethyl-2-(4-methyl-2-thienyl)imidazolidine

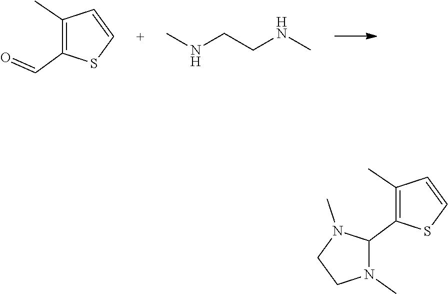

3-Methylthiophene-2-carbaldehyde (3.0 g, 23.8 mmol) and N,N′-dimethylethane-1,2-diamine (2.7 mL, 25.1 mmol) were dissolved in toluene (50 mL), a Dean Stark was attached and the reaction heated at 100° C. for 4 hrs. The reaction mixture was concentrated in vacuo and afforded the title compound (4.9 g, 89% yield) as a brown liquid.

[M+H]⁺=197.1

¹H NMR (DMSO-d6, 500 MHz) δ 2.12 (6H, s), 2.21 (3H, s), 2.49-2.51 (2H, m), 3.17-3.21 (2H, m), 3.74 (1H, d, J=0.8 Hz), 6.80 (1H, d, J=5.0 Hz), 7.37 (1H, dd, J=5.1, 0.7 Hz)

### 5-Formyl-4-methyl-thiophene-2-carboxylic Acid

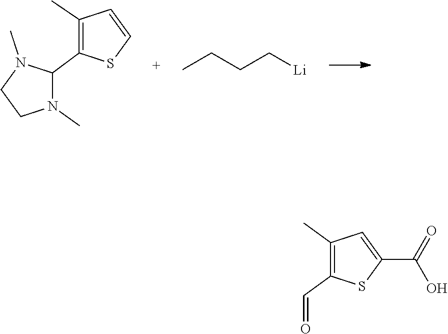

1,3-Dimethyl-2-(3-methyl-2-thienyl)imidazolidine (4.8 g, 24.5 mmol) and N,N,N′,N′-tetramethylethane-1,2-diamine (4.1 mL, 27.3 mmol) were stirred in THF (100 mL) and cooled to −78° C. under a nitrogen atmosphere. n-butyllithium (10.8 mL, 27 mmol) was added dropwise over 5 min and the reaction stirred at −78° C. for 2 hrs. The reaction was then poured onto crushed dry-ice and allowed to warm to rt under stirring for 2 hrs. The THF was evaporated in vacuo, the residue partitioned between aq. saturated NaHCO₃ (200 mL) and EtOAc (200 mL) and the aqueous layer acidified with conc. HCl. The aqueous was then extracted with EtOAc (2×100 mL), washed with brine (50 mL) and dried (Na₂SO₄). Organic mixture was concentrated in vacuo to afford the title compound (2.5 g, 58% yield) as a cream solid.

[M+H]⁺=170.9

¹H NMR (DMSO-d6, 500 MHz) δ 2.57 (3H, s), 7.67 (1H, s), 10.09 (1H, s), 13.69 (1H, s)

### N-[(1-Amino-6-isoquinolyl)methyl]-5-formyl-4-methyl-thiophene-2-carboxamide

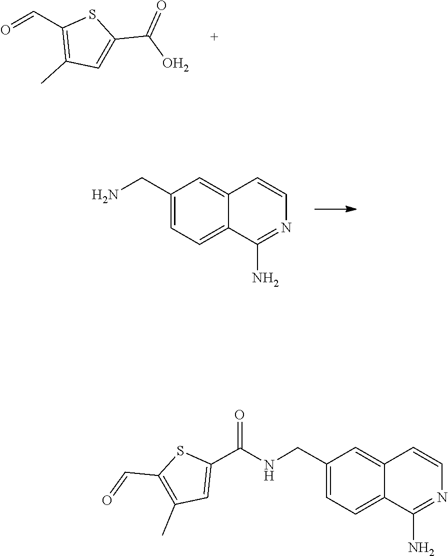 [SMILES: Cc1cc(C(=O)O)sc1C=O ; NCc1ccc2c(N)nccc2c1 ; Cc1cc(C(=O)NCc2ccc3c(N)nccc3c2)sc1C=O]

Following general procedure A, 5-formyl-4-methyl-thiophene-2-carboxylic acid (1 g, 5.88 mmol) was reacted with 6-(aminomethyl)isoquinolin-1-amine dihydrochloride (1.45 g, 5.88 mmol) to afford the title compound (100 mg, 5% yield).

[M+H]⁺=326

¹H NMR (DMSO-d6, 500 MHz) δ 2.56 (3H, s), 4.60 (2H, d, J=6.0 Hz), 6.91 (1H, d, J=6.0 Hz), 6.99 (2H, s), 7.43 (1H, dd, J=8.5, 1.7 Hz), 7.59 (1H, s), 7.74-7.77 (2H, m), 8.19 (1H, d, J=8.6 Hz), 9.40 (1H, t, J=6.1 Hz), 10.08 (1H, s)

### 5-Benzyl-4-chlorothiophene-2-carboxylic Acid

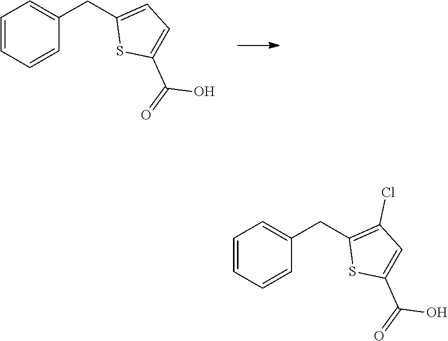 [SMILES: O=C(O)c1ccc(Cc2ccccc2)s1 ; O=C(O)c1cc(Cl)c(Cc2ccccc2)s1]

5-Benzylthiophene-2-carboxylic acid (50 mg, 0.23 mmol) in DMF (0.5 mL) was treated with NCS (35 mg, 0.26 mmol) and heated to 60° C. for 18 hrs. The reaction mixture was taken up in EtOAc (20 mL) and washed with water (2×10 mL) and brine (20 ml). The organic phase was dried (Na₂SO₄) and concentrated in vacuo. Flash chromatography (0-100% EtOAc in isohexane) afforded the title compound (10 mg, 14% yield) as a colourless glass.

### Methyl 3-acetamido-5-methylthiophene-2-carboxylate

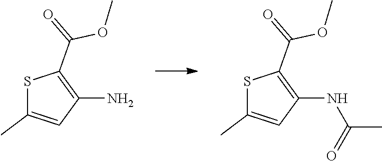

Acetic anhydride (3 mL, 31.8 mmol) was added to a solution of methyl 3-amino-5-methylthiophene-2-carboxylate (1.0 g, 5.84 mmol) in pyridine (1.5 mL, 18.55 mmol) and stirred at rt over the weekend. The reaction mixture was diluted with EtOAc (30 mL) and washed with 1 M HCl (2×20 mL). The organic layer was dried over MgSO₄, filtered and concentrated to an oily orange solid. The residue was purified by flash chromatography (0-50% EtOAc in iso-hexane) to afford the title compound (1.04 g, 4.69 mmol, 80% yield) as a waxy yellow solid.

[M+H]⁺=213.7

¹H NMR (500 MHz, Chloroform-d) δ 2.23 (s, 3H), 2.51 (d, J=1.0 Hz, 3H), 3.88 (s, 3H), 7.86 (d, J=1.2 Hz, 1H), 10.15 (s, 1H).

### tert-Butyl 4-((N-(2-(methoxycarbonyl)-5-methylthiophen-3-yl)acetamido)methyl)piperidine-1-carboxylate

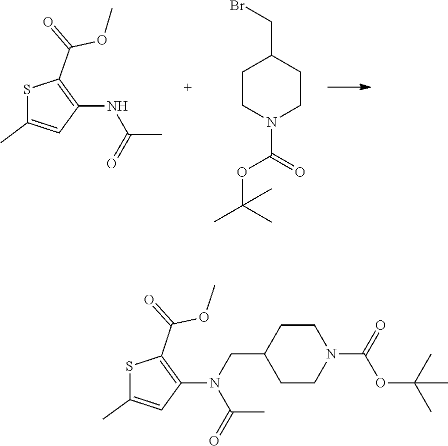 [SMILES: COC(=O)c1sc(C)cc1NC(C)=O ; CC(C)(C)OC(=O)N1CCC(CBr)CC1 ; COC(=O)c1sc(C)cc1N(CC1CCN(C(=O)OC(C)(C)C)CC1)C(C)=O]

Sodium hydride (60 wt % in mineral oil) (143 mg, 3.59 mmol) was added to a solution of methyl 3-acetamido-5-methylthiophene-2-carboxylate (765 mg, 3.59 mmol) in DMF (7 mL) at 0° C. and stirred for 10 min. The solution was warmed to rt and stirred for 30 min and tert-butyl 4-(bromomethyl)piperidine-1-carboxylate (1 g, 3.59 mmol) was added in one portion. The solution was heated to 60° C. and stirred for 18 hrs. The reaction mixture was poured into sat. NH₄Cl (50 mL) and extracted with EtOAc (100 mL). The phases were partitioned and the organic phase was washed with 1:1 brine:water (50 mL) followed by brine (50 mL). The organic phase was dried over MgSO₄, filtered and concentrated. The crude product was purified by flash chromatography (10-50% EtOAc/isohexane) to afford the title compound (650 mg, 35% yield) as a colourless glass.

[M+Na]⁺=433.1

### Methyl 5-methyl-3-(N-(piperidin-4-ylmethyl)acetamido)thiophene-2-carboxylate

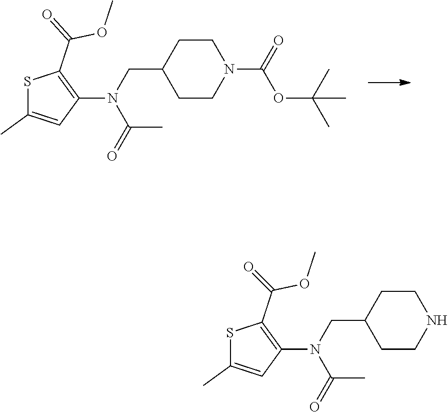

Following general procedure D, tert-butyl 4-((N-(2-(methoxycarbonyl)-5-methylthiophen-3-yl)acetamido)methyl)piperidine-1-carboxylate (620 mg, 1.51 mmol) was treated with TFA. Purification by flash chromatography (SOC, 7M NH₃ in MeOH) afforded the free base of the title compound (235 mg, 48% yield) as a colourless glass.

[M+H]⁺=311.1

### Methyl 5-methyl-3-(N-((1-methylpiperidin-4-yl)methyl)acetamido)thiophene-2-carboxylate

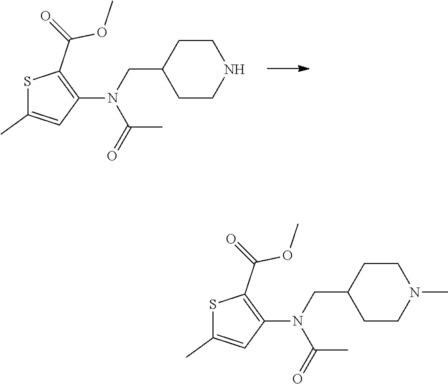

Following general procedure F (i), methyl 5-methyl-3-(N-(piperidin-4-ylmethyl)acetamido)thiophene-2-carboxylate (235 mg, 0.72 mmol) afforded the title compound (138 mg, 53% yield) as a colourless glass.

[M+H]⁺=325.1

### Methyl 4-methyl-3-(1-methylpiperidine-4-carboxamido)thiophene-2-carboxylate

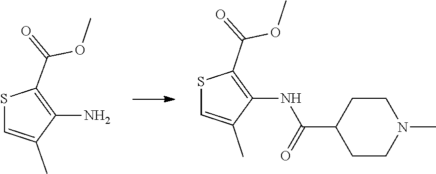 [SMILES: COC(=O)c1scc(C)c1N ; COC(=O)c1scc(C)c1NC(=O)C1CCN(C)CC1]

Propylphosphonic anhydride, (T3P) (50% in DMF) (1.6 mL, 2.69 mmol) was added to a solution of 1-methylpiperidine-4-carboxylic acid (100 mg, 0.698 mmol) and DIPEA (0.4 mL, 2.290 mmol) in DMF (2 mL) at rt, before adding methyl 3-amino-4-methylthiophene-2-carboxylate (120 mg, 0.698 mmol) and stirring the reaction at 100° C. for 18 hrs. The reaction mixture was cooled, diluted with EtOAc (25 mL) and washed with Na₂CO₃ (sat. aq., 30 mL). The basic aqueous layer was back extracted with EtOAc (3×20 mL). The combined organic extracts were washed with H₂O (2×20 mL) and brine (20 mL) dried (MgSO₄), filtered and concentrated under vacuum. The residue was purified by flash chromatography (0-10% MeOH (0.7 M NH₃)/DCM) to afford the title compound (110 mg, 53% yield) as a brown solid.

[M+H]⁺=297.1

### Methyl 3-acetamido-4-methylthiophene-2-carboxylate

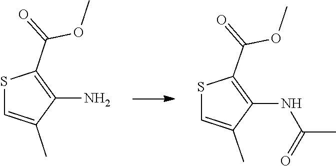

Acetic anhydride (10 mL, 106 mmol) was added to a solution of methyl 3-amino-4-methylthiophene-2-carboxylate (2.8 g, 16.35 mmol) in pyridine (5 mL) and stirred at rt for 65 hrs. The reaction was diluted with EtOAc (70 mL) and washed with HCl (1 M, 3×30 mL) before drying over MgSO₄, filtering and concentrating in vacuo. The crude product was purified by flash chromatography, (0-60% iso-hexane/EtOAc) to afford the title compound (2.3 g, 65% yield) as a white solid.

[M+H]⁺=214.2

¹H NMR (500 MHz, DMSO-d6) δ 9.61 (s, 1H), 7.51 (d, J=1.3 Hz, 1H), 3.76 (s, 3H), 2.05 (s, 3H), 2.04-2.03 (m, 3H)

### tert-Butyl 4-((N-(5-chloro-2-(methoxycarbonyl)-4-methylthiophen-3-yl)acetamido)methyl) piperidine-1-carboxylate

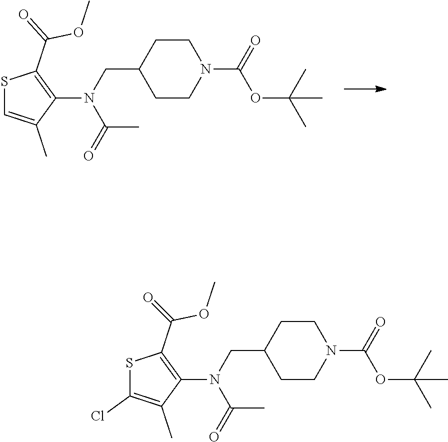 [SMILES: COC(=O)c1scc(C)c1N(CC1CCN(C(=O)OC(C)(C)C)CC1)C(C)=O ; COC(=O)c1sc(Cl)c(C)c1N(CC1CCN(C(=O)OC(C)(C)C)CC1)C(C)=O]

A solution of tert-butyl 4-((N-(2-(methoxycarbonyl)-4-methylthiophen-3-yl)acetamido)methyl) piperidine-1-carboxylate (1.70 g, 4.14 mmol) and NCS (0.66 g, 4.94 mmol) in DMF (25 mL) was heated to 40° C. under an atmosphere of N₂ for 7 hrs. The reaction was cooled and diluted with DCM (30 mL), washed with NaHCO₃ (sat., aq, 30 mL), H₂O (20 mL) and brine (20 mL), filtered through a phase separator and concentrated in vacuo. The crude product was purified by flash chromatography, (0-55% EtOAc/iso-hexane) to obtain the title compound (950 mg, 46% yield) as a white solid.

[M+Na]⁺=467.1

¹H NMR (500 MHz, DMSO-d6) δ 0.93-1.06 (m, 2H), 1.35-1.42 (m, 9H), 1.44-1.66 (m, 3H), 1.68-1.73 (m, 3H), 1.97-2.00 (m, 2H), 2.06 (s, 3H), 3.34-3.50 (m, 2H), 3.79 (s, 3H), 3.82-3.91 (m, 2H)

### Methyl 4,5-dimethyl-3-(N-((1-methylpiperidin-4-yl)methyl)acetamido)thiophene-2-carboxylate

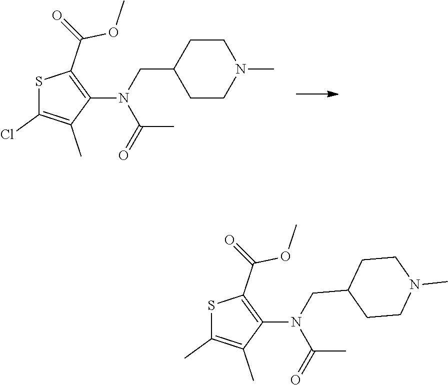

A mixture of methyl 5-chloro-4-methyl-3-(N-((1-methylpiperidin-4-yl)methyl)acetamido)thiophene-2-carboxylate (134 mg, 0.37 mmol), 2,4,6-trimethyl-1,3,5,2,4,6-trioxatriborinane (0.1 mL, 0.71 mmol), Pd(PPh₃)₄(43 mg, 0.04 mmol) and potassium carbonate (155 mg, 1.12 mmol) in 1,4-dioxane (5 mL) was stirred at 100° C. for 2 hrs and at rt for 16 hrs. The reaction was passed through a Celite pad and washed thoroughly with DCM (30 mL). The reaction mixture was diluted with EtOAc (25 mL), washed with H₂O (20 mL) and brine (20 mL) before passing through a phase separator and concentrating under reduced pressure. The crude product was purified by flash chromatography, (0-100% EtOAc/iso-hexane) to obtain the title compound (57 mg, 43% yield) as a colourless oil.

[M+H]⁺=339.1

¹H NMR (500 MHz, DMSO-d6) δ 1.51-1.61 (m, 3H), 1.65 (s, 3H), 1.67-1.76 (m, 3H), 1.97 (s, 3H), 2.10 (s, 3H), 2.42 (s, 3H), 2.63-2.76 (m, 2H), 3.14-3.21 (m, 1H), 3.36-3.42 (m, 2H), 3.75 (s, 3H).

### tert-Butyl 4-(hydroxy(5-(methoxycarbonyl)thiophen-2-yl)methyl)piperidine-1-carboxylate

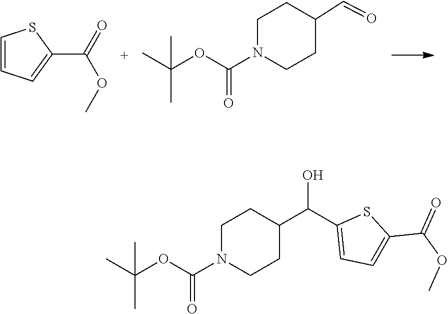

A solution of diisopropylamine (2.16 mL, 15.8 mmol) in THF (20 mL, 7.03 mmol) at −78° C. was treated with n-BuLi in hexanes (6.19 mL, 15.5 mmol) over the course of 1 min. The solution was warmed to rt for 10 min and then re-cooled to −78° C. Methyl thiophene-2-carboxylate (0.820 mL, 7.03 mmol) was added portionwise. The resulting mixture was stirred for 15 min at −78° C. before tert-butyl 4-formylpiperidine-1-carboxylate (1.88 g, 8.79 mmol) in THF (10 mL) was added portionwise. The reaction mixture was warmed to rt and left to stir for 18 hrs. The reaction mixture was quenched with sat. aq. NH₄Cl (100 mL) and extracted with EtOAc (2×100 ml). Combined organic extracts were dried (MgSO₄) and concentrated in vacuo. The residue was purified by flash chromatography (0-50% EtOAc in iso-hexane) to afford the title compound (995 mg, 38% yield) as a pale brown glass.

[M+H-Boc]⁺=256.6 ¹H NMR (500 MHz, Chloroform-d) δ 1.18-1.36 (m, 2H), 1.43 (d, J=3.0 Hz, 1H), 1.46 (s, 9H), 1.81 (tdt, J=11.3, 7.2, 3.6 Hz, 2H), 1.95 (dt, J=13.1, 2.9 Hz, 1H), 2.67 (dtd, J=20.0, 12.9, 2.9 Hz, 2H), 3.90 (s, 3H), 4.15 (dd, J=30.8, 13.2 Hz, 2H), 4.70 (d, J=7.0 Hz, 1H), 6.95 (d, J=3.8 Hz, 1H), 7.69 (d, J=3.8 Hz, 1H)

### Methyl 5-(piperidin-4-ylmethyl)thiophene-2-carboxylate

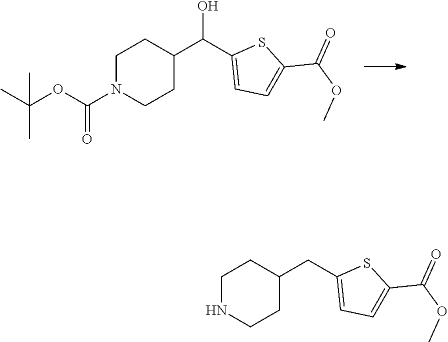 [SMILES: COC(=O)c1ccc(C(O)C2CCN(C(=O)OC(C)(C)C)CC2)s1 ; COC(=O)c1ccc(CC2CCNCC2)s1]

2,2,2-Trifluoroacetic acid (2.3 mL, 30.1 mmol) was added dropwise to a solution of tert-butyl 4-(hydroxy(5-(methoxycarbonyl)thiophen-2-yl)methyl)piperidine-1-carboxylate (444 mg, 1.25 mmol) and triethylsilane (1.2 mL, 7.51 mmol) in DCM (2 mL). The mixture was stirred at 60° C. for 24 hrs. The reaction mixture was concentrated in vacuo to afford the title compound (306 mg, 97% yield) as a colourless gum.

[M+H]⁺=240.3

¹H NMR (500 MHz, DMSO-d6) δ 1.04 (qd, J=12.1, 4.1 Hz, 2H), 1.51-1.64 (m, 3H), 2.39 (td, J=12.0, 2.4 Hz, 2H), 2.74 (d, J=6.8 Hz, 2H), 2.88 (dt, J=12.4, 3.4 Hz, 2H), 3.79 (s, 3H), 6.92-6.97 (m, 1H), 7.65 (d, J=3.8 Hz, 1H). NH not observed

### Methyl 5-((1-methylpiperidin-4-yl)methyl)thiophene-2-carboxylate

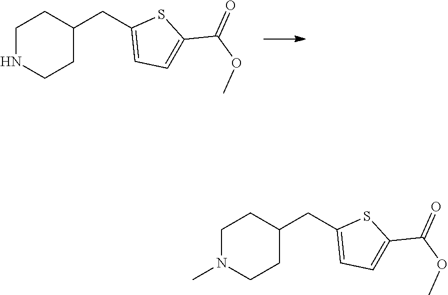 [SMILES: COC(=O)c1ccc(CC2CCNCC2)s1 ; COC(=O)c1ccc(CC2CCN(C)CC2)s1]

Following general procedure F, methyl 5-(piperidin-4-ylmethyl)thiophene-2-carboxylate (306 mg, 1.28 mmol) afforded the title compound (180 mg, 52% yield) as a pale yellow oil.

[M+H]⁺=254.3

¹H NMR (500 MHz, DMSO-d6) δ 1.19 (qd, J=11.9, 3.8 Hz, 2H), 1.47 (ttt, J=11.0, 7.3, 3.8 Hz, 1H), 1.55-1.61 (m, 2H), 1.78 (td, J=11.7, 2.5 Hz, 2H), 2.12 (s, 3H), 2.68-2.85 (m, 4H), 3.79 (s, 3H), 6.95 (d, J=3.7 Hz, 1H), 7.65 (d, J=3.7 Hz, 1H)

### Methyl 4-chloro-5-((1-methylpiperidin-4-yl)methyl)thiophene-2-carboxylate

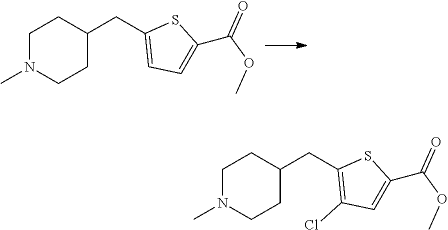 [SMILES: COC(=O)c1ccc(CC2CCN(C)CC2)s1 ; COC(=O)c1cc(Cl)c(CC2CCN(C)CC2)s1]

Sulfuryl chloride (0.046 mL, 0.568 mmol) in chloroform (1 mL) was added dropwise to a solution of methyl 5-((1-methylpiperidin-4-yl)methyl)thiophene-2-carboxylate (90 mg, 0.36 mmol) in chloroform (1 mL) and the mixture stirred at 40° C. for 60 min. The reaction mixture was diluted with DCM (15 mL), poured into sat. aq. Na₂CO₃(15 mL) and stirred for 1 min. The organic layer was separated and evaporated. Purification via flash chromatography (0-7% (0.7M NH₃ in MeOH) in DCM) afforded the title compound (56 mg, 49% yield) as a pale yellow gum.

[M+H]⁺=288.2

¹H NMR (500 MHz, DMSO-d6) δ 1.25 (ddt, J=11.3, 8.6, 3.5 Hz, 3H), 1.53-1.57 (m, 1H), 1.60 (s, 1H), 1.81 (t, J=11.8 Hz, 2H), 2.13 (d, J=1.3 Hz, 3H), 2.72 (s, 1H), 2.74 (s, 1H), 2.77 (d, J=6.9 Hz, 2H), 3.82 (s, 3H), 7.71 (s, 1H)

### 4-Chloro-5-((1-methylpiperidin-4-yl)methyl)thiophene-2-carboxylic Acid

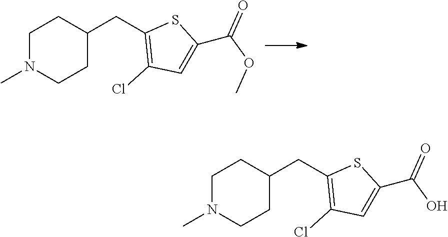 [SMILES: COC(=O)c1cc(Cl)c(CC2CCN(C)CC2)s1 ; CN1CCC(Cc2sc(C(=O)O)cc2Cl)CC1]

Following general procedure E, methyl 4-chloro-5-((1-methylpiperidin-4-yl)methyl)thiophene-2-carboxylate (56 mg, 0.195 mmol) afforded the title compound (83 mg, quantitative yield).

[M+H]⁺=274.1

### Methyl 5-(1-methyl-1,2,3,6-tetrahydropyridin-4-yl)thiophene-2-carboxylate

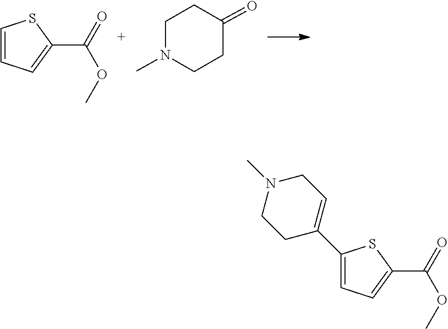

A solution of methyl thiophene-2-carboxylate (0.82 mL, 7.03 mmol) in THF (20 mL) was cooled to −78° C. then treated with LDA (2M in THF, hexanes, ethylbenzene) (3.6 mL, 7.20 mmol) was added dropwise with temperature maintained under −65° C. After addition was complete the reaction was stirred for 30 min then a solution of 1-methylpiperidin-4-one (0.95 mL, 7.72 mmol) in THF (2 mL) was added dropwise maintaining the temperature below −60° C. After addition was complete the reaction mixture was stirred for 15 min at −78° C. then allowed to warm to rt. Once at rt the reaction mixture was cooled to 0° C. then TFA (1 mL) was added and the reaction mixture was concentrated in vacuo. The product was dissolved in TFA (6 mL) and heated to 70° C. for 18 hrs. The reaction was concentrated and purified by flash chromatography (0-6% ((0.7M NH₃ in MeOH) in DCM) to afford the title compound (1.12 g, 54% yield) as a purple gum.

[M+H]⁺=238.1

### Methyl 5-(1-methylpiperidin-4-yl)thiophene-2-carboxylate

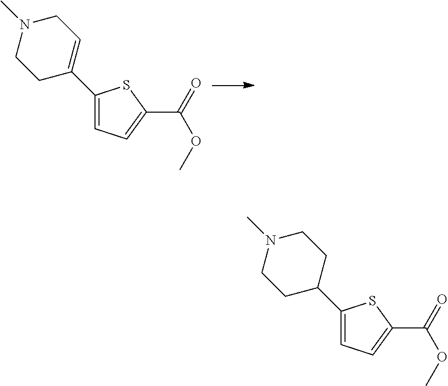 [SMILES: COC(=O)c1ccc(C2=CCN(C)CC2)s1 ; COC(=O)c1ccc(C2CCN(C)CC2)s1]

A solution of methyl 5-(1-methyl-1,2,3,6-tetrahydropyridin-4-yl)thiophene-2-carboxylate (1.12 g, 4.72 mmol) in MeOH (10 mL) was hydrogenated in the H-Cube (10% Pd/C, 30×4 mm, Full hydrogen, 40° C., 1 mL/min) for 4 hrs. The reaction mixture was concentrated and purified by flash chromatography (0-6% ((0.7M NH₃ in MeOH) in DCM) to afford the title compound (265 mg, 23% yield) as a brown oil.

[M+H]⁺=240.1

¹H NMR (500 MHz, DMSO-d6) δ 1.55-1.71 (m, 2H), 1.87-2.05 (m, 4H), 2.19 (s, 3H), 2.76-2.88 (m, 3H), 3.79 (s, 3H), 7.02 (d, J=3.8 Hz, 1H), 7.67 (d, J=3.8 Hz, 1H)

### Methyl 4-chloro-5-(1-methylpiperidin-4-yl)thiophene-2-carboxylate

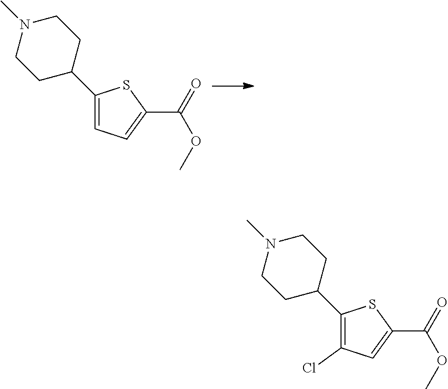 [SMILES: COC(=O)c1ccc(C2CCN(C)CC2)s1 ; COC(=O)c1cc(Cl)c(C2CCN(C)CC2)s1]

Sulfuryl chloride (180 μL, 2.22 mmol) in CHCl₃ (2 mL) was added dropwise to a solution of methyl 5-(1-methylpiperidin-4-yl)thiophene-2-carboxylate (160 mg, 0.67 mmol) in CHCl₃ (2 mL) and the mixture stirred at 50° C. for 8 hrs. The reaction mixture was partitioned between sat. Na₂CO₃ (20 mL) and DCM (20 mL). The aqueous phase was further extracted with DCM (2×20 mL), before the organic phases were combined, dried (MgSO₄), filtered and concentrated. The residue was purified by flash chromatography (0-5% ((0.7M NH₃ in MeOH) in DCM) to afford the title compound (36 mg, 19% yield) as a red solid.

[M+H]⁺=274.0

¹H NMR (500 MHz, DMSO-d6) δ 1.51-1.67 (m, 2H), 1.86-1.95 (m, 2H), 1.95-2.06 (m, 2H), 2.20 (s, 3H), 2.82-2.89 (m, 2H), 2.89-2.97 (m, 1H), 3.82 (s, 3H), 7.72 (s, 1H).

### 5-(1-Methylpiperidin-4-yl)thiophene-2-carboxylic Acid

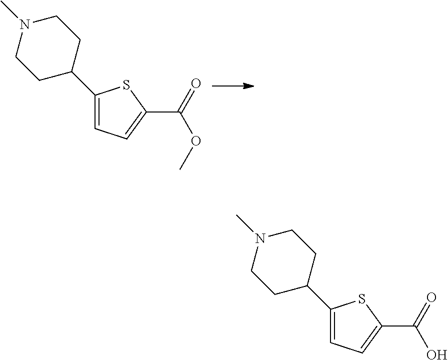 [SMILES: COC(=O)c1ccc(C2CCN(C)CC2)s1 ; CN1CCC(c2ccc(C(=O)O)s2)CC1]

Following general method (E), methyl 5-(1-methylpiperidin-4-yl)thiophene-2-carboxylate (31 mg, 0.13 mmol) afforded the title compound (27 mg, quantitative yield).

[M+H]⁺=226.1

### 4-Chloro-5-(1-methylpiperidin-4-yl)thiophene-2-carboxylic Acid

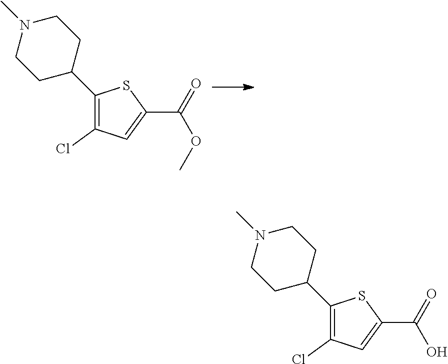 [SMILES: COC(=O)c1cc(Cl)c(C2CCN(C)CC2)s1 ; CN1CCC(c2sc(C(=O)O)cc2Cl)CC1]

Following general method (E), methyl 4-chloro-5-(1-methylpiperidin-4-yl)thiophene-2-carboxylate (35 mg, 0.13 mmol) afforded the title compound (30 mg, quantitative yield).

[M+H]⁺=260.0

### tert-Butyl 4-(2-(2-(methoxycarbonyl)-5-methylthiophen-3-yl)vinyl)piperidine-1-carboxylate

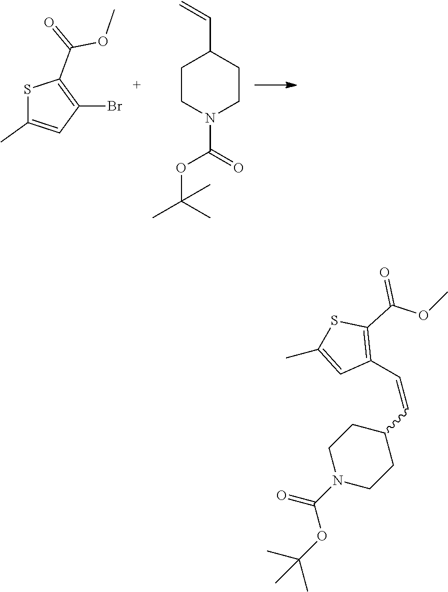

A solution of methyl 3-bromo-5-methylthiophene-2-carboxylate (100 mg, 0.425 mmol), tert-butyl 4-vinylpiperidine-1-carboxylate (180 mg, 0.851 mmol) and DIPEA (180 μL, 1.03 mmol) in DMF (1 mL) was degassed and purged with nitrogen at 40° C. then cataCXium Pd G2 (25 mg, 0.037 mmol) was added. The mixture was heated to 100° C. overnight. The reaction mixture was taken up in EtOAc (30 mL) and washed with 1M HCl (30 mL) and brine (30 mL). The organic layer was dried over MgSO₄, filtered and concentrated in vacuo. The crude product was purified by flash chromatography (0-20% EtOAc/isohexane) to afford the title compound (67 mg, 42% yield) as a colourless gum.

[M-Boc+H]⁺=266.1

### tert-Butyl 4-(2-(2-(methoxycarbonyl)-5-methylthiophen-3-yl)ethyl)piperidine-1-carboxylate

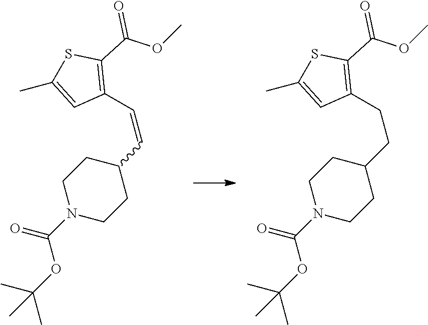 [SMILES: COC(=O)c1sc(C)cc1C=CC1CCN(C(=O)OC(C)(C)C)CC1 ; COC(=O)c1sc(C)cc1CCC1CCN(C(=O)OC(C)(C)C)CC1]

A solution of tert-butyl 4-(2-(2-(methoxycarbonyl)-5-methylthiophen-3-yl)vinyl)piperidine-1-carboxylate (280 mg, 0.766 mmol) in MeOH (40 mL) was hydrogenated in the H-Cube (10% Pd/C, 30×4 mm, 50 bar, 40° C., 1 mL/min). The reaction mixture was concentrated in vacuo and purified by flash chromatography (0-15% EtOAc/isohexane) to afford the title compound (163 mg, 55% yield) as a colourless gum.

[M-Boc+H]⁺=268.1

¹H NMR (500 MHz, DMSO-d6) δ 0.91-1.04 (m, 2H), 1.33-1.52 (m, 12H), 1.62-1.71 (m, 2H), 2.39-2.45 (m, 3H), 2.56-2.76 (m, 2H), 2.85-2.96 (m, 2H), 3.75 (s, 3H), 3.87-3.96 (m, 2H), 6.84 (d, J=1.2 Hz, 1H)

### tert-Butyl 4-((5-(methoxycarbonyl)thiophen-3-yl)ethynyl)piperidine-1-carboxylate

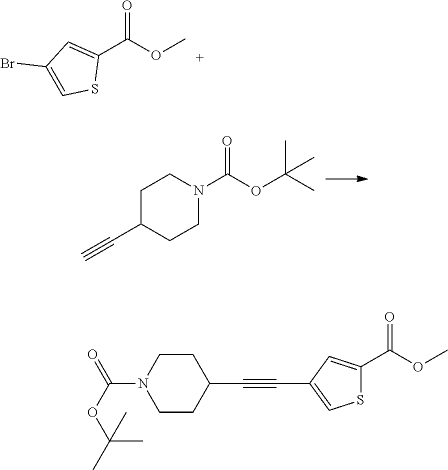

A solution of methyl 4-bromothiophene-2-carboxylate (500 mg, 2.26 mmol) and tert-butyl 4-ethynylpiperidine-1-carboxylate (500 mg, 2.39 mmol) in DMF (50 mL) was treated with Et₃N (500 μL, 3.59 mmol) followed by copper (1) iodide (100 mg, 0.53 mmol) and Pd(PPh₃)₂Cl₂ (200 mg, 0.29 mmol). The reaction mixture was heated to 80° C. for 5 hrs before being allowed to cool to rt. The reaction mixture was taken up in EtOAc (100 mL) and washed with water (100 mL) 1:1 brine:water (100 mL) and brine (100 mL). The organic phases were combined and dried (MgSO₄), filtered and concentrated. The residue was purified by flash chromatography (0-50% EtOAc in isohexane) to afford the title compound (455 mg, 52% yield) as a colourless gum.

[M-Boc+H]⁺=250.0

¹H NMR (500 MHz, DMSO-d6) δ 1.40 (s, 9H), 1.43-1.55 (m, 2H), 1.75-1.85 (m, 2H), 2.81-2.88 (m, 1H), 3.09-3.17 (m, 2H), 3.58-3.71 (m, 2H), 3.83 (s, 3H), 7.75 (d, J=1.5 Hz, 1H), 8.05 (d, J=1.5 Hz, 1H).

### Methyl 4-((1-(tert-butoxycarbonyl)piperidin-4-yl)ethynyl)thiazole-2-carboxylate

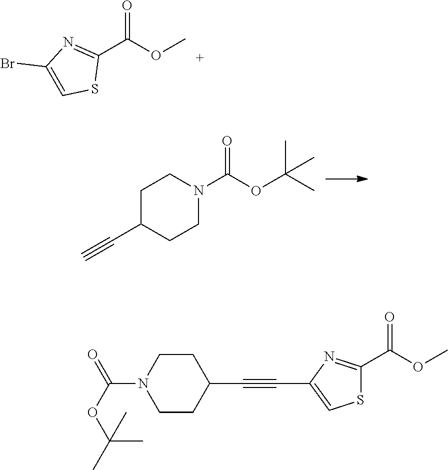

A solution of methyl 4-bromothiazole-2-carboxylate (500 mg, 2.25 mmol) and tert-butyl 4-ethynylpiperidine-1-carboxylate (500 mg, 2.39 mmol) in DMF (50 mL) was treated with Et₃N (500 μL, 3.59 mmol) followed by copper (1) iodide (100 mg, 0.53 mmol) and Pd(PPh₃)₂Cl₂ (200 mg, 0.29 mmol). The reaction mixture was heated to 80° C. for 5 hrs before being allowed to cool to rt. The reaction mixture was taken up in EtOAc (100 mL) and washed with water (100 mL) 1:1 brine:water (100 mL) and brine (100 mL). The organic phases were combined and dried (MgSO₄). The residue was purified by flash chromatography (0-50% EtOAc in isohexane) to afford the title compound (556 mg, 67% yield) as an orange gum.

[M−tBu+H]⁺=295.0

¹H NMR (500 MHz, DMSO-d6) δ 1.41 (s, 9H), 1.48-1.56 (m, 2H), 1.81-1.88 (m, 2H), 2.85-2.94 (m, 1H), 3.09-3.16 (m, 2H), 3.63-3.69 (m, 2H), 3.92 (s, 3H), 8.27 (s, 1H).

### tert-Butyl 4-((5-(methoxycarbonyl)thiophen-2-yl)ethynyl)piperidine-1-carboxylate

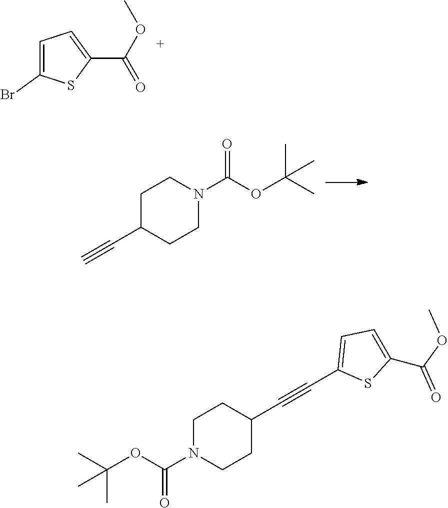

To a solution of methyl 5-bromothiophene-2-carboxylate (1 g, 4.52 mmol) and tert-butyl 4-ethynylpiperidine-1-carboxylate (1 g, 4.78 mmol) in DMF (20 mL) was treated with Et₃N (0.85 mL, 6.10 mmol) followed by copper (1) iodide (200 mg, 1.05 mmol) and Pd(PPh₃)₂Cl₂ (400 mg, 0.57 mmol). The reaction mixture was heated to 80° C. for 5 hrs before being allowed to cool to rt. The reaction mixture was taken up in EtOAc (100 mL) and washed with water (100 ml), 1:1 brine:water (100 mL) and brine (100 mL). The organic phase was dried (MgSO₄), filtered and concentrated in vacuo. The residue was purified by flash chromatography (0-50% EtOAc in isohexane) afforded the title compound (1.23 g, 70% yield) as a colourless glass.

[M−Boc+H]⁺=250.0

¹H NMR (500 MHz, DMSO-d6) δ 1.40 (s, 9H), 1.44-1.57 (m, 2H), 1.78-1.88 (m, 2H), 2.93 (tt, J=8.6, 3.9 Hz, 1H), 3.04-3.15 (m, 2H), 3.60-3.72 (m, 2H), 3.82 (s, 3H), 7.31 (d, J=3.9 Hz, 1H), 7.71 (d, J=3.9 Hz, 1H).

### Methyl 4-(piperidin-4-ylethynyl)thiophene-2-carboxylate

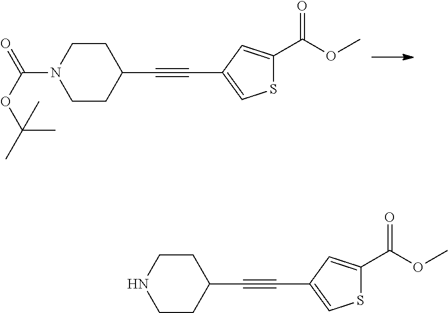 [SMILES: COC(=O)c1cc(C#CC2CCN(C(=O)OC(C)(C)C)CC2)cs1 ; COC(=O)c1cc(C#CC2CCNCC2)cs1]

Following general conditions (D), tert-butyl 4-((5-(methoxycarbonyl)thiophen-3-yl)ethynyl)piperidine-1-carboxylate (455 mg, 1.30 mmol) afforded the title compound (233 mg, 65% yield) as a colourless gum.

[M+H]⁺=250.0

¹H NMR (500 MHz, DMSO-d6) δ 1.43-1.51 (m, 2H), 1.73-1.80 (m, 2H), 2.52-2.57 (m, 2H), 2.63-2.72 (m, 1H), 2.84-2.93 (m, 2H), 3.83 (s, 3H), 7.72 (d, J=1.4 Hz, 1H), 8.02 (d, J=1.4 Hz, 1H), N—H not observed.

### 4-(Piperidin-4-ylethynyl)thiazole-2-carboxamide

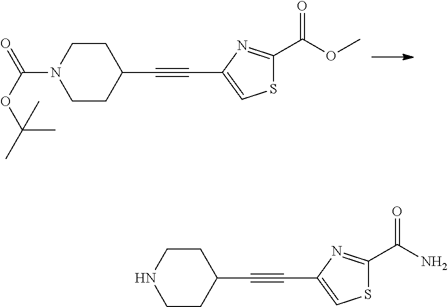 [SMILES: COC(=O)c1nc(C#CC2CCN(C(=O)OC(C)(C)C)CC2)cs1 ; NC(=O)c1nc(C#CC2CCNCC2)cs1]

Following general conditions (D), methyl 4-((1-(tert-butoxycarbonyl)piperidin-4-yl)ethynyl)thiazole-2-carboxylate (554 mg, 1.581 mmol) afforded the boc deprotected product which was loaded onto an SCX column with MeOH (20 mL). Elution with 7M NH₃ in MeOH (50 mL) and concentration afforded the title compound (233 mg, 60% yield) as a pink solid.

[M+H]⁺=236.0

¹H NMR (500 MHz, DMSO-d6) δ 1.41-1.55 (m, 2H), 1.75-1.84 (m, 2H), 2.53-2.58 (m, 2H), 2.68-2.78 (m, 1H), 2.87-2.95 (m, 2H), 7.89 (s, 1H), 8.11 (s, 1H), 8.31 (s, 1H), N—H not observed.

### Methyl 5-(piperidin-4-ylethynyl)thiophene-2-carboxylate

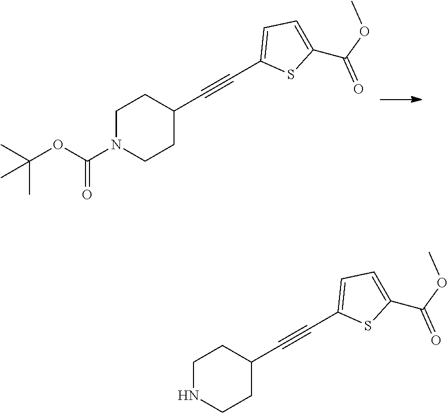 [SMILES: COC(=O)c1ccc(C#CC2CCN(C(=O)OC(C)(C)C)CC2)s1 ; COC(=O)c1ccc(C#CC2CCNCC2)s1]

Following general procedure D, tert-butyl 4-((5-(methoxycarbonyl)thiophen-2-yl)ethynyl)piperidine-1-carboxylate (1.29 g, 3.69 mmol) afforded the title compound (900 mg, 88% yield) as a yellow gum.

[M+H]⁺=250.0

¹H NMR (500 MHz, DMSO-d6) δ 1.44-1.56 (2H, m), 1.76-1.83 (2H, m), 2.53-2.61 (2H, m), 2.75-2.82 (1H, m), 2.87-2.94 (2H, m), 3.82 (3H, s), 7.28 (1H, d, J=3.9 Hz), 7.71 (1H, d, J=3.9 Hz), 1×N—H not observed.

### Methyl 4-((1-methylpiperidin-4-yl)ethynyl)thiophene-2-carboxylate

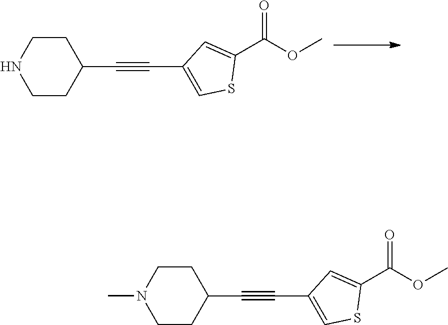

Following general conditions (F), methyl 4-(piperidin-4-ylethynyl)thiophene-2-carboxylate (233 mg, 0.94 mmol) afforded the title compound (103 mg, 41% yield) as a colourless glass.

[M+H]⁺=264.0

¹H NMR (500 MHz, DMSO-d6) δ 1.55-1.66 (m, 2H), 1.80-1.88 (m, 2H), 2.04-2.15 (m, 2H), 2.17 (s, 3H), 2.56-2.67 (m, 3H), 3.83 (s, 3H), 7.72 (d, J=1.5 Hz, 1H), 8.03 (d, J=1.5 Hz, 1H)

### 4-((1-Methylpiperidin-4-yl)ethynyl)thiazole-2-carboxamide

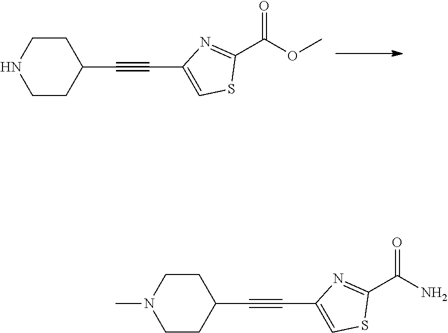

Following general conditions (F), 4-(piperidin-4-ylethynyl)thiazole-2-carboxamide (233 mg, 0.99 mmol) was subjected to reductive amination which after purification via SCX (eluent 7M NH₃ in MeOH) afforded the title compound (199 mg, 79% yield) as a red solid.

[M+H]⁺=250.0

¹H NMR (500 MHz, DMSO-d6) δ 1.55-1.66 (m, 2H), 1.80-1.91 (m, 2H), 1.98-2.12 (m, 2H), 2.16 (s, 3H), 2.58-2.67 (m, 3H), 7.86-7.91 (m, 1H), 8.11 (s, 1H), 8.30 (s, 1H)

### Methyl 5-((1-methylpiperidin-4-yl)ethynyl)thiophene-2-carboxylate

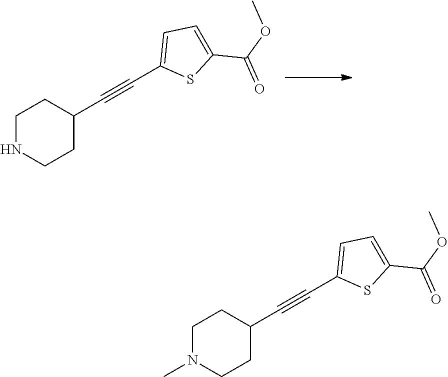 [SMILES: COC(=O)c1ccc(C#CC2CCNCC2)s1 ; COC(=O)c1ccc(C#CC2CCN(C)CC2)s1]

Following general procedure F, methyl 5-(piperidin-4-ylethynyl)thiophene-2-carboxylate (900 mg, 3.61 mmol) afforded the title compound (350 mg, 36% yield) as a colourless gum.

[M+H]⁺=264.1

¹H NMR (500 MHz, DMSO-d6) δ 1.51-1.67 (m, 2H), 1.78-1.89 (m, 2H), 2.02-2.13 (m, 2H), 2.15 (s, 3H), 2.54-2.63 (m, 2H), 2.63-2.73 (m, 1H), 3.82 (s, 3H), 7.29 (d, J=3.9 Hz, 1H), 7.71 (d, J=3.9 Hz, 1H)

### Methyl 4-(2-(1-methylpiperidin-4-yl)ethyl)thiophene-2-carboxylate

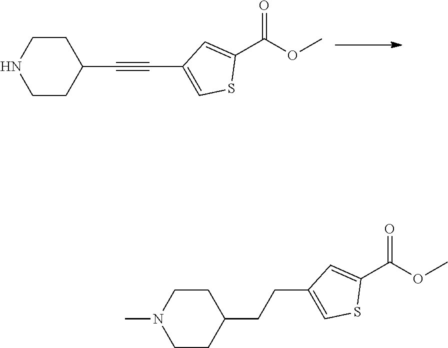

A solution of methyl 4-((1-methylpiperidin-4-yl)ethynyl)thiophene-2-carboxylate (103 mg, 0.39 mmol) in MeOH (10 mL) was hydrogenated in the H-Cube (10% Pd/C, 30×4 mm, 30 bar, 45° C., 1.5 mL/min) recirculating for 1.5 hrs. The reaction mixture was concentrated in vacuo and afforded the title compound (74 mg, 69% yield) as a colourless oil.

[M+H]⁺=268.1

¹H NMR (500 MHz, DMSO-d6) δ 1.10-1.20 (m, 3H), 1.47-1.54 (m, 2H), 1.61-1.67 (m, 2H), 1.74-1.83 (m, 2H), 2.13 (s, 3H), 2.57-2.64 (m, 2H), 2.69-2.77 (m, 2H), 3.80 (s, 3H), 7.58 (d, J=1.5 Hz, 1H), 7.68 (d, J=1.6 Hz, 1H)

### Methyl 5-(2-(1-methylpiperidin-4-yl)ethyl)thiophene-2-carboxylate

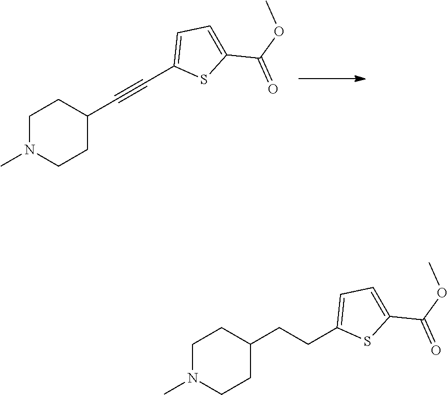 [SMILES: COC(=O)c1ccc(C#CC2CCN(C)CC2)s1 ; COC(=O)c1ccc(CCC2CCN(C)CC2)s1]

A solution of methyl 5-((1-methylpiperidin-4-yl)ethynyl)thiophene-2-carboxylate (300 mg, 1.14 mmol) in MeOH (10 mL) was hydrogenated in the H-Cube (10% Pd/C, 30×4 mm, Full hydrogen, 40° C., 1.5 mL/min) recirculating for 5 hrs. The reaction mixture was concentrated in vacuo to afford the title compound (264 mg, 85% yield) as a colourless glass.

[M+H]⁺=268.1

¹H NMR (500 MHz, DMSO-d6) δ 1.09-1.23 (m, 3H), 1.45-1.60 (m, 2H), 1.60-1.67 (m, 2H), 1.70-1.81 (m, 2H), 2.12 (s, 3H), 2.70-2.76 (m, 2H), 2.83-2.89 (m, 2H), 3.79 (s, 3H), 6.97 (d, J=3.8 Hz, 1H), 7.64 (d, J=3.8 Hz, 1H)

### Methyl 4-chloro-5-(2-(1-methylpiperidin-4-yl)ethyl)thiophene-2-carboxylate

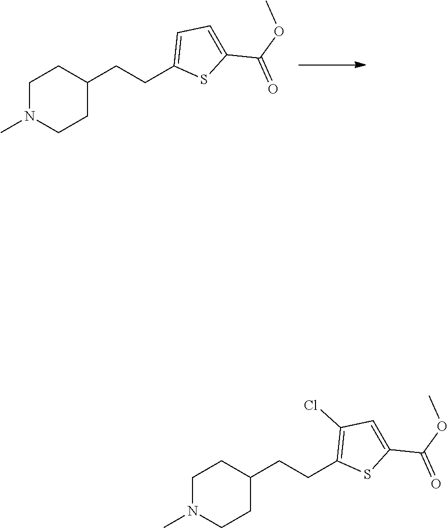 [SMILES: COC(=O)c1ccc(CCC2CCN(C)CC2)s1 ; COC(=O)c1cc(Cl)c(CCC2CCN(C)CC2)s1]

Sulfuryl chloride (120 μL, 1.48 mmol) was added dropwise to a solution of methyl 5-(2-(1-methylpiperidin-4-yl)ethyl)thiophene-2-carboxylate (264 mg, 0.99 mmol) in CHCl₃ (4 mL) and the mixture stirred at 60° C. for 2 hrs. The reaction mixture was taken up in sat. Na₂CO₃ (20 mL) and DCM (20 mL) was added. The phases were separated, and the aqueous phase was further extracted with DCM (2×20 ml). The organic phases were combined and dried (MgSO₄), filtered and concentrated and the residue purified by flash chromatography (0-5% (0.7M NH₃ in MeOH) in DCM) afforded the title compound (100 mg, 30% yield) as a red solid.

[M+H]⁺=302.0

¹H NMR (500 MHz, DMSO-d6) δ 1.12-1.22 (m, 2H), 1.49-1.58 (m, 2H), 1.62-1.69 (m, 3H), 1.74-1.84 (m, 2H), 2.13 (s, 3H), 2.69-2.77 (m, 2H), 2.79-2.87 (m, 2H), 3.82 (s, 3H), 7.71 (s, 1H).

### tert-Butyl 4-((2-(((1-aminoisoquinolin-6-yl)methyl)carbamoyl)thiazol-4-yl)ethynyl)piperidine-1-carboxylate

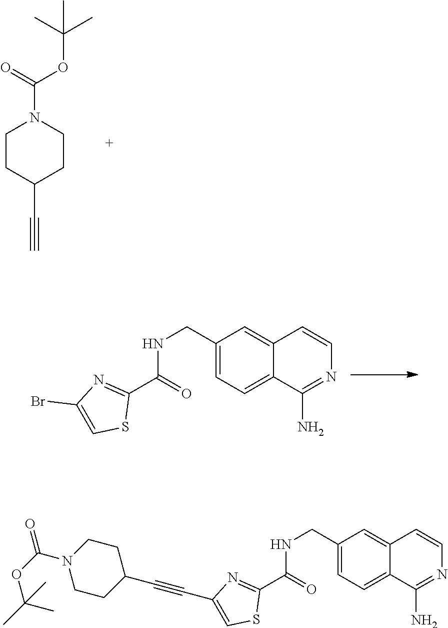 [SMILES: C#CC1CCN(C(=O)OC(C)(C)C)CC1 ; Nc1nccc2cc(CNC(=O)c3nc(Br)cs3)ccc12 ; CC(C)(C)OC(=O)N1CCC(C#Cc2csc(C(=O)NCc3ccc4c(N)nccc4c3)n2)CC1]

To a solution of N-((1-aminoisoquinolin-6-yl)methyl)-4-bromothiazole-2-carboxamide (200 mg, 0.55 mmol) and tert-butyl 4-ethynylpiperidine-1-carboxylate (115 mg, 0.55 mmol) in DMF (50 mL) was added Et₃N (200 μL, 1.44 mmol) followed by copper (1) iodide (20 mg, 0.11 mmol) and Pd(PPh₃)₂Cl₂ (40 mg, 0.06 mmol). The reaction was heated to 80° C. for 5 hrs before being allowed to cool to rt. The reaction mixture was taken up in EtOAc (50 mL) and washed with water (50 mL), 1:1 brine:water (50 mL) and brine (50 ml). The organic phase was dried (MgSO₄) and concentrated in vacuo. Purification by flash chromatography (0-10% (0.7M NH₃ in MeOH) in DCM) afforded the title compound (122 mg, 41% yield) as a colourless gum.

[M+H]⁺=492.2

¹H NMR (500 MHz, DMSO-d6) δ 1.40 (s, 9H), 1.45-1.55 (m, 2H), 1.80-1.88 (m, 2H), 2.84-2.93 (m, 1H), 3.04-3.15 (m, 2H), 3.65-3.71 (m, 2H), 4.57 (d, J=6.1 Hz, 2H), 6.77 (s, 2H), 6.88 (d, J=5.9 Hz, 1H), 7.42 (dd, J=8.7, 1.7 Hz, 1H), 7.56 (s, 1H), 7.76 (d, J=5.8 Hz, 1H), 8.14 (d, J=8.6 Hz, 1H), 8.18 (s, 1H), 9.65 (t, J=6.3 Hz, 1H)

### tert-Butyl 4-(2-(2-(((1-aminoisoquinolin-6-yl)methyl)carbamoyl)thiazol-4-yl)ethyl)piperidine-1-carboxylate

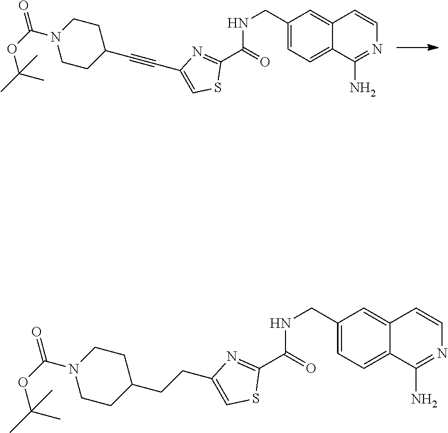 [SMILES: CC(C)(C)OC(=O)N1CCC(C#Cc2csc(C(=O)NCc3ccc4c(N)nccc4c3)n2)CC1 ; CC(C)(C)OC(=O)N1CCC(CCc2csc(C(=O)NCc3ccc4c(N)nccc4c3)n2)CC1]

A solution of tert-butyl 4-((2-(((1-aminoisoquinolin-6-yl)methyl)carbamoyl)thiazol-4-yl)ethynyl)piperidine-1-carboxylate (122 mg, 0.25 mmol) in MeOH (10 mL) was hydrogenated in the H-Cube (10% Pd/C, 30×4 mm, 30 bar, 45° C., 1.5 mL/min) recirculating for 5 hrs. The reaction mixture was concentrated in vacuo to afford the title compound (45 mg, 36% yield) as a colourless glass.

[M+H]⁺=496.2

¹H NMR (500 MHz, DMSO-d6) δ 0.95-1.06 (m, 2H), 1.37-1.47, (m, 1H), 1.39 (s, 9H), 1.59-1.73 (m, 4H), 2.60-2.75 (m, 2H), 2.77-2.83 (m, 2H), 3.87-3.98 (m, 2H), 4.58 (d, J=6.3 Hz, 2H), 6.71 (s, 2H), 6.86 (d, J=5.8 Hz, 1H), 7.42 (dd, J=8.6, 1.7 Hz, 1H), 7.56 (d, J=1.7 Hz, 1H), 7.65 (s, 1H), 7.76 (d, J=5.8 Hz, 1H), 8.14 (d, J=8.6 Hz, 1H), 9.35 (t, J=6.4 Hz, 1H).

### Methyl 1-(3-(1-methylpiperidin-4-yl)propyl)-H-pyrazole-3-carboxylate

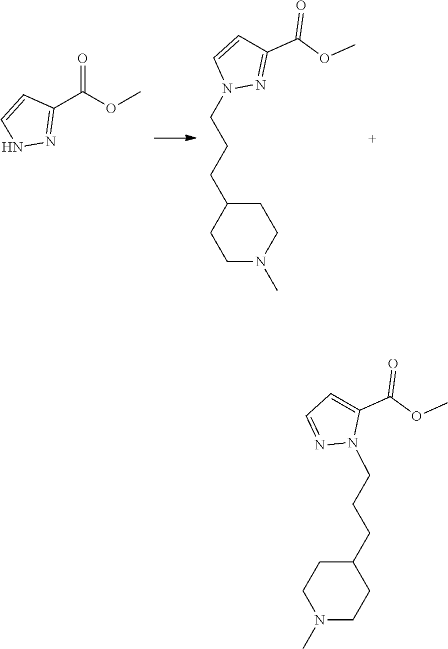

Following general method I (i), 3-(1-methylpiperidin-4-yl)propan-1-ol (500 mg, 3.18 mmol) was reacted with methyl 1H-pyrazole-3-carboxylate (308 mg, 2.45 mmol). Methyl 1-(3-(1-methylpiperidin-4-yl)propyl)-1H-pyrazole-3-carboxylate (41 mg, 6% yield) and methyl 1-(3-(1-methylpiperidin-4-yl)propyl)-1H-pyrazole-5-carboxylate (365 mg, 55%) were both isolated as colourless oils. Regioisomers were assigned using ¹H NMR experiments.

### Methyl 1-(3-(1-methylpiperidin-4-yl)propyl)-H-pyrazole-3-carboxylate

[M+H]⁺=266.1

¹H NMR (500 MHz, DMSO-d6) δ 0.98-1.20 (m, 5H), 1.48-1.62 (m, 2H), 1.72-1.84 (m, 4H), 2.11 (s, 3H), 2.67-2.75 (m, 2H), 3.78 (s, 3H), 4.15 (t, J=7.1 Hz, 2H), 6.73 (d, J=2.3 Hz, 1H), 7.87 (d, J=2.3 Hz, 1H).

### Methyl 1-(3-(1-methylpiperidin-4-yl)propyl)-H-pyrazole-5-carboxylate

[M+H]⁺=266.1

¹H NMR (500 MHz, DMSO-d6) δ 1.02-1.22 (m, 5H), 1.52-1.62 (m, 2H), 1.67-1.82 (m, 4H), 2.11 (s, 3H), 2.66-2.76 (m, 2H), 3.83 (s, 3H), 4.43-4.49 (m, 2H), 6.88 (d, J=2.0 Hz, 1H), 7.57 (d, J=2.0 Hz, 1H).

### Methyl 1-(4-ethoxybenzyl)-1H-pyrazole-5-carboxylate

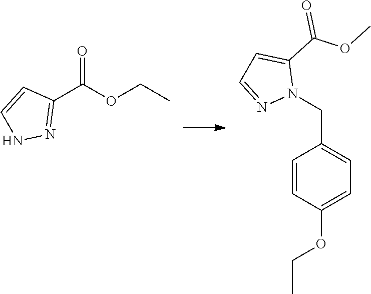

Following general method I (i), (4-ethoxyphenyl)methanol (272 mg, 1.78 mmol) was reacted with methyl 1H-pyrazole-3-carboxylate (150 mg, 1.19 mmol). The title compound was isolated (181 mg, 59%) as a clear, colourless oil. The desired regioisomer was assigned using ¹H NMR experiments.

[M+H]⁺=261.0

1H NMR (DMSO, 400 MHz): 1.29 (3H, t, J=7.0 Hz), 3.81 (3H, s), 3.97 (2H, q, J=7.0 Hz), 5.63 (2H, s), 6.83-6.87 (2H, m), 6.92 (1H, d, J=2.0 Hz), 7.10-7.13 (2H, m), 7.62 (1H, d, J=2.0 Hz)

### tert-Butyl 4-((3-chloro-5-(ethoxycarbonyl)-1H-pyrazol-1-yl)methyl)piperidine-1-carboxylate

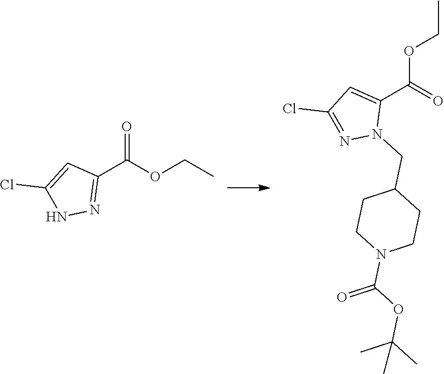 [SMILES: CCOC(=O)c1cc(Cl)[nH]n1 ; CCOC(=O)c1cc(Cl)nn1CC1CCN(C(=O)OC(C)(C)C)CC1]

Following general method G, ethyl 5-chloro-1H-pyrazole-3-carboxylate (100 mg, 0.573 mmol) was reacted with tert-butyl 4-(bromomethyl)piperidine-1-carboxylate (207 mg, 0.745 mmol) to afford the title product (128 mg, 60% yield).

### Methyl 1-(2-piperidin-4-yl)ethyl)-1H-pyrazole-3-carboxylate

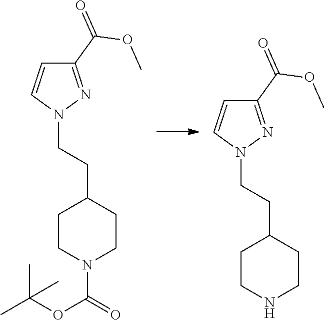 [SMILES: COC(=O)c1ccn(CCC2CCN(C(=O)OC(C)(C)C)CC2)n1 ; COC(=O)c1ccn(CCC2CCNCC2)n1]

Following general procedure D, tert-butyl 4-(2-(3-(methoxycarbonyl)-1H-pyrazol-1-yl)ethyl)piperidine-1-carboxylate (992 mg, 2.94 mmol) was deprotected to afford the title compound as the hydrochloride salt. The compound was free-based by washing with 200 mg PL-HCO₃ MP resin. The resin was filtered off and washed with MeOH (50 mL), solvent was removed in vacuo and the white oily residue was purified by flash chromatography (0-30% (10% NH₃ in MeOH) in DCM) to afford title compound (300 mg, 43% yield) as colourless oil.

[M+H]⁺=238.1

¹H NMR (DMSO-d6, 400 MHz) δ 1.00-1.15 (2H, m), 1.19-1.31 (1H, m), 1.61 (2H, d, J=12.2 Hz), 1.71 (2H, q, J=6.9 Hz), 2.39-2.48 (2H, m), 2.94 (2H, d, J=12.1 Hz), 3.78 (3H, s), 4.17-4.24 (2H, m), 6.73 (1H, d, J=2.3 Hz), 7.89 (1H, d, J=2.3 Hz) (NH proton not visible)

### Methyl 1-(2-(1-acetylpiperidin-4-yl)ethyl)-1H-pyrazole-3-carboxylate

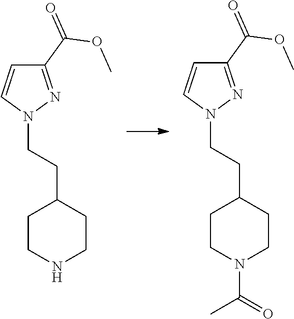 [SMILES: COC(=O)c1ccn(CCC2CCNCC2)n1 ; COC(=O)c1ccn(CCC2CCN(C(C)=O)CC2)n1]

A solution of methyl 1-(2-(piperidin-4-yl)ethyl)-1H-pyrazole-3-carboxylate (150 mg, 0.63 mmol) and triethylamine (264 μL, 1.90 mmol) in anhydrous DCM (5 mL) was cooled in an ice bath. Acyl chloride (49.4 μL, 0.70 mmol) was added dropwise. On completion of addition the ice bath was removed, and the mixture stirred at rt for 2 days. The reaction mixture was diluted with DCM (50 mL) and washed with water (10 mL), then brine (10 mL), dried over Na₂SO₄, filtered and concentrated to afford title compound (120 mg, 68% yield) as light yellow oil. Used without further purification.

[M+H]⁺=280.0

### Lithium 1-(2-(1-methylpiperidin-4-yl)ethyl)-1H-pyrazole-5-carboxylate

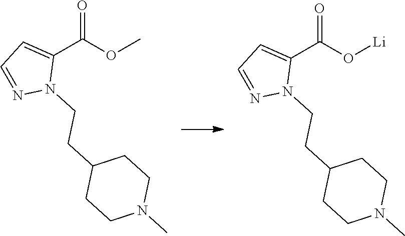

Following general method (E), methyl 1-(2-(1-methylpiperidin-4-yl)ethyl)-1H-pyrazole-5-carboxylate (50 mg, 0.20 mmol) was hydrolysed to afford the title compound (47 mg, quantitative yield).

### Methyl 3-(methoxymethyl)-1-(methylsulfonyl)-1H-pyrazole-4-carboxylate

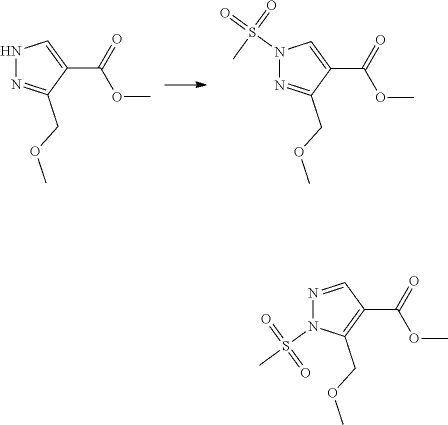

To a stirred solution of methyl 3-(methoxymethyl)-1H-pyrazole-4-carboxylate (3.0 g, 17.6 mmol) in DCM (60 mL) at 0° C. was added TEA (3.3 mL, 23.7 mmol) followed by methanesulfonyl chloride (1.5 mL, 19.2 mmol). The resulting mixture was stirred for 10 min then allowed to warm to rt and stirred for an additional 30 min. The reaction was diluted with DCM (50 mL) and quenched with NH₄Cl solution (100 mL). The aqueous layer was extracted with DCM (2×10 mL) and the combined organic layers were dried over MgSO₄, filtered and concentrated in vacuo. The residue was purified by flash chromatography (30-100% EtOAc in Hexanes) to afford the title compound (4.39 g, 97% yield) as a pale-yellow oil (as a 5:3 mix of regioisomers).

Major isomer: 1H NMR (DMSO, 500 MHz) δ 3.33 (3H, s), 3.65 (3H, s), 3.80 (3H, s), 4.63 (2H, s), 8.69 (1H, s).

Minor isomer: 1H NMR (DMSO, 500 MHz) δ 3.31 (3H, s), 3.61 (3H, s), 3.83 (3H, s), 4.93 (2H, s), 8.25 (1H, s)

### Methyl 1-(phenylsulfonyl)-1H-pyrazole-3-carboxylate

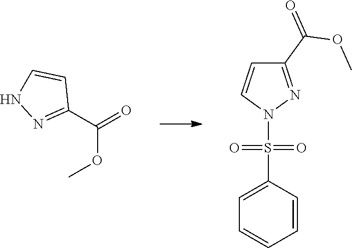

A solution of methyl 1H-pyrazole-3-carboxylate (500 mg, 3.965 mmol) in MeCN (10 mL) was cooled in an ice bath and benzenesulfonate chloride (0.531 mL, 4.163 mmol) added dropwise. On completion of addition the cooling bath was removed and the mixture was stirred at rt for 30 min and a white precipitate formed. The mixture was taken up in DCM (70 mL) and washed with water (50 mL). Organic layer was dried over Na₂SO₄, filtered and concentrated in vacuo. Crude residue was purified by flash chromatography (0-50% EtOAc in Pet. Ether) to afford the title product (997 mg, 94% yield) as white solid.

[M+H]⁺=266.9

### Ethyl 3-cyclopropyl-1-(methylsulfonyl)-1H-pyrazole-5-carboxylate

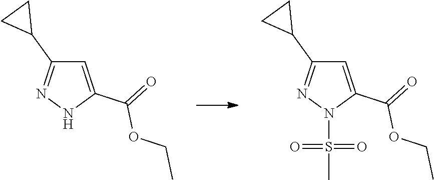 [SMILES: CCOC(=O)c1cc(C2CC2)n[nH]1 ; CCOC(=O)c1cc(C2CC2)nn1S(C)(=O)=O]

To a stirred solution of ethyl 3-cyclopropyl-1H-pyrazole-5-carboxylate (1 g, 5.55 mmol) in DCM (20 mL) at 0° C. was added TEA (1 mL, 7.17 mmol) followed by methanesulfonyl chloride (0.48 mL, 6.16 mmol). The resulting mixture was stirred for 10 min then allowed to warm to rt and stirred for an additional 30 min. The reaction was quenched with NH₄Cl aq. (30 mL), extracted with DCM (3×20 mL) and the combined organic extracts were washed with brine and concentrated in vacuo. The residual oil was purified by flash chromatography (30-100% EtOAc in Hexane) to afford a 6:1 mixture of regioisomers (1.4 g, 96% yield) as a white solid.

Major isomer: ¹H NMR (DMSO-d6, 500 MHz) δ: 0.82-0.92 (2H, m), 1.02-1.07 (2H, m), 1.30 (3H, t, J=7.1 Hz), 2.28-2.37 (1H, m), 3.65 (3H, s), 4.32 (2H, q, J=7.1), 6.65 (1H, s).

Minor isomer: ¹H NMR (DMSO-d6, 500 MHz) δ: 0.75-0.83 (2H, m), 0.96-1.01 (2H, m), 1.30 (3H, t, J=7.1 Hz), 1.96-2.04 (1H, m), 3.68 (3H, s), 4.32 (2H, q, J=7.1), 6.85 (1H, s).

[M+H]⁺=259.1

### Methyl 5-methyl-1-((1-(pyridin-4-yl)piperidin-4-yl)methyl)-1H-pyrazole-3-carboxylate

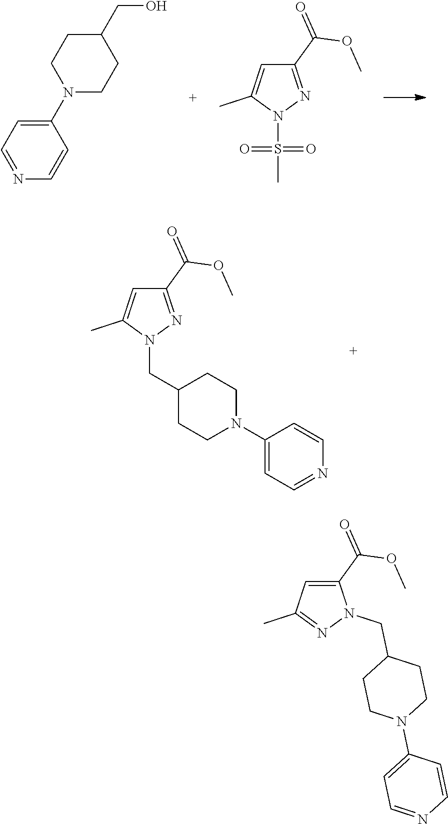

Following general method J, methyl 5-methyl-1-methylsulfonyl-pyrazole-3-carboxylate (0.93 g, 4.26 mmol) was reacted with (1-(pyridin-4-yl)piperidin-4-yl)methanol (650 mg, 3.38 mmol) to afford two regioisomers. The regioisomers were separated by flash chromatography (0-8% (1% NH₃ in MeOH) in DCM) to afford methyl 5-methyl-1-((1-(pyridin-4-yl)piperidin-4-yl)methyl)-1H-pyrazole-3-carboxylate (258 mg, 19% yield) and methyl 5-methyl-2-((1-(pyridin-4-yl)piperidin-4-yl)methyl)-1H-pyrazole-5-carboxylate (348 mg, 0.88 mmol, 26% yield) both as colourless gums. The regioisomers was assigned by ¹H NMR experiments.

### Methyl 5-methyl-1-((1-(pyridin-4-yl)piperidin-4-yl)methyl)-1H-pyrazole-3-carboxylate

[M+H]⁺=315.2

¹H NMR (DMSO-d6, 500 MHz) δ 1.19-1.31 (2H, m), 1.46-1.57 (2H, m), 2.08-2.15 (1H, m), 2.30 (3H, s), 2.78 (2H, td, J=12.9, 2.7 Hz), 3.77 (3H, s), 3.93 (2H, d, J=13.5 Hz), 4.01 (2H, d, J=7.3 Hz), 6.54 (1H, d, J=0.9 Hz), 6.77-6.80 (2H, m), 8.10-8.14 (2H, m).

### Methyl 5-methyl-1-((1-(pyridin-4-yl)piperidin-4-yl)methyl)-1H-pyrazole-5-carboxylate

[M+H]⁺=315.2

### Methyl 3-methyl-1-((1-(pyridin-4-yl)piperidin-4-yl)methyl)-1H-pyrazole-5-carboxylate

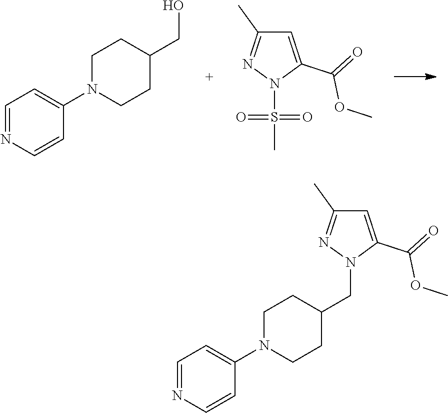

Following general method J, (1-(pyridin-4-yl)piperidin-4-yl)methanol (CAS 130658-67-2, 650 mg, 3.38 mmol) was reacted with methyl 3-methyl-1-(methylsulfonyl)-1H-pyrazole-5-carboxylate (930 mg, 4.26 mmol). The title compound was isolated as one of two regioisomers (314 mg, 26% yield) as a colourless gum. The desired regioisomer was determined by ¹H NMR experiments.

[M+H]⁺=315.2

### Ethyl 3-cyclopropyl-1-((1-(pyridin-4-yl)piperidin-4-yl)methyl)-1H-pyrazole-5-carboxylate

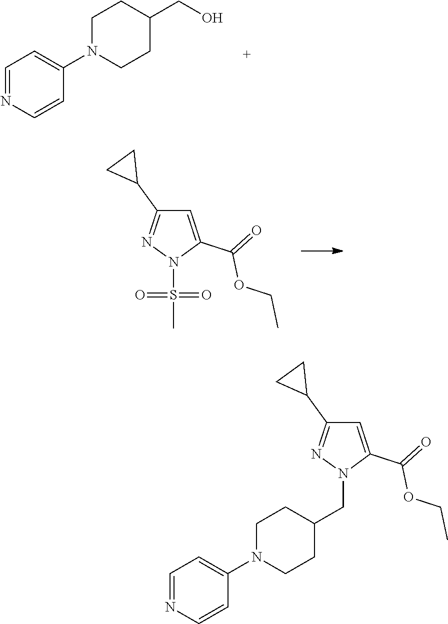

Following general method J, (1-(pyridin-4-yl)piperidin-4-yl)methanol (CAS 130658-67-2, 650 mg, 3.38 mmol) was reacted with ethyl 3-cyclopropyl-1-(methylsulfonyl)-1H-pyrazole-5-carboxylate (1.1 mg, 4.26 mmol). The title compound was isolated as one of two regioisomers (420 mg, 43% yield) as a clear, colourless oil. The desired regioisomer was assigned using ¹H NMR experiments.

¹H NMR (DMSO-d6, 500 MHz) δ 0.63-0.70 (2H, m), 0.82-0.92 (2H, m), 1.20 (2H, tt, J=12.3, 6.1 Hz), 1.28 (3H, t, J=7.1 Hz), 1.49 (2H, dd, J=13.7, 3.6 Hz), 1.90 (1H, tt, J=8.4, 5.0 Hz), 2.02-2.08 (1H, m), 2.76 (2H, td, J=12.8, 2.6 Hz), 3.90 (2H, dt, J=13.5, 3.2 Hz), 4.26 (2H, q, J=7.1 Hz), 4.31 (2H, d, J=7.2 Hz), 6.57 (1H, s), 6.74-6.80 (2H, m), 8.09-8.14 (2H, m)

[M+H]=355.1

### Ethyl 1-((tert-butoxycarbonyl)piperidin-4-yl)methyl)-1H-benzo[d]imidazole-2-carboxylate

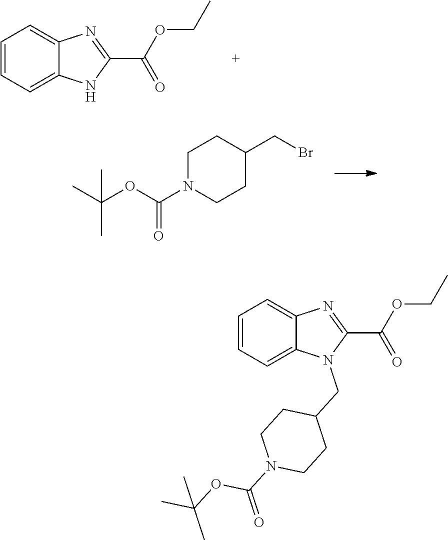 [SMILES: CCOC(=O)c1nc2ccccc2[nH]1 ; CC(C)(C)OC(=O)N1CCC(CBr)CC1 ; CCOC(=O)c1nc2ccccc2n1CC1CCN(C(=O)OC(C)(C)C)CC1]

Following general method G (i), ethyl-1H-benzo[d]imidazole-2-carboxylate (200 mg, 1.14 mmol) was reacted with 4-(bromomethyl)piperidine-1-carboxylate (379 mg, 1.36 mmol). Purification by flash chromatography (0-100% (10% NH₃ in MeOH) in DCM) afforded the title compound (290 mg, 66% yield).

[M+H]⁺=388.4

### Ethyl 1-((1-ethylpiperidine-4-yl)methyl)-1H-benzo[d]imidazole-2-carboxylate

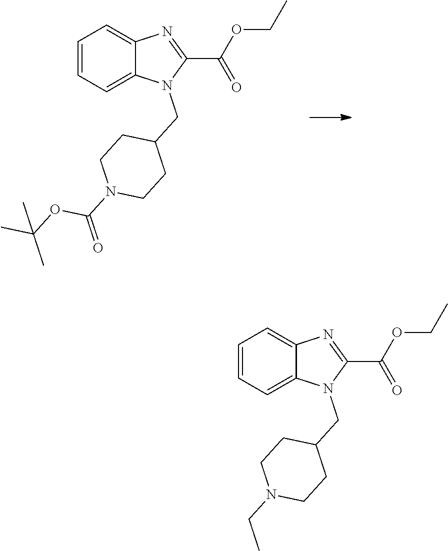 [SMILES: CCOC(=O)c1nc2ccccc2n1CC1CCN(C(=O)OC(C)(C)C)CC1 ; CCOC(=O)c1nc2ccccc2n1CC1CCN(CC)CC1]

Following general methods D (ii) and then F (ii), ethyl 1-((tert-butoxycarbonyl)piperidin-4-yl)methyl)-1H-benzo[d]imidazole-2-carboxylate (296 mg, 0.76 mmol) was converted to the title compound (139 mg, 59% yield).

[M+H]⁺=316.3

### Ethyl 1-((1-methylpiperidin-4-yl)methyl)-1H-indole-2-carboxylate

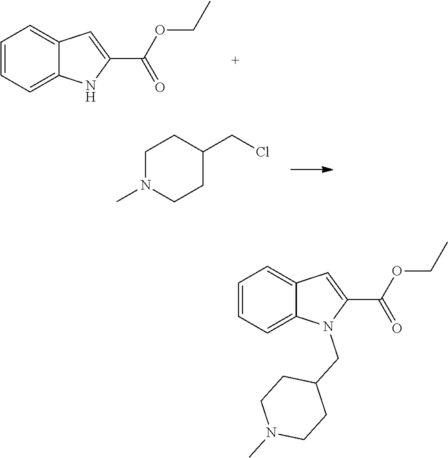

Following general method G (i), ethyl-1H-indole2-carboxylate (250 mg, 1.14 mmol) was reacted with 4-(chloromethyl)-1-methylpiperidinepiperidine (293 mg, 1.98 mmol) to afford the title compound (148 mg, 43% yield).

### Methyl 4-((1-methylpiperidin-4-yl)amino)benzo[b]thiophene-2-carboxylate

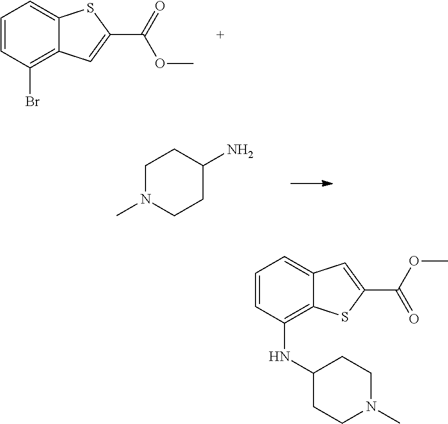

To a solution of methyl 4-bromobenzo[b]thiophene-2-carboxylate (300 mg, 1.11 mmol) in 1,4-dioxane (15 mL) was added 1-methylpiperidin-4-amine (0.14 mL, 1.11 mmol), BrettPhos Pd G3 (100 mg, 0.11 mmol) and sodium tert-butoxide (213 mg, 2.21 mmol). The reaction mixture was placed under N₂ and heated to 80° C. for 24 hrs. The reaction mixture was quenched with methanol (5 mL) and diluted with water (50 mL) and extracted into ethyl acetate (2×50 mL). The combined organic layers were washed with 1N HCl (50 mL). The acidified aqueous layer was washed with DCM (1×50 mL) and then basified to pH 10 with K₂CO₃.

The product was then extracted from the basic aqueous layer into ethyl acetate (2×50 mL), dried over Na₂SO₄, filtered and concentrated. Purification by flash chromatography (0-10% (0.7 M NH₃ in MeOH) in DCM)) afforded the title compound (61 mg, 17% yield) as a yellow solid.

[M+H]⁺=305.3

### Methyl 7-(((1-methylpiperidin-4-yl)methyl)amino)benzo[b]thiophene-2-carboxylate

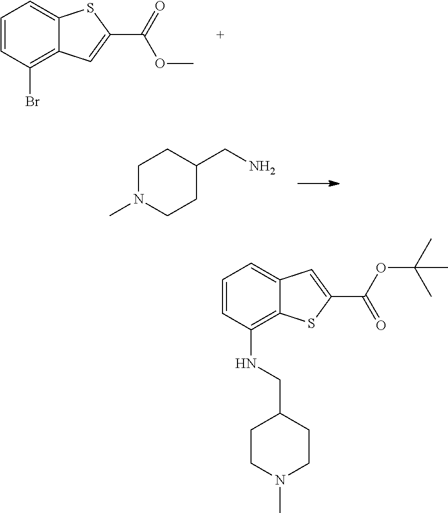

To a solution of methyl 4-bromobenzo[b]thiophene-2-carboxylate (300 mg, 1.11 mmol) in 1,4-dioxane (15 mL) was added (1-methylpiperidin-4-yl)methanamine (0.14 mL, 1.11 mmol), BrettPhos Pd G3 (100 mg, 0.11 mmol) and sodium tert-butoxide (213 mg, 2.21 mmol). The reaction mixture was placed under N₂ and heated to 80° C. for 24 hrs. The reaction mixture was quenched with methanol (5 mL) and diluted with water (50 mL) and extracted into ethyl acetate (2×50 mL). The combined organic layers were washed with 1N HCl (50 mL). The acidified aqueous layer was washed with DCM (1×50 mL) and then basified to pH 10 with K₂CO₃. The product was then extracted from the basic aqueous layer into ethyl acetate (2×50 mL), dried over Na₂SO₄, filtered and concentrated. The crude residue was purified by flash chromatography (0-10% (0.7 M NH₃ in MeOH) in DCM)) to afford the title compound (44 mg, 8% yield) as a yellow gum.

[M+H]⁺=361.5

### Synthesis of 2-(aminomethyl)thieno[3,2-c]pyridin-4-amine

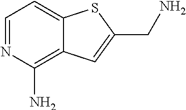 [SMILES: NCc1cc2c(N)nccc2s1]

### 4-Phenoxythieno[3,2-c]pyridine

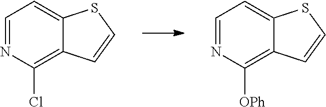 [SMILES: Clc1nccc2sccc12 ; c1ccc(Oc2nccc3sccc23)cc1]

A mixture of 4-chlorothieno[3,2-c]pyridine (10 g, 59.0 mmol) and phenol (36.6 g, 389 mmol) was warmed to 45° C. to form a homogeneous solution. KOH (5.6 g, 100 mmol) was added and the reaction heated to 140° C. for 18 hrs. The reaction mixture was cooled to 50° C. and diluted with 2N NaOH (250 mL), before being further cooled to rt and extracted with DCM (3×400 ml). The organic extract was washed with brine (100 ml), dried (MgSO₄), filtered and concentrated under vacuum to afford the title compound (13.25 g, 92% yield) as a dark brown crystalline solid.

[M+H]⁺=228.2

¹H NMR (500 MHz, DMSO-d6) δ 7.21-7.28 (m, 3H), 7.45 (dd, J=8.4, 7.3 Hz, 2H), 7.67 (d, J=5.5 Hz, 1H), 7.80 (d, J=5.6 Hz, 1H), 7.92 (dd, J=5.5, 4.3 Hz, 2H).

### Thieno[3,2-c]pyridin-4-amine

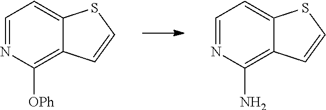 [SMILES: c1ccc(Oc2nccc3sccc23)cc1 ; Nc1nccc2sccc12]

4-phenoxythieno[3,2-c]pyridine (13.2 g, 58.1 mmol) and ammonium acetate (105 g, 1362 mmol) were mixed and heated to 150° C. After 72 hrs the reaction mixture was cooled to 50° C. and quenched with 2M NaOH (200 mL). The aqueous phase was then allowed to cool to room temperature and extracted with EtOAc (3×200 mL). The combined organic extracts were washed with brine (200 mL), dried (MgSO₄), filtered and concentrated in vacuo. The crude product was sonicated with 2M NaOH (100 mL). EtOAc (100 mL) was added and the organic layer separated. The aqueous layer was further extracted with EtOAc (3×100 mL). The combined organics were washed with brine (100 mL), dried (MgSO₄), filtered and concentrated in vacuo, to afford thieno[3,2-c]pyridin-4-amine (5.6 g, 63% yield) as a dark brown solid.

¹H NMR (500 MHz, DMSO-d6) δ 6.54 (s, 2H), 7.11-7.14 (m, 1H), 7.56 (d, J=5.5 Hz, 1H), 7.63-7.67 (m, 1H), 7.75 (d, J=5.7 Hz, 1H).

### N-(Thieno[3,2-c]pyridin-4-yl)benzamide

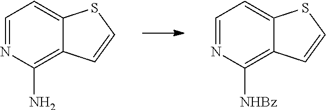

To a solution of thieno[3,2-c]pyridin-4-amine (5.6 g, 37.3 mmol) in pyridine (60 mL) was added benzoic anhydride (9.28 g, 41.0 mmol) at rt. The mixture was heated to 125° C. After 2 hrs the reaction was cooled to rt and concentrated in vacuo. The residue was partitioned between water (200 mL) and DCM (200 mL). The organic layer was separated and the aqueous layer was extracted with DCM (2×200 mL). The combined organics were washed with brine (100 mL), dried (MgSO₄), filtered and concentrated in vacuo. The residual oil was purified by flash chromatography (5% to 100% EtOAc in isohexane) to afford a thick yellow solid. The product was partitioned between DCM (100 mL) and Na₂CO₃ solution (aq., sat., 100 mL). The mixture was sonicated for 5 min and the layers separated. The aqueous layer was extracted with DCM (2×100 ml). The combined organic extracts were dried (Na₂SO₄) filtered and concentrated in vacuo to afford the title compound (6.62 g, 69% yield) as a yellow glass.

[M+H]⁺=255.2

### N-(2-Formylthieno[3,2-c]pyridin-4-yl)benzamide

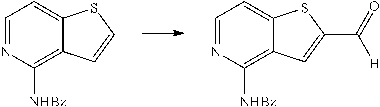

To a solution of N-(thieno[3,2-c]pyridin-4-yl)benzamide (6.6 g, 26.0 mmol) in THF (120 mL) at −78° C. was added LDA, 2M in THF/heptane/ethylbenzene (28.5 mL, 57.1 mmol) dropwise. After addition, the reaction mixture was stirred at −78° C. for 45 min. DMF (7 mL, 90 mmol) was added dropwise and the reaction warmed to rt and stirred for 18 hrs. The reaction was quenched with NH₄Cl (sat., aq., 100 ml). The aqueous layer was extracted with EtOAc (5×100 mL). The combined organic extracts were dried (Na₂SO₄), filtered and concentrated in vacuo. The crude product was purified by flash chromatography (5-100% THF in iso-hexane) to afford the title compound (4.62 g, 61% yield) as a pale yellow solid.

[M+H]⁺=283.2

### N-(2-(((2,4-Dimethoxybenzyl)amino)methyl)thieno[3,2-c]pyridin-4-yl)benzamide)

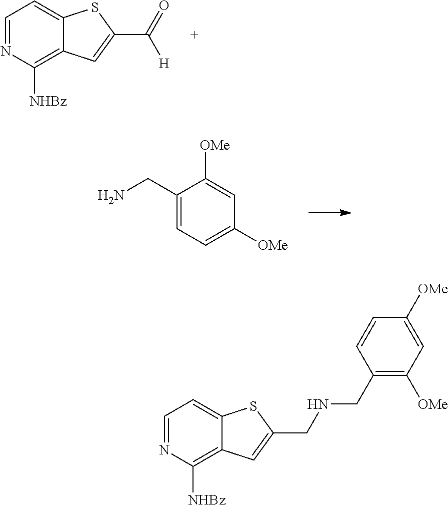

N-(2-formylthieno[3,2-c]pyridin-4-yl)benzamide (4.6 g, 16.29 mmol) and (2,4-dimethoxyphenyl)methanamine (3.27 g, 19.55 mmol) were mixed with AcOH (0.94 mL) and THF (110 ml). After 3 hrs, sodium triacetoxyborohydride (5.18 g, 24.44 mmol) was added. The reaction was stirred at rt for 3 hrs and then heated to 40° C. overnight. The reaction was quenched with NaHCO₃ (sat., aq., 100 ml). The organic layer was separated and the aqueous layer was extracted with EtOAc (3×100 ml). The combined organics were dried (Na₂SO₄), filtered and concentrated in vacuo. The residue was purified by flash chromatography (0-100% EtOAc in iso-hexane) to afford the title compound (3.9 g, 49% yield) as a pale yellow solid.

### 2-(Aminomethyl)thieno[3,2-c]pyridin-4-amine

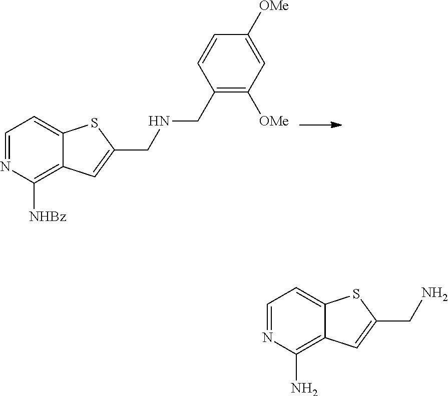

To a solution of N-(2-(((2,4-dimethoxybenzyl)amino)methyl)thieno[3,2-c]pyridin-4-yl)benzamide (650 mg, 1.5 mmol) in AcOH (6 mL) was added HCl (37 wt %, aq., 9 ml). The solution was heated to 100° C. in a sealed tube. The reaction was cooled to rt. The solvent and excess acid were removed in vacuo. The reaction mixture was partitioned in NaOH solution (aq., 2M, 150 mL) and EtOAc (150 ml). The aqueous phase was extracted with THF (200 mL×5). The combined organic extract was dried (Na₂SO₄), filtered and concentrated in vacuo to afford a dark red solid. The crude product was purified by reverse phase flash chromatography (0-50% MeCN in 10 mM ammonium bicarbonate) to afford the title compound (770 mg, 47% yield) as a pale red solid.

[M+H]⁺=180.2

1H NMR (500 MHz, DMSO-d6) δ 2.02 (s, 2H), 3.96 (d, J=1.3 Hz, 2H), 6.36 (s, 2H), 7.03 (d, J=5.7 Hz, 1H), 7.38-7.42 (m, 1H), 7.69 (d, J=5.6 Hz, 1H).

### Synthesis of tert-butyl (6-(aminomethyl)isoquinolin-1-yl)tert-butoxycarbonyl)carbamate

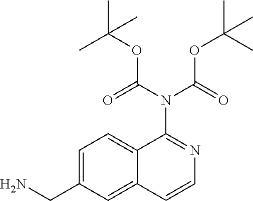 [SMILES: CC(C)(C)OC(=O)N(C(=O)OC(C)(C)C)c1nccc2cc(CN)ccc12]

### 2-Trimethylsilylethyl N-[(1-amino-6-isoquinolyl)methyl]carbamate

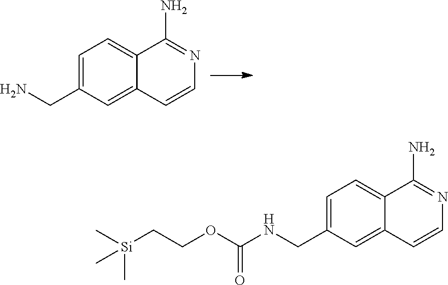 [SMILES: NCc1ccc2c(N)nccc2c1 ; C[Si](C)(C)CCOC(=O)NCc1ccc2c(N)nccc2c1]

6-(Aminomethyl)isoquinolin-1-amine dihydrochloride (synthesis described in WO2016083816, CAS 215454-95-8) (85 g, 345 mmol) was stirred in a mixture of water (0.446 L) and DMF (1.36 L). The reaction vessel was cooled in an ice-bath before the addition of triethylamine (87.4 g, 863 mmol) and (2,5-dioxopyrrolidin-1-yl) 2-trimethylsilylethyl carbonate (98.5 g, 380 mmol). The mixture was stirred at rt for 18 hrs. Solvents were removed under vacuum. The mixture was partitioned between EtOAc (450 mL), water (75 mL) and 2N NaOH (500 mL). The aqueous layer was extracted with further EtOAc (4×125 mL) and the combined organics washed with brine (100 mL), dried (Na₂SO₄), filtered and concentrated under vacuum. The residue was triturated with 2:1 Et₂O/Isohexane (375 mL) to afford the title compound (93.2 g, 82% yield) as a pale yellow powder.

[M+H]⁺=318.4

### tert-Butyl N-tert-butoxycarbonyl-N-[6-[(2-trimethylsilylethoxycarbonylamino)methyl]-1-isoquinolyl]carbamate

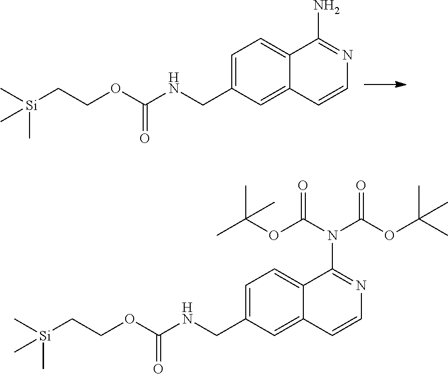 [SMILES: C[Si](C)(C)CCOC(=O)NCc1ccc2c(N)nccc2c1 ; CC(C)(C)OC(=O)N(C(=O)OC(C)(C)C)c1nccc2cc(CNC(=O)OCC[Si](C)(C)C)ccc12]

A mixture of di-tert-butyl dicarbonate (215 g, 986 mmol) and 2-trimethylsilylethyl N-[(1-amino-6-isoquinolyl)methyl]carbamate (31.3 g, 98.6 mmol) in anhydrous tert-butanol (283 mL) was heated at 66° C. for 48 hrs. Solvents were removed under vacuum. The crude material was purified by flash chromatography (0-50% EtOAc/Isohexane) to afford the title compound (33.9 g, 60% yield) as a sticky yellow gum.

[M+H]⁺=518.3

### tert-Butyl N-[6-(aminomethyl)-1-isoquinolyl]-N-tert-butoxycarbonyl-carbamate

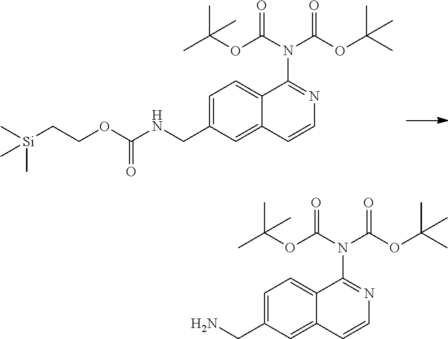 [SMILES: CC(C)(C)OC(=O)N(C(=O)OC(C)(C)C)c1nccc2cc(CNC(=O)OCC[Si](C)(C)C)ccc12 ; CC(C)(C)OC(=O)N(C(=O)OC(C)(C)C)c1nccc2cc(CN)ccc12]

A solution of tert-butyl N-tert-butoxycarbonyl-N-[6-[(2-trimethylsilylethoxycarbonylamino)methyl]-1-isoquinolyl]carbamate (31.9 g, 55.5 mmol) in THF (358 mL) was treated with tetra-n-butylammonium fluoride (185 mL, 185 mmol) and the mixture stirred at rt for 6 hrs. The residue was partitioned between EtOAc (1 L) and water (500 mL) containing brine (100 mL). The organic layer was washed with further water (150 mL) containing brine (50 mL). The aqueous was then extracted with further EtOAc (8×250 mL). The combined organics were dried (Na₂SO₄), filtered and concentrated. The residue was purified by flash chromatography (0 to 6% (1% NH₃ in MeOH) in DCM). The isolated solids were triturated with water (75 mL) for 3 hrs until a fine solid, then filtered and dried under vacuum in the presence of CaCl₂ to afford the title compound (12.9 g, 59% yield) as a yellow solid.

[M+H]⁺=374.2

### SPECIFIC EXAMPLES OF THE PRESENT INVENTION

### Example 2.11

### N-[(1-Aminoisoquinolin-6-yl)methyl]-4-chloro-5-({[(4-methanesulfonylphenyl)methyl]amino}methyl)thiophene-2-carboxamide

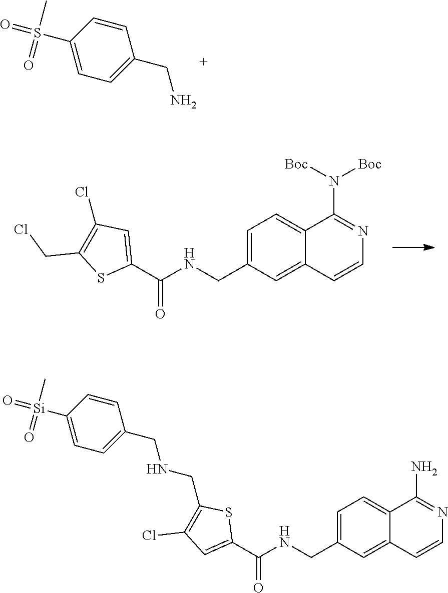

Following general methods C (i) and D, tert-butyl (tert-butoxycarbonyl) (6-((4-chloro-5-(chloromethyl)thiophene-2-carboxamido)methyl) isoquinolin-1-yl)carbamate (57 mg, 0.1 mmol) was reacted with (4-methanesulfonylphenyl)methanamine (0.4 mmol) to give the boc protected title compound which after treatment with TFA and purification by mass directed LCMS afforded the TFA salt of title compound (41 mg, 55% yield) as an off white solid.

[M+H]+=515.4

### Example 2.36

### N-[(1-Aminoisoquinolin-6-yl)methyl]-4-chloro-5-[({[1-(pyridin-4-yl)piperidin-4-yl]methyl}amino) methyl]thiophene-2-carboxamide

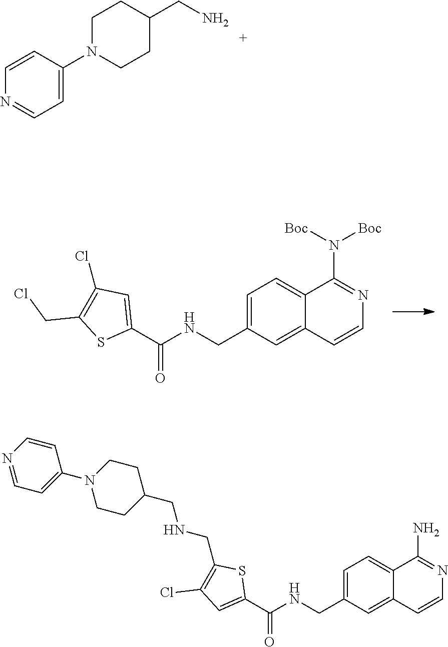 [SMILES: NCC1CCN(c2ccncc2)CC1 ; CC(C)(C)OC(=O)N(C(=O)OC(C)(C)C)c1nccc2cc(CNC(=O)c3cc(Cl)c(CCl)s3)ccc12 ; Nc1nccc2cc(CNC(=O)c3cc(Cl)c(CNCC4CCN(c5ccncc5)CC4)s3)ccc12]

Following general methods C (i) and D, tert-Butyl (tert-butoxycarbonyl) (6-((4-chloro-5-(chloromethyl)thiophene-2-carboxamido)methyl) isoquinolin-1-yl)carbamate (57 mg, 0.1 mmol) was reacted with [1-(pyridin-4-yl)piperidin-4-yl]methanamine (0.4 mmol) to give the boc protected title compound which after treatment with TFA and purification by mass directed LCMS afforded the TFA salt of title compound (43 mg, 49% yield) as an off white solid.

[M+H]⁺=521.6

### Example 5.18

### N-[(1-Amino-6-isoquinolyl)methyl]-4-chloro-5-[[(3R)-3-(3-pyridylamino)pyrrolidin-1-yl]methyl]thiophene-2-carboxamide

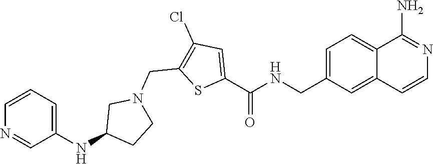 [SMILES: Nc1nccc2cc(CNC(=O)c3cc(Cl)c(CN4CC[C@@H](Nc5cccnc5)C4)s3)ccc12]

### tert-Butyl (3R)-3-(3-pyridylamino)pyrrolidine-1-carboxylate

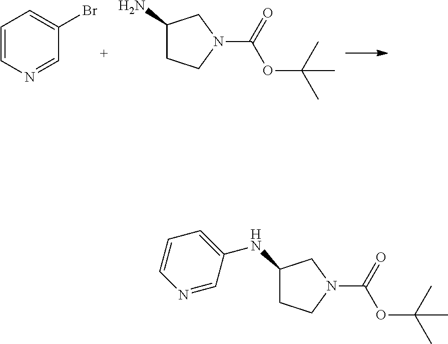 [SMILES: Brc1cccnc1 ; CC(C)(C)OC(=O)N1CC[C@@H](N)C1 ; CC(C)(C)OC(=O)N1CC[C@@H](Nc2cccnc2)C1]

To a stirred solution of 3-bromopyridine (120 μL, 1.25 mmol) in DMSO (1.5 mL) was added tert-butyl (3R)-3-aminopyrrolidine-1-carboxylate (300 μL, 1.77 mmol), caesium acetate (480 mg, 2.5 mmol) and copper (8 mg, 0.13 mmol) before degassing under N₂. The reaction was heated to 100° C. for 18 hrs before allowing to cool to rt. The reaction mixture was diluted with EtOAc (20 ml) and filtered through a silica gel plug, washing with EtOAc. The filtrate was washed with water (30 mL) and brine (20 mL) then dried via hydrophobic frit and concentrated in vacuo. The residue was purified by flash chromatography (50-100% EtOAc in hexane) to afford the title compound (112 mg, 34% yield) as a brown oil.

[M+H]⁺=264.1

### N-[(3R)-Pyrrolidin-3-yl]pyridin-3-amine

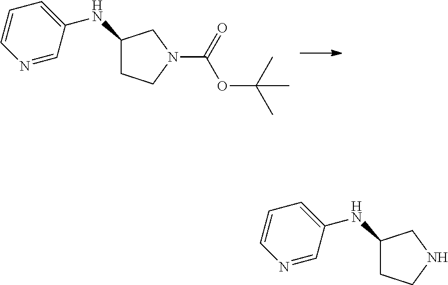 [SMILES: CC(C)(C)OC(=O)N1CC[C@@H](Nc2cccnc2)C1 ; c1cncc(N[C@@H]2CCNC2)c1]

Following general procedure D, tert-butyl (3R)-3-(3-pyridylamino)pyrrolidine-1-carboxylate (112 mg, 0.43 mmol) was deprotected to afford the title compound (quantitative yield) as a pale brown gum.

[M+H]⁺=164.1

### tert-Butyl (R)-(tert-butoxycarbonyl)(6-((4-chloro-5-((3-(pyridin-3-ylmethyl)pyrrolidin-1-yl)methyl) thiophene-2-carboxamido)methyl)isoquinolin-1-yl)carbamate

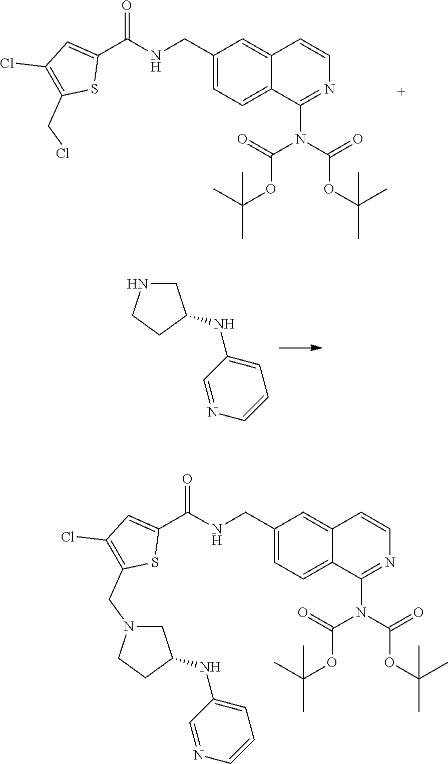 [SMILES: CC(C)(C)OC(=O)N(C(=O)OC(C)(C)C)c1nccc2cc(CNC(=O)c3cc(Cl)c(CCl)s3)ccc12 ; c1cncc(N[C@@H]2CCNC2)c1 ; CC(C)(C)OC(=O)N(C(=O)OC(C)(C)C)c1nccc2cc(CNC(=O)c3cc(Cl)c(CN4CC[C@@H](Nc5cccnc5)C4)s3)ccc12]

Following general procedure C (i), N-[(3R)-pyrrolidin-3-yl]pyridin-3-amine dihydrochloride (100 mg, 0.423 mmol) was alkylated to afford the title compound (62 mg, 42% yield) as a colourless oil.

[M+H]⁺=693.2

### N-[(1-Amino-6-isoquinolyl)methyl]-4-chloro-5-[[(3R)-3-(3-pyridylamino)pyrrolidin-1-yl]methyl]thiophene-2-carboxamide

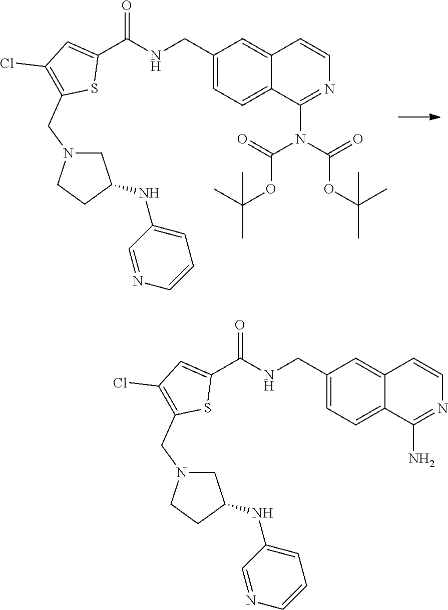 [SMILES: CC(C)(C)OC(=O)N(C(=O)OC(C)(C)C)c1nccc2cc(CNC(=O)c3cc(Cl)c(CN4CC[C@@H](Nc5cccnc5)C4)s3)ccc12 ; Nc1nccc2cc(CNC(=O)c3cc(Cl)c(CN4CC[C@@H](Nc5cccnc5)C4)s3)ccc12]

Following general procedure D, tert-butyl N-tert-butoxycarbonyl-N-[6-[[[4-chloro-5-[[(3R)-3-(3-pyridylamino)pyrrolidin-1-yl]methyl]thiophene-2-carbonyl]amino]methyl]-1-isoquinolyl]carbamate (62 mg, 0.089 mmol) was deprotected to afford the title compound (75 mg, quantitative yield) as an off-white solid.

[M+H]⁺=493.1

¹H NMR (MeOD, 500 MHz) δ 2.20 (1H, s), 2.70 (1H, d, J=20.8 Hz), 3.37-3.64 (2H, m), 3.70-3.99 (2H, m), 4.50 (1H, s), 4.81 (4H, s), 7.24 (1H, d, J=7.1 Hz), 7.58 (1H, d, J=7.0 Hz), 7.77-7.86 (4H, m), 7.87-7.91 (1H, m), 8.10 (1H, d, J=4.7 Hz), 8.18 (1H, d, J=2.5 Hz), 8.44 (1H, d, J=8.6 Hz).

### Example 5.19

### N-[1-Amino-6-isoquinolinyl)methyl]-4-chloro-5-(1,3-dihydropyrrolo[3,4-c]pyridine-2-ylmethyl)thiophene-2-carboxamidetrihydrochloride

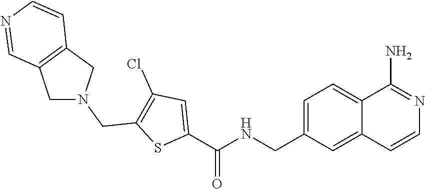 [SMILES: Nc1nccc2cc(CNC(=O)c3cc(Cl)c(CN4Cc5ccncc5C4)s3)ccc12]

### tert-Butyl N-tert-butoxycarbonyl-N-[6-[[[4-chloro-5-(1,3-dihydropyrrolo[3,4-c]pyridine-2-ylmethyl)thiophene-2-carbonyl]amino]methyl-1-isoquinolyl]carbamate

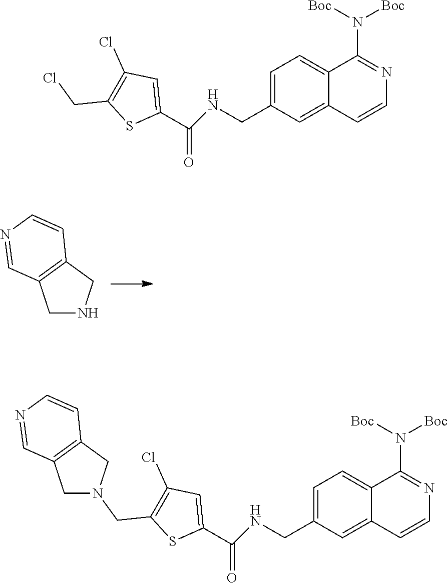 [SMILES: CC(C)(C)OC(=O)N(C(=O)OC(C)(C)C)c1nccc2cc(CNC(=O)c3cc(Cl)c(CCl)s3)ccc12 ; c1cc2c(cn1)CNC2 ; CC(C)(C)OC(=O)N(C(=O)OC(C)(C)C)c1nccc2cc(CNC(=O)c3cc(Cl)c(CN4Cc5ccncc5C4)s3)ccc12]

Following the general method C (i) tert-Butyl (tert-butoxycarbonyl) (6-((4-chloro-5-(chloromethyl)thiophene-2-carboxamido)methyl) isoquinolin-1-yl)carbamate was reacted with 2,3-dihydro-1H-pyrrolo[3,4-c]pyridine hydrochloride to afford the title product as light yellow solid (50 mg, 41% yield).

[M+H]⁺=650.2

### N-[1-Amino-6-isoquinolinyl)methyl]-4-chloro-5-(1,3-dihydropyrrolo[3,4-c]pyridine-2-ylmethyl)thiophene-2-carboxamide trihydrochloride

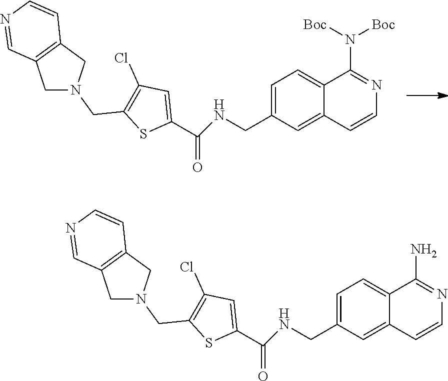 [SMILES: CC(C)(C)OC(=O)N(C(=O)OC(C)(C)C)c1nccc2cc(CNC(=O)c3cc(Cl)c(CN4Cc5ccncc5C4)s3)ccc12 ; Nc1nccc2cc(CNC(=O)c3cc(Cl)c(CN4Cc5ccncc5C4)s3)ccc12]

Boc group deprotection was carried out according to general method D (ii), using 4M HCl in Dioxane, to afford the title product as an off-white solid (49 mg, 100% yield).

[M+H]⁺=450.1

¹H NMR (MeOD, 500 MHz): 4.79 (2H, s), 4.83 (2H, s), 4.90 (2H, s), 4.95 (2H, s), 7.24 (1H, d, J=7.0 Hz), 7.58 (1H, d, J=7.0 Hz), 7.75-7.82 (2H, m), 7.89 (1H, s), 8.13 (1H, d, J=5.9 Hz), 8.44 (1H, d, J=8.7 Hz), 8.88 (1H, d, J=5.9 Hz), 8.92 (1H, s).

### Example 5.20

### N-[(1-Amino-6-isoquinolyl)methyl]-4-methyl-5-[[4-(4-pyridyl)piperazin-1-yl]methyl]thiophene-2-carboxamide

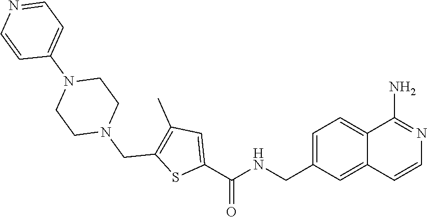 [SMILES: Cc1cc(C(=O)NCc2ccc3c(N)nccc3c2)sc1CN1CCN(c2ccncc2)CC1]

### 4-Methyl-5-[[4-(4-pyridyl)piperazin-1-yl]methyl]thiophene-2-carboxylic Acid

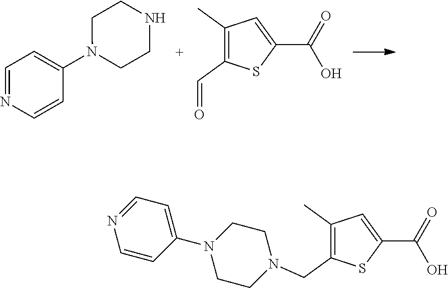 [SMILES: c1cc(N2CCNCC2)ccn1 ; Cc1cc(C(=O)O)sc1C=O ; Cc1cc(C(=O)O)sc1CN1CCN(c2ccncc2)CC1]

Following general procedure F, 5-formyl-4-methyl-thiophene-2-carboxylic acid (480 mg, 2.82 mmol) was reacted with 1-(4-pyridyl)piperazine (486 mg, 2.98 mmol) to afford the title compound (795 mg, 85% yield) as a white solid.

[M+H]⁺=318.2

¹H NMR (DMSO-d6, 500 MHz) δ 2.16 (3H, s), 2.52-2.58 (4H, m), 3.31-3.37 (4H, m), 3.66 (2H, s), 6.80-6.85 (2H, m), 7.43 (1H, s), 8.14-8.19 (2H, m)

### N-[(1-Amino-6-isoquinolyl)methyl]-4-methyl-5-[[4-(4-pyridyl)piperazin-1-yl]methyl]thiophene-2-carboxamide

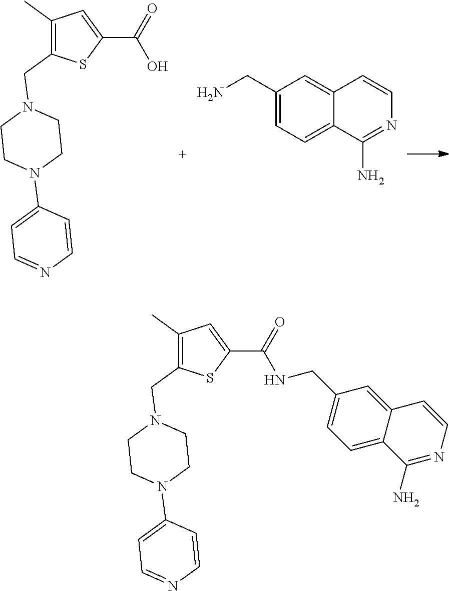 [SMILES: Cc1cc(C(=O)O)sc1CN1CCN(c2ccncc2)CC1 ; NCc1ccc2c(N)nccc2c1 ; Cc1cc(C(=O)NCc2ccc3c(N)nccc3c2)sc1CN1CCN(c2ccncc2)CC1]

Following general procedure A, 4-methyl-5-[[4-(4-pyridyl)piperazin-1-yl]methyl]thiophene-2-carboxylic acid (170 mg, 0.54 mmol) was reacted with 6-(aminomethyl)isoquinolin-1-amine dihydrochloride (133 mg, 0.54 mmol) to afford the title compound (190 mg, 73% yield) as an off white solid.

[M+H⁺]=473.3

¹H NMR (DMSO-d6, 500 MHz) δ (DMSO): 2.17 (3H, s), 2.55 (4H, t, J=5.1 Hz), 3.32 (4H, t, J=5.0 Hz), 3.66 (2H, s), 4.55 (2H, d, J=6.0 Hz), 6.72 (2H, s), 6.78-6.83 (2H, m), 6.87 (1H, d, J=5.8 Hz), 7.39 (1H, dd, J=8.6, 1.8 Hz), 7.56 (2H, d, J=9.2 Hz), 7.77 (1H, d, J=5.8 Hz), 8.12-8.18 (3H, m), 8.99 (1H, t, J=6.0 Hz).

### Example 5.25

### N-[(1-Amino-6-isoquinolyl)methyl]-4-methyl-5-[[3-(4-pyridyloxy)azetidin-1-yl]methyl]thiophene-2-carboxamide

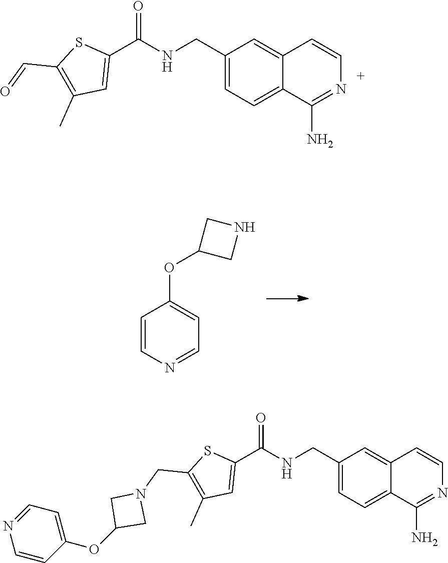 [SMILES: Cc1cc(C(=O)NCc2ccc3c(N)nccc3c2)sc1C=O ; c1cc(OC2CNC2)ccn1 ; Cc1cc(C(=O)NCc2ccc3c(N)nccc3c2)sc1CN1CC(Oc2ccncc2)C1]

Following general procedure F, 4-(azetidin-3-yloxy)pyridine (16.3 mg, 0.11 mmol) was reacted with N-[(1-amino-6-isoquinolyl)methyl]-5-formyl-4-methyl-thiophene-2-carboxamide (30 mg, 0.09 mmol), to afford the title compound (17 mg, 38% yield)

[M+H]⁺=460.0

¹H NMR (DMSO-d6, 500 MHz) δ: 2.14 (3H, s), 3.11-3.14 (2H, m), 3.77 (2H, s), 3.82 (2H, td, J=6.2, 1.8 Hz), 4.54 (2H, d, J=5.9 Hz), 4.95 (1H, t, J=5.6 Hz), 6.71 (2H, s), 6.84-6.89 (3H, m), 7.38 (1H, dd, J=8.7, 1.7 Hz), 7.53 (2H, d, J=6.7 Hz), 7.76 (1H, d, J=5.7 Hz), 8.13 (1H, d, J=8.6 Hz), 8.35-8.39 (2H, m), 8.96 (1H, t, J=6.0 Hz)

### Example 5.26

### N-((1-Aminoisoquinolin-6-yl)methyl)-4-methyl-5-((4-(pyrimidin-4-yl)piperazin-1-yl)methyl) thiophene-2-carboxamide

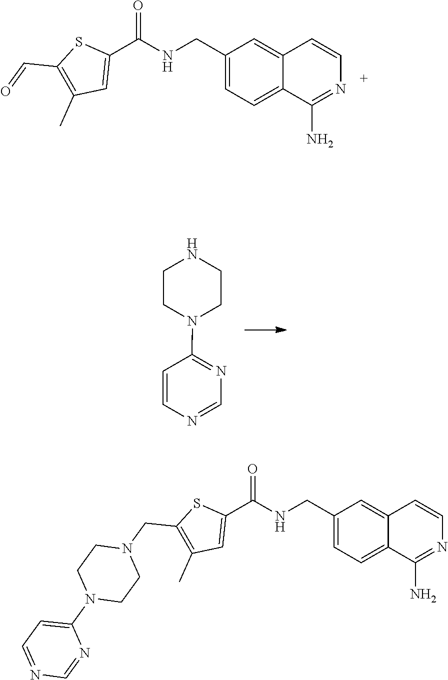 [SMILES: Cc1cc(C(=O)NCc2ccc3c(N)nccc3c2)sc1C=O ; c1cc(N2CCNCC2)ncn1 ; Cc1cc(C(=O)NCc2ccc3c(N)nccc3c2)sc1CN1CCN(c2ccncn2)CC1]

Following general procedure F (ii), 4-(piperazin-1-yl)pyrimidine (17.8 mg, 0.11 mmol) and N-[(1-amino-6-isoquinolyl)methyl]-5-formyl-4-methyl-thiophene-2-carboxamide (30 mg, 0.09 mmol) afforded the title compound (17 mg, 38% yield). [M+H]⁺=474.1

¹H NMR (DMSO-d6, 500 MHz) δ: 2.16 (3H, s), 2.51 (4H, t, J=5.3 Hz), 3.62 (4H, t, J=5.3 Hz), 3.65 (2H, s), 4.54 (2H, d, J=5.6 Hz), 6.70 (2H, d, J=4.9 Hz), 6.81 (1H, dd, J=6.3, 1.3 Hz), 6.86 (1H, d, J=5.8 Hz), 7.38 (1H, dd, J=8.6, 1.7 Hz), 7.55 (2H, d, J=8.1 Hz), 7.76 (1H, d, J=5.8 Hz), 8.13 (1H, d, J=8.6 Hz), 8.17 (1H, d, J=6.2 Hz), 8.46-8.50 (1H, m), 8.98 (1H, t, J=6.1 Hz)

### Example 7.03

### N-((1-Aminoisoquinolin-6-yl)methyl)-4-chloro-5-((4-(piperazin-1-yl)phenoxy)methyl)thiophene-2-carboxamide

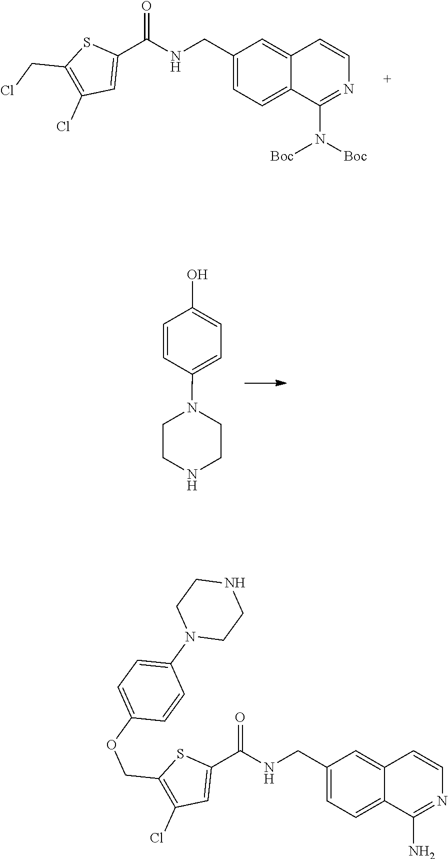 [SMILES: CC(C)(C)OC(=O)N(C(=O)OC(C)(C)C)c1nccc2cc(CNC(=O)c3cc(Cl)c(CCl)s3)ccc12 ; Oc1ccc(N2CCNCC2)cc1 ; Nc1nccc2cc(CNC(=O)c3cc(Cl)c(COc4ccc(N5CCNCC5)cc4)s3)ccc12]

Following general procedures B and D (i), 4-(piperazin-1-yl)phenol (35.6 mg, 0.2 mmol) and tert-butyl (tert-butoxycarbonyl)(6-((4-chloro-5-(chloromethyl)thiophene-2-carboxamido)methyl) isoquinolin-1-yl)carbamate (56.7 mg, 0.1 mmol) gave the boc protected title compound which after treatment with TFA afforded the TFA salt of the title compound (62.8 mg, 85% yield).

[M+H]⁺=508.6

### Example 25.15

### N-((1-Aminoisoquinolin-6-yl)methyl)-4-(2-(1-methylpiperidin-4-yl)ethyl)thiazole-2-carboxamide

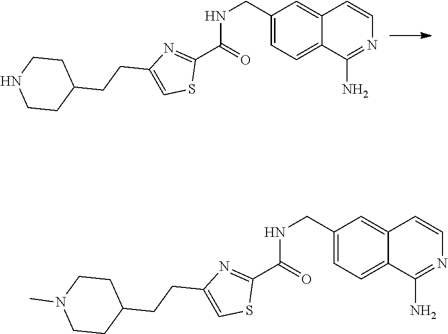 [SMILES: Nc1nccc2cc(CNC(=O)c3nc(CCC4CCNCC4)cs3)ccc12 ; CN1CCC(CCc2csc(C(=O)NCc3ccc4c(N)nccc4c3)n2)CC1]

Following general method F, N-((1-aminoisoquinolin-6-yl)methyl)-4-(2-(piperidin-4-yl)ethyl)thiazole-2-carboxamide (24 mg, 0.06 mmol) was reacted with paraformaldehyde (4 mg, 0.13 mmol) to afford the title compound (9 mg, 34% yield) as a colourless glass.

[M+H]⁺=410.2

¹H NMR (500 MHz, DMSO-d6) δ: 1.17-1.28 (3H, m), 1.59-1.66 (2H, m), 1.66-1.73 (2H, m), 1.92-2.04 (2H, m), 2.22 (3H, s), 2.76-2.87 (4H, m), 4.56-4.60 (2H, m), 6.73 (2H, s), 6.86 (1H, d, J=5.8 Hz), 7.42 (1H, dd, J=8.6, 1.7 Hz), 7.56 (1H, d, J=1.7 Hz), 7.64 (1H, s), 7.76 (1H, d, J=5.8 Hz), 8.14 (1H, d, J=8.6 Hz), 9.36 (1H, t, J=6.4 Hz).

### Example 25.101

### 4-Chloro-N-((4,6-dimethyl-1H-pyrrolo[2,3-b]pyridin-5-yl)methyl)-5-methylthiophene-2-carboxamide

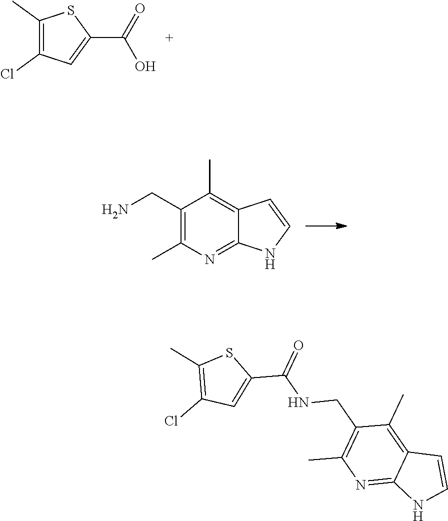

Following general method A (i), (4,6-dimethyl-1H-pyrrolo[2,3-b]pyridin-5-yl)methanamine (synthesis reported in a previous patent WO2014188211) (50 mg, 0.29 mmol) was reacted with 4-chloro-5-methylthiophene-2-carboxylic acid (50 mg, 0.28 mmol) which after purification by preparative HPLC (Waters, Basic (0.1% ammonium bicarbonate), 35-65% MeCN in Water) afforded the title compound (7 mg, 7% yield) as a beige solid.

[M+H]⁺=334.0

¹H NMR (500 MHz, DMSO-d6) δ 2.38 (s, 3H), 2.56 (s, 3H), 4.55 (d, J=4.7 Hz, 2H), 6.45 (dd, J=3.4, 1.5 Hz, 1H), 7.30 (dd, J=3.5, 2.1 Hz, 1H), 7.74 (d, J=1.4 Hz, 1H), 8.45 (t, J=4.7 Hz, 1H), 11.35 (s, 1H). Missing CH₃ under DMSO.

### Example 25.102

### N-(4-(Aminomethyl)-2,6-dimethylbenzyl)-4-chloro-5-methylthiophene-2-carboxamide

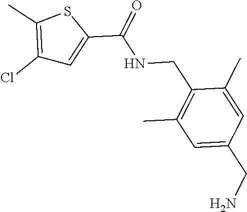 [SMILES: Cc1cc(CN)cc(C)c1CNC(=O)c1cc(Cl)c(C)s1]

### tert-Butyl 4-((4-chloro-5-methylthiophene-2-carboxamido)methyl)-3,5-dimethylbenzyl carbamate

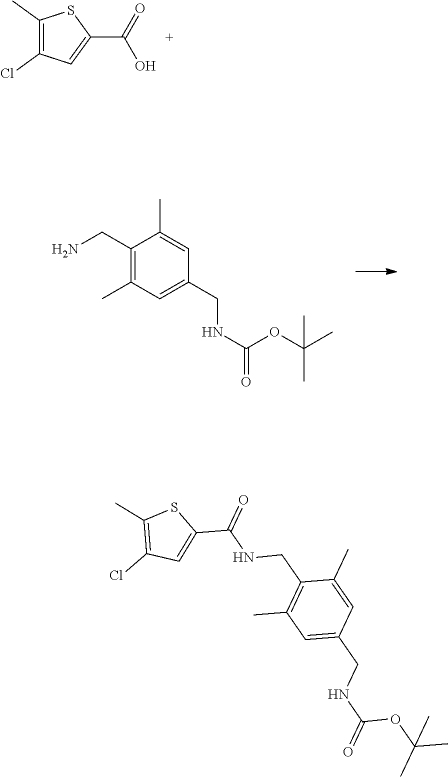

Following general method A (i), tert-butyl 4-(aminomethyl)-3,5-dimethylbenzylcarbamate (synthesis reported in WO2014108679, CAS 1618647-97-4) (75 mg, 0.28 mmol) was reacted with 4-chloro-5-methylthiophene-2-carboxylic acid (50 mg, 0.28 mmol) which after purification by flash chromatography (0-50% EtOAc/isohexane) afforded the title compound (49 mg, 39% yield) as a pale white solid.

[M+H]⁺=421.1

¹H NMR (500 MHz, DMSO-d6) δ 1.39 (s, 9H), 2.31 (s, 6H), 2.38 (s, 3H), 4.04 (d, J=6.2 Hz, 2H), 4.40 (d, J=4.7 Hz, 2H), 6.90 (s, 2H), 7.33 (t, J=6.2 Hz, 1H), 7.76 (s, 1H), 8.43 (t, J=4.6 Hz, 1H).

### N-(4-(Aminomethyl)-2,6-dimethylbenzyl)-4-chloro-5-methythiophene-2-carboxamide

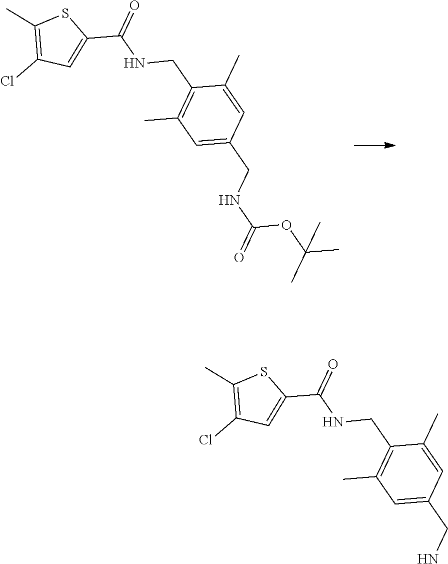

Following general method D (ii), tert-butyl 4-((4-chloro-5-methylthiophene-2-carboxamido)methyl)-3,5-dimethylbenzylcarbamate (45 mg, 0.106 mmol) was deprotected which after purification by preparative HPLC (Waters, Basic (0.1% ammonium bicarbonate), 20-50% MeCN in Water) afforded the title compound (22 mg, 62% yield) as a pale white solid.

[M+H]⁺=323.3

¹H NMR (500 MHz, DMSO-d6) δ: 2.31 (6H, s), 2.37 (3H, s), 3.62 (2H, s), 4.40 (2H, d, J=4.7 Hz), 6.98 (2H, s), 7.76 (1H, s), 8.42 (1H, t, J=4.8 Hz), NH₂ was not observed

### Example 25.103

### N-((4-aminothieno[3,2-c]pyridin-2-yl)methyl)-4-chloro-5-methylthiophene-2-carboxamide

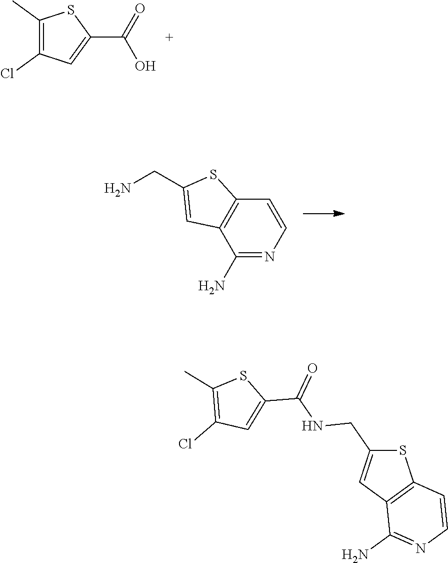

Following general method A (i), 2-(aminomethyl)thieno[3,2-c]pyridin-4-amine (26 mg, 0.145 mmol) was reacted with 4-chloro-5-methylthiophene-2-carboxylic acid (25 mg, 0.142 mmol) which after purification by prep HPLC (Waters, Basic (0.1% ammonium bicarbonate), 20-50% MeCN in Water) afforded the title compound (10.1 mg, 21% yield) as a white solid.

[M+H]⁺=338.2

¹H NMR (500 MHz, DMSO-d6) δ: 2.40 (3H, s), 4.64 (2H, d, J=6.1 Hz), 6.49 (2H, s), 7.03 (1H, d, J=5.7 Hz), 7.51 (1H, s), 7.69-7.75 (2H, m), 9.25 (1H, t, J=5.9 Hz).

### Example 25.104

### N-((1-Amino-5-fluoroisoquinolin-6-yl)methyl)-4-chloro-5-methylthiophene-2-carboxamide

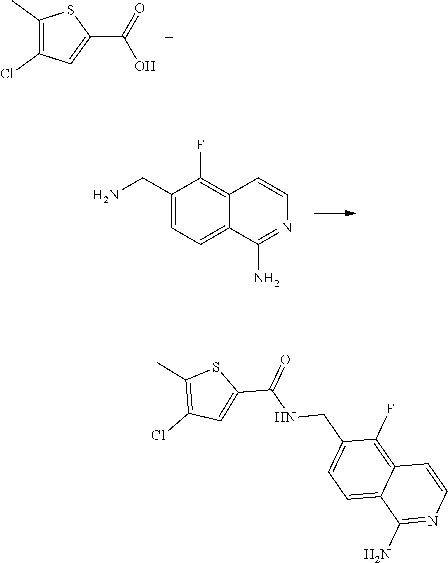

Following general method A (i), 6-(aminomethyl)-5-fluoroisoquinolin-1-amine dihydrochloride (synthesis reported in a previous patent WO2016083816) (67 mg, 0.254 mmol) was reacted with 4-chloro-5-methylthiophene-2-carboxylic acid (45 mg, 0.254 mmol) which after purification by prep HPLC (Waters, Basic (0.1% ammonium bicarbonate), 35-65% MeCN in Water) afforded the title compound (6.84 mg, 8% yield) as a white solid.

[M+H]⁺=350.3

¹H NMR (500 MHz, DMSO-d6) δ: 2.40 (3H, s), 4.62 (2H, d, J=5.7 Hz), 6.95 (2H, s), 6.97 (1H, dd, J=5.9, 0.9 Hz), 7.37-7.44 (1H, m), 7.75 (1H, s), 7.88 (1H, d, J=5.9 Hz), 8.00 (1H, d, J=8.6 Hz), 9.12 (1H, t, J=5.8 Hz).

### Example 25.105

### N-[(6-Amino-2,4-dimethylpyridin-3-yl)methyl]-4-chloro-5-methylthiophene-2-carboxamide

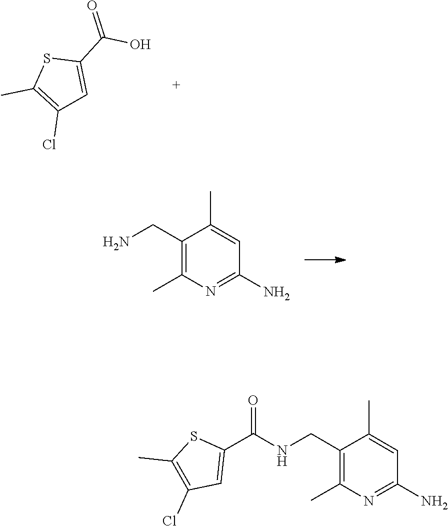

4-Chloro-5-methylthiophene-2-carboxylic acid (50 mg, 0.28 mmol) and 5-(aminomethyl)-4,6-dimethylpyridin-2-amine dihydrochloride (CAS 199296-47-4) (70 mg, 0.31 mmol) were dissolved in DCM (25 mL) and HOBt (52 mg, 0.34 mmol), triethylamine (198 μL, 1.42 mmol) and EDC (76 mg, 0.40 mmol) added and stirred at rt for 18 hrs. The reaction mixture was diluted with DCM (50 mL) and washed with water (25 mL) and brine (20 ml). The organic extracts were combined, dried over MgSO₄ filtered and concentrated in vacuo. The residue was purified by prep HPLC. (2-60% MeCN in (0.1% formic acid in water)) to afford the title compound (52 mg, 52%) as an off white solid.

[M+H]⁺=310.0

¹H NMR (DMSO, 400 MHz): 2.15 (3H, s), 2.29 (3H, s), 2.37 (3H, s), 4.29 (2H, d, J=4.7 Hz), 5.69 (2H, s), 6.12 (1H, s), 7.76 (1H, s), 8.20 (1H, s), 8.36 (1H, t, J=4.5 Hz)

### Example 25.203

### N-((1-Aminoisoquinolin-6-yl)methyl)-4-chlorobenzo[b]thiophene-2-carboxamide

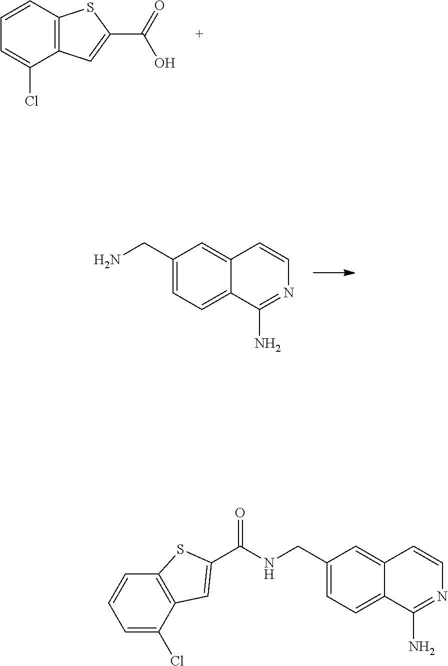 [SMILES: O=C(O)c1cc2c(Cl)cccc2s1 ; NCc1ccc2c(N)nccc2c1 ; Nc1nccc2cc(CNC(=O)c3cc4c(Cl)cccc4s3)ccc12]

4-chlorobenzo[b]thiophene-2-carboxylic acid (86 mg, 0.4 mmol) and 6-(aminomethyl)isoquinolin-1-amine (70.3 mg, 0.41 mmol) using general conditions A(i) afforded the title compound (68 mg, 44% yield) as yellow gum.

[M+H]⁺=368.3

¹H NMR (DMSO-d6, 500 MHz) δ:4.64 (2H, d, J=5.9 Hz), 6.75 (2H, s), 6.89 (1H, d, J=5.8 Hz), 7.45 (1H, dd, J=8.6, 1.8 Hz), 7.47-7.52 (1H, m), 7.56 (1H, dd, J=7.7, 0.9 Hz), 7.62 (1H, s), 7.78 (1H, d, J=5.8 Hz), 8.05 (1H, d, J=8.1 Hz), 8.17 (1H, d, J=8.6 Hz), 8.34 (1H, s), 9.60 (1H, t, J=6.0 Hz)

### Example 26.05

### N-((1-Aminoisoquinolin-6-yl)methyl)-5-methyl-3-(((1-methylpiperidin-4-yl)methyl)amino) thiophene-2-carboxamide

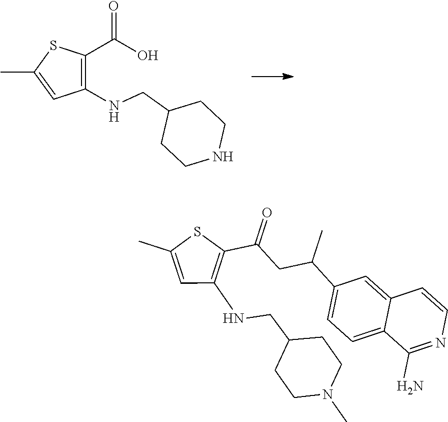 [SMILES: Cc1cc(NCC2CCNCC2)c(C(=O)O)s1 ; Cc1cc(NCC2CCN(C)CC2)c(C(=O)CC(C)c2ccc3c(N)nccc3c2)s1]

Following general procedure A (i), 5-methyl-3-(((1-methylpiperidin-4-yl)methyl)amino)thiophene-2-carboxylic acid (60 mg, 0.07 mmol) was reacted with and 6-(aminomethyl)isoquinolin-1-amine, 2HCl (80 mg, 0.33 mmol) to afford the title compound (5 mg, 14% yield) as a colourless glass.

[M+H]⁺=424.1

¹H NMR (500 MHz, DMSO-d6): 1.12-1.22 (1H, m), 1.32-1.44 (1H, m), 1.55-1.66 (2H, m), 1.74-1.83 (2H, m), 2.12 (2H, s), 2.39 (3H, d, J=1.0 Hz), 2.71-2.77 (2H, m), 3.05 (2H, t, J=6.5 Hz), 4.46 (2H, d, J=6.0 Hz), 6.60 (1H, d, J=1.2 Hz), 6.67-6.73 (2H, m), 6.84 (1H, d, J=5.8 Hz), 7.37 (1H, dd, J=8.6, 1.7 Hz), 7.45-7.51 (2H, m), 7.73-7.79 (1H, m), 7.93 (1H, t, J=6.0 Hz), 8.07-8.17 (1H, m).

### Example 26.10

### N-((1-Aminoisoquinolin-6-yl)methyl)-4-chloro-5-methyl-3-(((1-methylpiperidin-4-yl)methyl)amino) thiophene-2-carboxamide

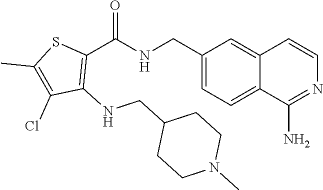 [SMILES: Cc1sc(C(=O)NCc2ccc3c(N)nccc3c2)c(NCC2CCN(C)CC2)c1Cl]

### Methyl methyl 4-chloro-5-methyl-3-(N-((1-methylpiperidin-4-yl)methyl)acetamido)thiophene-2-carboxylate

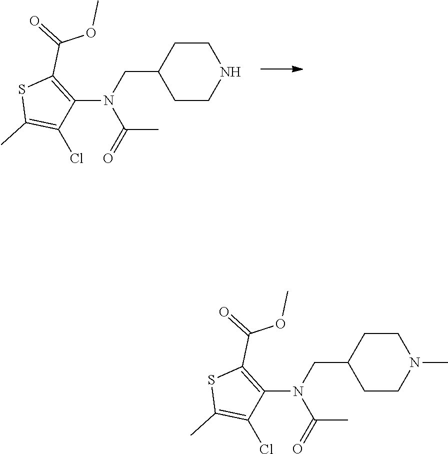

Following general procedure F (i), methyl 4-chloro-5-methyl-3-(N-(piperidin-4-ylmethyl)acetamido)thiophene-2-carboxylate (190 mg, 0.55 mmol) which after purification by SCX (eluting with 7M ammonia in MeOH) afforded the title compound (150 mg, 68% yield) as a pale yellow gum.

[M+H]⁺=359.6

¹H NMR (500 MHz, DMSO-d6) δ 1.06-1.27 (m, 3H), 1.54-1.67 (m, 2H), 1.70 (s, 4H), 1.71 (s, 1H), 2.10 (s, 3H), 2.68 (dd, J=10.8, 4.5 Hz, 2H), 3.26-3.33 (m, 3H), 3.57 (dd, J=13.8, 7.2 Hz, 1H), 3.80 (s, 3H), 5.76 (s, 1H).

### Methyl 4-chloro-5-methyl-3-(((1-methylpiperidin-4-yl)methyl)amino)thiophene-2-carboxylate

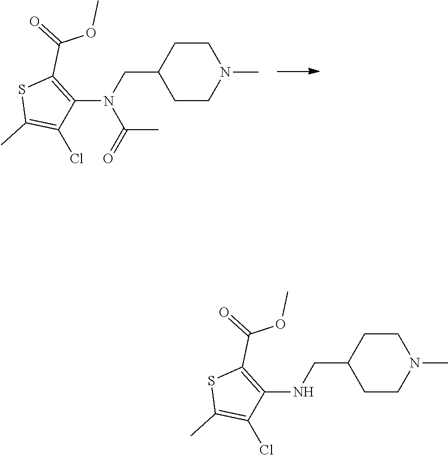

Following general procedure H, methyl methyl 4-chloro-5-methyl-3-(N-((1-methylpiperidin-4-yl)methyl)acetamido)thiophene-2-carboxylate (150 mg, 0.42 mmol) afforded the title compound (52 mg, 37% yield) as a pale yellow gum.

[M+H]⁺=317.2

¹H NMR (500 MHz, DMSO-d6) δ 1.19 (qd, J=12.1, 3.9 Hz, 2H), 1.40 (ddp, J=10.9, 6.9, 3.6, 3.2 Hz, 1H), 1.58-1.70 (m, 2H), 1.79 (td, J=11.6, 2.5 Hz, 2H), 2.13 (s, 3H), 2.34 (s, 3H), 2.74 (dt, J=11.9, 3.3 Hz, 2H), 3.46 (t, J=6.6 Hz, 2H), 3.74 (s, 3H), 6.86 (t, J=6.5 Hz, 1H).

### 4-Chloro-5-methyl-3-(((1-methylpiperidin-4-yl)methyl)amino)thiophene-2-carboxylic acid lithium salt

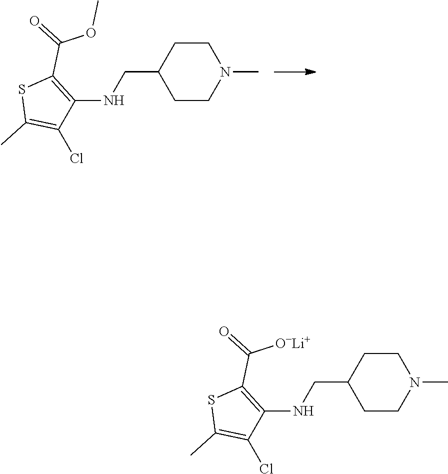

Following general procedure E, methyl 4-chloro-5-methyl-3-(((1-methylpiperidin-4-yl)methyl)amino)thiophene-2-carboxylate (52 mg, 0.16 mmol) was hydrolysed to afford the title compound (57 mg, quantitative yield).

[M+H]⁺=303.1/305.1

### N-((1-Aminoisoquinolin-6-yl)methyl)-4-chloro-5-methyl-3-(((1-methylpiperidin-4-yl)methyl)amino) thiophene-2-carboxamide

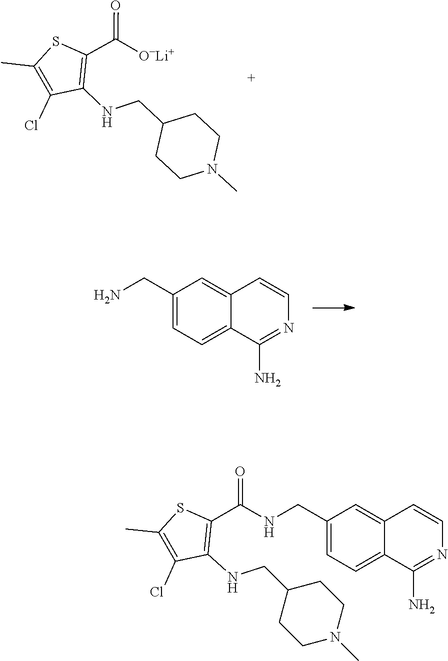

Following general procedure A (i), 4-Chloro-5-methyl-3-(((1-methylpiperidin-4-yl)methyl)amino)thiophene-2-carboxylic acid lithium salt (57 mg, 0.16 mmol) was reacted with and 6-(aminomethyl)isoquinolin-1-amine dihydrochloride (41 mg, 0.17 mmol) to afford the title compound (34 mg, 44% yield) as a colourless glass.

[M+H]⁺=458.3/460.4

¹H NMR (500 MHz, DMSO-d6) δ: 1.05-1.17 (2H, m), 1.24-1.34 (1H, m), 1.54-1.60 (2H, m), 1.67-1.75 (2H, m), 2.09 (3H, s), 2.36 (3H, s), 2.63-2.69 (2H, m), 3.24 (2H, t, J=6.7 Hz), 4.50 (2H, d, J=5.8 Hz), 6.71 (2H, s), 6.84 (1H, d, J=5.8 Hz), 7.17 (1H, t, J=6.7 Hz), 7.37 (1H, dd, J=8.7, 1.7 Hz), 7.50 (1H, d, J=1.7 Hz), 7.76 (1H, d, J=5.8 Hz), 8.13 (1H, d, J=8.6 Hz), 8.46 (1H, t, J=6.0 Hz).

### Example 26.16

### N-((1-Aminoisoquinolin-6-yl)methyl)-4-(((1-methylpiperidin-4-yl)methyl)amino)thiazole-5-carboxamide

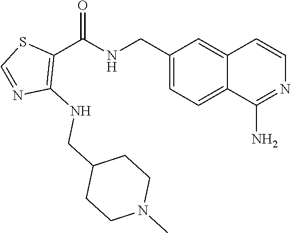 [SMILES: CN1CCC(CNc2ncsc2C(=O)NCc2ccc3c(N)nccc3c2)CC1]

### Methyl 4-(((1-(tert-butoxycarbonyl)piperidin-4-yl)methyl)amino)thiazole-5-carboxylate

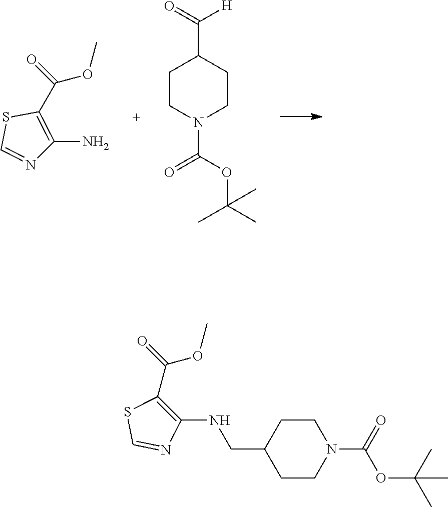

Following general method F (ii), tert-butyl 4-formylpiperidine-1-carboxylate (539 mg, 2.53 mmol) was reacted with methyl 4-aminothiazole-5-carboxylate (200 mg, 1.264 mmol) which after purification by flash chromatography (0-80% MeCN/10 mM ammonium bicarbonate) afforded the title compound (183 mg, 40% yield) as a white solid.

[M(−t-Bu)+H]⁺=300.1

¹H NMR (500 MHz, DMSO-d6) δ 0.97-1.08 (m, 2H), 1.39 (s, 9H), 1.59-1.64 (m, 1H), 1.67-1.80 (m, 2H), 2.58-2.74 (m, 2H), 3.40 (t, J=6.6 Hz, 2H), 3.72-3.76 (m, 3H), 3.86-3.98 (m, 2H), 7.08 (t, J=6.4 Hz, 1H), 9.02 (s, 1H).

### Methyl 4-((piperidin-4-ylmethyl)amino)thiazole-5-carboxylate

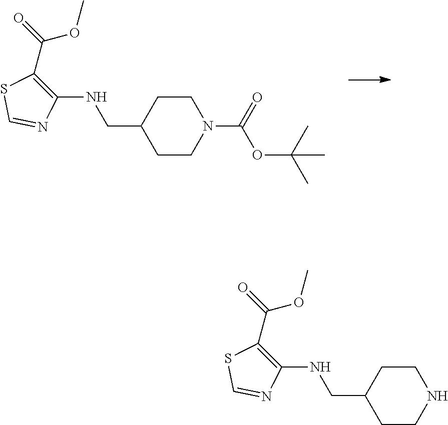 [SMILES: COC(=O)c1scnc1NCC1CCN(C(=O)OC(C)(C)C)CC1 ; COC(=O)c1scnc1NCC1CCNCC1]

Following general procedure D, methyl 4-(((1-(tert-butoxycarbonyl)piperidin-4-yl)methyl)amino)thiazole-5-carboxylate (183 mg, 0.52 mmol) afforded the title compound (130 mg, 94% yield) as a colourless oil.

[M+H]⁺=256.1

¹H NMR (500 MHz, DMSO-d6) δ 0.96-1.09 (m, 2H), 1.50-1.69 (m, 3H), 2.33-2.44 (m, 2H), 2.85-2.94 (m, 2H), 3.16-3.19 (m, 2H), 3.34-3.40 (m, 2H), 3.74 (s, 2H), 7.00-7.09 (m, 1H), 8.99-9.07 (m, 1H).

### Methyl 4-(((1-methylpiperidin-4-ylmethyl)amino)thiazole-5-carboxylate

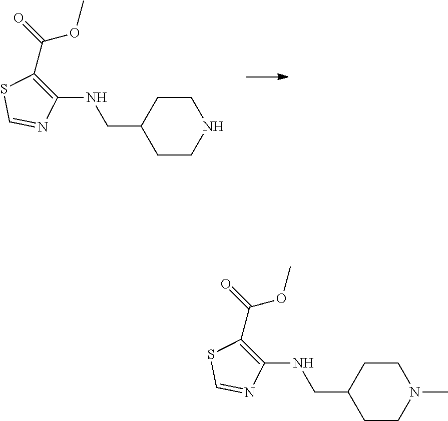 [SMILES: COC(=O)c1scnc1NCC1CCNCC1 ; COC(=O)c1scnc1NCC1CCN(C)CC1]

Following general procedure F (i), methyl 4-((piperidin-4-ylmethyl)amino)thiazole-5-carboxylate (130 mg, 0.51 mmol) afforded the title compound (108 mg, 75% yield) as a colourless oil.

[M+H]⁺=270.1

¹H NMR (500 MHz, DMSO-d6) δ 1.11-1.24 (m, 2H), 1.44-1.53 (m, 1H), 1.56-1.64 (m, 2H), 1.73-1.84 (m, 2H), 2.12 (s, 3H), 2.70-2.77 (m, 2H), 3.37-3.43 (m, 2H), 3.74 (s, 3H), 7.04 (t, J=6.3 Hz, 1H), 9.02 (s, 1H).

### 4-(((1-Methylpiperidin-4-ylmethyl)amino)thiazole-5-carboxylic Acid Lithium Salt

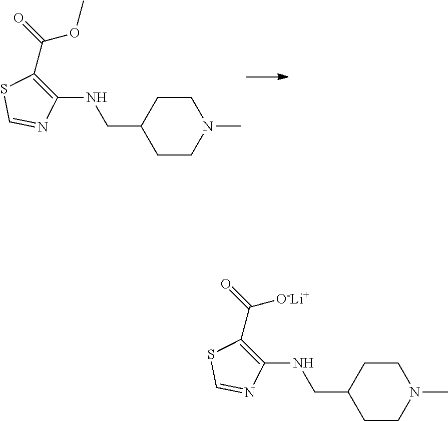

Following general method E, methyl 4-(((1-methylpiperidin-4-ylmethyl)amino)thiazole-5-carboxylate (108 mg, 0.40 mmol) was hydrolysed to afford the title compound (105 mg, quantitative yield).

[M+H]⁺=256.1

### N-((1-Aminoisoquinolin-6-yl)methyl)-4-(((1-methylpiperidin-4-yl)methyl)amino)thiazole-5-carboxamide

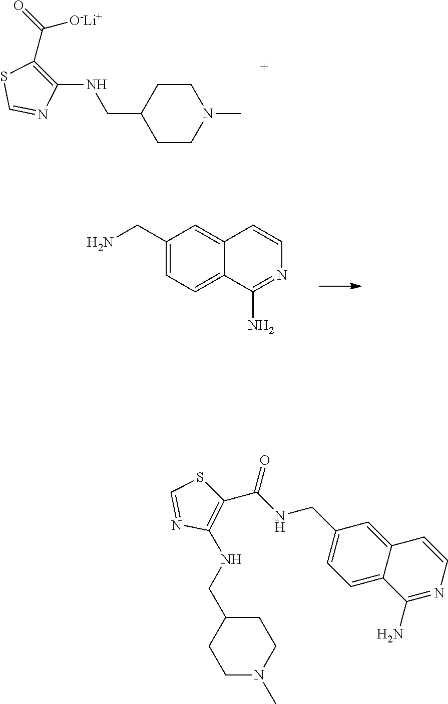

Following general method A, 4-(((1-methylpiperidin-4-ylmethyl)amino)thiazole-5-carboxylic acid lithium salt (105 mg, 0.40 mmol) was reacted with 6-(aminomethyl)isoquinolin-1-amine, 2HCl (100 mg, 0.406 mmol) to afford the title compound (43 mg, 95% yield).

[M+H]⁺=411.1

¹H NMR (500 MHz, DMSO-d6) δ: 1.13-1.26 (2H, m), 1.40-1.51 (1H, m), 1.57-1.65 (2H, m), 1.74-1.84 (2H, m), 2.13 (3H, s), 2.71-2.77 (2H, m), 3.33-3.36 (2H, m), 4.48-4.53 (2H, m), 6.67-6.73 (2H, m), 6.83-6.87 (1H, m), 7.36-7.40 (1H, m), 7.50-7.55 (2H, m), 7.75-7.78 (1H, m), 8.11-8.15 (1H, m), 8.39 (1H, t, J=6.0 Hz), 8.90 (1H, s).

### Example 35.04

### N-((1-Aminoisoquinolin-6-yl)methyl)-3-chloro-1-(2-(1-methylpiperidin-4-yl)ethyl)-1H-pyrazole-5-carboxamide

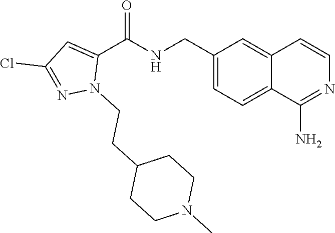 [SMILES: CN1CCC(CCn2nc(Cl)cc2C(=O)NCc2ccc3c(N)nccc3c2)CC1]

### Methyl 3-chloro-1-(2-(piperidin-4-yl)ethyl)-1H-pyrazole-5-carboxylate

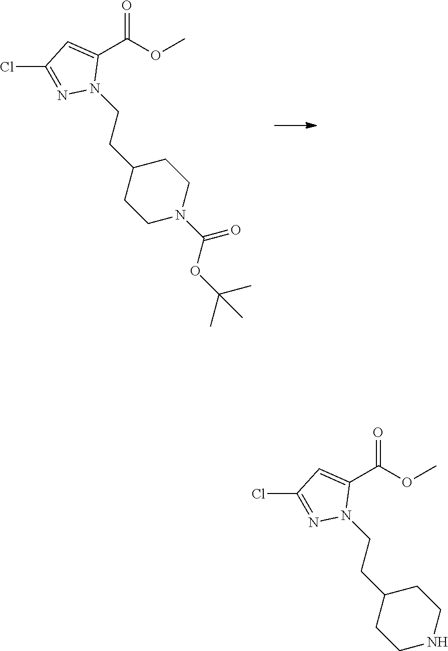 [SMILES: COC(=O)c1cc(Cl)nn1CCC1CCN(C(=O)OC(C)(C)C)CC1 ; COC(=O)c1cc(Cl)nn1CCC1CCNCC1]

Following general method D, tert-butyl 4-(2-(3-chloro-5-(methoxycarbonyl)-1H-pyrazol-1-yl)ethyl)piperidine-1-carboxylate (617 mg, 1.66 mmol) was reacted with 4M HCl in Dioxane to afford the title compound (451 mg, 100% yield) as colourless gum.

[M+H]⁺=272.1/274.1

### Methyl 3-chloro-1-(2-(1-methylpiperidin-4-yl)ethyl)-1H-pyrazole-5-carboxylate

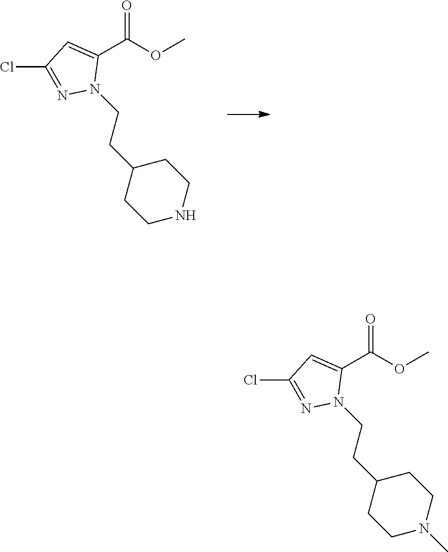 [SMILES: COC(=O)c1cc(Cl)nn1CCC1CCNCC1 ; COC(=O)c1cc(Cl)nn1CCC1CCN(C)CC1]

Polymer supported cyanoborohydride 2 mmol/g (3.32 g, 6.64 mmol) was added to a solution of methyl 3-chloro-1-(2-(piperidin-4-yl)ethyl)-1H-pyrazole-5-carboxylate (451 mg, 1.66 mmol), Formaldehyde aq 37% and acetic acid (47 μL, 0.830 mmol) in MeOH (2 mL). The mixture was stirred for 4 hrs then filtered and the filtrate concentrated in vacuo. The residue was purified by flash chromatography (0-100% (10% NH₃ in MeOH) in DCM) to afford title compound (390 mg, 82% yield) as colourless oil.

[M+H]⁺=286.4/288.0

### 3-Chloro-1-(2-(1-methylpiperidin-4-yl)ethyl)-1H-pyrazole-5-carboxylic Acid

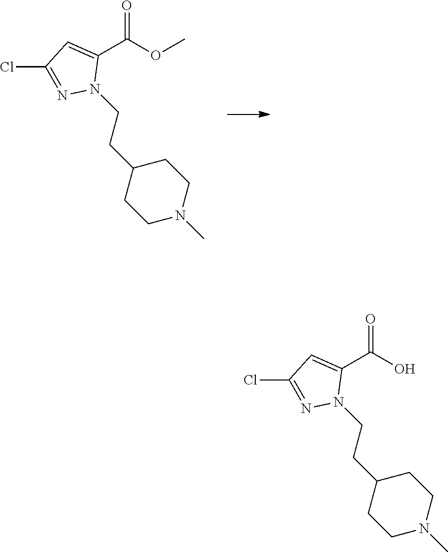 [SMILES: COC(=O)c1cc(Cl)nn1CCC1CCN(C)CC1 ; CN1CCC(CCn2nc(Cl)cc2C(=O)O)CC1]

Following general method (E), methyl 3-chloro-1-(2-(1-methylpiperidin-4-yl)ethyl)-1H-pyrazole-5-carboxylate (180 mg, 0.63 mmol) was hydrolysed to afford the title compound (273 mg, quantitative yield) as a colourless glass.

[M+H]⁺=272.1

### N-((1-Aminoisoquinolin-6-yl)methyl)-3-chloro-1-(2-(1-methylpiperidin-4-yl)ethyl)-1H-pyrazole-5-carboxamide

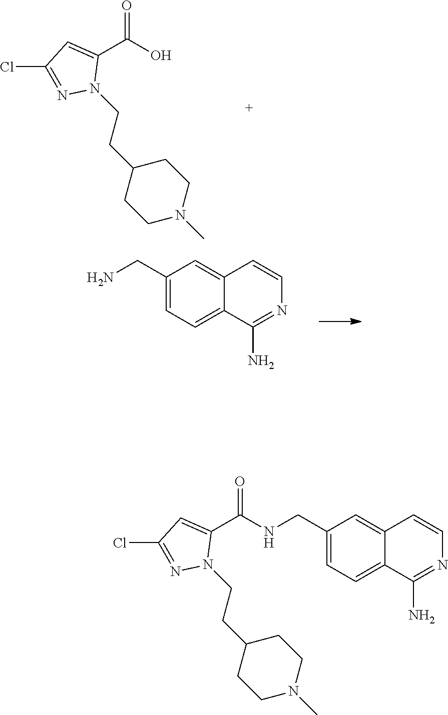

Following general method A (i), 3-chloro-1-(2-(1-methylpiperidin-4-yl)ethyl)-1H-pyrazole-5-carboxylic acid (120 mg, 0.27 mmol) was reacted with 6-(aminomethyl)isoquinolin-1-amine dihydrochloride (65 mg, 0.27 mmol) to afford the title compound (83 mg, 73% yield) as a colourless glass.

[M+H]⁺=427.1

¹H NMR (400 MHz, DMSO): 1.16-1.02 (3H, m), 1.72-1.51 (6H, m), 2.06-2.05 (3H, m), 2.66-2.60 (2H, m), 4.48 (2H, t, J=7.3 Hz), 4.56 (2H, d, J=5.9 Hz), 6.74-6.71 (2H, m), 6.86 (1H, d, J=5.7 Hz), 6.94 (1H, s), 7.39 (1H, dd, J=1.7, 8.6 Hz), 7.55 (1H, s), 7.77 (1H, d, J=5.8 Hz), 8.16-8.13 (1H, m), 9.22 (1H, t, J=5.9 Hz).

### Example 46.01

### N-((4-aminothieno[3,2-c]pyridin-2-yl)methyl)-1-(2-(1-methylpiperidin-4-yl)ethyl)-1H-pyrazole-5-carboxamide

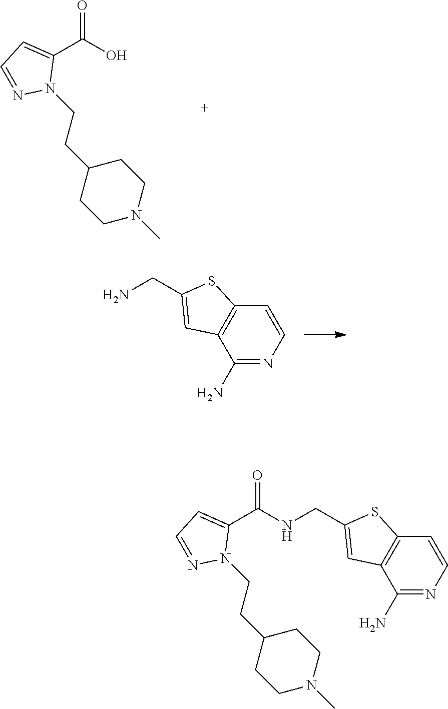

Following general method A (i), lithium 1-(2-(1-methylpiperidin-4-yl)ethyl)-1H-pyrazole-5-carboxylate (47 mg, 0.20 mmol) was reacted with 2-(aminomethyl)thieno[3,2-c]pyridin-4-amine (36 mg, 0.20 mmol) to afford the title compound (25 mg, 31% yield) as a beige glass.

[M+H]⁺=399.4

¹H NMR (500 MHz, DMSO-d6) δ: 1.02-1.17 (3H, m), 1.54-1.60 (2H, m), 1.60-1.73 (4H, m), 2.06 (3H, s), 2.61-2.67 (2H, m), 4.54 (2H, t, J=7.2 Hz), 4.63 (2H, d, J=5.6 Hz), 6.49 (2H, s), 6.86 (1H, d, J=2.1 Hz), 7.02 (1H, dd, J=5.6, 0.8 Hz), 7.49 (1H, d, J=2.0 Hz), 7.51 (1H, d, J=1.0 Hz), 7.71 (1H, d, J=5.6 Hz), 9.20 (1H, t, J=6.0 Hz).

### Example 51.02

### Ethyl 5-formyl-4-methyl-1H-pyrrole-2-carboxylate

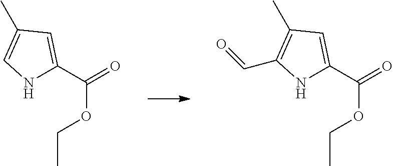

To an ice-cooled solution of DMF (0.51 mL, 6.59 mmol) in anhydrous DCM (20 mL) phosphorus oxychloride (0.61 mL, 6.54 mmol) was added. The mixture was warmed to rt and stirred for 30 min, then re-cooled in an ice bath and treated with ethyl 4-methyl-1H-pyrrole-2-carboxylate (0.5 g, 3.26 mmol) portion wise. The mixture was subsequently warmed to 40° C. for 4 hrs, then quenched by the slow addition of 2M NaOH (10 mL). The mixture was stirred for 30 min (still at acidic pH), then the organic layer collected. The aqueous was extracted with further DCM (2×20 mL) and the combined organic layers washed with brine (20 mL), dried over Na₂SO₄, filtered and concentrated in vacuo to afford title product (539 mg, 82% yield) as a pink oil which crystallized on standing.

[M+H]⁺=182.1

¹HNMR (DMSO-d6, 500 MHz) δ: 1.29 (3H, t, J=7.1 Hz), 2.29 (3H, s), 4.28 (2H, q, J=7.1 Hz), 6.70 (1H, s), 9.79 (1H, s), 12.70 (1H, s)

### Ethyl 1-ethyl-5-formyl-4-methyl-1H-pyrrole-2-carboxylate

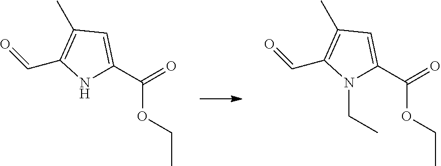

A solution of ethyl 5-formyl-4-methyl-1H-pyrrole-2-carboxylate (535 mg, 2.95 mmol) and iodoethane (0.47 mL, 5.90 mmol) in anhydrous DMF (6 mL) was cooled in an ice bath under nitrogen atmosphere, then treated portion wise with 60% sodium hydride (236 mg, 5.9 mmol). The cooling bath was removed, and the mixture allowed to warm to rt and stirred for 3 days. The mixture was quenched with water (30 mL) and acidified to pH 4 with 1M HCl. The aqueous layer was extracted with EtOAc (2×30 ml). The combined organic layers were washed with brine (2×20 ml), dried (MgSO₄), filtered and concentrated in vacuo. The residue was purified by flash chromatography (0 to 30% EtOAc in Isohexane) to afford title compound (113 mg, 18% yield) as a colourless oil.

[M+H]⁺=210.2

¹HNMR (DMSO-d6, 500 MHz) δ: 1.20-1.33 (6H, m), 2.31 (3H, s), 4.27 (2H, q, J=7.1 Hz), 4.68 (2H, q, J=7.0 Hz), 6.74 (1H, s), 9.89 (1H, s).

### Ethyl 1-ethyl-4-methyl-5-((4-(pyridin-4-yl)piperazin-1-yl)methyl)-1H-pyrrole-2-carboxylate

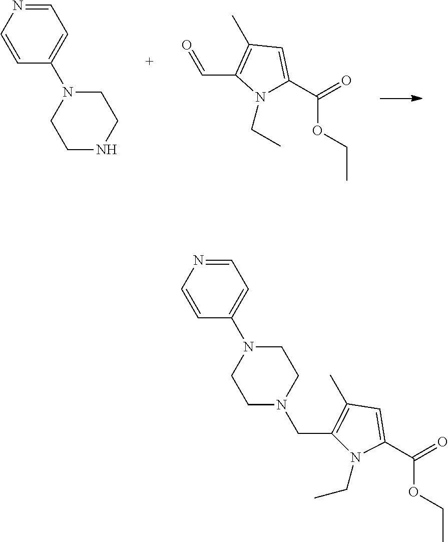 [SMILES: c1cc(N2CCNCC2)ccn1 ; CCOC(=O)c1cc(C)c(C=O)n1CC ; CCOC(=O)c1cc(C)c(CN2CCN(c3ccncc3)CC2)n1CC]

Following general method F, ethyl 1-ethyl-5-formyl-4-methyl-pyrrole-2-carboxylate (110 mg, 0.526 mmol) was reacted with 1-(4-pyridyl)piperazine (103 mg, 0.631 mmol) to afford title compound (49 mg, 24% yield) as a colourless gum.

[M+H]⁺=357.3

### 1-Ethyl-4-methyl-5-[[4-(4-pyridyl)piperazin-1-yl]methyl]pyrrole-2-carboxylic Acid

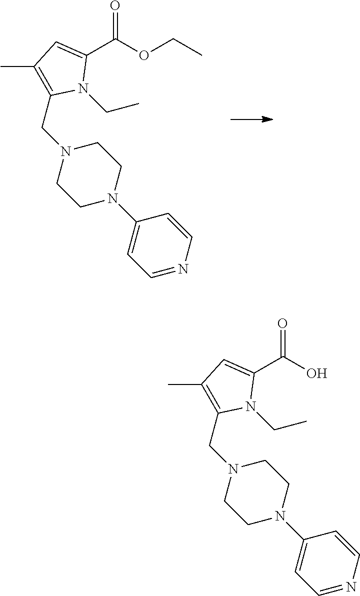 [SMILES: CCOC(=O)c1cc(C)c(CN2CCN(c3ccncc3)CC2)n1CC ; CCn1c(C(=O)O)cc(C)c1CN1CCN(c2ccncc2)CC1]

Following general method (E), ethyl 1-ethyl-4-methyl-5-[[4-(4-pyridyl)piperazin-1-yl]methyl]pyrrole-2-carboxylate (45 mg, 0.13 mmol) was hydrolysed to afford the title compound (32 mg, 71% yield) as a white solid.

[M+H]⁺=329.3

¹H NMR (DMSO-d6, 500 MHz) δ: 1.25 (3H, t, J=6.9 Hz), 1.99 (3H, s), 2.46 (4H, t, J=5.0 Hz), 3.27 (4H, t, J=5.1 Hz), 3.45 (2H, s), 4.34 (2H, q, J=6.9 Hz), 6.63 (1H, s), 6.74-6.83 (2H, in), 8.06-8.19 (2H, in). Acid proton visible but extremely broad at ^{˜}11.5 ppm

### N-[(1-Amino-6-isoquinolyl)methyl]-1-ethyl-4-methyl-5-[[4-(4-pyridyl)piperazin-1-yl]methyl]pyrrole-2-carboxamide

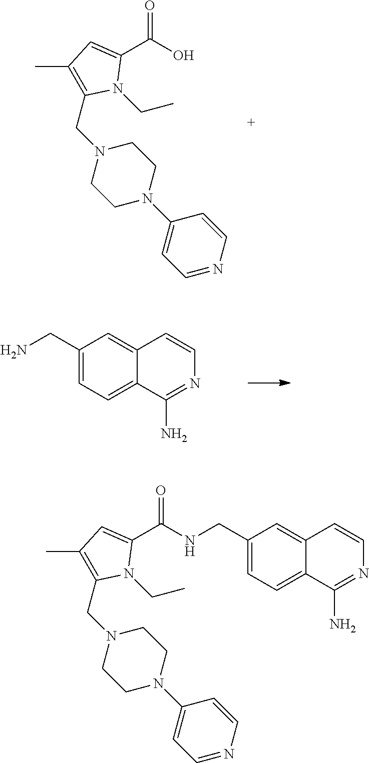 [SMILES: CCn1c(C(=O)O)cc(C)c1CN1CCN(c2ccncc2)CC1 ; NCc1ccc2c(N)nccc2c1 ; CCn1c(C(=O)NCc2ccc3c(N)nccc3c2)cc(C)c1CN1CCN(c2ccncc2)CC1]

Following general method (A (i)), 1-ethyl-4-methyl-5-[[4-(4-pyridyl)piperazin-1-yl]methyl]pyrrole-2-carboxylic acid (30 mg, 0.09 mmol) was reacted with 6-(aminomethyl)isoquinolin-1-amine dihydrochloride (25 mg, 0.10 mmol) to afford the title compound (28 mg, 62% yield) as an off white solid.

[M+H]⁺=484.4

¹H NMR (DMSO-d6, 500 MHz) δ: 1.23 (3H, t, J=6.9 Hz), 2.00 (3H, s), 2.42-2.49 (4H, m), 3.24-3.31 (4H, m), 3.44 (2H, s), 4.37 (2H, q, J=6.9 Hz), 4.51 (2H, d, J=6.1 Hz), 6.67 (1H, s), 6.71 (2H, d, J=5.2 Hz), 6.76-6.82 (2H, m), 6.85 (1H, d, J=5.8 Hz), 7.38 (1H, dd, J=8.6, 1.8 Hz), 7.52 (1H, s), 7.75 (1H, d, J=5.8 Hz), 8.07-8.21 (3H, m), 8.52 (1H, t, J=6.1 Hz)

### Example 51.05

### N-((1-Aminoisoquinolin-6-yl)methyl)-5-chloro-1-(2-(1-methylpiperidin-4-yl)ethyl)-1H-pyrazole-3-carboxamide

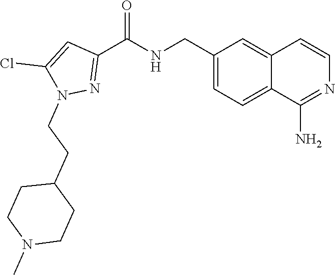 [SMILES: CN1CCC(CCn2nc(C(=O)NCc3ccc4c(N)nccc4c3)cc2Cl)CC1]

### Methyl 5-chloro-1-(2-(piperidin-4-yl)ethyl)-1H-pyrazole-3-carboxylate

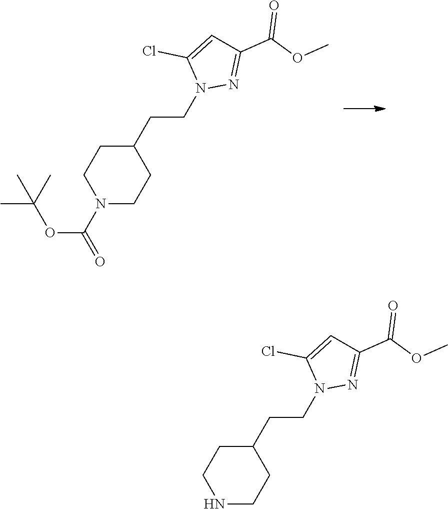 [SMILES: COC(=O)c1cc(Cl)n(CCC2CCN(C(=O)OC(C)(C)C)CC2)n1 ; COC(=O)c1cc(Cl)n(CCC2CCNCC2)n1]

Following general method D, tert-butyl 4-(2-(5-chloro-3-(methoxycarbonyl)-1H-pyrazol-1-yl)ethyl)piperidine-1-carboxylate (617 mg, 1.66 mmol) was reacted with TFA to afford the title compound (234 mg 41% yield).

[M+H]⁺=272.1

### Methyl 5-chloro-1-(2-(1-methylpiperidin-4-yl)ethyl)-1H-pyrazole-3-carboxylate

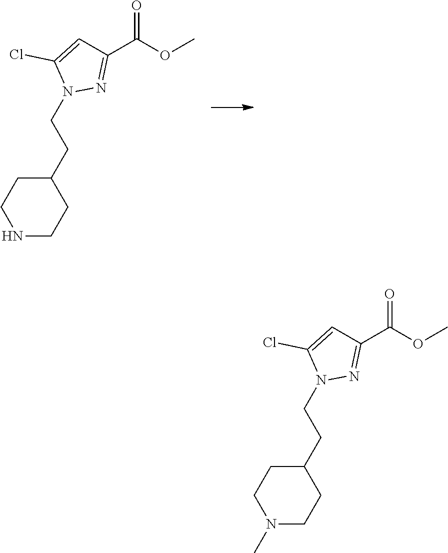 [SMILES: COC(=O)c1cc(Cl)n(CCC2CCNCC2)n1 ; COC(=O)c1cc(Cl)n(CCC2CCN(C)CC2)n1]

Following general method (F), methyl 5-chloro-1-(2-(piperidin-4-yl)ethyl)-1H-pyrazole-3-carboxylate (234 mg, 0.39 mmol) was reacted to afford the title compound (68 mg, 22% yield) as a brown solid.

[M+H]⁺=286.0

### Lithium 5-chloro-1-(2-(1-methylpiperidin-4-yl)ethyl)-1H-pyrazole-3-carboxylate

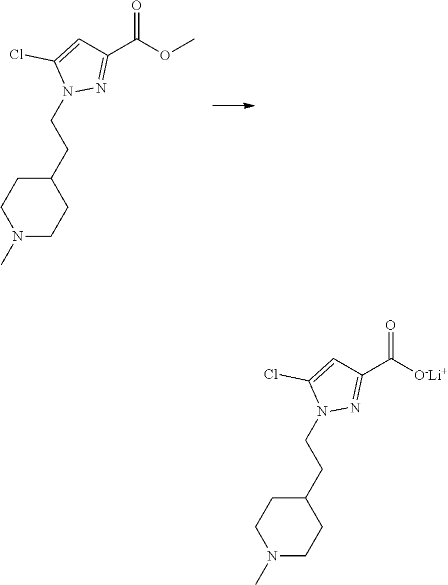

Following general method (E), methyl 5-chloro-1-(2-(1-methylpiperidin-4-yl)ethyl)-1H-pyrazole-3-carboxylate (68 mg, 0.21 mmol) was hydrolysed to afford the title compound (58 mg, quantitative).

### N-((1-Aminoisoquinolin-6-yl)methyl)-5-chloro-1-(2-(1-methylpiperidin-4-yl)ethyl)-1H-pyrazole-3-carboxamide

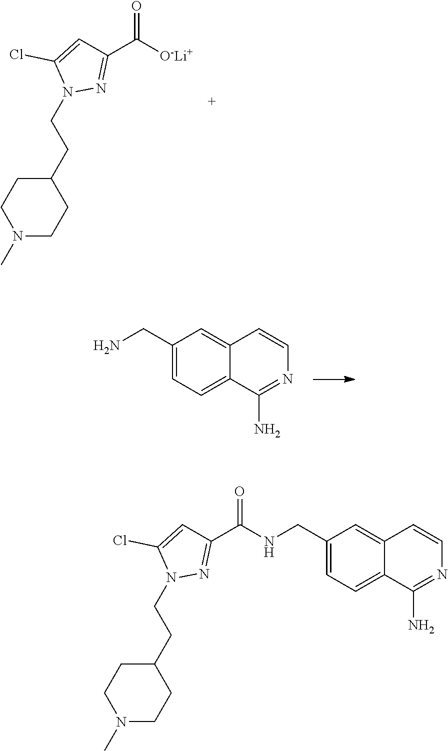

Following general method (A (i)), lithium 5-chloro-1-(2-(1-methylpiperidin-4-yl)ethyl)-1H-pyrazole-3-carboxylate (58 mg, 0.21 mmol) was reacted with 6-(aminomethyl)isoquinolin-1-amine dihydrochloride (55 mg, 0.22 mmol) to afford the title compound (13 mg, 13% yield) as a colourless glass.

[M+H]⁺=427.2

¹H NMR (500 MHz, DMSO-d6) δ: 1.13-1.26 (3H, m), 1.59-1.84 (6H, m), 2.12 (3H, s), 2.69-2.76 (2H, m), 4.16-4.24 (2H, m), 4.54 (2H, d, J=6.2 Hz), 6.71 (2H, s), 6.82 (1H, s), 6.84 (1H, d, J=5.8 Hz), 7.39 (1H, dd, J=8.6, 1.7 Hz), 7.48-7.55 (1H, m), 7.76 (1H, d, J=5.8 Hz), 8.13 (1H, d, J=8.6 Hz), 8.86 (1H, t, J=6.2 Hz)

### Example 69.01

### N-((3-Chloro-1H-pyrrolo[2,3-b]pyridin-5-yl)methyl)-1-((6-fluoro-2-methyl-1,2,3,4-tetrahydroisoquinolin-7-yl)methyl)-5-(methoxymethyl)-1H-pyrazole-4-carboxamide

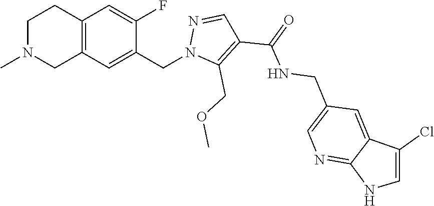 [SMILES: COCc1c(C(=O)NCc2cnc3[nH]cc(Cl)c3c2)cnn1Cc1cc2c(cc1F)CCN(C)C2]

### tert-Butyl 7-bromo-6-fluoro-3,4-dihydroisoquinoline-2(1H)-carboxylate

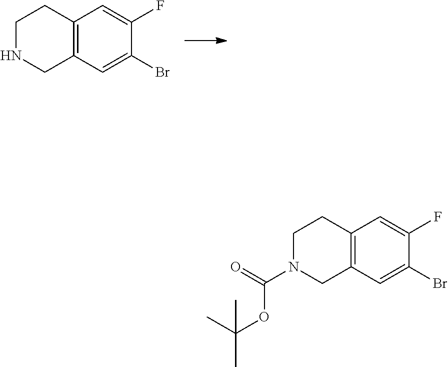 [SMILES: Fc1cc2c(cc1Br)CNCC2 ; CC(C)(C)OC(=O)N1CCc2cc(F)c(Br)cc2C1]

Et₃N (227 μL, 1.63 mmol) was added to a suspension of 7-bromo-6-fluoro-1,2,3,4-tetrahydroisoquinoline (335 mg, 1.46 mmol) and di-tert-butyl dicarbonate (0.637 g, 2.92 mmol) in THF (8 mL). The reaction mixture was stirred at rt for 18 hrs. The mixture was diluted with EtOAc (20 mL) and water (10 mL). The organic layer was separated, the aqueous layer was extracted with EtOAc (2×20 mL). The organic layers were combined, washed with brine (10 ml) dried over Na₂SO₄, filtered and concentrated in vacuo. The residue was purified by flash chromatography (0 to 20% EtOAc in Hexanes) to afford a white solid identified as title compound (520 mg, 97% yield).

[M-tBu+H]⁺=273.8/275.8

¹H NMR (DMSO-d6, 500 MHz) δ 1.42 (9H, s), 2.74 (2H, t, J=5.9 Hz), 3.52 (2H, t, J=6.0 Hz), 4.47 (2H, s), 7.20 (1H, d, J=9.6 Hz), 7.57 (1H, d, J=7.1 Hz)

### tert-Butyl 6-fluoro-7-(hydroxymethyl)-3,4-dihydroisoquinoline-2(1H)-carboxylate

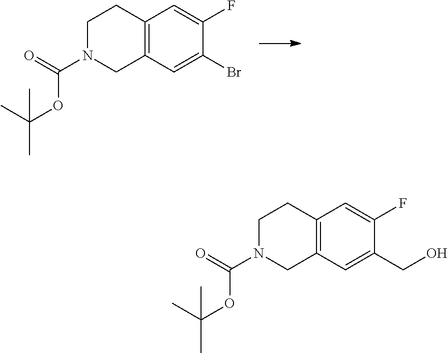 [SMILES: CC(C)(C)OC(=O)N1CCc2cc(F)c(Br)cc2C1 ; CC(C)(C)OC(=O)N1CCc2cc(F)c(CO)cc2C1]

To a solution of tert-butyl 7-bromo-6-fluoro-3,4-dihydroisoquinoline-2(1H)-carboxylate (260 mg, 0.79 mmol) in THF (3 mL) at −78° C. was added n-butyllithium (455 μL, 1.14 mmol) and the solution was stirred for 1 hour. Following this time, N,N-dimethylformamide (156 μL, 2.01 mmol) was added and the mixture, stirred at −78° C. for 30 min. Acetic acid (13 μL, 0.065 mmol) was added and the reaction allowed to warm to rt for 20 min. The mixture was partitioned between 1M HCl (5 mL) and DCM (3×5 mL). Organic layers were combined, dried over Na₂SO₄, filtered, concentrated in vacuo to afford 190 mg of an aldehyde intermediate which was then dissolved in THF (1 mL), MeOH (1 mL) and water (1 mL) then NaBH₄ (32.5 mg, 0.859 mmol) was added. After 20 min, DCM (5 mL) and 1M HCl (5 mL) were added to the mixture. Organic layer was separated and concentrated in vacuo. Flash chromatography (0 to 30% EtOAc in isohexane) afforded title compound (100 mg, 38% yield) as a colourless oil.

1H NMR (DMSO-d6, 500 MHz) δ 1.42 (9H, s), 2.75 (2H, t, J=6.0 Hz), 3.49-3.54 (2H, m), 4.41-4.47 (2H, m), 4.47-4.52 (2H, m), 5.19 (1H, t, J=5.7 Hz), 6.95 (1H, d, J=10.7 Hz), 7.22 (1H, d, J=7.5 Hz)

[M-tBu+H]⁺=209.1

### tert-Butyl 6-fluoro-7-((4-(methoxycarbonyl)-5-(methoxymethyl)-1H-pyrazol-1-yl)methyl)-3,4-dihydroisoquinoline-2(1H)-carboxylate

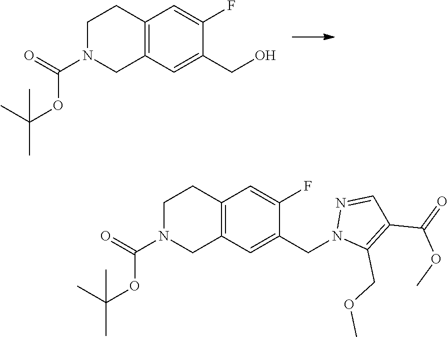 [SMILES: CC(C)(C)OC(=O)N1CCc2cc(F)c(CO)cc2C1 ; COCc1c(C(=O)OC)cnn1Cc1cc2c(cc1F)CCN(C(=O)OC(C)(C)C)C2]

Following general method J, tert-butyl 6-fluoro-7-(hydroxymethyl)-3,4-dihydroisoquinoline-2(1H)-carboxylate (295 mg, 0.95 mmol) was reacted with methyl 3-(methoxymethyl)-1-(methylsulfonyl)-1H-pyrazole-4-carboxylate (296 mg, 1.19 mmol) to afford the title compound (89 mg, 24% yield) as pale-yellow oil.

[M+H]⁺=434.4

¹H NMR (DMSO-d6, 500 MHz) δ 1.40 (9H, s), 2.70-2.78 (2H, m), 3.26 (3H, s), 3.47-3.55 (2H, m), 3.74-3.77 (3H, m), 4.37-4.41 (2H, m), 4.43-4.51 (2H, m), 4.83 (2H, s), 6.96 (1H, d, J=7.1 Hz), 7.04 (1H, d, J=10.7 Hz), 7.88 (1H, s).

### 6-Fluoro-7-((4-(methoxycarbonyl)-5-(methoxymethyl)-1H-pyrazol-1-yl)methyl)-1,2,3,4-tetrahydroisoquinolin-2-ium chloride

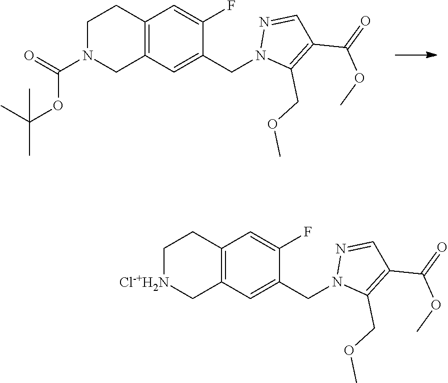

Following general method D, tert-butyl 6-fluoro-7-((4-(methoxycarbonyl)-5-(methoxymethyl)-1H-pyrazol-1-yl)methyl)-3,4-dihydroisoquinoline-2(1H)-carboxylate (84 mg, 0.16 mmol) was deprotected to afford the title compound (105 mg, 87% yield) as an off-white solid.

[M+H]⁺=334.3

### Methyl 1-((6-fluoro-2-methyl-1,2,3,4-tetrahydroisoquinolin-7-yl)methyl)-5-(methoxymethyl)-1H-pyrazole-4-carboxylate

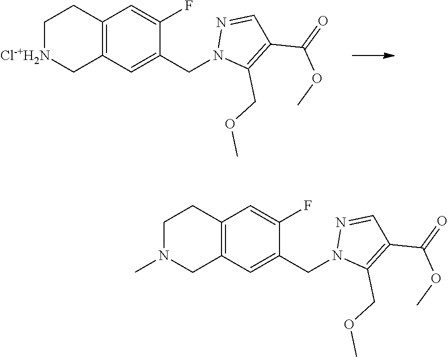

To a stirred solution of 6-fluoro-7-((4-(methoxycarbonyl)-5-(methoxymethyl)-1H-pyrazol-1-yl)methyl)-1,2,3,4-tetrahydroisoquinolin-2-ium chloride (105 mg, 0.18 mmol) in DCM (3 mL) was added TEA (104 μL, 0.75 mmol) and the mixture stirred for 30 min at rt. Formaldehyde solution (37% aq.) (64 μL, 2.13 mmol) was added and the mixture stirred for a further 30 min before the addition of sodium triacetoxyborohydride (83.2 mg, 0.39 mmol). The resulting solution was stirred at rt for 18 hrs. The reaction was diluted with DCM (10 mL) and washed with NaHCO₃ solution (10 mL). The aqueous layer was extracted with DCM (10 mL) and the combined organic layers washed with NaHCO₃ solution (5 mL) and brine (5 mL), then dried via dried over Na₂SO₄ and concentrated in vacuo affording the title compound (84 mg, 91% yield) as a yellow oil.

[M+H]⁺=348.3

¹H NMR (DMSO-d6, 500 MHz) δ 1.91 (3H, s), 2.33 (3H, s), 2.58-2.62 (2H, m), 2.81 (2H, t, J=6.0 Hz), 2.99-3.06 (2H, m), 3.25 (3H, s), 4.81 (2H, s), 5.35-5.37 (2H, m), 6.81 (1H, d, J=7.6 Hz), 6.98 (1H, d, J=10.8 Hz), 7.88 (1H, s).

### Lithium 1-((6-fluoro-2-methyl-1,2,3,4-tetrahydroisoquinolin-7-yl)methyl)-5-(methoxymethyl)-1H-pyrazole-4-carboxylate

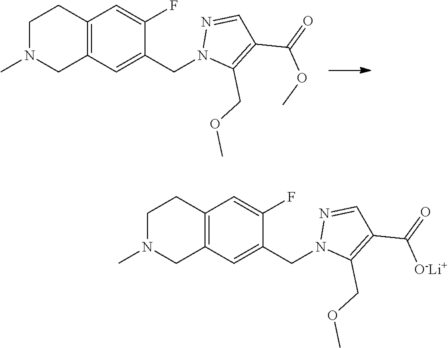

Following general method F, Methyl 1-((6-fluoro-2-methyl-1,2,3,4-tetrahydroisoquinolin-7-yl)methyl)-5-(methoxymethyl)-1H-pyrazole-4-carboxylate (84 mg, 0.16 mmol) was treated with lithium hydroxide (7 mg, 0.29 mmol) to afford title product (82 mg, quantitative yield) as orange gum.

[M+H]⁺=334.3

### N-((3-Chloro-1H-pyrrolo[2,3-b]pyridin-5-yl)methyl)-1-((6-fluoro-2-methyl-1,2,3,4-tetrahydroisoquinolin-7-yl)methyl)-5-(methoxymethyl)-1H-pyrazole-4-carboxamide

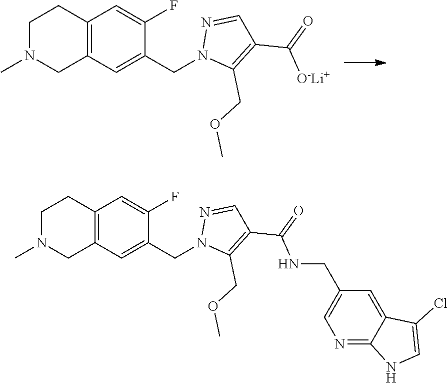

Following general method A, Lithium 1-((6-fluoro-2-methyl-1,2,3,4-tetrahydroisoquinolin-7-yl)methyl)-5-(methoxymethyl)-1H-pyrazole-4-carboxylate (41 mg, 0.08 mmol) was reacted with (3-chloro-1H-pyrrolo[2,3-b]pyridine-5-yl)methanamine dihydrochloride (synthesised as described within WO2016083816, CAS 754173-67-6) (23 mg, 0.09 mmol), [[(E)-(1-cyano-2-ethoxy-2-oxo-ethylidene)amino]oxy-morpholino-methylene]-dimethyl-ammonium hexa-fluorophosphate (COMU, 40 mg, 0.09 mmol) and DIPEA (0.08 mL, 0.46 mmol). The title product was obtained as light yellow solid (19 mg, 42% yield).

[M+H]⁺=497.3/499.3

¹H NMR (DMSO-d6, 500 MHz) δ: 2.28 (3H, s), 2.50-2.54 (2H, m), 2.75-2.80 (2H, m), 3.22 (3H, s), 3.34 (2H, s), 4.52 (2H, d, J=5.6 Hz), 4.85 (2H, s), 5.32 (2H, s), 6.76 (1H, d, J=7.7 Hz), 6.95 (1H, d, J=11.1 Hz), 7.65 (1H, s), 7.83 (1H, s), 7.97 (1H, s), 8.28 (1H, s), 8.68-8.72 (1H, m), 11.93 (1H, br. s)

### Example 82.01

### 2-((1-Aminoisoquinolin-6-yl)methyl)-4-(((1-methylpiperidin-4-yl)methyl)amino)-1,2-dihydro-3H-pyrrolo[3,4-c]pyridin-3-one

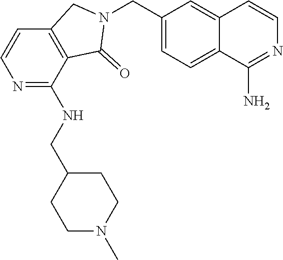 [SMILES: CN1CCC(CNc2nccc3c2C(=O)N(Cc2ccc4c(N)nccc4c2)C3)CC1]

### 2-Fluoro-N,N-diisopropyinicotinamide

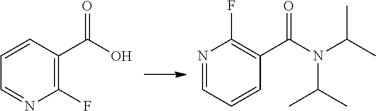 [SMILES: O=C(O)c1cccnc1F ; CC(C)N(C(=O)c1cccnc1F)C(C)C]

To 2-fluoro-3-pyridinecarboxylic acid (1 g, 7.09 mmol) in DCM (70 mL) was added oxalyl chloride (1.2 ml, 14.2 mmol) and a catalytic amount of DMF (0.1 mL) and the reaction stirred at rt for 5 hrs. The solvent was removed in vacuo and fresh DCM added. The reaction was cooled to 0° C. and DIPEA (3.1 ml, 17.7 mmol) and diisopropylamine (1.2 ml, 8.50 mmol) were added. The reaction mixture was warmed to rt and stirred for 48 hrs. Flash chromatography (0-100% EtOAc in cyclohexane) afforded the title compound (1.43 g, 90% yield) as an off white solid.

[M+H]⁺=225.0

### 2-Fluoro-4-formyl-N,N-diisopropyinicotinamide

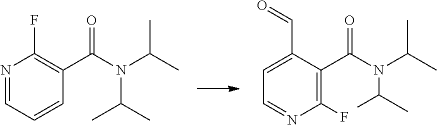 [SMILES: CC(C)N(C(=O)c1cccnc1F)C(C)C ; CC(C)N(C(=O)c1c(C=O)ccnc1F)C(C)C]

To a solution of diisopropylamine (0.89 ml, 6.38 mmol) in dry THF (50 ml) at −70° C. was added n-butyllithium (2.6 ml, 6.38 mmol) and stirred for 20 min. A solution of 2-fluoro-N,N-diisopropylnicotinamide (1.43 g, 6.38 mmol) in THF (15 ml) was then added whilst keeping the temperature below −70° C. The mixture was stirred for 60 min before dry DMF (1.5 ml, 19.13 mmol) was added whilst holding the reaction at −70° C. for 5 min. The reaction was then warmed to rt and stirred for 60 min. The reaction mixture was quenched with saturated NH₄Cl (25 mL), extracted with EtOAc (35 ml), washed with brine (25 ml), dried (Na₂SO₄) and concentrated in vacuo. Flash chromatography (0-100% EtOAc in cyclohexane) afforded the title compound (1.27 g, 79% yield) as an off white solid.

[M+H]⁺=253.0

### 4-((((1-Aminoisoquinolin-6-yl)methyl)amino)methyl)-2-fluoro-N,N-diisopropyinicotinamide

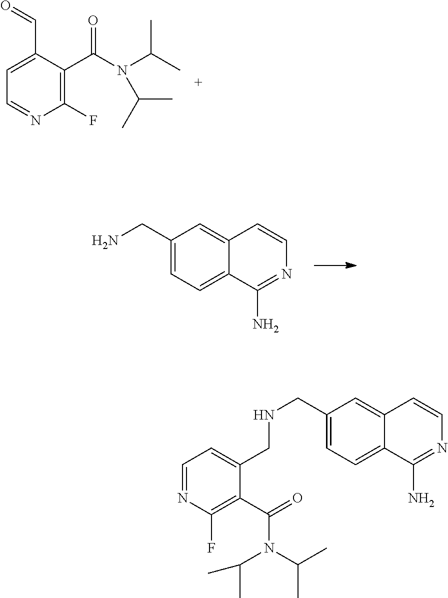 [SMILES: CC(C)N(C(=O)c1c(C=O)ccnc1F)C(C)C ; NCc1ccc2c(N)nccc2c1 ; CC(C)N(C(=O)c1c(CNCc2ccc3c(N)nccc3c2)ccnc1F)C(C)C]

Following general method F, 2-fluoro-4-formyl-N,N-diisopropylnicotinamide (1.27 g, 3.22 mmol) was reacted with 6-(aminomethyl)isoquinolin-1-amine (610 mg, 3.54 mmol) to afford the title compound (980 mg, 74% yield) as a pale, yellow solid.

[M+H]⁺=410.2

### 2-((1-aminoisoquinolin-6-yl)methyl)-4-(((1-methylpiperidin-4-yl)methyl)amino)-1,2-dihydro-3H-pyrrolo[3,4-c]pyridin-3-one

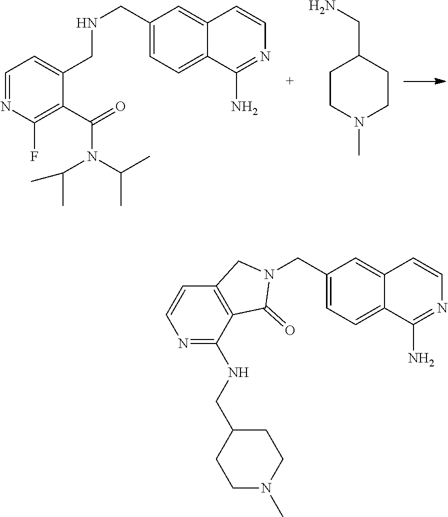

A mixture of 4-((((1-aminoisoquinolin-6-yl)methyl)amino)methyl)-2-fluoro-N,N-diisopropyinicotinamide (200 mg, 0.49 mmol), (1-Methyl-4-piperidinyl)methanamine (69 mg, 0.54 mmol) and DIPEA (170 μL, 0.98 mmol) in NMP (4 mL) was sealed and heated at 250° C. for 12 hrs. The mixture was diluted with MeOH and concentrated in vacuo. Purification by prep HPLC (10-98% A to B, A=0.1% NH₄OH in water, B=0.1% NH₄OH in MeCN) afforded the title compound (20 mg, 10% yield) as an off white solid.

[M+H]⁺=417.1

¹H NMR (DMSO-d6, 500 MHz) δ: 1.28-1.16 (2H, m), 1.59-1.49 (1H, m), 1.65 (2H, d, J=12.4 Hz), 1.80 (2H, dt, J=2.4, 11.6 Hz), 2.13 (3H, s), 2.78-2.71 (2H, m), 3.38 (2H, t, J=6.5 Hz), 4.35 (2H, s), 4.78 (2H, s), 6.68 (1H, d, J=5.1 Hz), 6.75 (2H, s), 6.92-6.86 (2H, m), 7.34 (1H, dd, J=1.7, 8.6 Hz), 7.55 (1H, d, J=1.1 Hz), 7.77 (1H, d, J=5.9 Hz), 8.12 (1H, d, J=5.4 Hz), 8.16 (1H, d, J=8.7 Hz).

TABLE 13
¹H NMR data of examples (solvent d6 DMSO unless otherwise indicated)

| Ex. No. | NMR write-up |
|---|---|
| 2.01 | 2.22 (6H, s), 3.62 (2H, s), 4.57 (2H, d, J = 5.9 Hz), 6.82 (2H, br.s), 6.69 (1H, d, J = 5.8 Hz), 7.41 |
| | (1H, d, J = 8.4 Hz), 7.57 (1H, s), 7.75-7.78 (2H, m), 8.16 (1H, d, J = 8.6 Hz), 9.17 (1H, t, J = 5.7 Hz) |
| 2.58 | 2.00-2.10 (2H, m), 2.44-2.49 (2H, m), 3.12 (1H, br.s), 3.72 (2H, s), 3.77-3.85 (4H, m), 4.57 |
| | (2H, d, J = 5.9 Hz), 6.73 (2H, s), 6.87 (1H, d, J = 6.1Hz), 7.34 (2H, d, J = 8.6Hz), 7.39 (1H, dd, J = |
| | 8.6, 1.8Hz), 7.56 (1H, s), 7.60 (2H, d, J = 8.6 Hz), 7.75 (1H, s), 7.76 (1H, d, J = 5.8 Hz), 8.15 |
| | (1H, d, J = 8.6 Hz), 9.14 (1H, t, J = 6.0 Hz) |
| 5.12 | 2.56-2.63 (4H, m), 3.28-3.38 (4H, m), 3.77 (2H, s), 4.58 (2H, d, J = 5.9Hz), 6.73 (2H, s), 6.79- |
| | 6.84 (2H, m), 6.88 (1H, d, J = 5.9, 0.7Hz), 7.40 (1H, dd, J = 8.6, 1.8Hz), 7.57 (1H, d, J = |
| | 1.7Hz), 7.78 (1H, d, J = 5.8Hz), 7.79 (1H, s), 8.13-8.18 (3H, m), 9.19 (1H, t, J = 6.0, 6.0Hz) |
| 5.16 | ¹H NMR at 90° C. 2.98 (4H, br.s), 3.56 (4H, br.s), 4.13 (2H, s), 4.68 (2H, d, J = 5.9 Hz), 7.21 |
| | (1H, d, J = 7.0 Hz), 7.64-7.71 (2H, m), 7.75 (1H, dd, J = 8.6, 1.7 Hz), 7.85-7.89 (3H, m), 8.15 |
| | (1H, dd, J = 5.1, 1.0 Hz), 8.39 (1H, d, J = 2.9 Hz), 8.58 (1H, d, J = 8.6 Hz), 9.02 (2H, s), 9.25 (1H, |
| | t, J = 5.9 Hz), 13.34 (1H, s). |
| 5.17 | ¹H NMR at 90° C. 3.07 (4H, s), 3.38-4.20 (4H, m), 4.27 (2H, s), 4.69 (2H, d, J = 5.8 Hz), 6.79 |
| | (1H, dd, J = 7.0, 5.3 Hz), 7.01 (1H, d, J = 8.7 Hz), 7.21 (1H, d, J = 7.0 Hz), 7.67 (1H, d, J = 6.9 |
| | Hz), 7.68-7.73 (1H, m), 7.75 (1H, dd, J = 8.7, 1.7 Hz), 7.86 (1H, s), 7.89 (1H, s), 8.12 (1H, dd, J = |
| | 5.4, 1.8 Hz), 8.58 (1H, d, J = 8.6 Hz), 9.01 (2H, s), 9.27 (1H, d, J = 7.1 Hz). |
| 5.18 | (MeOD) 2.20 (1H, s), 2.70 (1H, d, J = 20.8 Hz), 3.37-3.64 (2H, m), 3.70-3.99 (2H, m), 4.50 |
| | (1H, s), 4.81 (4H, s), 7.24 (1H, d, J = 7.1 Hz), 7.58 (1H, d, J = 7.0 Hz), 7.77-7.86 (4H, m), 7.87- |
| | 7.91 (1H, m), 8.10 (1H, d, J = 4.7 Hz), 8.18 (1H, d, J = 2.5 Hz), 8.44 (1H, d, J = 8.6 Hz). |
| 5.19 | (MeOD) 4.79 (2H, s), 4.83 (2H, s), 4.90 (2H, s), 4.95 (2H, s), 7.24 (1H, d, J = 7.0 Hz), 7.58 (1H, |
| | d, J = 7.0 Hz), 7.75-7.82 (2H, m), 7.89 (1H, s), 8.13 (1H, d, J = 5.9 Hz), 8.44 (1H, d, J = 8.7 Hz), |
| | 8.88 (1H, d, J = 5.9 Hz), 8.92 (1H, s). |
| 5.20 | 2.17 (3H, s), 2.55 (4H, t, J = 5.1 Hz), 3.32 (4H, t, J = 5.0 Hz), 3.66 (2H, s), 4.55 (2H, d, J = 6.0 |
| | Hz), 6.72 (2H, s), 6.78-6.83 (2H, m), 6.87 (1H, d, J = 5.8 Hz), 7.39 (1H, dd, J = 8.6, 1.8 Hz), |
| | 7.56 (2H, d, J = 9.2 Hz), 7.77 (1H, d, J = 5.8 Hz), 8.12-8.18 (3H, m), 8.99 (1H, t, J = 6.0 Hz). |
| 5.21 | (MeOD) 2.18 (1H, s), 2.54-2.79 (1H, br.s), 3.45-3.64 (2H, m), 3.71-3.93 (2H, m), 4.48 (1H, |
| | s), 4.71-4.83 (4H, m), 7.24 (1H, d, J = 7.0 Hz), 7.58 (1H, d, J = 7.1 Hz), 7.75-7.84 (4H, m), |
| | 7.89 (1H, s), 8.09 (1H, s), 8.16 (1H, s), 8.44 (1H, d, J = 8.6 Hz). |
| 5.22 | ¹H NMR at 90° C.: 3.1-3.17 (4H, m), 4.06 (2H, s), 4.18 (2H, s), 4.67 (2H, d, J = 5.8 Hz), 7.20 |
| | (1H, d, J = 7.0 Hz), 7.63 (1H, d, J = 5.7 Hz), 7.67 (1H, d, J = 6.9 Hz), 7.74 (1H, dd, J = 8.6, 1.7 |
| | Hz), 7.85 (1H, d, J = 1.8 Hz), 7.89 (1H, s), 8.54 (1H, d, J = 5.6 Hz), 8.56-8.61 (2H, m), 9.05 |
| | (2H, s), 9.27 (1H, t, J = 5.9 Hz). |
| 5.23 | 1.86-1.92 (1H, m), 2.41-2.47 (1H, m), 2.55-2.61 (5H, m), 2.76-2.82 (1H, m), 3.32-3.43 |
| | (4H, m), 3.76 (2H, s), 5.35-5.41 (1H, m), 5.82 (2H, d, J = 5.9 Hz), 6.45 (1H, d, J = 5.1 Hz), 6.80- |
| | 6.85 (2H, m), 7.74-7.80 (2H, m), 8.14-8.19 (2H, m), 8.81 (1H, d, J = 8.4 Hz). |
| 5.24 | 2.16-2.26 (1H, m), 2.31 (3H, s), 2.57-2.67 (1H, m), 3.15-3.29 (2H, m), 3.56 (2H, s), 3.63- |
| | 3.71 (2H, m), 4.02-4.11 (1H, m), 4.12-4.21 (1H, m), 4.52 (2H, s), 4.64 (2H, d, J = 6.2 Hz), 7.05- |
| | 7.13 (1H, m), 7.20-7.28 (1H, m), 7.23 (1H, d, J = 7.0 Hz), 7.69 (1H, d, J = 7.0 Hz), 7.72 (1H, |
| | dd, J = 8.6, 1.7 Hz), 7.76 (1H, s), 7.83 (1H, s), 8.32 (2H, d, J = 7.2 Hz), 8.61 (1H, d, J = 8.6 Hz), |
| | 9.20 (2H, s), 9.48 (1H, s), 11.71 (1H, s), 13.41 (1H, s), 13.96 (1H, s). |
| 5.25 | 2.14 (3H, s), 3.11-3.14 (2H, m), 3.77 (2H, s), 3.82 (2H, td, J = 6.2, 1.8 Hz), 4.54 (2H, d, J = 5.9 |
| | Hz), 4.95 (1H, t, J = 5.6 Hz), 6.71 (2H, s), 6.84-6.89 (3H, m), 7.38 (1H, dd, J = 8.7, 1.7 Hz), |
| | 7.53 (2H, d, J = 6.7 Hz), 7.76 (1H, d, J = 5.7 Hz), 8.13 (1H, d, J = 8.6 Hz), 8.35-8.39 (2H, m), |
| | 8.96 (1H, t, J = 6.0 Hz) |
| 5.26 | 2.16 (3H, s), 2.51 (4H, t, J = 5.3 Hz), 3.62 (4H, t, J = 5.3 Hz), 3.65 (2H, s), 4.54 (2H, d, J = 5.6 |
| | Hz), 6.70 (2H, d, J = 4.9 Hz), 6.81 (1H, dd, J = 6.3, 1.3 Hz), 6.86 (1H, d, J = 5.8 Hz), 7.38 (1H, |
| | dd, J = 8.6, 1.7 Hz), 7.55 (2H, d, J = 8.1 Hz), 7.76 (1H, d, J = 5.8 Hz), 8.13 (1H, d, J = 8.6 Hz), |
| | 8.17 (1H, d, J = 6.2 Hz), 8.46-8.50 (1H, m), 8.98 (1H, t, J = 6.1 Hz) |
| 5.27 | 2.16 (3H, s), 2.51 (4H, t, J = 5.0 Hz), 3.23 (4H, t, J = 5.0 Hz), 3.26 (3H, s), 3.63 (2H, s), 4.54 (2H, |
| | d, J = 5.6 Hz), 5.49 (1H, d, J = 2.7 Hz), 6.06 (1H, dd, J = 7.7, 2.8 Hz), 6.71 (2H, d, J = 5.0 Hz), |
| | 6.86 (1H, d, J = 5.8 Hz), 7.37-7.42 (2H, m), 7.53-7.56 (2H, m), 7.76 (1H, d, J = 5.8 Hz), 8.13 |
| | (1H, d, J = 8.6 Hz), 8.98 (1H, t, J = 6.0 Hz) |
| 5.28 | 2.16 (3H, s), 2.54 (4H, t, J = 5.0 Hz), 2.83 (4H, t, J = 4.7 Hz), 3.63 (2H, s), 3.71 (3H, s), 4.54 (2H, |
| | d, J = 5.5 Hz), 6.70 (2H, d, J = 5.0 Hz), 6.86 (1H, d, J = 5.8 Hz), 7.11 (1H, s), 7.21 (1H, s), 7.38 |
| | (1H, dd, J = 8.6, 1.7 Hz), 7.51-7.56 (2H, m), 7.76 (1H, d, J = 5.8 Hz), 8.13 (1H, d, J = 8.6 Hz), |
| | 8.97 (1H, t, J = 6.0 Hz) |
| 5.29 | 2.16 (3H, s), 3.23 (4H, t, 7 = 5.0 Hz), 3.64 (2H, s), 4.54 (2H, d, 7 = 6.0 Hz), 5.38 (1H, d, 7 = 2.5 Hz), |
| | 6.00 (1H, dd, 7 = 7.6, 2.6 Hz), 6.71 (2H, s), 6.86 (1H, d, 7 = 5.8 Hz), 7.09 (1H, d, 7 = 7.5 Hz), 7.38 |
| | (1H, dd, 7 = 8.6, 1.7 Hz), 7.53-7.56 (2H, m), 7.76 (1H, d, 7 = 5.8 Hz), 8.13 (1H, d, 7 = 8.6 Hz), 8.98 |
| | (1H, t, 7 = 6.0 Hz), 10.59 (1H, s) |
| 25.01 | 4.59 (2H, d, J = 5.9 Hz), 6.75 (2H, s), 6.88 (1H, d, J = 5.8 Hz), 7.40 (1H, dd, J = 8.6, 1.5 Hz), |
| | 7.57 (1H, s), 7.77 (1H, d, J = 5.8 Hz), 7.84-7.85 (2H, m), 8.15 (1H, d, J = 8.6 Hz), 9.23 |
| | (1H, t, J = 5.9 Hz). |
| 25.02 | 2.40 (3H, s), 4.56 (2H, d, J = 5.9 Hz), 6.76 (2H, s), 6.87 (1H, d, J = 5.8 Hz), 7.38 (1H, dd, |
| | J = 8.6, 1.6 Hz), 7.55 (1H, s), 7.76 (1H, d, J = 5.8 Hz), 7.80 (1H, s), 8.15 (1H, d, J = 8.6 Hz), |
| | 9.17 (1H, t, J = 5.9 Hz). |
| 25.03 | 4.58 (2H, d, J = 5.9 Hz), 6.94 (1H, d, J = 5.8 Hz), 7.14 (2H, br.s), 7.45 (1H, d, J = 8.5 Hz), 7.55 |
| | (1H, d, J = 1.7 Hz), 7.60 (1H, br.s), 7.74 (1H, d, J = 6.0 Hz), 8.09 (1H, d, J = 1.8 Hz), 8.21 (1H, d, |
| | J = 8.9 Hz), 9.01 (1H, t, J = 5.7 Hz) |
| 25.04 | 2.40 (3H, s), 2.50 (2H, d, J = 3.6 Hz), 6.78 (2H, br.s), 6.88 (1H, d, J = 5.8 Hz), 7.39 (1H, d, J = |
| | 8.4 Hz), 7.55 (1H, s), 7.75-7.77 (2H, m), 8.15 (1H, d, J = 8.4 Hz), 9.13 (1H, t, J = 5.6 Hz) |
| 25.07 | 2.59 (3H, d, J = 4.7 Hz), 4.68 ( 2H, d, J = 5.8 Hz), 7.24 (1H, d, J = 7.0 Hz), 7.67 (1H, d, J = 6.9 |
| | Hz), 7.72 (1H, dd, J = 1.4, 8.7 Hz), 7.85 (1H, s), 8.00 (1H, s), 8.19 (1H, dd, J = 4.7, 9.6 Hz), 8.51 |
| | (1H, d, J = 8.6 Hz), 8.91 (2H, s), 9.57 (1H, t, J = 5.9 Hz) |
| 25.08 | 4.15 (2H, s), 4.56 (2H, s), 6.72 (2H, s), 6.87 (1H, d, J = 5.8 Hz), 7.19-7.42 (6H, m), |
| | 7.54 (1H, s), 7.76 (1H, d, J = 5.8 Hz), 7.80 (1H, s), 8.14 (1H, d, J = 8.6 Hz), 9.17 (1H, s). |
| 25.09 | 1.56-1.68 (2H, m), 1.86-1.95 (2H, m), 1.95-2.02 (2H, m), 2.19 (2H, s), 2.73-2.88 (3H, m), |
| | 4.56 (2H, d, J = 5.9 Hz), 6.71 (2H, s), 6.86 (1H, d, J = 5.8 Hz), 6.94 (1H, d, J = 3.8 Hz), 7.39 (1H, |
| | dd, J = 8.6, 1.7 Hz), 7.54 (1H, d, J = 1.7 Hz), 7.67 (1H, d, J = 3.8 Hz), 7.76 (1H, d, J = 5.8 Hz), |
| | 8.14 (1H, d, J = 8.6 Hz), 9.03 (1H, t, J = 6.0 Hz). |
| 25.10 | 1.18-1.29 (2H, m), 1.48-1.52 (1H, m), 1.56-1.62 (2H, m), 1.75-1.83 (2H, m), 2.12 (3H, s), |
| | 2.69-2.76 (4H, m), 4.57 (2H, d, J = 5.9 Hz), 6.72 (2H, s), 6.88 (1H, d, J = 5.8 Hz), 7.39 (1H, dd, |
| | J = 8.6, 1.8 Hz), 7.56 (1H, s), 7.75-7.79 (2H, m), 8.15 (1H, d, J = 8.6 Hz), 9.16 (1H, t, J = 6.0 Hz). |
| 25.11 | 1.12-1.23 (3H, m), 1.50-1.57 (2H, m), 1.61-1.70 (2H, m), 1.74-1.82 (2H, m), 2.12 (3H, s), |
| | 2.68-2.77 (2H, m), 2.77-2.84 (2H, m), 4.57 (2H, d, J = 5.8 Hz), 6.72 (2H, s), 6.88 (1H, d, J = |
| | 5.8 Hz), 7.39 (1H, dd, J = 8.6, 1.8 Hz), 7.56 (1H, d, J = 1.7 Hz), 7.75-7.78 (2H, m), 8.15 (1H, d, |
| | J = 8.6 Hz), 9.15 (1H, t, J = 6.0 Hz). |
| 25.12 | 1.14-1.21 (3H, m), 1.46-1.56 (2H, m), 1.61-1.67 (2H, m), 1.74-1.83 (2H, m), 2.12 (3H, s), |
| | 2.56-2.61 (2H, m), 2.68-2.74 (2H, m), 4.56 (2H, d, J = 5.8 Hz), 6.72 (2H, s), 6.87 (1H, d, J = |
| | 5.8 Hz), 7.38-7.41 (2H, m), 7.55 (1H, d, J = 1.7 Hz), 7.71 (1H, d, J = 1.4 Hz), 7.77 (1H, d, J = |
| | 5.8 Hz), 8.14 (1H, d, J = 8.6 Hz), 9.04 (1H, t, J = 6.0 Hz). |
| 25.13 | 4.59 (2H, d, J = 5.9 Hz), 6.73 (2H, s), 6.88 (1H, d, J = 5.8 Hz), 7.40 (1H, dd, J = 8.6, 1.8 Hz), |
| | 7.57 (1H, d, J = 1.8 Hz), 7.77 (1H, d, J = 5.8 Hz), 7.89 (1H, d, J = 1.5 Hz), 7.94 (1H, d, J = 1.4 |
| | Hz), 8.15 (1H, d, J = 8.6 Hz), 9.24 (1H, t, J = 6.0 Hz). |
| 25.14 | 0.96-1.11 (2H, m), 1.29-1.38 (1H, m), 1.54-1.68 (4H, m), 2.39-2.47 (2H, m), 2.73-2.84 |
| | (2H, m), 2.88-2.96 (2H, m), 4.58 (2H, d, J = 6.3 Hz), 6.71 (2H, s), 6.86 (1H, d, J = 5.8 Hz), 7.42 |
| | (1H, dd, J = 8.6, 1.7 Hz), 7.56 (1H, d, J = 1.7 Hz), 7.64 (1H, s), 7.77 (1H, d, J = 5.8 Hz), 8.14 (1H, |
| | d, J = 8.6 Hz), 9.36 (1H, t, J = 6.4 Hz), N-H not observed. |
| 25.15 | 1.17-1.28 (3H, m), 1.59-1.66 (2H, m), 1.66-1.73 (2H, m), 1.92-2.04 (2H, m), 2.22 (3H, s), |
| | 2.76-2.87 (4H, m), 4.56-4.60 (2H, m), 6.73 (2H, s), 6.86 (1H, d, J = 5.8 Hz), 7.42 (1H, dd, J = |
| | 8.6, 1.7 Hz), 7.56 (1H, d, J = 1.7 Hz), 7.64 (1H, s), 7.76 (1H, d, J = 5.8 Hz), 8.14 (1H, d, J = 8.6 |
| | Hz), 9.36 (1H, t, J = 6.4 Hz). |
| 25.16 | 1.28-1.45 (2H, m), 1.84-1.92 (2H, m), 1.97 (2H, t, J = 11.3 Hz), 2.15 (3H, s), 2.63-2.78 (2H, m), |
| | 2.93-3.04 (1H, m), 4.55 (2H, d, J = 5.9 Hz), 5.41 (1H, d, J = 7.9 Hz), 6.19 (1H, d, J = 1.6 |
| | Hz), 6.71 (2H, s), 6.86 (1H, d, J = 5.8 Hz), 7.30 (1H, d, J = 1.7 Hz), 7.38 (1H, dd, J = 8.6, 1.8 Hz), |
| | 7.53 (1H, s), 7.77 (1H, d, J = 5.8 Hz), 8.14 (1H, d, J = 8.6 Hz), 8.97 (1H, t, J = 6.1 Hz). |
| 25.17 | 1.18 (2H, qd, J = 12.1, 4.0 Hz), 1.43-1.54 (1H, m), 1.67-1.74 (2H, m), 1.82 (2H, td, J = 11.7, |
| | 2.5 Hz), 2.14 (2H, s), 2.71-2.79 (2H, m), 2.83 (2H, t, J = 6.3 Hz), 4.54 (2H, d, J = 6.0 Hz), 5.59 |
| | (1H, t, J = 5.9 Hz), 6.14 (1H, d, J = 1.6 Hz), 6.72 (2H, s), 6.86 (1H, d, J = 5.8 Hz), 7.30 (1H, d, J = |
| | 1.7 Hz), 7.38 (1H, dd, J = 8.6, 1.7 Hz), 7.53 (1H, d, J = 1.7 Hz), 7.77 (1H, d, J = 5.8 Hz), 8.14 |
| | (1H, d, J = 8.6 Hz), 8.98 (1H, t, J = 6.0 Hz). |
| 25.101 | 2.38 (3H, s), 2.51 (3H, s), 2.56 (3H, s), 4.55 (2H, d, J = 4.7 Hz), 6.45 (1H, dd, J = 3.4, 1.5 Hz), |
| | 7.30 (1H, dd, J = 3.5, 2.1 Hz), 7.74 (1H, c, J = 1.4 Hz), 8.45 (1H, t, J = 4.7 Hz), 11.35 (1H, s). |
| 25.102 | 2.31 (6H, s), 2.37 (3H, s), 3.62 (2H, s), 4.40 (2H, d, J = 4.7 Hz), 6.98 (2H, s), 7.76 (1H, s), 8.42 |
| | (1H, t, J = 4.8 Hz), NH2 was not observed |
| 25.103 | 2.40 (3H, s), 4.64 (2H, d, 7 = 6.1 Hz), 6.49 (2H, s), 7.03 (1H, d, 7 = 5.7 Hz), 7.51 (1H, s), 7.69- |
| | 7.75 (2H, m), 9.25 (1H, t, 7 = 5.9 Hz). |
| 25.104 | 2.40 (3H, s), 4.62 (2H, d, J = 5.7 Hz), 6.95 (2H, s), 6.97 (1H, dd, J = 5.9, 0.9 Hz), 7.37-7.44 |
| | (1H, m), 7.75 (1H, s), 7.88 (1H, d, J = 5.9 Hz), 8.00 (1H, d, J = 8.6 Hz), 9.12 (1H, t, J = 5.8 Hz). |
| 25.105 | 2.15 (3H, s), 2.29 (3H, s), 2.37 (3H, s), 4.29 (2H, d, J= 4.7 Hz), 5.69 (2H, s), 6.12 (1H, s), 7.76 |
| | (1H, s), 8.20 (1H, s), 8.36 (1H, t, J = 4.5 Hz) |
| 25.201 | 1.54-1.42 (2H, m), 1.63 (2H, d, J = 13.4 Hz), 2.14-2.03 (1H, m), 2.72-2.68 (3H, m), 2.86- |
| | 2.80 (2H, m), 3.35 (2H, d, J = 12.3 Hz), 4.57 (2H, d, J = 6.9 Hz), 4.72-4.68 (2H, m), 7.15 (1H, t, |
| | J = 7.5 Hz), 7.35-7.24 (3H, m), 7.78-7.61 (4H, m), 7.86 (1H, s), 8.57-8.53 (1H, m), 9.06 (1H, s), |
| | 9.20 (1H, d, J = 2.9 Hz), 9.31 (1H, t, J = 6.0 Hz), 13.24-13.20 (1H, m). |
| 25.202 | 0.96-0.91 (3H, m), 1.39-1.21 (4H, m), 1.66 (2H, dd, J = 9.3, 11.4 Hz), 1.86-1.77 (1H, m), |
| | 2.26-2.19 (2H, m), 2.81-2.72 (2H, m), 4.65-4.58 (4H, m), 6.73 (2H, s), 6.87-6.84 (1H, m), |
| | 7.48-7.31 (3H, m), 7.59 (1H, s), 7.78-7.74 (3H, m), 8.18-8.14 (1H, m), 9.67 (1H, t, J = 6.3 Hz) |
| 25.203 | 4.64 (2H, d, J = 5.9 Hz), 6.75 (2H, s), 6.89 (1H, d, J = 5.8 Hz), 7.45 (1H, dd, J = 8.6, 1.8 Hz), |
| | 7.47-7.52 (1H, m), 7.56 (1H, dd, J = 7.7, 0.9 Hz), 7.62 (1H, s), 7.78 (1H, d, J = 5.8 Hz), 8.05 |
| | (1H, d, J = 8.1 Hz), 8.17 (1H, d, J = 8.6 Hz), 8.34 (1H, s), 9.60 (1H, t, J = 6.0 Hz) |
| 25.204 | 4.65 (2H, d, J = 5.9 Hz), 6.73 (2H, s), 6.88 (1H, d, J = 5.8 Hz), 7.44 (1H, dd, J = 8.6, 1.7 Hz), |
| | 7.51 (1H, dd, J = 7.8 Hz), 7.59-7.64 (2H, m), 7.78 (1H, d, J = 5.8 Hz), 7.98 (1H, dd, J = 8.0, 0.9 |
| | Hz), 8.17 (1H, d, J = 8.6 Hz), 8.27 (1H, s), 9.52 (1H, t, J = 6.0 Hz). |
| 25.205 | 4.63 (2H, d, J = 5.9 Hz), 6.77 (2H, s), 6.89 (1H, d, J = 5.8 Hz), 7.33-7.41 (1H, m), 7.44 (1H, dd, |
| | J = 8.6, 1.8 Hz), 7.60 (1H, d, J = 1.7 Hz), 7.77 (1H, d, J = 5.8 Hz), 7.82 (1H, dd, J = 9.7, 2.6 Hz), |
| | 8.09 (1H, dd, J = 8.9, 4.9 Hz), 8.13-8.20 (2H, m), 9.48 (1H, t, J = 6.0 Hz). |
| 25.206 | 4.64 (2H, d, J = 5.9 Hz), 6.73 (2H, s), 6.89 (1H, d, J = 5.7 Hz), 7.24-7.31 (1H, m), 7.44 (1H, dd, |
| | J = 8.6, 1.8 Hz), 7.47-7.55 (1H, m), 7.61 (1H, d, J = 1.7 Hz), 7.78 (1H, d, J = 5.8 Hz), 7.90 |
| | (1H, d, J = 8.1 Hz), 8.17 (1H, d, J = 8.6 Hz), 8.31 (1H, d, J = 0.8 Hz), 9.51 (1H, t, J = 6.0 Hz). |
| 25.207 | 4.65 (2H, d, J = 5.8 Hz), 6.72 (2H, s), 6.87 (1H, d, J = 5.8 Hz), 7.13 (1H, d, J = 7.5 Hz), 7.21- |
| | 7.15 (1H, m), 7.33 (1H, s), 7.46-7.39 (2H, m), 7.59 (1H, s), 7.77 (1H, d, J = 5.8 Hz), 8.16 |
| | (1H, d, J = 8.6 Hz), 9.29 (1H, s), 12.03 (1H, s) |
| 25.208 | 1.53 (2H, d, J = 11.3 Hz), 1.95 (2H, d, J = 12.9 Hz), 2.03 (2H, t, J = 11.4 Hz), 2.18 (3H, s), 2.78 |
| | (2H, d, J = 11.1 Hz), 4.60 (2H, d, J = 5.7 Hz), 5.79 (1H, d, J = 7.7 Hz), 6.50 (1H, d, J = 7.9 Hz), |
| | 6.73 (2H, s), 6.87 (1H, d, J = 5.9 Hz), 7.13 (1H, d, J = 8.0 Hz), 7.22 (1H, t, J = 7.9 Hz), 7.44 (1H, |
| | dd, J = 8.1, 1.7 Hz), 7.59 (1H, s, 1H), 7.77 (1H, d, J = 5.8 Hz), 8.16 (1H, d, J = 8.6 Hz), |
| | 8.33 (1H, s), 9.11 (1H, d, J = 6.0 Hz) |
| 25.209 | 1.31-1.18 (2H, m), 1.65-1.59 (1H, m), 1.86-1.73 (4H, m), 2.14 (3H, s), 2.81-2.72 (2H, m), |
| | 3.07-3.00 (2H, m), 4.61 (2H, d, J = 5.8 Hz), 6.09-6.05 (1H, m), 6.44 (1H, d, J = 7.9 Hz), 6.73 |
| | (2H, s), 6.87 (1H, d, J = 5.9 Hz), 7.13 (1H, d, J = 7.9 Hz), 7.25-7.20 (1H, m), 7.45-7.42 (1H, |
| | m), 7.59 (1H, s), 7.77 (1H, d, J = 5.7 Hz), 8.16 (1H, d, J = 8.6 Hz), 8.32 (1H, s), 9.07 (1H, s) |
| 25.210 | 4.64 (2H, d, J = 5.8 Hz), 6.73 (2H, s), 6.89 (1H, d, J = 5.8 Hz), 7.40 (1H, m), 7.45 (1H, dd, J = |
| | 8.6, 1.7 Hz), 7.62 (1H, d, J = 1.7 Hz), 7.70 (1H, d, J = 7.6 Hz), 7.78 (1H, d, J = 5.8 Hz), 8.08 |
| | (1H, d, J = 8.1 Hz), 8.17 (1H, d, J = 8.6 Hz), 8.30 (1H, s), 9.63 (1H, t, J = 5.9 Hz). |
| 26.01 | 1.13-1.01 (2H, m), 1.37-1.25 (1H, m), 1.55 (2H, d, J = 11.4 Hz), 1.70-1.61 (2H, m), 2.06 |
| | (3H, s), 2.61 (2H, d, J = 11.4 Hz), 2.80-2.75 (2H, m), 3.96 (3H, s), 4.62-4.52 (3H, m), 6.74- |
| | 6.71 (2H, m), 6.85 (1H, d, J = 5.7 Hz), 7.23 (1H, s), 7.42 (1H, dd, J = 1.7, 8.6 Hz), 7.59 (1H, s), |
| | 7.77 (1H, d, J = 5.7 Hz), 8.16-8.13 (1H, m), 8.70 (1H, t, J = 5.7 Hz). |
| 26.02 | (d4-MeOH) 1.29-1.51 (2H, m), 1.58-1.64 (2H, m), 1.84-1.96 (1H, m, 1H), 2.20 (3H, s), 2.34- |
| | 2.45 (2H, m), 2.51 (3H, s), 3.06-3.17 (2H, m), 4.35 (2H, d, J = 7.2 Hz), 4.74 (2H, s), 6.99- |
| | 7.02 (1H, m), 7.56 (1H, dd, J = 8.6, 1.8 Hz), 7.71 (1H, s), 7.73 (1H, d, J = 6.1 Hz), 8.13 |
| | (1H, d, J = 8.6 Hz). |
| 26.03 | (d4-MeOH) 1.08-1.25 (2H, m), 1.26-1.54 (3H, m), 1.62-1.79 (2H, m), 1.88 (2H, s), 2.00 |
| | (3H, s), 2.13 (3H, d, J = 1.1 Hz), 2.52-2.66 (2H, m), 2.98-3.07 (1H, m), 3.82 (1H, dd, J = 13.7, |
| | 7.9 Hz), 4.56 (1H, d, J = 15.2 Hz), 4.68 (1H, d, J = 15.1 Hz), 6.99 (1H, d, J = 6.0 Hz), 7.40 (1H, d, |
| | J = 1.1 Hz), 7.55 (1H, dd, J = 8.6, 1.8 Hz), 7.69 (1H, s), 7.74 (1H, d, J = 5.9 Hz), 8.11 |
| | (1H, d, J = 8.5 Hz). |
| 26.04 | 1.12-1.27 (2H, m), 1.46-1.73 (3H, m), 2.06-2.26 (3H, m), 2.42 (3H, d, J = 1.0 Hz), 2.60- |
| | 2.80 (2H, m), 2.85-2.97 (2H, m), 4.00 (2H, d, J = 6.7 Hz), 4.60 (2H, d, J = 5.7 Hz), 6.76 (2H, s), |
| | 6.86 (1H, d, J = 5.8 Hz), 6.91 (1H, d, J = 1.1 Hz), 7.40 (1H, dd, J = 8.6, 1.8 Hz), 7.57 (1H, d, J = |
| | 1.7 Hz), 7.67 (1H, t, J = 5.5 Hz), 7.78 (1H, d, J = 5.8 Hz), 8.16 (1H, d, J = 8.6 Hz). |
| 26.05 | 1.12-1.22 (1H, m), 1.32-1.44 (1H, m), 1.55-1.66 (2H, m), 1.74-1.83 (2H, m), 2.12 (2H, s), |
| | 2.39 (3H, d, J = 1.0 Hz), 2.71-2.77 (2H, m), 3.05 (2H, t, J = 6.5 Hz), 4.46 (2H, d, J = 6.0 Hz), |
| | 6.60 (1H, d, J = 1.2 Hz), 6.67-6.73 (2H, m), 6.84 (1H, d, J = 5.8 Hz), 7.37 (1H, dd, J = 8.6, 1.7 |
| | Hz), 7.45-7.51 (2H, m), 7.73-7.79 (1H, m), 7.93 (1H, t, J = 6.0 Hz), 8.07-8.17 (1H, m). |
| 26.06 | 0.96-1.09 (2H, m),1.19-1.31 (1H, m), 1.45-1.69 (4H, m), 1.79 (3H, s), 2.02 (3H, s), 2.48 |
| | (3H, s), 2.54-2.63 (2H, m), 3.14-3.25 (1H, m), 3.47-3.64 (1H, m), 4.50 (2H, d, J = 5.9 Hz), |
| | 6.72 (2H, s), 6.83 (1H, d, J = 5.8 Hz), 6.86 (1H, s), 7.37 (1H, dd, J = 8.6, 1.7 Hz), 7.51 (1H, d, J = |
| | 1.7 Hz), 7.77 (1H, d, J = 5.8 Hz), 8.14 (1H, d, J = 8.6 Hz), 8.59 (1H, t, J = 6.1 Hz). |
| 26.07 | 1.49-1.57 (2H, m), 1.57-1.66 (2H, m), 1.71-1.81 (2H, m), 2.00 (3H, s), 2.09 (3H, s), 2.19- |
| | 2.26 (1H, m), 2.62-2.70 (2H, m), 4.52-4.61 (2H, m), 6.73 (2H, s), 6.81-6.88 (1H, m), 7.34- |
| | 7.41 (2H, m), 7.56 (1H, s), 7.74-7.81 (1H, m), 8.11-8.17 (1H, m), 8.23 (1H, s), 9.45 (1H, s). |
| 26.08 | 1.01-1.09 (1H, m), 1.12-1.22 (1H, m), 1.39-1.48 (2H, m), 1.51-1.57 (1H, m), 1.60 (2H, s), |
| | 1.77 (3H, s), 1.95 (3H, s), 2.01 (3H, s), 2.54-2.60 (1H, m), 3.01-3.10 (1H, m), 3.51-3.62 |
| | (1H, m), 4.45-4.57 (2H, m), 6.72 (2H, s), 6.82-6.88 (1H, m), 7.38-7.42 (1H, m), 7.53-7.56 |
| | (1H, m), 7.75-7.79 (1H, m), 8.11-8.18 (1H, m), 8.92 (1H, t, J = 5.9 Hz). |
| 26.09 | 1.05-1.16 (2H, m), 1.21-1.32 (1H, m), 1.53-1.63 (2H, m), 1.64-1.73 (2H, m), 2.08 (3H, s), |
| | 2.15 (3H, s), 2.60-2.68 (2H, m), 3.03-3.07 (2H, m), 4.44-4.51 (2H, m), 6.67-6.73 (2H, m), |
| | 6.82-6.86 (1H, m), 7.21 (1H, t, J = 6.5 Hz), 7.33-7.40 (1H, m), 7.47-7.52 (1H, m), 7.72- |
| | 7.79 (1H, m), 8.09-8.17 (1H, m), 8.46 (1H, t, J = 6.0 Hz). |
| 26.10 | 1.05-1.17 (2H, m), 1.24-1.34 (1H, m), 1.54-1.60 (2H, m), 1.67-1.75 (2H, m), 2.09 (3H, s), |
| | 2.36 (3H, s), 2.63-2.69 (2H, m), 3.24 (2H, t, J = 6.7 Hz), 4.50 (2H, d, J = 5.8 Hz), 6.71 (2H, s), |
| | 6.84 (1H, d, J = 5.8 Hz), 7.17 (1H, t, J = 6.7 Hz), 7.37 (1H, dd, J = 8.7, 1.7 Hz), 7.50 (1H, d, J = |
| | 1.7 Hz), 7.76 (1H, d, J = 5.8 Hz), 8.13 (1H, d, J = 8.6 Hz), 8.46 (1H, t, J = 6.0 Hz). |
| 26.11 | (d4-MeOH) 1.23-1.12 (2H, m), 1.38-1.24 (3H, m), 1.73-1.63 (2H, m), 2.11 (3H, s), 2.18 |
| | (3H, s), 2.36 (3H, s), 2.75-2.68 (2H, m), 3.00-2.95 (2H, m), 4.64 (2H, s), 6.97 (1H, d, J = 6.0 Hz), |
| | 7.51 (1H, dd, J = 8.6, 1.8 Hz), 7.73 (1H, d, J = 6.0 Hz), 7.64 (1H, s), 8.09 (1H, d, J = 8.7 Hz) |
| | 4 × N-H not observed. |
| 26.12 | 1.01-1.12 (3H, m), 1.37-1.47 (2H, m), 1.52-1.59 (2H, m), 1.64-1.75 (2H, m), 2.08 (3H, s), |
| | 2.43 (3H, s), 2.64 (2H, d, J = 10.0 Hz), 2.81 (2H, dd, J = 9.3, 6.6 Hz), 4.51 (2H, d, J = 5.8 Hz), |
| | 6.67-6.76 (3H, m), 6.84 (1H, d, J = 5.8 Hz), 7.39 (1H, dd, J = 8.4, 1.7 Hz), 7.52 (1H, s), 7.77 |
| | (1H, d, J = 5.8 Hz), 8.14 (1H, d, J = 8.6 Hz), 8.49 (1H, t, J = 6.1 Hz). |
| 26.13 | 1.12-1.27 (2H, m), 1.59-1.68 (2H, m), 1.72-1.87 (2H, m), 2.12 (3H, s), 2.70-2.77 (2H, m), |
| | 3.05-3.14 (3H, m), 4.45-4.54 (2H, m), 6.63-6.76 (2H, m), 6.80-6.88 (2H, m), 7.34-7.41 |
| | (1H, m), 7.47 (1H, t, J = 6.3 Hz), 7.49-7.55 (2H, m), 7.71-7.79 (1H, m), 8.07-8.13 (1H, m), |
| | 8.16 (1H, t, J = 6.0 Hz). |
| 26.14 | 0.96-1.11 (2H, m), 1.42-1.52 (1H, m), 1.55-1.65(2H, m), 2.34-2.47(6H, m), 2.90-2.99 |
| | (2H, m), 3.03-3.12 (2H, m), 4.43-4.58 (2H, m), 6.44 (1H, t, J = 6.6 Hz),6.68-6.76 (2H, m), |
| | 6.81-6.93 (1H, m), 7.35-7.43 (1H, m), 7.51-7.54(1H, m), 7.74-7.80(1H, m), 8.08-8.20 |
| | (1H, m), 8.90 (1H, t, J = 5.9 Hz). |
| 26.15 | (d4-MeOH) 1.12-1.23 (2H, m), 1.28-1.38 (1H, m), 1.61-1.68 (2H, m), 1.75-1.86 (2H, m), |
| | 2.19 (3H, s), 2.46 (3H, s), 2.70-2.75 (2H, m), 3.03-3.08 (2H, m), 4.65-4.67 (2H, m), 6.95- |
| | 6.99 (1H, m), 7.50-7.54 (1H, m), 7.65-7.68 (1H, m), 7.72-7.75 (1H, m), 8.08-8.12 (1H, m). |
| 26.16 | 1.13-1.26 (2H, m), 1.40-1.51 (1H, m), 1.57-1.65 (2H, m), 1.74-1.84 (2H, m), 2.13 (3H, s), |
| | 2.71-2.77 (2H, m), 3.33-3.36 (2H, m), 4.48-4.53 (2H, m), 6.67-6.73 (2H, m), 6.83-6.87 |
| | (1H, m), 7.36-7.40 (1H, m), 7.50-7.55 (2H, m), 7.75-7.78 (1H, m), 8.11-8.15 (1H, m), 8.39 |
| | (1H, t, J = 6.0 Hz), 8.90 (1H, s). |
| 35.01 | 1.08-0.97 (2H, m), 1.39-1.38 (11H, m), 2.01-1.92 (1H, m), 2.72-2.57 (2H, m), 3.87 (2H, d, |
| | J = 11.9 Hz), 4.39 (2H, d, J = 7.0 Hz), 4.65 (2H, d, J = 5.9 Hz), 7.02 (1H, s), 7.19 (1H, d, J = 6.8 |
| | Hz), 7.70-7.66 (2H, m), 7.80 (1H, s), 8.54-8.45 (3H, m), 9.32 (1H, t, J = 6.0 Hz). |
| 35.02 | 1.41-1.29 (2H, m), 1.61 (2H, d, J = 13.2 Hz), 2.11-2.04 (1H, m), 2.77 (2H, dd, J = 10.7, 12.7 |
| | Hz), 3.21 (2H, d, J = 12.7 Hz), 4.36 (2H, d, J = 7.0 Hz), 4.63- |
| | 4.61 (2H, m), 6.97 (1H, s), 7.20 (1H, d, J = 7.1 Hz), 7.57 (1H, d, J = 7.1 Hz), |
| | 7.71-7.66 (1H, m), 7.79 (1H, s), 8.44-8.40 (1H, m). |
| 35.03 | 1.06 (3H, t, J = 7.1 Hz), 1.28 (2H, q, J = 11.4 Hz), 1.59-1.48 (2H, m), 1.92-1.92 (1H, m), 2.35- |
| | 2.28 (1H, m), 2.72-2.58 (1H, m), 2.96-2.89 (1H, m), 3.19-3.07 (2H, m), 4.45-4.40 (2H, |
| | m), 4.60-4.57 (2H, m), 6.77 (2H, s), 6.88 (1H, d, J = 5.8 Hz), 7.01 (1H, s), 7.43-7.39 (1H, m), |
| | 7.57 (1H, s), 7.80-7.77 (1H, m), 8.19-8.15 (1H, m), 9.26 (1H, t, J = 6.0 Hz). |
| 35.04 | 1.16-1.02 (3H, m), 1.72-1.51 (6H, m), 2.06-2.05 (3H, m), 2.66-2.60 (2H, m), 4.48 (2H, t, J = |
| | 7.3 Hz), 4.56 (2H, d, J = 5.9 Hz), 6.74-6.71 (2H, m), 6.86 (1H, d, J = 5.7 Hz), 6.94 (1H, s), |
| | 7.39 (1H, dd, J = 1.7, 8.6 Hz), 7.55 (1H, s), 7.77 (1H, d, J = 5.8 Hz), 8.16-8.13 (1H, m), 9.22 |
| | (1H, t, J = 5.9 Hz). |
| 35.05 | 0.60-0.66 (2H, m), 0.84-0.94 (2H, m), 1.04-1.18 (2H, m), 1.45 (2H, dd, J = 13.4, 3.6 Hz), |
| | 1.89 (1H, tt, J = 8.4, 5.0 Hz), 2.01 (1H, ddt, J = 11.3, 7.8, 3.8 Hz), 2.73 (2H, td, J = 12.8, 2.6 |
| | Hz), 3.78-3.85 (2H, m), 4.34 (2H, d, J = 7.1 Hz), 4.53 (2H, d, J = 6.0 Hz), 6.60 (1H, s), 6.65- |
| | 6.70 (2H, m), 6.75 (2H, s), 6.86 (1H, dd, J = 6.0, 0.8 Hz), 7.38 (1H, dd, J = 8.6, 1.8 Hz), 7.53 |
| | (1H, d, J = 1.7 Hz), 7.77 (1H, d, J = 5.8 Hz), 8.05-8.10 (2H, m), 8.16 (1H, d, J = 8.6 Hz), 9.04 |
| | (1H, t, J = 6.1 Hz). |
| 35.06 | 1.03-1.18 (2H, m), 1.39-1.51 (2H, m), 1.97-2.09 (1H, m), 2.18 (3H, s), 2.66-2.80 (2H, m), |
| | 3.74-3.89 (2H, m), 4.35 (2H, d, J = 7.1 Hz), 4.53 (2H, d, J = 6.0 Hz), 6.61-6.68 (2H, m), 6.69 |
| | (1H, s), 6.74 (2H, s), 6.85 (1H, d, J = 5.8 Hz), 7.38 (1H, dd, J = 8.6, 1.7 Hz), 7.52 (1H, s), 7.76 |
| | (1H, d, J = 5.8 Hz), 8.01-8.09 (2H, m), 8.15 (1H, d, J = 8.6 Hz), 9.07 (1H, t, J = 6.1 Hz) |
| 35.07 | 0.99-1.17 (3H, m), 1.54-1.67 (4H, m), 1.71-1.82 (2H, m), 2.11 (3H, s), 2.65-2.72 (2H, m), |
| | 4.53 (2H, t, J = 7.2 Hz), 4.57 (2H, d, J = 6.0 Hz), 6.73 (2H, s), 6.86 (1H, d, J = 5.8 Hz), 6.91 (1H, |
| | d, J = 2.0 Hz), 7.40 (1H, dd, J = 8.6, 1.8 Hz), 7.50 (1H, d, J = 2.0 Hz), 7.55 (1H, d, J = 1.7 Hz), |
| | 7.77 (1H, d, J = 5.8 Hz), 8.15 (1H, d, J = 8.6 Hz), 9.11 (1H, t, J = 6.1 Hz) |
| 35.08 | 0.92-1.14 (5H, m), 1.49-1.56 (2H, m), 1.65-1.85 (4H, m), 2.12 (3H, s), 2.65-2.73 (2H, m), |
| | 4.47 (2H, t, J = 7.1 Hz), 4.56 (2H, d, J = 6.0 Hz), 6.73 (2H, s), 6.85 (1H, d, J = 5.8 Hz), 6.90 (1H, |
| | d, J = 2.0 Hz), 7.40 (1H, dd, J = 8.6, 1.8 Hz), 7.49 (1H, d, J = 2.0 Hz), 7.54 (1H, s), 7.77 |
| | (1H, d, J = 5.8 Hz), 8.15 (1H, d, J = 8.6 Hz), 9.11 (1H, t, J = 6.1 Hz). |
| 35.09 | 1.29 (3H, t, J = 6.9 Hz), 3.94 (2H, q, J = 7.0 Hz), 4.56 (2H, d, J = 6.0 Hz), 5.65 (2H, s), 6.75- |
| | 6.83 (5H, m), 6.93 (1H, d, J = 2.0 Hz), 7.09 (2H, d, J = 8.7 Hz), 7.38 (1H, dd, J = 8.6, 1.6 Hz), |
| | 7.50 (1H, s), 7.53 (1H, d, J = 2.0 Hz), 7.76 (1H, d, J = 5.8 Hz), 8.15 (1H, d, J = 8.6 Hz), |
| | 8.25 (1H, s), 9.14 (1H, t, J = 6.1 Hz) |
| 46.01 | 1.02-1.17 (3H, m), 1.54-1.60 (2H, m), 1.60-1.73 (4H, m), 2.06 (3H, s), 2.61-2.67 (2H, m), |
| | 4.54 (2H, t, J = 7.2 Hz), 4.63 (2H, d, J = 5.6 Hz), 6.49 (2H, s), 6.86 (1H, d, J = 2.1 Hz), 7.02 (1H, |
| | dd, J = 5.6, 0.8 Hz), 7.49 (1H, d, J = 2.0 Hz), 7.51 (1H, d, J = 1.0 Hz), 7.71 (1H, d, J = 5.6 Hz), |
| | 9.20 (1H, t, J = 6.0 Hz). |
| 51.01 | 1.52-1.57 (2H, m), 2.09 (6H, br.s), 2.57 (4H, br.s), 3.42 (3H, br.s), 3.97 (2H, t, J = 7.2 Hz), 4.52 |
| | (2H, d, J = 5.4 Hz), 6.61 (1H, s), 6.81 (2H, br.s), 6.87 (1H, d, J = 5.4 Hz), 7.33-7.46 (6H, m), |
| | 7.54 (1H, s), 7.74 (1H, d, J = 5.7 Hz), 8.14 (1H, d, J = 8.4 Hz), 8.31 (1H, t, J = 5.7 Hz) |
| 51.02 | 1.23 (3H, t, J = 6.9 Hz), 2.00 (3H, s), 2.42-2.49 (4H, m), 3.24-3.31 (4H, m), 3.44 (2H, s), 4.37 |
| | (2H, q, J = 6.9 Hz), 4.51 (2H, d, J = 6.1 Hz), 6.67 (1H, s), 6.71 (2H, d, J = 5.2 Hz), 6.76-6.82 |
| | (2H, m), 6.85 (1H, d, J = 5.8 Hz), 7.38 (1H, dd, J = 8.6, 1.8 Hz), 7.52 (1H, s), 7.75 (1H, d, J = 5.8 |
| | Hz), 8.07-8.21 (3H, m), 8.52 (1H, t, J = 6.1 Hz) |
| 51.03 | 1.15 (3H, t, J = 7.6 Hz), 2.52-2.60 (6H, m), 3.28-3.34 (4H, m), 3.67 (2H, s), 4.56 (2H, d, J = |
| | 5.9 Hz), 6.73 (2H, s), 6.78-6.83 (2H, m), 6.87 (1H, d, J = 5.8 Hz), 7.40 (1H, dd, J = 8.6, 1.7 Hz), |
| | 7.56 (1H, d, J = 1.7 Hz), 7.65 (1H, s), 7.77 (1H, d, J = 5.8 Hz), 8.12-8.18 (3H, m), |
| | 8.99 (1H, t, J = 6.0 Hz) |
| 51.04 | 1.20-1.34 (2H, m), 1.50-1.60 (2H, m), 2.10-2.20 (1H, m), 2.30 (3H, s), 2.73-2.85 (2H, m), |
| | 3.93 (2H, d, J = 13.1 Hz), 3.99 (2H, d, J = 7.3 Hz), 4.53 (2H, d, J = 6.3 Hz), 6.46 (1H, s), 6.71 |
| | (2H, s), 6.75-6.81 (2H, m), 6.84 (1H, d, J = 5.8 Hz), 7.40 (1H, dd, J = 8.6, 1.8 Hz), 7.52 (1H, s), |
| | 7.76 (1H, d, J = 5.8 Hz), 8.06-8.16 (3H, m), 8.61 (1H, t, J = 6.3 Hz) |
| 51.05 | 1.13-1.26 (3H, m), 1.59-1.84 (6H, m), 2.12 (3H, s), 2.69-2.76 (2H, m), 4.16-4.24 (2H, m), |
| | 4.54 (2H, d, J = 6.2 Hz), 6.71 (2H, s), 6.82 (1H, s), 6.84 (1H, d, J = 5.8 Hz), 7.39 |
| | (1H, dd, J = 8.6, 1.7 Hz), 7.48-7.55 (1H, m), 7.76 (1H, d, J = 5.8 Hz), 8.13 (1H, d, J = 8.6 Hz), |
| | 8.86 (1H, t, J = 6.2 Hz) |
| 51.06 | 1.09-1.22 (3H, m), 1.59-1.65 (2H, m), 1.70-1.80 (4H, m), 2.11 (3H, s), 2.67-2.74 (2H, m), |
| | 4.19 (2H, t, J = 7.4 Hz), 4.54 (2H, d, J = 6.3 Hz), 6.64 (1H, d, J = 2.3 Hz), 6.70 (2H, s), 6.84 (1H, |
| | d, J = 5.8 Hz), 7.40 (1H, dd, J = 8.6, 1.7 Hz), 7.52 (1H, s), 7.76 (1H, d, J = 5.8 Hz), 7.84 |
| | (1H, d, J = 2.3 Hz), 8.12 (1H, d, J = 8.6 Hz), 8.70 (1H, t, J = 6.3 Hz) |
| 51.07 | 1.02-1.19 (5H, m), 1.54-1.62 (2H, m), 1.74-1.86 (4H, m), 2.11 (3H, s), 2.67-2.74 (2H, m), |
| | 4.14 (2H, t, J = 7.1 Hz), 4.54 (2H, d, J = 6.3 Hz), 6.65 (1H, d, J = 2.3 Hz), 6.70 (2H, s), 6.84 (1H, |
| | d, J = 5.8 Hz), 7.40 (1H, dd, J = 8.6, 1.7 Hz), 7.51-7.54 (1H, m), 7.76 (1H, d, J = 5.8 Hz), 7.83 |
| | (1H, d, J = 2.3 Hz), 8.12 (1H, d, J = 8.6 Hz), 8.70 (1H, t, J = 6.3 Hz) |
| 51.08 | 0.89-1.02 (1H, m), 1.02-1.15 (1H, m), 1.36-1.48 (1H, m), 1.64-1.72 (2H, m), 1.72-1.81 |
| | (2H, m), 1.96 (3H, s), 2.39-2.48 (1H, m), 2.89-2.99 (1H, m), 3.75 (1H, d, J = 13.2 Hz), 4.21 |
| | (2H, t, J = 7.3 Hz), 4.32 (1H, d, J = 12.9 Hz), 4.54 (2H, d, J = 6.2 Hz), 6.65 (1H, d, J = 2.3 Hz), |
| | 6.76 (2H, s), 6.84 (1H, d, J = 5.8 Hz), 7.40 (1H, dd, J = 8.6, 1.6 Hz), 7.50-7.54 (1H, m), 7.75 |
| | (1H, d, J = 5.8 Hz), 7.85 (1H, d, J = 2.3 Hz), 8.12 (1H, d, J = 8.6 Hz), 8.21 (1H, s), |
| | 8.71 (1H, t, J = 6.3 Hz) |
| 69.01 | 2.28 (3H, s), 2.50-2.54 (2H, m), 2.75-2.80 (2H, m), 3.22 (3H, s), 3.34 (2H, s), 4.52 (2H, d, J = |
| | 5.6 Hz), 4.85 (2H, s), 5.32 (2H, s), 6.76 (1H, d, J = 7.7 Hz), 6.95 (1H, d, J = 11.1 Hz), 7.65 |
| | (1H, s), 7.83 (1H, s), 7.97 (1H, s), 8.28 (1H, s), 8.68-8.72 (1H, m), 11.93 (1H, br. s) |
| 82.01 | 1.28-1.16 (2H, m), 1.59-1.49 (1H, m), 1.65 (2H, d, J = 12.4 Hz), 1.80 (2H, dt, J = 2.4, 11.6 |
| | Hz), 2.13 (3H, s), 2.78-2.71 (2H, m), 3.38 (2H, t, J = 6.5 Hz), 4.35 (2H, s), 4.78 (2H, s), 6.68 |
| | (1H, d, J = 5.1 Hz), 6.75 (2H, s), 6.92-6.86 (2H, m), 7.34 (1H, dd, J = 1.7, 8.6 Hz), 7.55 (1H, d, |
| | J = 1.1 Hz), 7.77 (1H, d, J = 5.9 Hz), 8.12 (1H, d, J = 5.4 Hz), 8.16 (1H, d, J = 8.7 Hz). |

### Biological Methods

Determination of the % Inhibition for FXIIa

Factor XIIa inhibitory activity in vitro was determined using standard published methods (see e.g. Shori et al., Biochem. Pharmacol., 1992, 43, 1209; Baeriswyl et al., ACS Chem. Biol., 2015, 10 (8) 1861; Bouckaert et al., European Journal of Medicinal Chemistry 110 (2016) 181). Human Factor XIIa (Enzyme Research Laboratories) was incubated at 25° C. with the fluorogenic substrate H-DPro-Phe-Arg-AFC and various concentrations of the test compound. Residual enzyme activity (initial rate of reaction) was determined by measuring the change in optical absorbance at 410 nm and the IC50 value for the test compound was determined.

Data acquired from this assay are shown in Table 14 using the following scale:

| Category | IC₅₀ (nM) |
|---|---|
| A | <1,000 |
| B | 1,000-3,000 |
| C | 3,000-10,000 |
| D | 10,000-40,000 |
| E | 40,000-70,000 |

TABLE 14
Human FXIIa data, molecular weight and LCMS data

| | Human | | |
|---|---|---|---|
| Example | FXIIa | Molecular | LCMS |
| number | IC50 (nM) | weight | Mass Ion |
| 2.01 | D | 374.1 | 375.2 |
| 2.02 | D | 436.1 | 437.3 |
| 2.03 | D | 422.1 | 423.3 |
| 2.04 | C | 457.2 | 458.1 |
| 2.05 | D | 466.1 | 467.4 |
| 2.06 | D | 466.1 | 467.4 |
| 2.07 | D | 466.1 | 467.4 |
| 2.08 | D | 479.2 | 480.5 |
| 2.09 | D | 404.1 | 405.4 |
| 2.10 | D | 444.1 | 445.4 |
| 2.11 | C | 514.1 | 515.4 |
| 2.12 | C | 514.1 | 515.4 |
| 2.13 | D | 514.1 | 515.4 |
| 2.14 | D | 479.2 | 480.5 |
| 2.15 | D | 461.1 | 462.4 |
| 2.16 | C | 417.1 | 418.4 |
| 2.17 | D | 430.1 | 431.4 |
| 2.18 | D | 459.1 | 460.4 |
| 2.19 | C | 472.2 | 473.5 |
| 2.20 | C | 457.2 | 458.4 |
| 2.21 | D | 466.1 | 467.4 |
| 2.22 | D | 480.1 | 481.4 |
| 2.23 | D | 440.1 | 441.4 |
| 2.24 | D | 500.2 | 501.5 |
| 2.25 | D | 507.1 | 508.5 |
| 2.26 | D | 416.1 | 417.5 |
| 2.27 | D | 431.1 | 432.4 |
| 2.28 | D | 480.1 | 481.4 |
| 2.29 | D | 505.2 | 506.1 |
| 2.30 | D | 437.1 | 438.4 |
| 2.31 | C | 437.1 | 438.4 |
| 2.32 | C | 437.1 | 438.4 |
| 2.33 | C | 431.2 | 432.5 |
| 2.34 | C | 514.2 | 515.6 |
| 2.35 | C | 523.2 | 524.5 |
| 2.36 | B | 520.2 | 521.6 |
| 2.37 | C | 486.2 | 487.6 |
| 2.38 | D | 457.1 | 458.4 |
| 2.39 | D | 534.2 | 535.5 |
| 2.40 | C | 533.2 | 534.6 |
| 2.41 | D | 533.2 | 534.5 |
| 2.42 | D | 519.1 | 520.5 |
| 2.43 | D | 471.1 | 472.5 |
| 2.44 | C | 534.2 | 535.6 |
| 2.45 | C | 551.2 | 552.6 |
| 2.46 | D | 444.1 | 445.5 |
| 2.47 | D | 457.2 | 458.5 |
| 2.48 | D | 452.1 | 453.5 |
| 2.49 | C | 500.1 | 501.5 |
| 2.50 | D | 461.1 | 462.0 |
| 2.51 | D | 445.1 | 446.0 |
| 2.52 | C | 537.2 | 538.2 |
| 2.53 | C | 499.2 | 500.1 |
| 2.54 | C | 441.1 | 442.1 |
| 2.55 | D | 479.1 | 480.4 |
| 2.56 | C | 473.2 | 474.5 |
| 2.57 | C | 533.2 | 534.4 |
| 2.58 | C | 519.1 | 520.3 |
| 5.01 | D | 506.2 | 507.3 |
| 5.02 | C | 443.2 | 444.5 |
| 5.03 | D | 416.1 | 417.4 |
| 5.04 | D | 429.1 | 430.5 |
| 5.05 | D | 457.1 | 458.4 |
| 5.06 | D | 457.2 | 458.5 |
| 5.07 | D | 493.1 | 494.5 |
| 5.08 | D | 436.1 | 437.4 |
| 5.09 | D | 448.1 | 449.5 |
| 5.10 | C | 463.1 | 464.5 |
| 5.11 | D | 402.1 | 403.4 |
| 5.12 | C | 492.1 | 492.7 |
| 5.13 | D | 525.2 | 526.6 |
| 5.14 | D | 472.1 | 473.1 |
| 5.15 | D | 493.1 | 494.4 |
| 5.16 | C | 492.1 | 493.3 |
| 5.17 | D | 492.1 | 493.3 |
| 5.18 | D | 492.1 | 493.1 |
| 5.19 | C | 449.1 | 450.1 |
| 5.20 | D | 472.2 | 473.3 |
| 5.21 | D | 492.1 | 493.1 |
| 5.22 | C | 463.1 | 464.2 |
| 5.23 | D | 468.1 | 469.2 |
| 5.24 | C | 486.2 | 487.3 |
| 5.25 | D | 459.2 | 460.0 |
| 5.26 | D | 473.2 | 474.1 |
| 5.27 | D | 502.2 | 503.2 |
| 5.28 | D | 475.2 | 476.2 |
| 5.29 | D | 488.2 | 489.4 |
| 7.01 | D | 501.1 | 502.4 |
| 7.02 | D | 477.1 | 478.4 |
| 7.03 | B | 507.1 | 508.6 |
| 7.04 | D | 537.2 | 538.6 |
| 7.05 | B | 507.1 | 508.6 |
| 7.06 | C | 460.1 | 461.3 |
| 7.07 | C | 458.2 | 459.4 |
| 7.08 | B | 458.2 | 459.3 |
| 7.09 | C | 508.1 | 509.2 |
| 7.10 | D | 438.1 | 439.3 |
| 7.11 | D | 515.1 | 516.5 |
| 7.12 | D | 459.1 | 460.5 |
| 7.13 | C | 445.1 | 446.4 |
| 7.14 | C | 444.1 | 445.5 |
| 7.15 | C | 418.1 | 419.5 |
| 7.16 | C | 441.1 | 442.4 |
| 7.17 | D | 494.2 | 495.5 |
| 7.18 | D | 534.2 | 535.6 |
| 7.19 | C | 438.1 | 439.4 |
| 7.20 | C | 438.1 | 439.4 |
| 7.21 | C | 439.1 | 440.4 |
| 7.22 | B | 460.2 | 461.5 |
| 7.23 | B | 472.2 | 473.5 |
| 7.24 | D | 466.1 | 467.5 |
| 7.25 | C | 474.1 | 475.5 |
| 7.26 | B | 446.2 | 447.5 |
| 7.27 | C | 469.1 | 470.5 |
| 7.28 | D | 455.1 | 456.4 |
| 7.29 | D | 466.1 | 467.5 |
| 7.30 | D | 480.1 | 481.4 |
| 7.31 | B | 432.1 | 433.5 |
| 25.01 | D | 317.0 | 317.7 |
| 25.02 | C | 375.0 | 375.7/377.7 |
| 25.03 | D | 317.0 | 318.2 |
| 25.04 | C | 331.1 | 332.2 |
| 25.05 | D | 423.1 | 424.0 |
| 25.06 | D | 453.1 | 454.0 |
| 25.07 | B | 454.0 | 455.1/457.1 |
| 25.08 | D | 407.1 | 408.0 |
| 25.09 | D | 380.2 | 381.3 |
| 25.10 | C | 428.1 | 429.4 |
| 25.11 | B | 442.2 | 443.1 |
| 25.12 | C | 408.2 | 409.2 |
| 25.13 | C | 361.0 | 362.0 |
| 25.14 | B | 395.2 | 396.1 |
| 25.15 | A | 409.2 | 410.2 |
| 25.16 | D | 395.2 | 396.5 |
| 25.17 | D | 409.2 | 410.4 |
| 25.101 | D | 333.1 | 334.0 |
| 25.102 | C | 322.1 | 323.3 |
| 25.103 | D | 337.0 | 338.2 |
| 25.104 | D | 349.1 | 350.3 |
| 25.105 | E | 309.1 | 310.0 |
| 25.201 | C | 427.2 | 428.4 |
| 25.202 | B | 442.2 | 443.3 |
| 25.203 | B | 367.1 | 368.3 |
| 25.204 | D | 367.1 | 368.0 |
| 25.205 | C | 351.1 | 352.0 |
| 25.206 | D | 351.1 | 352.0 |
| 25.207 | B | 350.1 | 351.0 |
| 25.208 | C | 445.2 | 446.4 |
| 25.209 | C | 459.2 | 460.4 |
| 25.210 | A | 412.3 | 412.2/414.2 |
| 26.01 | C | 407.2 | 408.1 |
| 26.02 | D | 407.2 | 408.5 |
| 26.03 | D | 465.2 | 466.2 |
| 26.04 | D | 424.2 | 425.4 |
| 26.05 | B | 423.2 | 424.1 |
| 26.06 | D | 465.2 | 466.2 |
| 26.07 | D | 437.2 | 438.1 |
| 26.08 | D | 499.2 | 500.1 |
| 26.09 | B | 457.2 | 458.3 |
| 26.10 | B | 457.2 | 458.3 |
| 26.11 | C | 437.2 | 438.0 |
| 26.12 | C | 422.2 | 423.0 |
| 26.13 | C | 409.2 | 410.4 |
| 26.14 | D | 410.2 | 411.1 |
| 26.15 | C | 424.2 | 425.2 |
| 26.16 | B | 410.2 | 411.1 |
| 35.01 | D | 498.2 | 499.4 |
| 35.02 | C | 398.2 | 399.3 |
| 35.03 | C | 426.2 | 427.4 |
| 35.04 | A | 426.2 | 427.1 |
| 35.05 | C | 481.3 | 482.3 |
| 35.06 | D | 455.2 | 456.4 |
| 35.07 | B | 392.2 | 393.2 |
| 35.08 | B | 406.2 | 407.2 |
| 35.09 | D | 401.2 | 402.1 |
| 46.01 | D | 398.2 | 399.4 |
| 51.01 | D | 483.3 | 484.0 |
| 51.02 | D | 483.3 | 484.4 |
| 51.03 | C | 486.2 | 487.3 |
| 51.04 | C | 455.2 | 456.4 |
| 51.05 | A | 426.2 | 427.2 |
| 51.06 | B | 392.2 | 393.2 |
| 51.07 | B | 406.2 | 407.2 |
| 51.08 | D | 420.2 | 421.2 |
| 69.01 | B | 496.2 | 497.3 |
| 82.01 | D | 416.2 | 417.1 |

Determination of the % Inhibition for FXIa

FXIa inhibitory activity in vitro was determined using standard published methods (see e.g. Johansen et al., Int. J. Tiss. Reac. 1986, 8, 185; Shori et al., Biochem. Pharmacol., 1992, 43, 1209; Stürzebecher et al., Biol. Chem. Hoppe-Seyler, 1992, 373, 1025). Human FXIa (Enzyme Research Laboratories) was incubated at 25° C. with the fluorogenic substrate Z-Gly-Pro-Arg-AFC and various concentrations of the test compound. Residual enzyme activity (initial rate of reaction) was determined by measuring the change in fluorescence at 410 nm and the IC50 value for the test compound was determined.

TABLE 15
Selectivity; FXIa data

| Ex. No. | Human FXIa IC50 (nM) |
|---|---|
| 2.36 | 76,400 |
| 2.58 | >40,000 |
| 5.12 | >40,000 |
| 5.16 | >40,000 |
| 5.17 | >40,000 |
| 5.18 | 37,700 |
| 5.19 | 29,300 |
| 5.20 | >40,000 |
| 5.21 | >40,000 |
| 5.22 | >40,000 |
| 5.23 | 8,350 |
| 5.24 | >40,000 |
| 5.25 | >40,000 |
| 5.26 | >40,000 |
| 5.27 | >40,000 |
| 5.28 | >40,000 |
| 5.29 | 38,500 |
| 25.07 | >40,000 |
| 25.08 | >40,000 |
| 25.09 | >40,000 |
| 25.10 | >40,000 |
| 25.102 | >40,000 |
| 25.201 | >40,000 |
| 25.202 | >40,000 |
| 25.203 | >40,000 |
| 25.207 | >40,000 |
| 26.01 | >40,000 |
| 26.02 | >40,000 |
| 26.03 | >40,000 |
| 26.04 | >40,000 |
| 26.05 | >40,000 |
| 26.06 | >40,000 |
| 26.07 | >40,000 |
| 26.08 | >40,000 |
| 26.09 | >40,000 |
| 26.10 | >40,000 |
| 35.01 | >40,000 |
| 35.02 | >40,000 |
| 35.03 | >40,000 |
| 35.04 | >40,000 |
| 35.05 | 31,700 |
| 35.06 | >40,000 |
| 35.07 | >40,000 |
| 51.02 | 12,400 |
| 51.03 | 20,500 |
| 51.04 | >40,000 |
| 51.05 | >40,000 |
| 69.01 | >40,000 |
| 82.01 | >40,000 |

### NUMBERED EMBODIMENTS

1. A compound of formula (I) or (Ia),

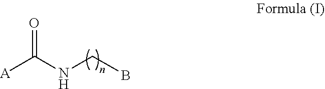

wherein:
n is 0, 1, or 2;
A is (i) a 5-membered heteroaryl of formula (II),

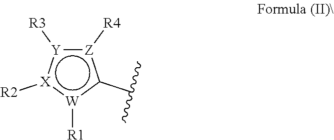

wherein W is S;
Z is C or N;
X and Y are C;
R1 is absent;
R4 is absent or H;
R2 are R3 are independently selected from H, halo, alkyl, —SO₂NR13R14, —(CH₂)₀₋₃heterocyclyl, —(CH₂)₀₋₃NR12(CH₂)₀₋₃(heterocyclyl), and —(CH₂)₀₋₃aryl; and
wherein one of R2 or R3 is not H; or
wherein W is S;
X, Y and Z are C;
R1 is absent;
R3 is halo or alkyl;
R4 is H, halo, or alkyl; and
R2 is selected from —(CH₂)₀₋₃NR13R14, —(CH₂)₀₋₃NR12(CH₂)₀₋₃(aryl), —(CH₂)₀₋₃NR12(CH₂)₀₋₃(heterocyclyl), —(CH₂)₀₋₃—O—(CH₂)₀₋₃(aryl), —(CH₂)₀₋₃—O—(CH₂)₀₋₃(heterocyclyl), —(CH₂)₀₋₃—O—(CH₂)₀₋₃(heteroaryl), —(CH₂)₀₋₃—O—(CH₂)₁₋₄NR13R14, and —(CH₂)₀₋₃heterocyclyl; or
wherein X, Y and Z are independently N, C or S;
wherein at least one of X, Y and Z is N or S;
W is C;
R3 and R4 are independently absent or independently selected from H, alkyl and halo;
R2 is selected from H, halo, alkyl, and cycloalkyl; and
R1 is selected from —(CH₂)₀₋₃NR12(CH₂)₀₋₃(heterocyclyl), —(CH₂)₀₋₃NR12CO(CH₂)₀₋₃(heterocyclyl), —(CH₂)₀₋₃—O—(CH₂)₀₋₃(heterocyclyl), and —(CH₂)₀₋₃heterocyclyl; or
wherein Y and Z are N;
W and X are C;
R1 and R2 are selected from H, halo, alkyl, cycloalkyl, and —(CH₂)₀₋₃aryl;
R3 and R4 are independently absent or independently selected from —(CH₂)₀₋₃heterocyclyl, and —(CH₂)₀₋₃aryl; and
wherein at least one of R3 or R4 is selected from —(CH₂)₀₋₃heterocyclyl, and —(CH₂)₀₋₃aryl; or
wherein Y or Z are independently C, N or S;
wherein at least one of Y and Z is N or S;
W and X are C;
R1 is H;
R2 is selected from H, alkyl, aryl, and halo;
R4 is absent, or selected from H and alkyl; and
R3 is (CH₂)₀₋₃(heterocyclyl); or
wherein Y and X are independently C or N;
wherein at least one of Y or X is N;
W and Z are C;
R1 and R4 are independently selected from H, alkyl, and halo; and
one of R2 and R3 is absent and the other of R2 and R3 is

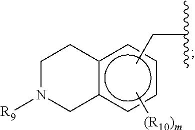

m is 0, 1, 2, or 3;
R9 is selected from H and alkyl;
Each R10 is independently selected from alkyl and halo; or
A is (ii) a 9-membered heteroaromatic bicycle of formula (III)

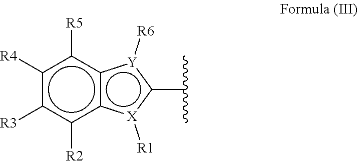

wherein X and Y are independently selected from C, N or S;
wherein at least one of X and Y is N or S;
wherein R1 and R6 are independently absent or independently selected from H and —(CH₂)₀₋₃heterocyclyl;
wherein R2 is selected from H, halo, —(CH₂)₀₋₃NR12(CH₂)₀₋₃(heterocyclyl), and —(CH₂)₀₋₃heterocyclyl;
R3, R4, and R5 are independently selected from H, alkyl and halo; and
wherein at least one of R2, R3, R4, R5 is not absent or H;

or,

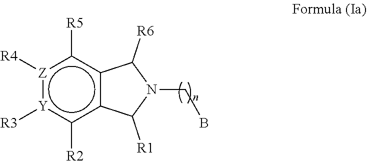

wherein n is 0, 1, or 2;
wherein Z and Y and independently selected from C and N;
wherein R6 is selected from H and alkyl;
wherein R4 and R5 are independently absent, or independently selected from H, alkyl, and halo; and
wherein one of R2 and R5 is —(CH₂)₀₋₃NR12(CH₂)₀₋₃(heterocyclyl), and the other of R2 and R5 is selected from H, alkyl, and halo;

B is:
(i) a fused 6,5- or 6,6-heteroaromatic bicyclic ring, containing N and, optionally, one or two additional heteroatoms independently selected from N, O and S;
wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, CF₃ and —NR13R14;
wherein the 6,5-heteroaromatic bicyclic ring may be attached via the 6- or 5-membered ring; or
(ii) phenyl substituted with —(CH₂)₁₋₃NH₂ and two groups selected from methyl, ethyl and propyl; or
(iii) pyridine substituted with NH₂ and two groups selected from methyl, ethyl and propyl;
(iv) a fused 6,5- or 6,6-bicyclic ring containing N and containing an aromatic ring fused to a non-aromatic ring and, optionally, one or two additional heteroatoms independently selected from N, O and S;
wherein the fused 6,5- or 6,6-bicyclic ring may be optionally substituted with 1, 2, or 3 substituents selected from alkyl, alkoxy, OH, halo, CN, —COOR13, —CONR13R14, CF₃ and —NR13R14;
wherein the 6,5-bicyclic ring may be attached via the 6- or 5-membered ring;

alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, —N(R12)₂ and fluoro;

alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₁-C₆)alkoxy, OH,

—NR13R14, —NHCOCH₃, —CO(heterocyclyl^{b}), —COOR13, —CONR13R14, CN, CF₃, halo, oxo, and heterocyclyl^{b};

alkyl^{b} is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1 or 2 substituents independently selected from (C₁-C₆)alkoxy, OH, —N(R12)₂,

—NHCOCH₃, CF₃, halo, oxo, cyclopropane, —O(aryl^{b}), aryl^{b}, and heterocyclyl^{b};

alkylene is a bivalent linear saturated hydrocarbon having 1 to 5 carbon atoms (C₁-C₅); alkylene may optionally be substituted with 1 or 2 substituents independently selected from alkyl, (C₁-C₆)alkoxy, OH, CN, CF₃, and halo;

aryl is phenyl, biphenyl or naphthyl; aryl may be optionally substituted with 1, 2 or 3 substituents independently selected from alkyl, alkoxy, OH, —SO₂CH₃, halo, CN, —(CH₂)₀₋₃O-heteroaryl^{b}, aryl^{b}, —O-aryl^{b}, —(CH₂)₀₋₃heterocyclyl, —(CH₂)₁₋₃aryl, —(CH₂)₀₋₃-heteroaryl^{b}, —COOR13, —CONR13R14, —(CH₂)₀₋₃—NR13R14, OCF₃ and CF₃; or two adjacent carbon ring atoms on the aryl may be optionally linked by a heteroalkylene to form a non-aromatic ring containing 5, 6, or 7 ring members; or optionally wherein two adjacent ring atoms on aryl are linked to form a 5- or 6-membered aromatic ring containing 1 or 2 heteroatoms that are selected from N, NR8, S, and O;

aryl^{b} is phenyl, biphenyl or naphthyl, which may be optionally substituted with 1, 2 or 3 substituents independently selected from methyl, ethyl, propyl, isopropyl, alkoxy, OH, —SO₂CH₃, N(R12)₂, halo, CN, and CF₃; or two adjacent carbon ring atoms on the aryl may be optionally linked by a heteroalkylene to form a non-aromatic ring containing 5, 6, or 7 ring members;

cycloalkyl is a monocyclic saturated hydrocarbon ring of between 3 and 6 carbon atoms (C₃-C₆); cycloalkyl may optionally be substituted with 1 or 2 substituents independently selected from alkyl, (C₁-C₆)alkoxy, OH, CN, CF₃, and halo;

halo is F, Cl, Br, or I;

heteroalkylene is a bivalent linear saturated hydrocarbon having 2 to 5 carbon atoms (C₂-C₅), wherein 1 or 2 of the 2 to 5 carbon atoms are replaced with NR8, S, or O; heteroalkylene may optionally be substituted with 1 or 2 substituents independently selected from alkyl (C₁-C₆)alkoxy, OH, CN, CF₃, and halo;

heteroaryl is a 5- or 6-membered carbon-containing aromatic ring containing 1, 2, 3, or 4 ring members that are selected from N, NR8, S, and O; heteroaryl may be optionally substituted with 1, 2 or 3 substituents independently selected from alkyl, alkoxy, aryl^{b}, OH, OCF₃, halo, heterocyclyl^{b}, CN, and CF₃;

heteroaryl^{b} is a 5- or 6-membered carbon-containing aromatic ring containing one, two or three ring members that are selected from N, NR8, S, and O; heteroaryl^{b} may be optionally substituted with 1, 2 or 3 substituents independently selected from methyl, ethyl, propyl, isopropyl, alkoxy, OH, OCF₃, halo, CN, and CF₃;

heterocyclyl is a 4-, 5-, 6-, or 7-membered carbon-containing non-aromatic ring containing one or two ring members that are selected from N, NR8, S, SO, SO₂ and O; heterocyclyl may be optionally substituted with 1, 2, 3, or 4 substituents independently selected from alkyl^{b}, alkoxy, OH, OCF₃, halo, oxo, CN, —NR13R14, —O(aryl^{b}), —O(heteroaryl^{b}) and CF₃; or optionally wherein two ring atoms on heterocyclyl are linked with an alkylene to form a non-aromatic ring containing 5, 6, or 7 ring members; or optionally wherein two adjacent ring atoms on heterocyclyl are linked to form a 5- or 6-membered aromatic ring containing 1 or 2 heteroatoms that are selected from N, NR8, S, and O; or optionally wherein a carbon ring atom on heterocyclyl is substituted with a heteroalkylene such that the carbon ring atom on heterocyclyl together with the heteroalkylene forms a heterocyclyl^{b} that is spiro to ring heterocyclyl;

heterocyclyl^{b} is a 4-, 5-, 6-, or 7-membered carbon-containing non-aromatic ring containing one or two ring members that are selected from N, NR12, S, SO, SO₂ and O; heterocyclyl may be optionally substituted with 1, 2, 3, or 4 substituents independently selected from methyl, ethyl, propyl, isopropyl, alkoxy, OH, OCF₃, halo, oxo, CN, and CF₃;

R13 and R14 are independently selected from H, —SO₂CH₃, alkyl^{b}, heteroaryl^{b}, and cycloalkyl; or R13 and R14 together with the nitrogen atom to which they are attached form a carbon-containing 4-, 5-, 6- or 7-membered heterocylic ring, optionally containing an additional heteroatom selected from N, NR8, S, SO, SO₂, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃; or R13 and R14 together with the nitrogen atom to which they are attached form a carbon-containing 5- or 6-membered heterocylic ring, which is fused to an aryl^{b} or a heteroaryl^{b};

R8 is independently selected from H, —SO₂CH₃, alkyl^{b}, —(CH₂)₀₋₃aryl^{b}, —(CH₂)₀₋₃heteroaryl^{b}, —(CH₂)₀₋₃cycloalkyl, and —(CH₂)₀₋₃heterocyclyl^{b}; or R8 is a carbon-containing 4-, 5-, 6- or 7-membered heterocylic ring containing 1, 2 or 3 heteroatoms independently selected from N, N12, S, SO, SO₂, and O, which may be saturated or unsaturated with 1 or 2 double bonds and which may be optionally mono- or di-substituted with substituents independently selected from oxo, alkyl^{b}, alkoxy, OH, halo, —SO₂CH₃, and CF₃;

R12 is independently selected from H, —SO₂CH₃, —COCH₃, methyl, ethyl, propyl, isopropyl, and cycloalkyl;

and tautomers, isomers, stereoisomers (including enantiomers, diastereoisomers and racemic and scalemic mixtures thereof), deuterated isotopes, and pharmaceutically acceptable salts and/or solvates thereof.

2. A compound of formula (I) or (Ia) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein n is 0.

3. A compound of formula (I) or (Ia) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein n is 1.

4. A compound of formula (I) or (Ia) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein n is 2.

5. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 5-membered heteroaryl of formula (II),

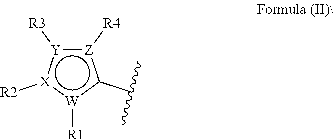

wherein W is S;
Z is C or N;
X and Y are C;
R1 is absent;
R4 is absent or H;
R2 are R3 are independently selected from H, halo, alkyl, —SO₂NR13R14, —(CH₂)₀₋₃heterocyclyl, —(CH₂)₀₋₃NR12(CH₂)₀₋₃(heterocyclyl), and —(CH₂)₀₋₃aryl; and
wherein one of R2 or R3 are not H.

6. A compound of formula (I) according to numbered embodiment 5, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is C.

7. A compound of formula (I) according to numbered embodiment 5, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is N.

8. A compound of formula (I) according to numbered embodiment 7, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is absent.

9. A compound of formula (I) or (Ia) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.

10. A compound of formula (I) according to any of numbered embodiments 5 to 9, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein at least one of R2 or R3 is (i) halo, or (ii) selected from —(CH₂)₀₋₃heterocyclyl and —(CH₂)₀₋₃NR12(CH₂)₀₋₃(heterocyclyl).

11. A compound of formula (I) according to numbered embodiment 10, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein at least one of R2 or R3 is halo.

12. A compound of formula (I) according to numbered embodiment 10, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein at least one of R2 or R3 is selected from —(CH₂)₀₋₃heterocyclyl and —(CH₂)₀₋₃NR12(CH₂)₀₋₃(heterocyclyl).

13. A compound of formula (I) according to numbered embodiment 12, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein at least one of R2 or R3 is —(CH₂)₀₋₃heterocyclyl.

14. A compound of formula (I) according to numbered embodiment 12, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein at least one of R2 or R3 is —(CH₂)₀₋₃NR12(CH₂)₀₋₃(heterocyclyl).

15. A compound of formula (I) according to any of numbered embodiments 1 to 4, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 5-membered heteroaryl of formula (II),

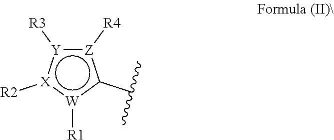

wherein W is S;
X, Y and Z are C;
R1 is absent;
R3 is halo or alkyl;
R4 is H, halo, or alkyl; and
R2 is selected from —(CH₂)₀₋₃NR13R14, —(CH₂)₀₋₃NR12(CH₂)₀₋₃(aryl), —(CH₂)₀₋₃NR12(CH₂)₀₋₃(heterocyclyl), —(CH₂)₀₋₃—O—(CH₂)₀₋₃(aryl), —(CH₂)₀₋₃—O—(CH₂)₀₋₃(heterocyclyl), —(CH₂)₀₋₃—O—(CH₂)₀₋₃(heteroaryl), —(CH₂)₀₋₃—O—(CH₂)₁₋₄NR13R14, and —(CH₂)₀₋₃heterocyclyl.

16. A compound of formula (I) according to numbered embodiment 15, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is halo.

17. A compound of formula (I) according to numbered embodiment 15, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is alkyl.

18. A compound of formula (I) according to any of numbered embodiments 15 to 17, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.

19. A compound of formula (I) according to any of numbered embodiments 15 to 17, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is halo.

20. A compound of formula (I) according to any of numbered embodiments 15 to 17, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkyl.

21. A compound of formula (I) according to any of numbered embodiments 15 to 20, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃NR13R14.

22. A compound of formula (I) according to any of numbered embodiments 15 to 20, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃NR12(CH₂)₀₋₃(aryl).

23. A compound of formula (I) according to any of numbered embodiments 15 to 20, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃NR12(CH₂)₀₋₃(heterocyclyl).

24. A compound of formula (I) according to any of numbered embodiments 15 to 20, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃O—(CH₂)₀₋₃(aryl).

25. A compound of formula (I) according to any of numbered embodiments 15 to 20, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃—O—(CH₂)₀₋₃(heterocyclyl).

26. A compound of formula (I) according to any of numbered embodiments 15 to 20, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃—O—(CH₂)₀₋₃(heteroaryl).

27. A compound of formula (I) according to any of numbered embodiments 15 to 20, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃—O—(CH₂)₁₋₄NR13R14.

28. A compound of formula (I) according to any of numbered embodiments 15 to 20, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃heterocyclyl.

29. A compound of formula (I) according to any of numbered embodiments 1 to 4, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 5-membered heteroaryl of formula (II),

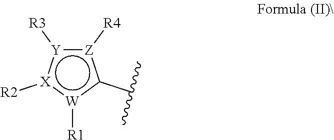

wherein X, Y and Z are independently N, C or S;
wherein at least one of X, Y and Z is N or S;
W is C;
R3 and R4 are independently absent or independently selected from H, alkyl and halo;
R2 is selected from H, halo, alkyl, and cycloalkyl; and
R1 is selected from —(CH₂)₀₋₃NR12(CH₂)₀₋₃(heterocyclyl), —(CH₂)₀₋₃—O—(CH₂)₀₋₃(heterocyclyl), and —(CH₂)₀₋₃heterocyclyl.

30. A compound of formula (I) according to numbered embodiment 29, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z and Y are both N, and X is C.

31. A compound of formula (I) according to numbered embodiment 29, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is S, and X and Y are both C.

32. A compound of formula (I) according to numbered embodiment 29, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is N, Z is S, and X is C.

33. A compound of formula (I) according to numbered embodiment 29, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is S, X is N, and Y is C.

34. A compound of formula (I) according to any of numbered embodiments 29 to 30, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H or alkyl.

35. A compound of formula (I) according to numbered embodiment 34, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.

36. A compound of formula (I) according to numbered embodiment 34, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkyl.

37. A compound of formula (I) according to any of numbered embodiments 29 or 31, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein one of R2 or R3 is halo or alkyl.

38. A compound of formula (I) according to numbered embodiment 37, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 and R3 are independently alkyl or halo.

39. A compound of formula (I) according to any of numbered embodiments 37 to 38, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is halo, preferably Cl.

40. A compound of formula (I) according to any of numbered embodiments 37 to 39, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is halo, preferably Cl.

41. A compound of formula (I) according to any of numbered embodiments 29 or 32, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is H or alkyl.

42. A compound of formula (I) according to numbered embodiment 41, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is H.

43. A compound of formula (I) according to numbered embodiment 41, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is alkyl.

44. A compound of formula (I) according to any of numbered embodiments 29 or 32, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is cycloalkyl.

45. A compound of formula (I) according to any of numbered embodiments 29 or 33, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is H or alkyl.

46. A compound of formula (I) according to numbered embodiment 44, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is H.

47. A compound of formula (I) according to numbered embodiment 44, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is alkyl.

48. A compound of formula (I) according to any of numbered embodiments 29 or 33, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is halo, preferably Cl.

49. A compound of formula (I) according to any of numbered embodiments 29 to 48, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is selected from —NR12(CH₂)₀₋₃(heterocyclyl), —O—(CH₂)₀₋₃(heterocyclyl), and —(CH₂)₀₋₃heterocyclyl.

50. A compound of formula (I) according to any of numbered embodiments 29 to 48 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is —(CH₂)₀₋₃NR12(CH₂)₀₋₃(heterocyclyl), preferably —NR12(CH₂)₀₋₃(heterocyclyl).

51. A compound of formula (I) according to any of numbered embodiments 29 to 50, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein when present, R12 is H or —COCH₃.

52. A compound of formula (I) according to any of numbered embodiments 29 to 50, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is —(CH₂)₀₋₃—O—(CH₂)₀₋₃(heterocyclyl), preferably —O—(CH₂)₀₋₃(heterocyclyl).

53. A compound of formula (I) according to any of numbered embodiments 29 to 50, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is —(CH₂)₀₋₃heterocyclyl.

54. A compound of formula (I) according to any of numbered embodiments 29 to 53, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R1 is piperidinyl.

55. A compound of formula (I) according to any of numbered embodiments 1 to 4, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 5-membered heteroaryl of formula (II),

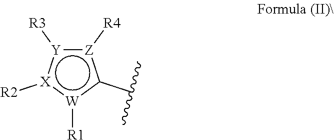

wherein Y and Z are N;
W and X are C;
R1 and R2 are selected from H, halo, alkyl, cycloalkyl, and —(CH₂)₀₋₃aryl;
R3 and R4 are independently absent or independently selected from —(CH₂)₀₋₃heterocyclyl, and —(CH₂)₀₋₃aryl; and
wherein at least one of R3 or R4 is selected from —(CH₂)₀₋₃heterocyclyl, and —(CH₂)₀₋₃aryl.

56. A compound of formula (I) according to numbered embodiment 55, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is H.

57. A compound of formula (I) according to numbered embodiment 55, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is halo.

58. A compound of formula (I) according to numbered embodiment 55, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is alkyl.

59. A compound of formula (I) according to numbered embodiment 55, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is cycloalkyl.

60. A compound of formula (I) according to numbered embodiment 55, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is —(CH₂)₀₋₃aryl.

61. A compound of formula (I) according to any of numbered embodiments 55 to 60, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is H.

62. A compound of formula (I) according to any of numbered embodiments 55 to 60, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is halo.

63. A compound of formula (I) according to any of numbered embodiments 55 to 60, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is alkyl.

64. A compound of formula (I) according to any of numbered embodiments 55 to 60, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is cycloalkyl.

65. A compound of formula (I) according to any of numbered embodiments 55 to 60, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃aryl.

66. A compound of formula (I) according to any of numbered embodiments 55 to 65, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is absent and R4 is —(CH₂)₀₋₃heterocyclyl.

67 A compound of formula (I) according to any of numbered embodiments 55 to 65, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is absent and R4 is —(CH₂)₀₋₃aryl.

68. A compound of formula (I) according to any of numbered embodiments 55 to 65, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is absent and R3 is —(CH₂)₀₋₃heterocyclyl.

69. A compound of formula (I) according to any of numbered embodiments 55 to 65, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is absent and R3 is —(CH₂)₀₋₃aryl.

70. A compound of formula (I) according to any of numbered embodiments 55 to 69, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when present, the heterocyclyl on R3 or R4 is piperidinyl.

71. A compound of formula (I) according to any of numbered embodiments 1 to 4, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 5-membered heteroaryl of formula (II),

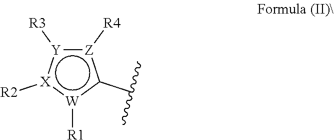

wherein Y or Z are independently C, N or S;
wherein at least one of Y and Z is N or S;
W and X are C;
R1 is H;
R2 is selected from H, alkyl, aryl, and halo;
R4 is absent, or selected from H and alkyl; and
R3 is (CH₂)₀₋₃(heterocyclyl).

72. A compound of formula (I) according to numbered embodiment 71, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is C and Y is N.

73. A compound of formula (I) according to numbered embodiment 72, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is S and Y is C.

74. A compound of formula (I) according to numbered embodiment 72, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y and Z are both N.

75. A compound of formula (I) according to any of numbered embodiments 71 to 72, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.

76. A compound of formula (I) according to any of numbered embodiments 71 to 72, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkyl.

77. A compound of formula (I) according to any of numbered embodiments 71 to 76, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the heterocyclyl on R3 is piperidinyl, piperazinyl, or morpholinyl.

78. A compound of formula (I) according to any of numbered embodiments 71 to 77, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is H.

79. A compound of formula (I) according to any of numbered embodiments 71 to 77, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is alkyl.

80. A compound of formula (I) according to any of numbered embodiments 71 to 77, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is aryl.

81. A compound of formula (I) according to any of numbered embodiments 71 to 77, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is halo, preferably Cl.

82. A compound of formula (I) according to any of numbered embodiments 1 to 4, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 5-membered heteroaryl of formula (II),

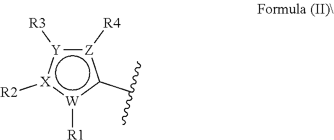

wherein Y and X are independently C or N;
wherein at least one of Y or X is N;
W and Z are C;
R1 and R4 are independently selected from H, alkyl, and halo; and one of R2 and R3 is absent and the other of R2 and R3 is selected from: one of R2 and
R3 is absent and the other of R2 and R3 is selected from:

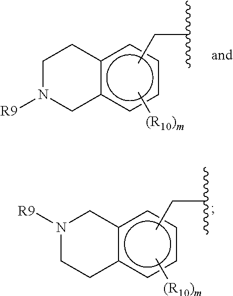

m is 0, 1, 2, or 3;
R9 is selected from H and alkyl;
Each R10 is independently selected from alkyl and halo.

83. A compound of formula (I) according to numbered embodiment 82, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is N.

84. A compound of formula (I) according to any of numbered embodiments 82 to 83, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is N.

85. A compound of formula (I) according to any of numbered embodiments 82, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is N.

86. A compound of formula (I) according to any of numbered embodiments 82 to 85, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is H.

87. A compound of formula (I) according to any of numbered embodiments 82 to 85, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is alkyl.

88. A compound of formula (I) according to any of numbered embodiments 82 to 85 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is halo.

89. A compound of formula (I) according to any of numbered embodiments 82 to 88, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.

90. A compound of formula (I) according to any of numbered embodiments 82 to 88, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkyl.

91. A compound of formula (I) according to any of numbered embodiments 82 to 88, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is halo.

92. A compound of formula (I) according to any of numbered embodiments 86 to 91, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein one of R2 and R3 is absent and the other of R2 and R3 is

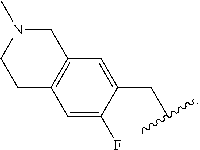 [SMILES: *Cc1cc2c(cc1F)CCN(C)C2]

93. A compound of formula (I) according to any of numbered embodiments 1 to 4, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is (ii) a 9-membered bicycle of formula (III) comprising an aromatic 6-membered ring fused to a 5-membered ring,

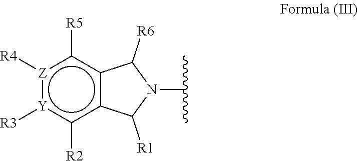

wherein X and Y are independently selected from C, N or S;
wherein at least one of X and Y is N or S;
wherein R1 and R6 are independently absent or independently selected from H and —(CH₂)₀₋₃heterocyclyl;
wherein R2 is selected from H, halo, —(CH₂)₀₋₃NR12(CH₂)₀₋₃(heterocyclyl), and —(CH₂)₀₋₃heterocyclyl;
R3, R4, and R5 are independently selected from H, alkyl and halo; and wherein at least one of R1, R2, R3, R4, R5 and R6 is not H.

94. A compound of formula (I) according to numbered embodiment 93, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is N.

95. A compound of formula (I) according to any of numbered embodiments 93 to 94, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is N.

96. A compound of formula (I) according to any of numbered embodiments 93 and 95, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is C.

97. A compound of formula (I) according to any of numbered embodiments 93 and 94, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is C.

98. A compound of formula (I) according to any of numbered embodiments 93, 94 and 96, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is S.

99. A compound of formula (I) according to any of numbered embodiments 93, 95 and 97, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is S.

100. A compound of formula (I) according to any of numbered embodiments 93 to 98, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is —(CH₂)₀₋₃heterocyclyl, preferably wherein the heterocyclyl on R1 is piperidinyl.

101. A compound of formula (I) according to any of numbered embodiments 93 to 98, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is H.

102. A compound of formula (I) according to any of numbered embodiments 93 to 101, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃NR12(CH₂)₀₋₃(heterocyclyl), preferably —NR12(CH₂)₀₋₃(heterocyclyl).

103. A compound of formula (I) according to any of numbered embodiments 93 to 102, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is H.

104. A compound of formula (I) according to any of numbered embodiments 93 to 103, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is H.

105. A compound of formula (I) according to any of numbered embodiments 93 to 103, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is alkyl.

106. A compound of formula (I) according to any of numbered embodiments 93 to 103, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R3 is halo.

107. A compound of formula (I) according to any of numbered embodiments 93 to 106, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.

108. A compound of formula (I) according to any of numbered embodiments 93 to 106, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkyl.

109. A compound of formula (I) according to any of numbered embodiments 93 to 106, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is halo.

110. A compound of formula (I) according to any of numbered embodiments 93 to 109, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is H.

111. A compound of formula (I) according to any of numbered embodiments 93 to 109, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is alkyl.

112. A compound of formula (I) according to any of numbered embodiments 93 to 109, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is halo.

113. A compound of formula (Ia) according to any of numbered embodiments 1 to 4, or a tautomer, isomer, stereoisomer (including an enantiomer, a diastereoisomer and a racemic and scalemic mixture thereof), a deuterated isotope, and a pharmaceutically acceptable salt and/or solvate thereof,

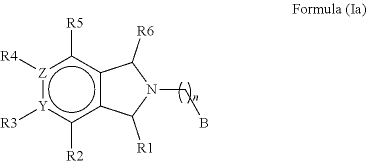

wherein n is 0, 1, or 2;
wherein Z and Y and independently selected from C and N;
wherein R6 is selected from H and alkyl;
wherein R4 and R5 are independently absent, or independently selected from H, alkyl, and halo; and
wherein one of R2 and R5 is —(CH₂)₀₋₃NR12(CH₂)₀₋₃(heterocyclyl), and the other of R2 and R5 is selected from H, alkyl, and halo.

114. A compound of formula (Ia) according to numbered embodiment 113, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is C.

115. A compound of formula (Ia) according to numbered embodiment 113, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Z is N.

116. A compound of formula (Ia) according to any of numbered embodiments 113 and 115, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is C.

117. A compound of formula (Ia) according to numbered embodiment 113 to 115, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is N.

118. A compound of formula (Ia) according to numbered embodiment 113 to 117, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is H.

119. A compound of formula (Ia) according to numbered embodiment 113 to 117, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R6 is alkyl.

120. A compound of formula (Ia) according to numbered embodiment 113 to 119, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is H.

121. A compound of formula (Ia) according to numbered embodiment 113 to 119, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is alkyl.

122. A compound of formula (Ia) according to numbered embodiment 113 to 119, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R4 is halo.

123. A compound of formula (Ia) according to numbered embodiment 113 to 122, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is H.

124. A compound of formula (Ia) according to numbered embodiment 113 to 122, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is alkyl.

125. A compound of formula (Ia) according to numbered embodiment 113 to 122, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is halo.

126. A compound of formula (Ia) according to numbered embodiment 113 to 119, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃NR12(CH₂)₀₋₃(heterocyclyl) and R5 is H.

127. A compound of formula (Ia) according to numbered embodiment 113 to 119, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃NR12(CH₂)₀₋₃heterocyclyl) and R5 is alkyl.

128. A compound of formula (Ia) according to numbered embodiment 113 to 119, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is —(CH₂)₀₋₃NR12(CH₂)₀₋₃(heterocyclyl) and R5 is halo.

129. A compound of formula (Ia) according to numbered embodiment 113 to 119, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —(CH₂)₀₋₃NR12(CH₂)₀₋₃(heterocyclyl) and R2 is H.

130. A compound of formula (Ia) according to numbered embodiment 113 to 119, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —(CH₂)₀₋₃NR12(CH₂)₀₋₃(heterocyclyl) and R2 is alkyl.

131. A compound of formula (Ia) according to numbered embodiment 113 to 119, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R5 is —(CH₂)₀₋₃NR12(CH₂)₀₋₃(heterocyclyl) and R2 is halo.

132. A compound of formula (I) or (Ia) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is a fused 6,5- or 6,6-heteroaromatic bicyclic ring, containing N and, optionally, one or two additional heteroatoms independently selected from N, O and S;
wherein the fused 6,5- or 6,6-heteroaromatic bicyclic ring may be optionally substituted with 1, 2, or 3 substituents selected from alkyl, alkoxy, OH, halo, CN, —COOR13, —CONR13R14, —CF₃ and —NR13R14;
wherein the 6,5-heteroaromatic bicyclic ring may be attached via the 6- or 5-membered ring.

133. A compound of formula (I) or (Ia) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is isoqunilolinyl.

134. A compound of formula (I) or (Ia) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is isoqunilolinyl substituted with —NR13R14, preferably —NH₂.

135. A compound of formula (I) or (Ia) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is substituted with halo.

136. A compound of formula (I) or (Ia) according to numbered embodiment 132, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is indole.

137. A compound of formula (I) or (Ia) according to numbered embodiment 136, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is indole substituted with halo, preferably Cl.

138. A compound of formula (I) or (Ia) according to numbered embodiment 136, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is indole substituted twice with alkyl, preferably twice with methyl.

139. A compound of formula (I) or (Ia) according to numbered embodiment 132, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is 5-azathianaphthenyl.

140. A compound of formula (I) or (Ia) according to numbered embodiment 139, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is 5-azathianaphthenyl substituted with —NR13R14, preferably —NH₂.

141. A compound of formula (I) or (Ia) according to any of numbered embodiments 1 to 131, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl substituted with —(CH₂)₁₋₃NH₂ and two groups selected from methyl, ethyl and propyl.

142. A compound of formula (I) or (Ia) according to numbered embodiment 141, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is phenyl substituted with —CH₂NH₂ and two methyl groups.

143. A compound of formula (I) or (Ia) according to any of numbered embodiments 1 to 131, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is pyridine substituted with NH₂ and two groups selected from methyl, ethyl and propyl.

144. A compound of formula (I) or (Ia) according to any of numbered embodiments 143, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is pyridine substituted with NH₂ and two methyl groups.

145. A compound of formula (I) or (Ia) according to any of numbered embodiments 1 to 131, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is a fused 6,5- or 6,6-bicyclic ring containing N and containing an aromatic ring fused to a non-aromatic ring and, optionally, one or two additional heteroatoms independently selected from N, O and S; wherein the fused 6,5- or 6,6-bicyclic ring may be optionally substituted with 1, 2, or 3 substituents selected from alkyl, alkoxy, OH, halo, CN, —COOR13, —CONR13R14, CF₃ and —NR13R14; wherein the 6,5-bicyclic ring may be attached via the 6- or 5-membered ring.

146. A compound of formula (I) or (Ia) according to numbered embodiment 145, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the 6,5-bicyclic ring is attached via the 5-membered ring.

147. A compound of formula (I) or (Ia) according to numbered embodiment 145, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the 6,5-bicyclic ring is attached via the 6-membered ring.

148. A compound of formula (I) or (Ia) according to any of numbered embodiments 145 to 147, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the 5-membered ring is cyclopropane.

149. A compound of formula (I) or (Ia) according to any of numbered embodiments 145 to 148, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the 5-membered ring is pyridine.

150. A compound of formula (I) or (Ia) according to numbered embodiment 149, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the pyridine is substituted with —NR13R14.

151. A compound of formula (I) or (Ia) according to numbered embodiment 150, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the pyridine is substituted with —NH₂.

152. A compound selected from any of Tables 1 to 12, and pharmaceutically acceptable salts, solvates, or solvates of salts thereof.

153. A compound according to any preceding claim selected from examples: 25.15, 25.21, 35.04, 51.05, 2.36, 7.03, 7.05, 7.08, 7.22, 7.23, 7.26, 7.31, 25.07, 25.11, 25.14, 25.202, 25.203, 25.207, 26.05, 26.09, 26.1, 26.16, 35.07, 35.08, 51.06, 51.07, 69.01; and pharmaceutically acceptable salts, solvates, or solvates of salts thereof.

154. A compound according to any preceding claim selected from examples: 25.15, 25.21, 35.04, 51.05; and pharmaceutically acceptable salts, solvates, or solvates of salts thereof.

155. A compound according to any preceding numbered embodiment.

156. A pharmaceutically acceptable salt according to any of numbered embodiments 1 to 155.

157. A pharmaceutically acceptable solvate according to any of numbered embodiments 1 to 155.

158. A pharmaceutically acceptable solvate of a salt according to any of numbered embodiments 1 to 155.

159. A pharmaceutical composition comprising:
(i) a compound according to numbered embodiment 155, the pharmaceutically acceptable salt according to numbered embodiment 156, the pharmaceutically acceptable solvate according to numbered embodiment 157, or the pharmaceutically acceptable solvate of a salt according to numbered embodiment 158; and
(ii) at least one pharmaceutically acceptable excipient.

160. A compound as defined in numbered embodiment 155, a pharmaceutically acceptable salt according to numbered embodiment 156, a pharmaceutically acceptable solvate according to numbered embodiment 157, a pharmaceutically acceptable solvate of a salt according to numbered embodiment 158, or a pharmaceutical composition as defined in numbered embodiment 159, for use in medicine.

161. The use of a compound as defined in numbered embodiment 155, a pharmaceutically acceptable salt according to numbered embodiment 156, a pharmaceutically acceptable solvate according to numbered embodiment 157, a pharmaceutically acceptable solvate of a salt according to numbered embodiment 158, or a pharmaceutical composition as defined in numbered embodiment 159, in the manufacture of a medicament for the treatment or prevention of a disease or condition in which Factor XIIa activity is implicated.

162. A method of treatment of a disease or condition in which Factor XIIa activity is implicated comprising administration to a subject in need thereof a therapeutically effective amount of a compound as defined in numbered embodiment 155, a pharmaceutically acceptable salt according to numbered embodiment 156, a pharmaceutically acceptable solvate according to numbered embodiment 157, a pharmaceutically acceptable solvate of a salt according to numbered embodiment 158, or a pharmaceutical composition as defined in numbered embodiment 159.

163. A compound as defined in numbered embodiment 155, a pharmaceutically acceptable salt according to numbered embodiment 156, a pharmaceutically acceptable solvate according to numbered embodiment 157, a pharmaceutically acceptable solvate of a salt according to numbered embodiment 158, or a pharmaceutical composition as defined in numbered embodiment 159, for use in a method of treatment of a disease or condition in which Factor XIIa activity is implicated.

164. The use of numbered embodiment 161, the method of numbered embodiment 162, or a compound, a pharmaceutically acceptable salt, a pharmaceutically acceptable solvate, a pharmaceutically acceptable solvate of a salt, or a pharmaceutical composition for use as defined in numbered embodiment 163, wherein, the disease or condition in which Factor XIIa activity is implicated is a bradykinin-mediated angioedema.

165. The use of numbered embodiment 164, the method of numbered embodiment 164, or a compound, a pharmaceutically acceptable salt, a pharmaceutically acceptable solvate, a pharmaceutically acceptable solvate of a salt, or a pharmaceutical composition for use as defined in numbered embodiment 164, wherein the bradykinin-mediated angioedema is hereditary angioedema.

166. The use of numbered embodiment 164, the method of numbered embodiment 164, or a compound, a pharmaceutically acceptable salt, a pharmaceutically acceptable solvate, a pharmaceutically acceptable solvate of a salt, or a pharmaceutical composition for use as defined in numbered embodiment 164, wherein the bradykinin-mediated angioedema is non hereditary.

167. The use of numbered embodiment 161, the method of numbered embodiment 162, or a compound, a pharmaceutically acceptable salt, a pharmaceutically acceptable solvate, a pharmaceutically acceptable solvate of a salt, or a pharmaceutical composition for use as defined in numbered embodiment 163, wherein the disease or condition in which Factor XIIa activity is implicated is selected from vascular hyperpermeability; stroke including ischemic stroke and haemorrhagic accidents; retinal edema; diabetic retinopathy; DME; retinal vein occlusion; and AMD.

168. The use of numbered embodiment 161, the method of numbered embodiment 162, or a compound, a pharmaceutically acceptable salt, a pharmaceutically acceptable solvate, a pharmaceutically acceptable solvate of a salt, or a pharmaceutical composition for use as defined in numbered embodiment 163, wherein, the disease or condition in which Factor XIIa activity is implicated is a thrombotic disorder.

169. The use of numbered embodiment 168, the method of numbered embodiment 168, or a compound, a pharmaceutically acceptable salt, a pharmaceutically acceptable solvate, a pharmaceutically acceptable solvate of a salt, or a pharmaceutical composition for use as defined in numbered embodiment 168, wherein the thrombotic disorder is thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget Schroetter syndrome and Budd-Chari syndrome; and atherosclerosis.

170. The use of numbered embodiment 161, the method of numbered embodiment 162, or a compound, a pharmaceutically acceptable salt, a pharmaceutically acceptable solvate, a pharmaceutically acceptable solvate of a salt, or a pharmaceutical composition for use as defined in numbered embodiment 163, wherein, the disease or condition in which Factor XIIa activity is implicated is selected from neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

171. The use of any of numbered embodiments 161 or 164 to 170, the method of any of numbered embodiments 161 or 164 to 170, or a compound, a pharmaceutically acceptable salt, a pharmaceutically acceptable solvate, a pharmaceutically acceptable solvate of a salt, or a pharmaceutical composition for use as defined in any of numbered embodiments 161 or 164 to 170, wherein the compound targets FXIIa.

1. A compound of formula (I) or (Ia), 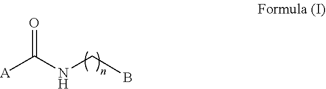
wherein:
n is 0, 1, or 2;
A is (i) a 5-membered heteroaryl of formula (II), 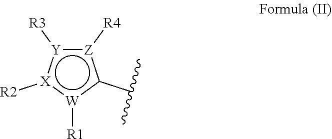
wherein:
(a) W is S;
Z is C or N;
X and Y are C;
R1 is absent;
R4 is absent or H;
R2 are R3 are independently selected from H, halo, alkyl, —SO₂NR13R14, —(CH₂)₀₋₃heterocyclyl, —(CH₂)₀₋₃NR12(CH₂)₀₋₃(heterocyclyl), or —(CH₂)₀₋₃aryl; and
wherein one of R2 or R3 is not H; or
(b) W is S;
X, Y and Z are C;
R1 is absent;
R3 is halo or alkyl;
R4 is H, halo, or alkyl; and
R2 is selected from —(CH₂)₀₋₃NR13R14, —(CH₂)₀₋₃NR12(CH₂)₀₋₃(aryl), —(CH₂)₀₋₃NR12(CH₂)₀₋₃(heterocyclyl), —(CH₂)₀₋₃—O—(CH₂)₀₋₃(aryl), —(CH₂)₀₋₃—O—(CH₂)₀₋₃(heterocyclyl), —(CH₂)₀₋₃—O—(CH₂)₀₋₃(heteroaryl), —(CH₂)₀₋₃—O—(CH₂)₁₋₄NR13R14, or —(CH₂)₀₋₃heterocyclyl; or
(c) X, Y and Z are independently N, C or S;
wherein at least one of X, Y and Z is N or S;
W is C;
R3 and R4 are independently absent, H, alkyl or halo;
R2 is selected from H, halo, alkyl, or cycloalkyl; and
R1 is selected from —(CH₂)₀₋₃NR12(CH₂)₀₋₃(heterocyclyl), —(CH₂)₀₋₃NR12CO(CH₂)₀₋₃(heterocyclyl), —(CH₂)₀₋₃—O—(CH₂)₀₋₃(heterocyclyl), or —(CH₂)₀₋₃heterocyclyl; or
(d) Y and Z are N;
W and X are C;
R1 and R2 are selected from H, halo, alkyl, cycloalkyl, or —(CH₂)₀₋₃aryl;
R3 and R4 are independently absent, —(CH₂)₀₋₃heterocyclyl, or —(CH₂)₀₋₃aryl; and
wherein at least one of R3 or R4 is selected from —(CH₂)₀₋₃heterocyclyl, or —(CH₂)₀₋₃aryl; or
(e) Y or Z are independently C, N or S;
at least one of Y and Z is N or S;
W and X are C;
R1 is H;
R2 is selected from H, alkyl, aryl, or halo;
R4 is absent, H, or alkyl; and
R3 is (CH₂)₀₋₃(heterocyclyl); or
(f) Y and X are independently C or N;
wherein at least one of Y or X is N;
W and Z are C;
R1 and R4 are independently selected from H, alkyl, or halo; and
one of R2 and R3 is absent and the other of R2 and R3 is 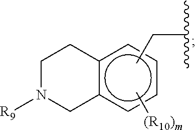
m is 0, 1, 2, or 3;
R9 is selected from H or alkyl;
Each R10 is independently selected from alkyl or halo; or
A is (ii) a 9-membered heteroaromatic bicycle of formula (III) 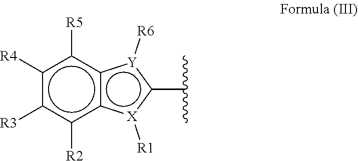
wherein:
X and Y are independently selected from C, N or S;
at least one of X and Y is N or S;
R1 and R6 are independently absent, H, or —(CH₂)₀₋₃heterocyclyl;
R2 is selected from H, halo, —(CH₂)₀₋₃NR12(CH₂)₀₋₃(heterocyclyl), or —(CH₂)₀₋₃heterocyclyl;
R3, R4, and R5 are independently selected from H, alkyl or halo; and
at least one of R2, R3, R4, R5 is not absent or H;
or, 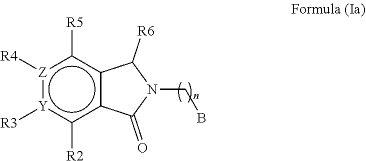
wherein:
n is 0, 1, or 2;
Z and Y are independently selected from C or N;
R6 is selected from H or alkyl;
R4 and R5 are independently absent, H, alkyl, or halo; and
one of R2 and R5 is —(CH₂)₀₋₃NR12(CH₂)₀₋₃(heterocyclyl), and the other of R2 and R5 is selected from H, alkyl, or halo;
B is:
(i) a fused 6,5- or 6,6-heteroaromatic bicyclic ring, containing N and, optionally, one or two additional heteroatoms independently selected from N, O or S;
wherein the fused 6,5- or 6,6-heteroaromatic bicyclic ring is optionally substituted with 1, 2, or 3 substituents selected from alkyl, alkoxy, OH, halo, CN, —COOR13, —CONR13R14, CF₃ or —NR13R14;
wherein the 6,5-heteroaromatic bicyclic ring is attached via the 6- or 5-membered ring; or
(ii) phenyl substituted with —(CH₂)₁₋₃NH₂ and two groups selected from methyl, ethyl or propyl; or
(iii) pyridine substituted with NH₂ and two groups selected from methyl, ethyl or propyl;
(iv) a fused 6,5- or 6,6-bicyclic ring containing N and containing an aromatic ring fused to a non-aromatic ring and, optionally, one or two additional heteroatoms independently selected from N, O or S;
wherein the fused 6,5- or 6,6-bicyclic ring is optionally substituted with 1, 2, or 3 substituents selected from alkyl, alkoxy, OH, halo, CN, —COOR13, —CONR13R14, CF₃ or —NR13R14;
wherein the 6,5-bicyclic ring is attached via the 6- or 5-membered ring;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy is optionally substituted with 1 or 2 substituents independently selected from OH, CN, CF₃, —N(R12)₂ or fluoro;
alkyl is a linear saturated hydrocarbon having up to 10 carbon atoms (C₁-C₁₀) or a branched saturated hydrocarbon of between 3 and 10 carbon atoms (C₃-C₁₀); alkyl is optionally substituted with 1 or 2 substituents independently selected from (C₁-C₆)alkoxy, OH, —NR13R14, —NHCOCH₃, —CO(heterocyclyl^{b}), —COOR13, —CONR13R14, CN, CF₃, halo, oxo, or heterocyclyl^{b};
alkyl^{b} is a linear saturated hydrocarbon having up to 10 carbon atoms (C₁-C₁₀) or a branched saturated hydrocarbon of between 3 and 10 carbon atoms (C₃-C₁₀); alkyl is optionally substituted with 1 or 2 substituents independently selected from (C₁-C₆)alkoxy, OH, —N(R12)₂, —NHCOCH₃, CF₃, halo, oxo, cyclopropane, —O(aryl^{b}), aryl^{b}, or heterocyclyl^{b};
alkylene is a bivalent linear saturated hydrocarbon having 1 to 5 carbon atoms (C₁-C₅); alkylene is optionally substituted with 1 or 2 substituents independently selected from alkyl, (C₁-C₆)alkoxy, OH, CN, CF₃, or halo;
aryl is phenyl, biphenyl or naphthyl; aryl is optionally substituted with 1, 2 or 3 substituents independently selected from alkyl, alkoxy, OH, —SO₂CH₃, halo, CN, —(CH₂)₀₋₃—O-heteroaryl^{b}, aryl^{b}, —O-aryl^{b}, —(CH₂)₀₋₃-heterocyclyl^{b}, —(CH₂)₁₋₃-aryl^{b}, —(CH₂)₀₋₃-heteroaryl^{b}, —COOR13, —CONR13R14, —(CH₂)₀₋₃—NR13R14, OCF₃ or CF₃; or two adjacent carbon ring atoms on the aryl are optionally linked by a heteroalkylene to form a non-aromatic ring containing 5, 6, or 7 ring members; or optionally wherein two adjacent ring atoms on aryl are linked to form a 5- or 6-membered aromatic ring containing 1 or 2 heteroatoms that are selected from N, NR8, S, or O;
aryl^{b} is phenyl, biphenyl or naphthyl, which is optionally substituted with 1, 2 or 3 substituents independently selected from methyl, ethyl, propyl, isopropyl, alkoxy, OH, —SO₂CH₃, N(R12)₂, halo, CN, or CF₃; or two adjacent carbon ring atoms on the aryl are optionally linked by a heteroalkylene to form a non-aromatic ring containing 5, 6, or 7 ring members;
cycloalkyl is a monocyclic saturated hydrocarbon ring of between 3 and 6 carbon atoms (C₃-C₆); cycloalkyl is optionally substituted with 1 or 2 substituents independently selected from alkyl^{b}, (C₁-C₆)alkoxy, OH, CN, CF₃, or halo;
halo is F, Cl, Br, or I;
heteroalkylene is a bivalent linear saturated hydrocarbon having 2 to 5 carbon atoms (C₂-C₅), wherein 1 or 2 of the 2 to 5 carbon atoms are replaced with NR8, S, or O; heteroalkylene is optionally substituted with 1 or 2 substituents independently selected from alkyl (C₁-C₆)alkoxy, OH, CN, CF₃, or halo;
heteroaryl is a 5- or 6-membered carbon-containing aromatic ring containing 1, 2, 3, or 4 ring members that are selected from N, NR8, S, or O; heteroaryl is optionally substituted with 1, 2 or 3 substituents independently selected from alkyl, alkoxy, aryl^{b}, OH, OCF₃, halo, heterocyclyl^{b}, CN, or CF₃;
heteroaryl^{b} is a 5- or 6-membered carbon-containing aromatic ring containing one, two or three ring members that are selected from N, NR8, S, or O; heteroaryl^{b} is optionally substituted with 1, 2 or 3 substituents independently selected from methyl, ethyl, propyl, isopropyl, alkoxy, OH, OCF₃, halo, CN, or CF₃;
heterocyclyl is a 4-, 5-, 6-, or 7-membered carbon-containing non-aromatic ring containing one or two ring members that are selected from N, NR8, S, SO, SO₂ or O; heterocyclyl is optionally substituted with 1, 2, 3, or 4 substituents independently selected from alkyl^{b}, alkoxy, OH, OCF₃, halo, oxo, CN, —NR13R14, —O(aryl^{b}), —O(heteroaryl^{b}) or CF₃; or optionally wherein two ring atoms on heterocyclyl are linked with an alkylene to form a non-aromatic ring containing 5, 6, or 7 ring members; or optionally wherein two adjacent ring atoms on heterocyclyl are linked to form a 5- or 6-membered aromatic ring containing 1 or 2 heteroatoms that are selected from N, NR8, S, or O; or optionally wherein a carbon ring atom on heterocyclyl is substituted with a heteroalkylene such that the carbon ring atom on heterocyclyl together with the heteroalkylene forms a heterocyclyl^{b} that is spiro to ring heterocyclyl;
heterocyclyl^{b} is a 4-, 5-, 6-, or 7-membered carbon-containing non-aromatic ring containing one or two ring members that are selected from N, NR12, S, SO, SO₂ or O; heterocyclyl^{b} is optionally substituted with 1, 2, 3, or 4 substituents independently selected from methyl, ethyl, propyl, isopropyl, alkoxy, OH, OCF₃, halo, oxo, CN, or CF₃;
R13 and R14 are independently selected from H, —SO₂CH₃, alkyl^{b}, heteroaryl^{b}, or cycloalkyl; or R13 and R14 together with the nitrogen atom to which they are attached form a carbon-containing 4-, 5-, 6- or 7-membered heterocyclic ring, optionally containing an additional heteroatom selected from N, NR8, S, SO, SO₂, or O, which is saturated or unsaturated with 1 or 2 double bonds and is optionally mono- or di-substituted with substituents independently selected from oxo, alkyl^{b}, alkoxy, OH, halo, —SO₂CH₃, or CF₃; or R13 and R14 together with the nitrogen atom to which they are attached form a carbon-containing 5- or 6-membered heterocyclic ring, which is fused to an aryl^{b} or a heteroaryl^{b};
R8 is independently selected from H, —SO₂CH₃, alkyl^{b}, —(CH₂)₀₋₃aryl^{b}, —(CH₂)₀₋₃heteroaryl^{b}, —(CH₂)₀₋₃cycloalkyl, or —(CH₂)₀₋₃heterocyclyl^{b}; or R8 is a carbon-containing 4-, 5-, 6- or 7-membered heterocyclic ring containing 1, 2 or 3 heteroatoms independently selected from N, N12, S, SO, SO₂, or O, which is saturated or unsaturated with 1 or 2 double bonds and is optionally mono- or di-substituted with substituents independently selected from oxo, alkyl^{b}, alkoxy, OH, halo, —SO₂CH₃, or CF₃;
R12 is independently selected from H, —SO₂CH₃, —COCH₃, methyl, ethyl, propyl, isopropyl, or cycloalkyl;
or a tautomer, isomer, stereoisomer, deuterated isotope, pharmaceutically acceptable salt or solvate thereof.

2. The compound of formula (I) according to claim 1, or a tautomer, isomer, stereoisomer, a deuterated isotope, or a pharmaceutically acceptable salt and/or solvate thereof, wherein A is a 5-membered heteroaryl of formula (II), 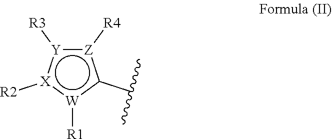
wherein:
W is S;
Z is C or N;
X and Y are C;
R1 is absent;
R4 is absent or H;
R2 are R3 are independently selected from H, halo, alkyl, —SO₂NR13R14, —(CH₂)₀₋₃heterocyclyl, —(CH₂)₀₋₃NR12(CH₂)₀₋₃(heterocyclyl), or —(CH₂)₀₋₃aryl; and
wherein one of R2 or R3 is not H.

3. The compound of formula (I) according to claim 2, or a tautomer, isomer, stereoisomer, a deuterated isotope, a pharmaceutically acceptable salt and/or solvate thereof, wherein at least one of R2 and R3 is (i) halo, or (ii) selected from —(CH₂)₀₋₃heterocyclyl, —(CH₂)₀₋₃NR12(CH₂)₀₋₃(heterocyclyl), or —(CH₂)₀₋₃aryl.

4. The compound of formula (I) according to claim 1, or a tautomer, isomer, stereoisomer, a deuterated isotope, a pharmaceutically acceptable salt, and/or solvate thereof, wherein R2 is alkyl and R3 is halo.

5. The compound of formula (I) according to claim 1, or a tautomer, isomer, stereoisomer, a deuterated isotope, a pharmaceutically acceptable salt and/or solvate thereof, wherein R2 is H and R3 is —(CH₂)₀₋₃heterocyclyl.

6. The compound of formula (I) according to claim 1, or a tautomer, isomer, stereoisomer, a deuterated isotope, a pharmaceutically acceptable salt and/or solvate thereof, wherein A is a 5-membered heteroaryl of formula (II), 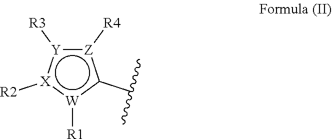
wherein:
W is S;
X, Y and Z are C;
R1 is absent;
R3 is halo or alkyl;
R4 is H, halo, or alkyl; and
R2 is selected from —(CH₂)₀₋₃NR13R14, —(CH₂)₀₋₃NR12(CH₂)₀₋₃(aryl), —(CH₂)₀₋₃NR12(CH₂)₀₋₃(heterocyclyl), —(CH₂)₀₋₃—O—(CH₂)₀₋₃(aryl), —(CH₂)₀₋₃—O—(CH₂)₀₋₃(heterocyclyl), —(CH₂)₀₋₃—O—(CH₂)₀₋₃(heteroaryl), —(CH₂)₀₋₃—O—(CH₂)₁₋₄NR13R14, or —(CH₂)₀₋₃heterocyclyl.

7. The compound of formula (I) according to claim 6, or a tautomer, isomer, stereoisomer, a deuterated isotope, a pharmaceutically acceptable salt and/or solvate thereof, wherein R3 is halo.

8. The compound of formula (I) according to claim 6, or a tautomer, isomer, stereoisomer, a deuterated isotope, and a pharmaceutically acceptable salt and/or solvate thereof, wherein R3 is alkyl.

9. The compound of formula (I) according to claim 6, or a tautomer, isomer, stereoisomer, a deuterated isotope, a pharmaceutically acceptable salt and/or solvate thereof, wherein R4 is H and R2 is —(CH₂)₀₋₃NR13R14.

10. The compound of formula (I) according to claim 1, or a tautomer, isomer, stereoisomer, a deuterated isotope, a pharmaceutically acceptable salt and/or solvate thereof, wherein A is a 5-membered heteroaryl of formula (II), 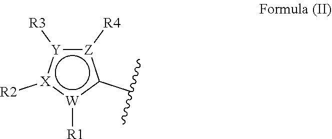
wherein:
X, Y and Z are independently N, C or S;
wherein at least one of X, Y and Z is N or S;
W is C:
R3 and R4 are independently absent, H, alkyl or halo;
R2 is selected from H, halo, alkyl, or cycloalkyl; and
R1 is selected from —(CH₂)₀₋₃NR12(CH₂)₀₋₃(heterocyclyl), —(CH₂)₀₋₃NR12CO(CH₂)₀₋₃(heterocyclyl), —(CH₂)₀₋₃—O—(CH₂)₀₋₃(heterocyclyl), or —(CH₂)₀₋₃heterocyclyl.

11. The compound of formula (I) according to claim 10, or a tautomer, isomer, stereoisomer, a deuterated isotope, a pharmaceutically acceptable salt and/or solvate thereof, wherein Z is S, and Y and X and C.

12. The compound of formula (I) according to claim 10, or a tautomer, isomer, stereoisomer, a deuterated isotope, a pharmaceutically acceptable salt and/or solvate thereof, wherein Z is S, Y is C, and X is N.

13. The compound of formula (I) according to claim 10, or a tautomer, isomer, stereoisomer, a deuterated isotope, a pharmaceutically acceptable salt and/or solvate thereof, wherein R1 is —(CH₂)₀₋₃NR12(CH₂)₀₋₃(heterocyclyl).

14. The compound of formula (I) according to claim 1, or a tautomer, isomer, stereoisomer, a deuterated isotope, a pharmaceutically acceptable salt and/or solvate thereof, wherein A is a 5-membered heteroaryl of formula (II), 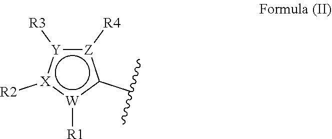
wherein:
Y and Z are N;
W and X are C;
R1 and R2 are selected from H, halo, alkyl, cycloalkyl, or —(CH₂)₀₋₃aryl;
R3 and R4 are independently absent, —(CH₂)₀₋₃heterocyclyl, or —(CH₂)₀₋₃aryl; and
wherein at least one of R3 or R4 is selected from —(CH₂)₀₋₃heterocyclyl, or —(CH₂)₀₋₃aryl.

15. The compound of formula (I) according to claim 14, or a tautomer, isomer, stereoisomer, a deuterated isotope, a pharmaceutically acceptable salt and/or solvate thereof, wherein R2 is halo.

16. The compound of formula (I) according to claim 14, or a tautomer, isomer, stereoisomer, a deuterated isotope, a pharmaceutically acceptable salt and/or solvate thereof, wherein R2 is H.

17. The compound of formula (I) according to claim 14, or a tautomer, isomer, stereoisomer, a deuterated isotope, a pharmaceutically acceptable salt and/or solvate thereof, wherein R4 is —(CH₂)₀₋₃heterocyclyl.

18. The compound of formula (I) according to claim 1, or a tautomer, isomer, stereoisomer, a deuterated isotope, a pharmaceutically acceptable salt and/or solvate thereof, wherein A is a 5-membered heteroaryl of formula (II), 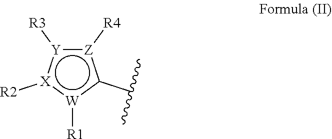
wherein:
Y or Z are independently C, N or S;
wherein at least one of Y and Z is N or S;
W and X are C;
R1 is H;
R2 is selected from H, alkyl, aryl, or halo;
R4 is absent, H or alkyl; and
R3 is —(CH₂)₀₋₃(heterocyclyl).

19. The compound of formula (I) according to claim 18, or a tautomer, isomer, stereoisomer, a deuterated isotope, a pharmaceutically acceptable salt and/or solvate thereof, wherein Z is N.

20. The compound of formula (I) according to claim 18, or a tautomer, isomer, stereoisomer, a deuterated isotope, a pharmaceutically acceptable salt and/or solvate thereof, wherein Y is N.

21. The compound of formula (I) according to claim 18, or a tautomer, isomer, stereoisomer, a deuterated isotope, a pharmaceutically acceptable salt and/or solvate thereof, wherein R3 is —(CH₂)₀₋₃(heterocyclyl).

22. The compound of formula (I) according to claim 1, or a tautomer, isomer, stereoisomer, a deuterated isotope, a pharmaceutically acceptable salt and/or solvate thereof, wherein A is a 5-membered heteroaryl of formula (II), 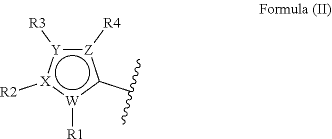
wherein:
Y and X are independently C or N;
wherein at least one of Y or X is N;
W and Z are C;
R1 and R4 are independently selected from H, alkyl, or halo; and
one of R2 and R3 is absent and the other of R2 and R3 is 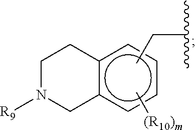
m is 0, 1, 2, or 3;
R9 is selected from H or alkyl;
Each R10 is independently selected from alkyl or halo.

23. The compound of formula (I) according to claim 22, or a tautomer, isomer, stereoisomer, a deuterated isotope, a pharmaceutically acceptable salt and/or solvate thereof, wherein one of R2 and R3 is absent and the other of R2 and R3 is

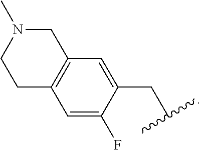 [SMILES: *Cc1cc2c(cc1F)CCN(C)C2]

24. The compound of formula (I) according to claim 22, or a tautomer, isomer, stereoisomer, a deuterated isotope, a pharmaceutically acceptable salt and/or solvate thereof, wherein R1 is alkyl, preferably —CH₂OCH₃.

25. The compound of formula (I) according to claim 1, or a tautomer, isomer, stereoisomer, a deuterated isotope, a pharmaceutically acceptable salt and/or solvate thereof, wherein A is a 9-membered heteroaromatic bicycle of formula (III) 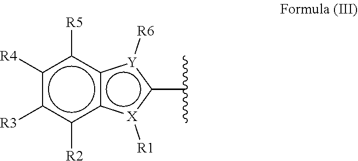
wherein:
X and Y are independently selected from C, N or S;
wherein at least one of X and Y is N or S;
R1 and R6 are independently absent, H or —(CH₂)₀₋₃heterocyclyl;
R2 is selected from H, halo, —(CH₂)₀₋₃NR12(CH₂)₀₋₃(heterocyclyl), or —(CH₂)₀₋₃heterocyclyl;
R3, R4, and R5 are independently selected from H, alkyl or halo; and
wherein at least one of R1, R2, R3, R4, R5 and R6 is not H.

26. The compound of formula (I) according to claim 25, or a tautomer, isomer, stereoisomer, a deuterated isotope, a pharmaceutically acceptable salt and/or solvate thereof, wherein Y is S.

27. The compound of formula (I) according to claim 25, or a tautomer, isomer, stereoisomer, a deuterated isotope, a pharmaceutically acceptable salt and/or solvate thereof, wherein Y is N.

28. The compound of formula (I) according to claim 25, or a tautomer, isomer, stereoisomer, a deuterated isotope, a pharmaceutically acceptable salt and/or solvate thereof, wherein R2 is chloro.

29. The compound of formula (I) according to claim 1, or a tautomer, isomer, stereoisomer, a deuterated isotope, a pharmaceutically acceptable salt and/or solvate thereof, wherein A is a compound of formula (Ia), 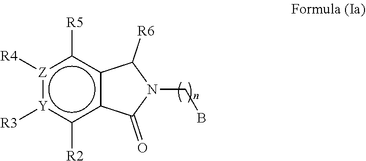
wherein:
n is 0, 1, or 2;
Z and Y and independently selected from C or N;
R6 is selected from H or alkyl;
R4 and R5 are independently absent, H, alkyl, or halo; and
one of R2 and R5 is —(CH₂)₀₋₃NR12(CH₂)₀₋₃(heterocyclyl), and the other of R2 and R5 is selected from H, alkyl, or halo.

30. The compound of formula (I) according to claim 29, or a tautomer, isomer, stereoisomer, a deuterated isotope, a pharmaceutically acceptable salt and/or solvate thereof, wherein R2 is —(CH₂)₀₋₃NR12(CH₂)₀₋₃(heterocyclyl).

31. A compound selected from: 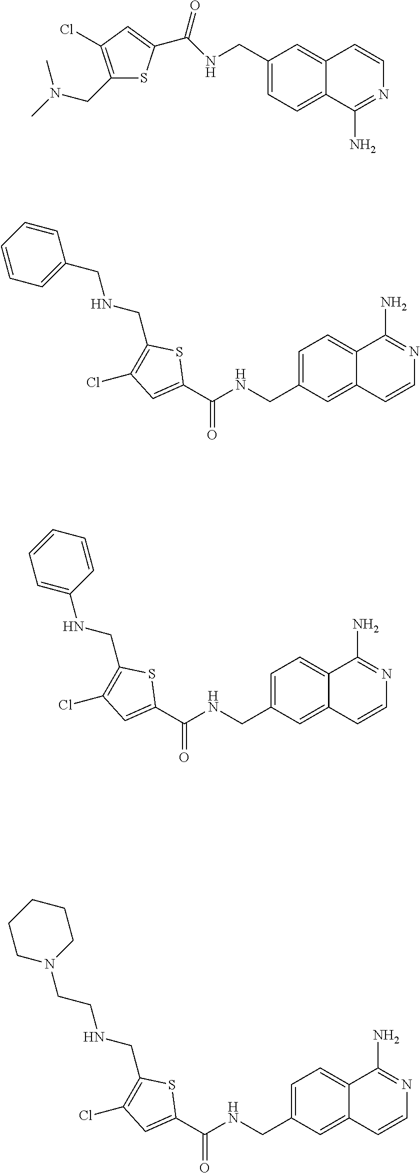 [SMILES: CN(C)Cc1sc(C(=O)NCc2ccc3c(N)nccc3c2)cc1Cl ; Nc1nccc2cc(CNC(=O)c3cc(Cl)c(CNCc4ccccc4)s3)ccc12 ; Nc1nccc2cc(CNC(=O)c3cc(Cl)c(CNc4ccccc4)s3)ccc12 ; Nc1nccc2cc(CNC(=O)c3cc(Cl)c(CNCCN4CCCCC4)s3)ccc12] 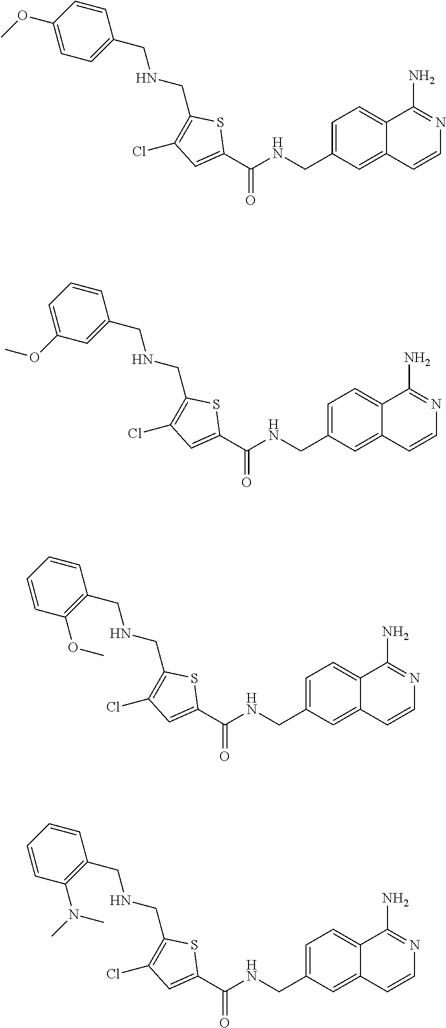 [SMILES: COc1ccc(CNCc2sc(C(=O)NCc3ccc4c(N)nccc4c3)cc2Cl)cc1 ; COc1cccc(CNCc2sc(C(=O)NCc3ccc4c(N)nccc4c3)cc2Cl)c1 ; COc1ccccc1CNCc1sc(C(=O)NCc2ccc3c(N)nccc3c2)cc1Cl ; CN(C)c1ccccc1CNCc1sc(C(=O)NCc2ccc3c(N)nccc3c2)cc1Cl] 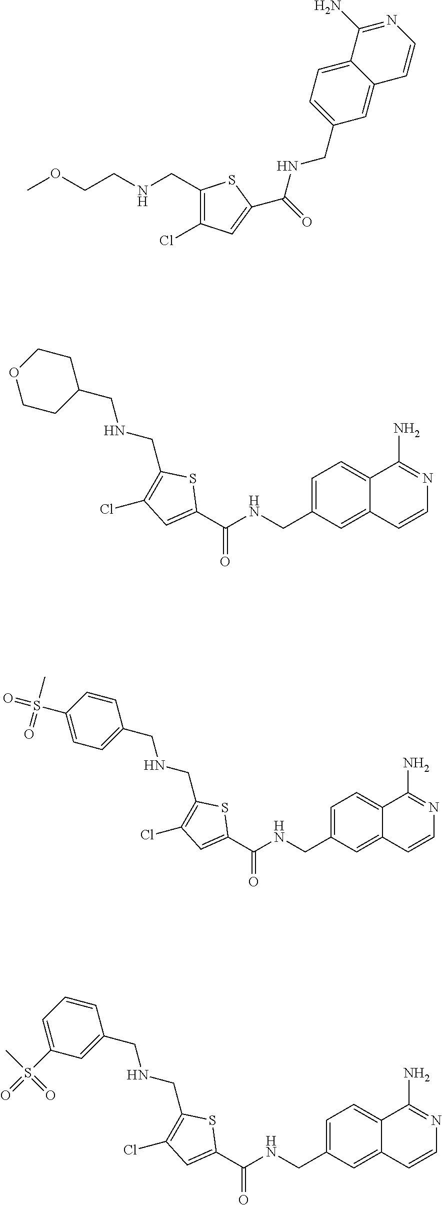 [SMILES: COCCNCc1sc(C(=O)NCc2ccc3c(N)nccc3c2)cc1Cl ; Nc1nccc2cc(CNC(=O)c3cc(Cl)c(CNCC4CCOCC4)s3)ccc12 ; CS(=O)(=O)c1ccc(CNCc2sc(C(=O)NCc3ccc4c(N)nccc4c3)cc2Cl)cc1 ; CS(=O)(=O)c1cccc(CNCc2sc(C(=O)NCc3ccc4c(N)nccc4c3)cc2Cl)c1] 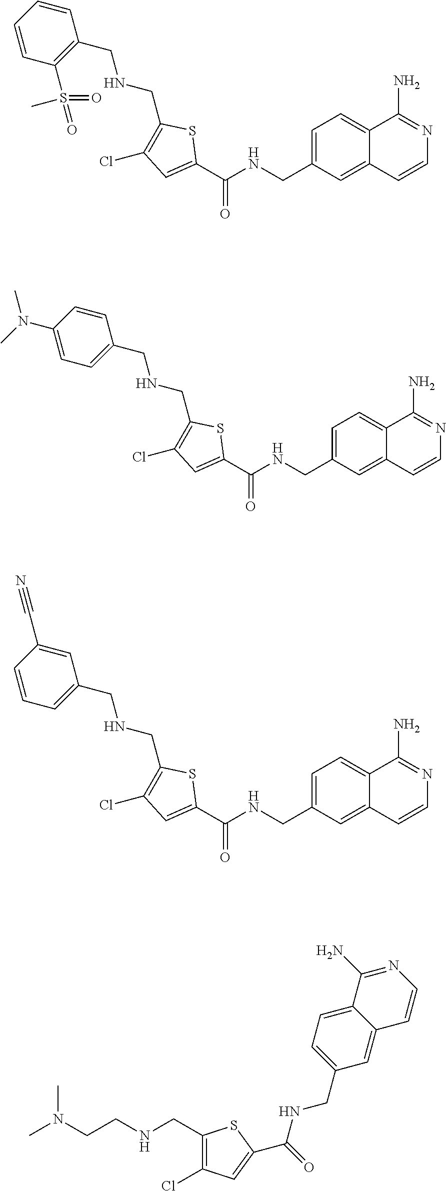 [SMILES: CS(=O)(=O)c1ccccc1CNCc1sc(C(=O)NCc2ccc3c(N)nccc3c2)cc1Cl ; CN(C)c1ccc(CNCc2sc(C(=O)NCc3ccc4c(N)nccc4c3)cc2Cl)cc1 ; N#Cc1cccc(CNCc2sc(C(=O)NCc3ccc4c(N)nccc4c3)cc2Cl)c1 ; CN(C)CCNCc1sc(C(=O)NCc2ccc3c(N)nccc3c2)cc1Cl] 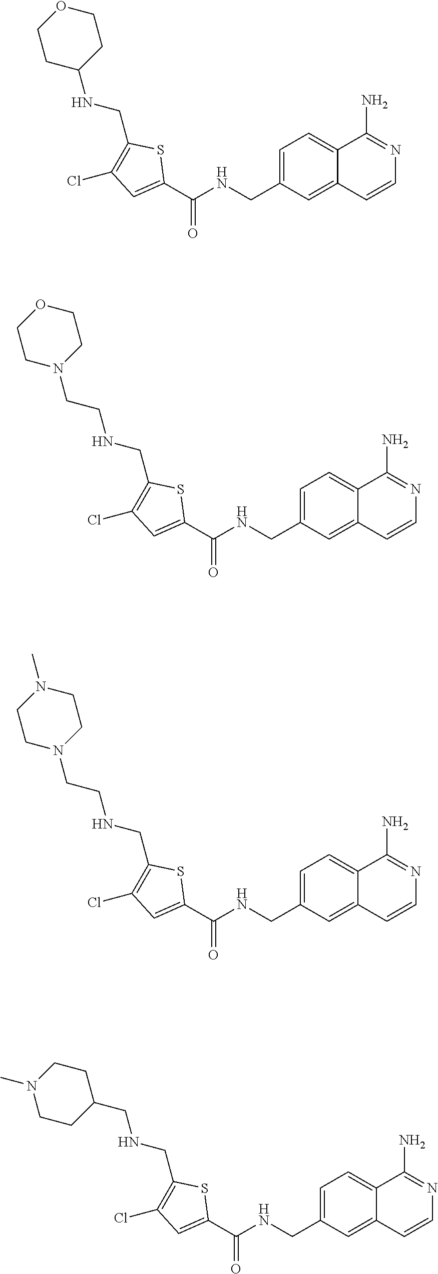 [SMILES: Nc1nccc2cc(CNC(=O)c3cc(Cl)c(CNC4CCOCC4)s3)ccc12 ; Nc1nccc2cc(CNC(=O)c3cc(Cl)c(CNCCN4CCOCC4)s3)ccc12 ; CN1CCN(CCNCc2sc(C(=O)NCc3ccc4c(N)nccc4c3)cc2Cl)CC1 ; CN1CCC(CNCc2sc(C(=O)NCc3ccc4c(N)nccc4c3)cc2Cl)CC1] 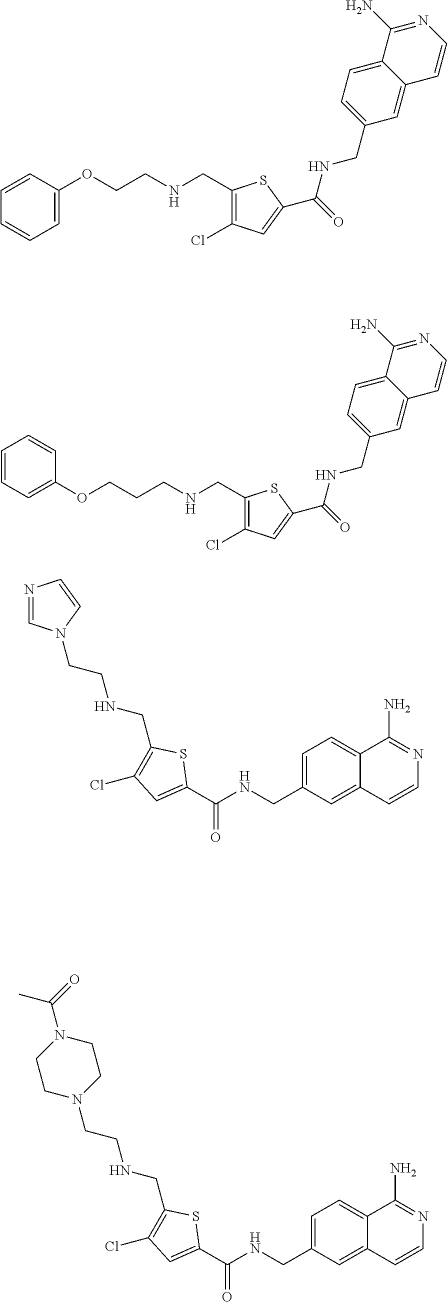 [SMILES: Nc1nccc2cc(CNC(=O)c3cc(Cl)c(CNCCOc4ccccc4)s3)ccc12 ; Nc1nccc2cc(CNC(=O)c3cc(Cl)c(CNCCCOc4ccccc4)s3)ccc12 ; Nc1nccc2cc(CNC(=O)c3cc(Cl)c(CNCCn4ccnc4)s3)ccc12 ; CC(=O)N1CCN(CCNCc2sc(C(=O)NCc3ccc4c(N)nccc4c3)cc2Cl)CC1] 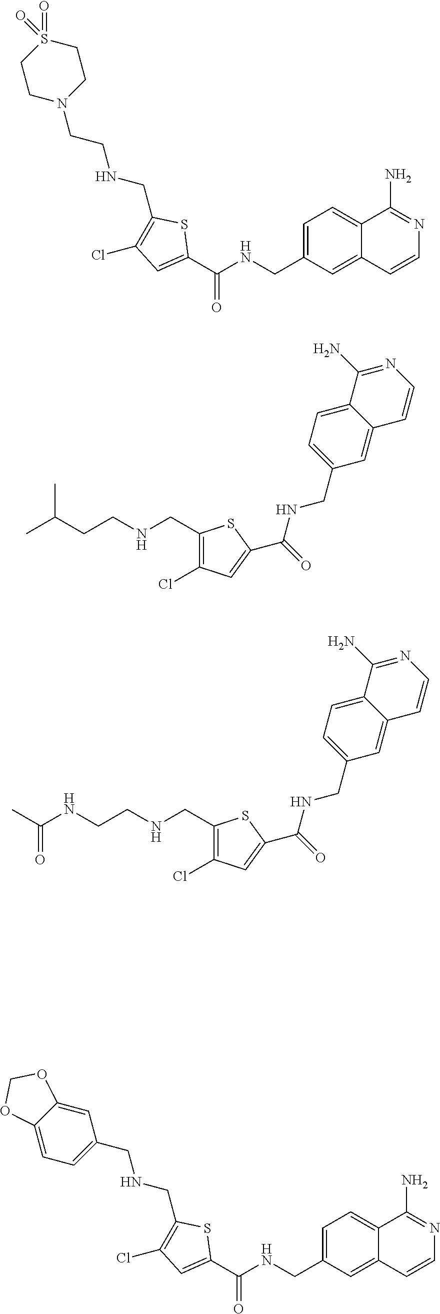 [SMILES: Nc1nccc2cc(CNC(=O)c3cc(Cl)c(CNCCN4CCS(=O)(=O)CC4)s3)ccc12 ; CC(C)CCNCc1sc(C(=O)NCc2ccc3c(N)nccc3c2)cc1Cl ; CC(=O)NCCNCc1sc(C(=O)NCc2ccc3c(N)nccc3c2)cc1Cl ; Nc1nccc2cc(CNC(=O)c3cc(Cl)c(CNCc4ccc5c(c4)OCO5)s3)ccc12] 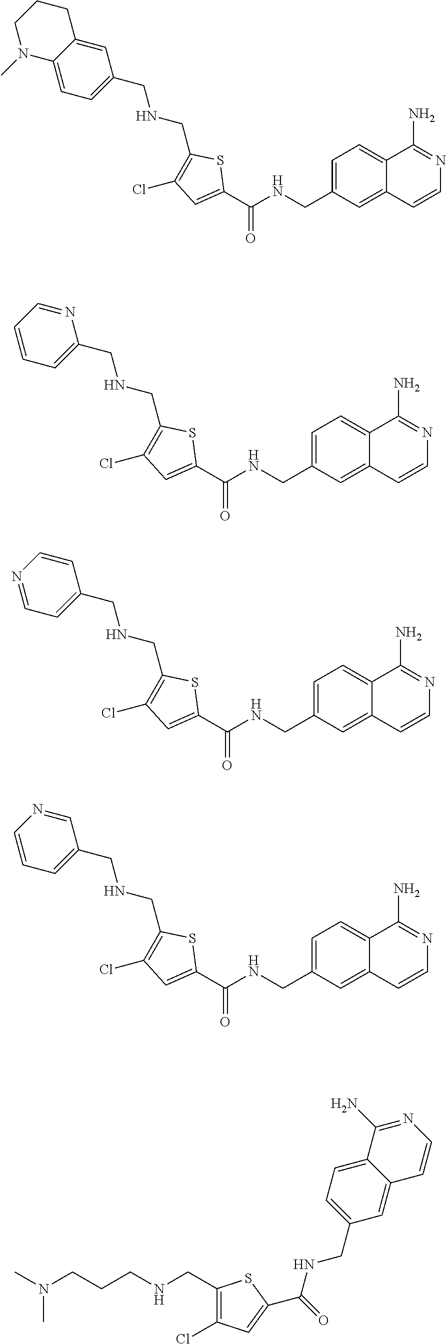 [SMILES: CN1CCCc2cc(CNCc3sc(C(=O)NCc4ccc5c(N)nccc5c4)cc3Cl)ccc21 ; Nc1nccc2cc(CNC(=O)c3cc(Cl)c(CNCc4ccccn4)s3)ccc12 ; Nc1nccc2cc(CNC(=O)c3cc(Cl)c(CNCc4ccncc4)s3)ccc12 ; Nc1nccc2cc(CNC(=O)c3cc(Cl)c(CNCc4cccnc4)s3)ccc12 ; CN(C)CCCNCc1sc(C(=O)NCc2ccc3c(N)nccc3c2)cc1Cl] 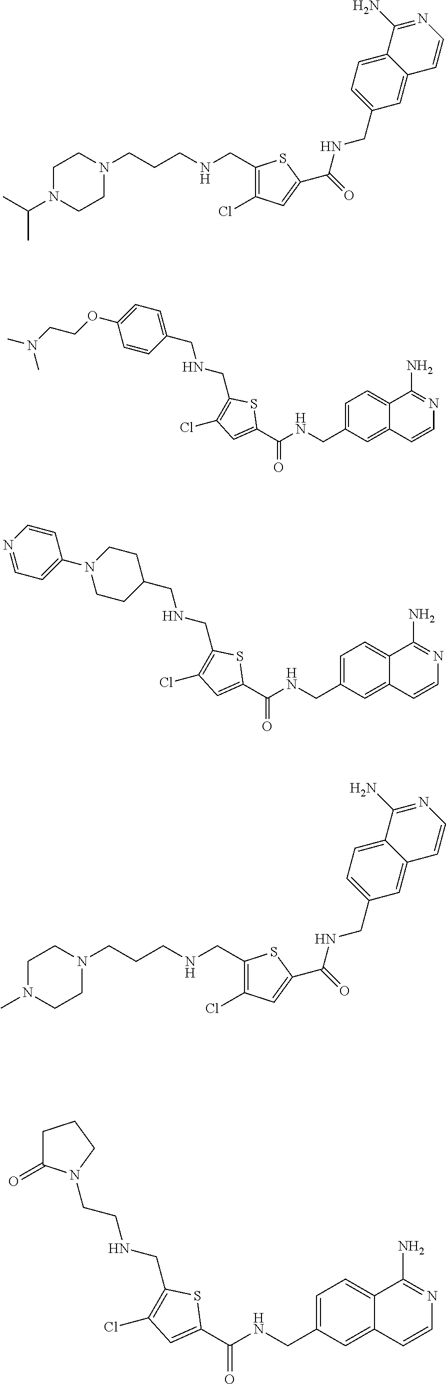 [SMILES: CC(C)N1CCN(CCCNCc2sc(C(=O)NCc3ccc4c(N)nccc4c3)cc2Cl)CC1 ; CN(C)CCOc1ccc(CNCc2sc(C(=O)NCc3ccc4c(N)nccc4c3)cc2Cl)cc1 ; Nc1nccc2cc(CNC(=O)c3cc(Cl)c(CNCC4CCN(c5ccncc5)CC4)s3)ccc12 ; CN1CCN(CCCNCc2sc(C(=O)NCc3ccc4c(N)nccc4c3)cc2Cl)CC1 ; Nc1nccc2cc(CNC(=O)c3cc(Cl)c(CNCCN4CCCC4=O)s3)ccc12] 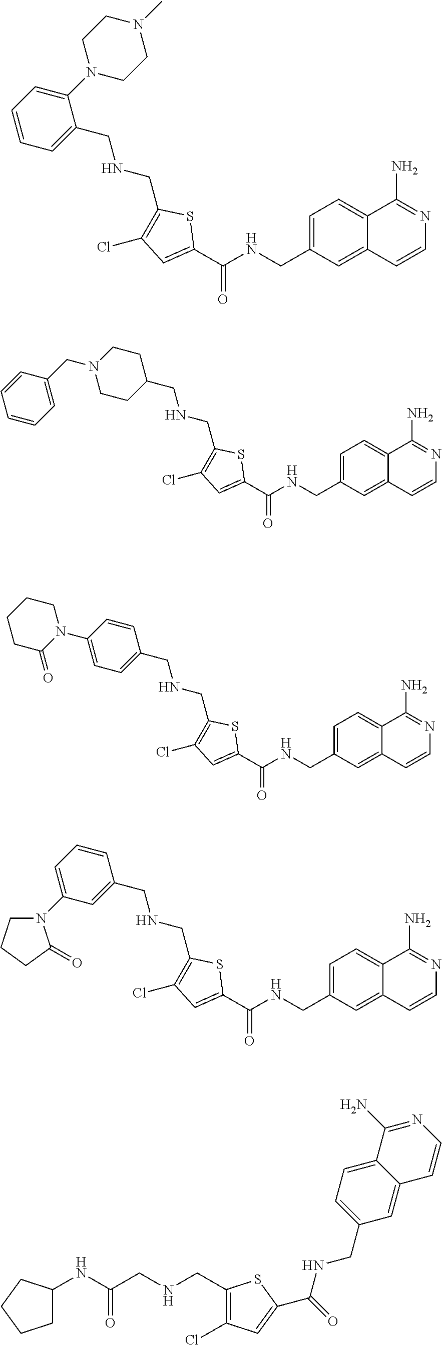 [SMILES: CN1CCN(c2ccccc2CNCc2sc(C(=O)NCc3ccc4c(N)nccc4c3)cc2Cl)CC1 ; Nc1nccc2cc(CNC(=O)c3cc(Cl)c(CNCC4CCN(Cc5ccccc5)CC4)s3)ccc12 ; Nc1nccc2cc(CNC(=O)c3cc(Cl)c(CNCc4ccc(N5CCCCC5=O)cc4)s3)ccc12 ; Nc1nccc2cc(CNC(=O)c3cc(Cl)c(CNCc4cccc(N5CCCC5=O)c4)s3)ccc12 ; Nc1nccc2cc(CNC(=O)c3cc(Cl)c(CNCC(=O)NC4CCCC4)s3)ccc12] 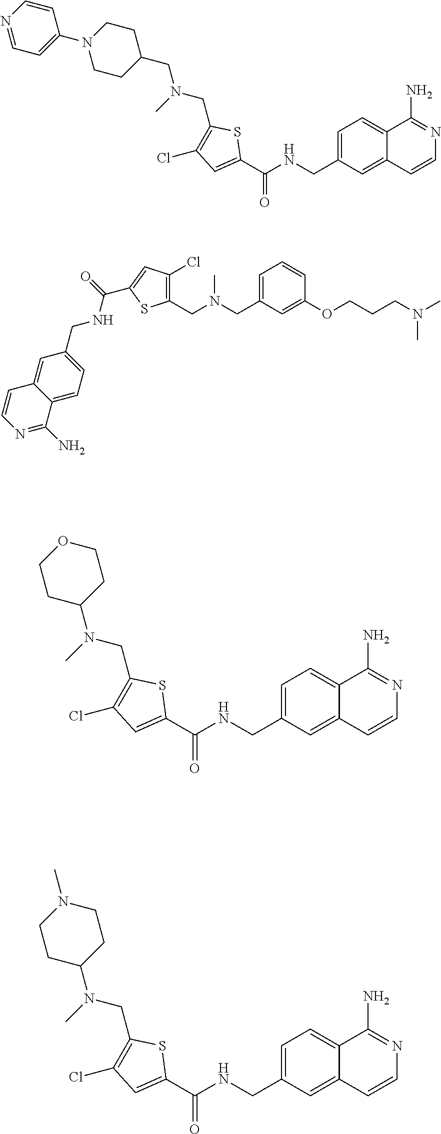 [SMILES: CN(Cc1sc(C(=O)NCc2ccc3c(N)nccc3c2)cc1Cl)CC1CCN(c2ccncc2)CC1 ; CN(C)CCCOc1cccc(CN(C)Cc2sc(C(=O)NCc3ccc4c(N)nccc4c3)cc2Cl)c1 ; CN(Cc1sc(C(=O)NCc2ccc3c(N)nccc3c2)cc1Cl)C1CCOCC1 ; CN1CCC(N(C)Cc2sc(C(=O)NCc3ccc4c(N)nccc4c3)cc2Cl)CC1] 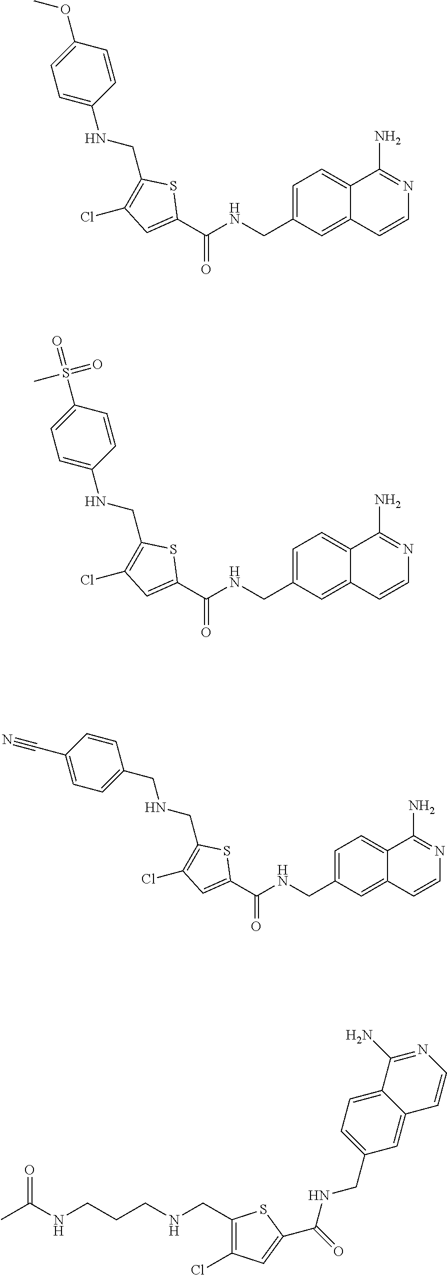 [SMILES: COc1ccc(NCc2sc(C(=O)NCc3ccc4c(N)nccc4c3)cc2Cl)cc1 ; CS(=O)(=O)c1ccc(NCc2sc(C(=O)NCc3ccc4c(N)nccc4c3)cc2Cl)cc1 ; N#Cc1ccc(CNCc2sc(C(=O)NCc3ccc4c(N)nccc4c3)cc2Cl)cc1 ; CC(=O)NCCCNCc1sc(C(=O)NCc2ccc3c(N)nccc3c2)cc1Cl] 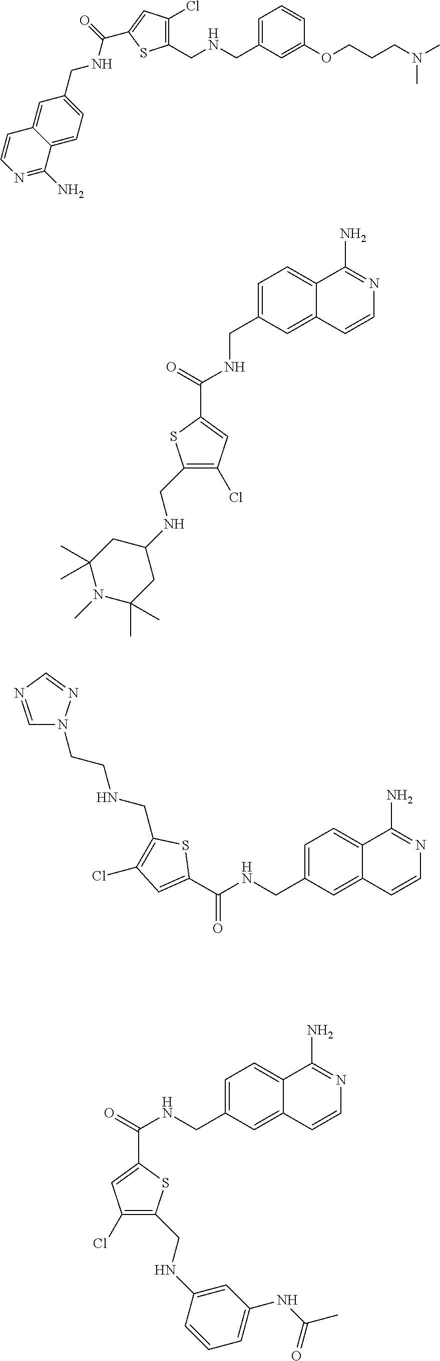 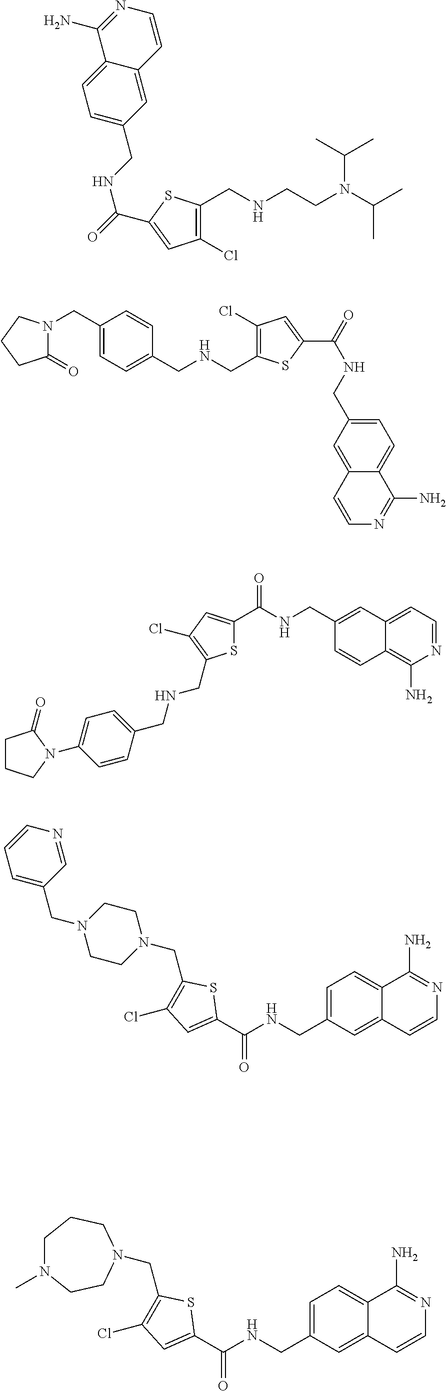 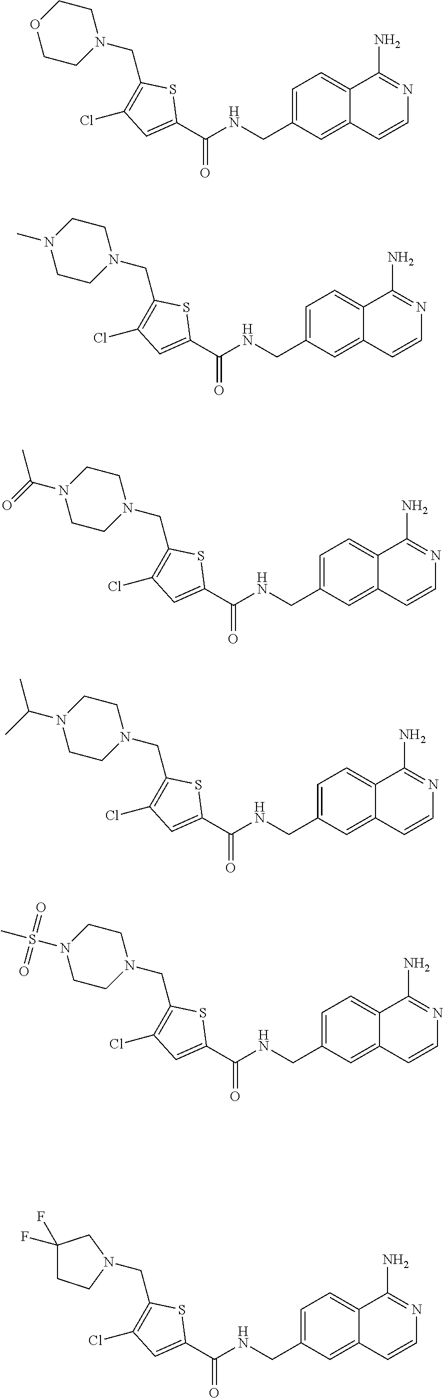 [SMILES: Nc1nccc2cc(CNC(=O)c3cc(Cl)c(CN4CCOCC4)s3)ccc12 ; CN1CCN(Cc2sc(C(=O)NCc3ccc4c(N)nccc4c3)cc2Cl)CC1 ; CC(=O)N1CCN(Cc2sc(C(=O)NCc3ccc4c(N)nccc4c3)cc2Cl)CC1 ; CC(C)N1CCN(Cc2sc(C(=O)NCc3ccc4c(N)nccc4c3)cc2Cl)CC1 ; CS(=O)(=O)N1CCN(Cc2sc(C(=O)NCc3ccc4c(N)nccc4c3)cc2Cl)CC1 ; Nc1nccc2cc(CNC(=O)c3cc(Cl)c(CN4CCC(F)(F)C4)s3)ccc12] 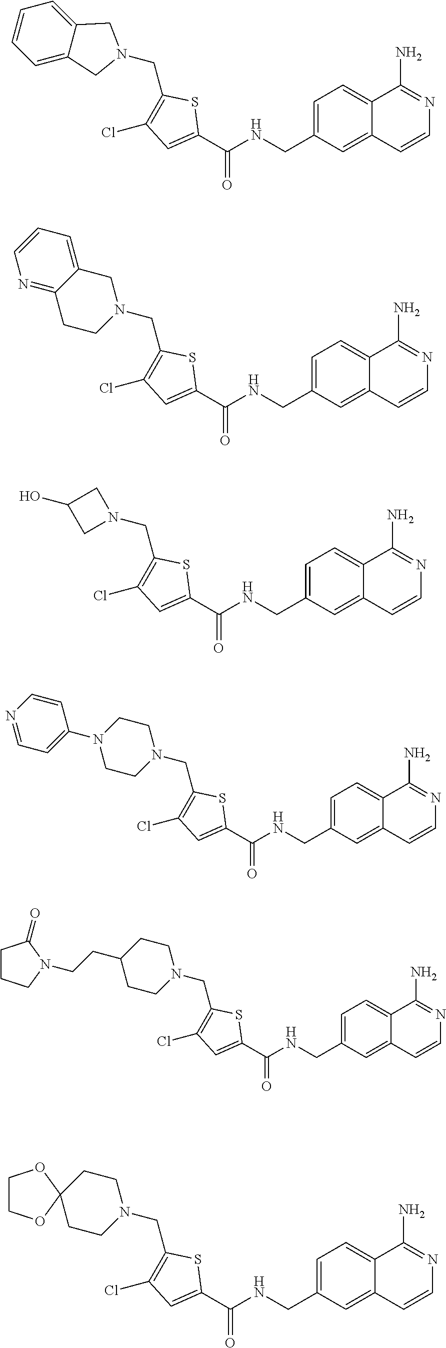 [SMILES: Nc1nccc2cc(CNC(=O)c3cc(Cl)c(CN4Cc5ccccc5C4)s3)ccc12 ; Nc1nccc2cc(CNC(=O)c3cc(Cl)c(CN4CCc5ncccc5C4)s3)ccc12 ; Nc1nccc2cc(CNC(=O)c3cc(Cl)c(CN4CC(O)C4)s3)ccc12 ; Nc1nccc2cc(CNC(=O)c3cc(Cl)c(CN4CCN(c5ccncc5)CC4)s3)ccc12 ; Nc1nccc2cc(CNC(=O)c3cc(Cl)c(CN4CCC(CCN5CCCC5=O)CC4)s3)ccc12 ; Nc1nccc2cc(CNC(=O)c3cc(Cl)c(CN4CCC5(CC4)OCCO5)s3)ccc12] 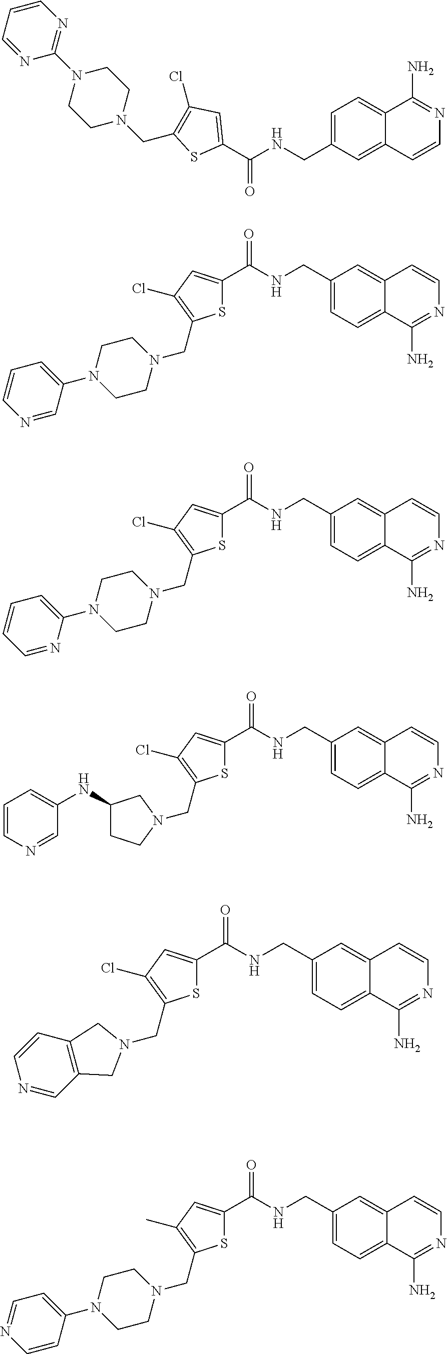 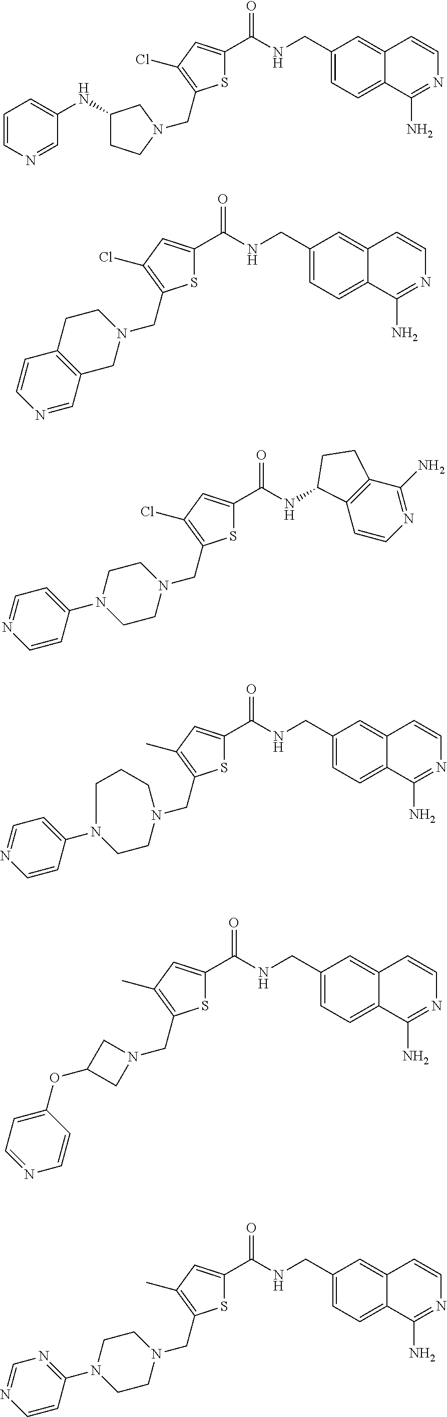 [SMILES: Nc1nccc2cc(CNC(=O)c3cc(Cl)c(CN4CC[C@H](Nc5cccnc5)C4)s3)ccc12 ; Nc1nccc2cc(CNC(=O)c3cc(Cl)c(CN4CCc5ccncc5C4)s3)ccc12 ; Nc1nccc2c1CC[C@H]2NC(=O)c1cc(Cl)c(CN2CCN(c3ccncc3)CC2)s1 ; Cc1cc(C(=O)NCc2ccc3c(N)nccc3c2)sc1CN1CCCN(c2ccncc2)CC1 ; Cc1cc(C(=O)NCc2ccc3c(N)nccc3c2)sc1CN1CC(Oc2ccncc2)C1 ; Cc1cc(C(=O)NCc2ccc3c(N)nccc3c2)sc1CN1CCN(c2ccncn2)CC1] 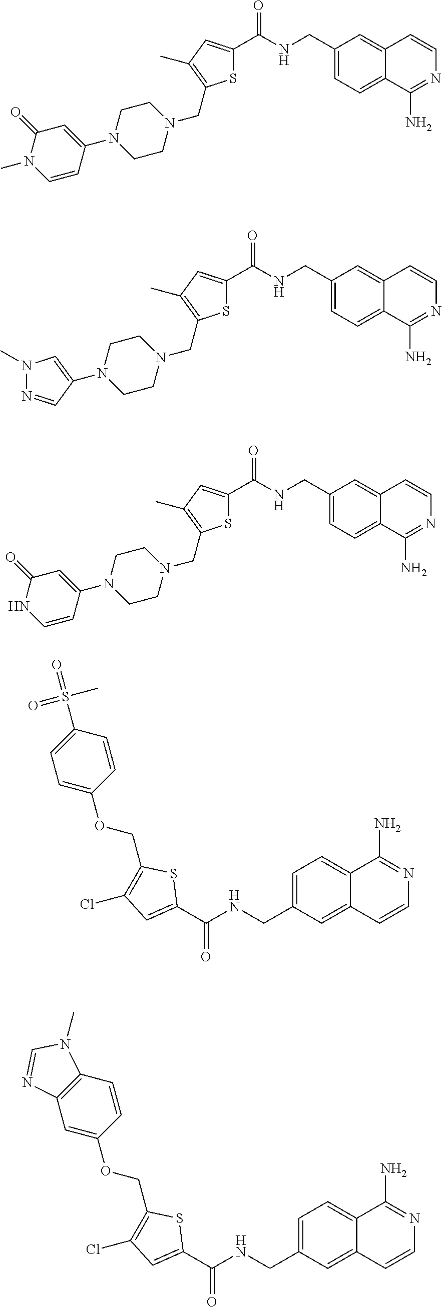 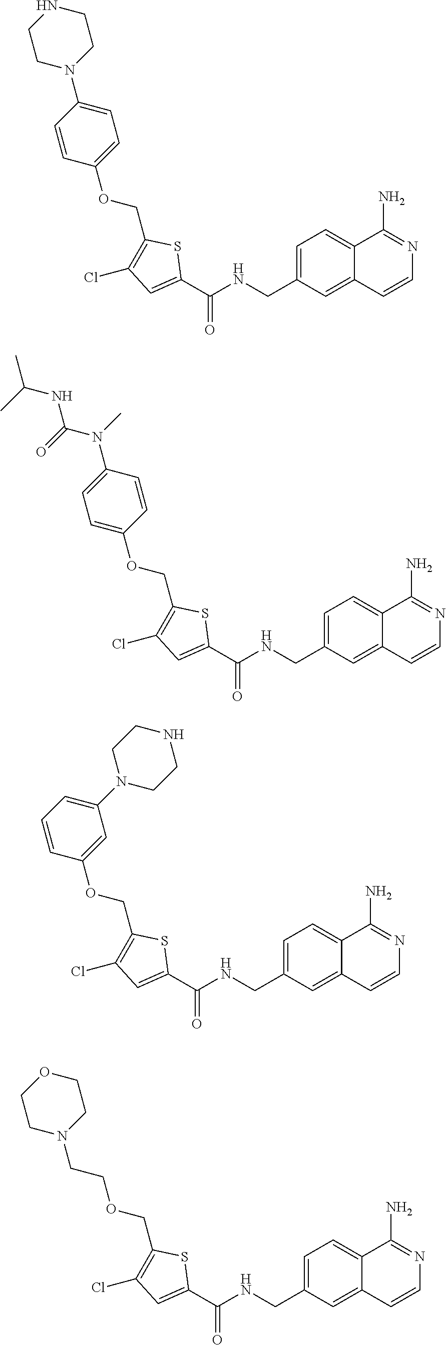 [SMILES: Nc1nccc2cc(CNC(=O)c3cc(Cl)c(COc4ccc(N5CCNCC5)cc4)s3)ccc12 ; CC(C)NC(=O)N(C)c1ccc(OCc2sc(C(=O)NCc3ccc4c(N)nccc4c3)cc2Cl)cc1 ; Nc1nccc2cc(CNC(=O)c3cc(Cl)c(COc4cccc(N5CCNCC5)c4)s3)ccc12 ; Nc1nccc2cc(CNC(=O)c3cc(Cl)c(COCCN4CCOCC4)s3)ccc12] 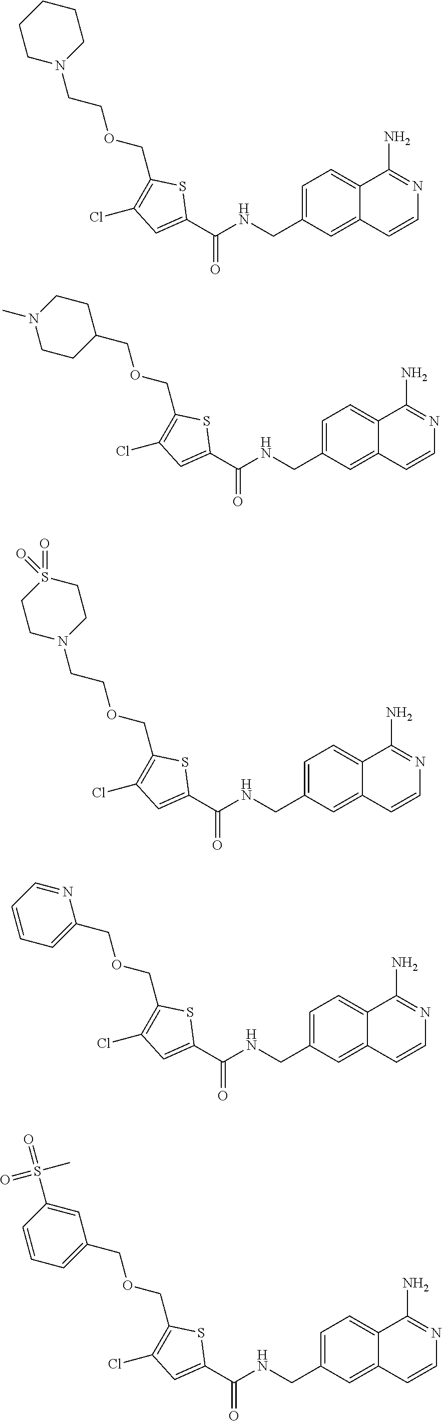 [SMILES: Nc1nccc2cc(CNC(=O)c3cc(Cl)c(COCCN4CCCCC4)s3)ccc12 ; CN1CCC(COCc2sc(C(=O)NCc3ccc4c(N)nccc4c3)cc2Cl)CC1 ; Nc1nccc2cc(CNC(=O)c3cc(Cl)c(COCCN4CCS(=O)(=O)CC4)s3)ccc12 ; Nc1nccc2cc(CNC(=O)c3cc(Cl)c(COCc4ccccn4)s3)ccc12 ; CS(=O)(=O)c1cccc(COCc2sc(C(=O)NCc3ccc4c(N)nccc4c3)cc2Cl)c1] 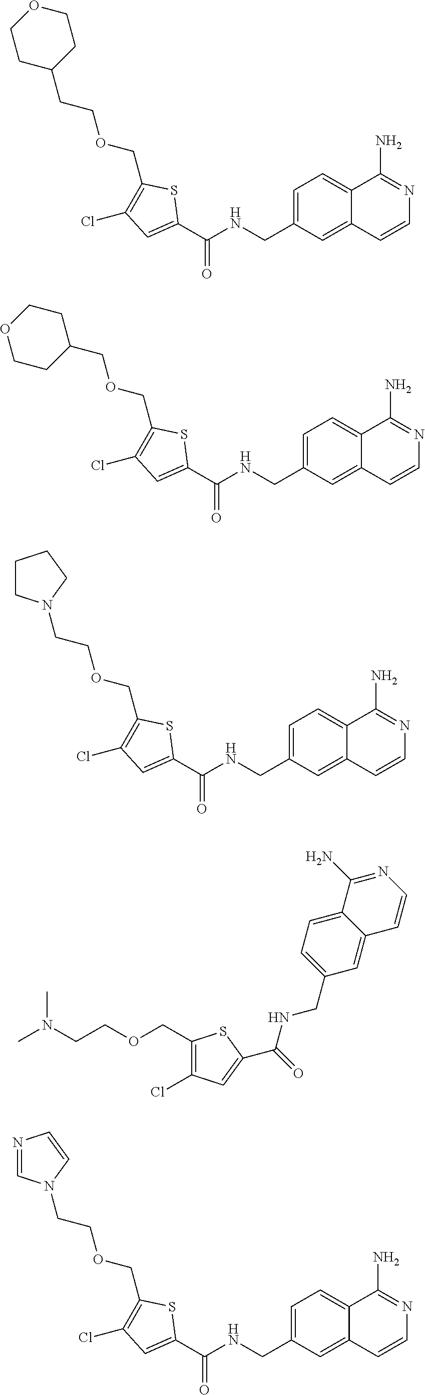 [SMILES: Nc1nccc2cc(CNC(=O)c3cc(Cl)c(COCCC4CCOCC4)s3)ccc12 ; Nc1nccc2cc(CNC(=O)c3cc(Cl)c(COCC4CCOCC4)s3)ccc12 ; Nc1nccc2cc(CNC(=O)c3cc(Cl)c(COCCN4CCCC4)s3)ccc12 ; CN(C)CCOCc1sc(C(=O)NCc2ccc3c(N)nccc3c2)cc1Cl ; Nc1nccc2cc(CNC(=O)c3cc(Cl)c(COCCn4ccnc4)s3)ccc12] 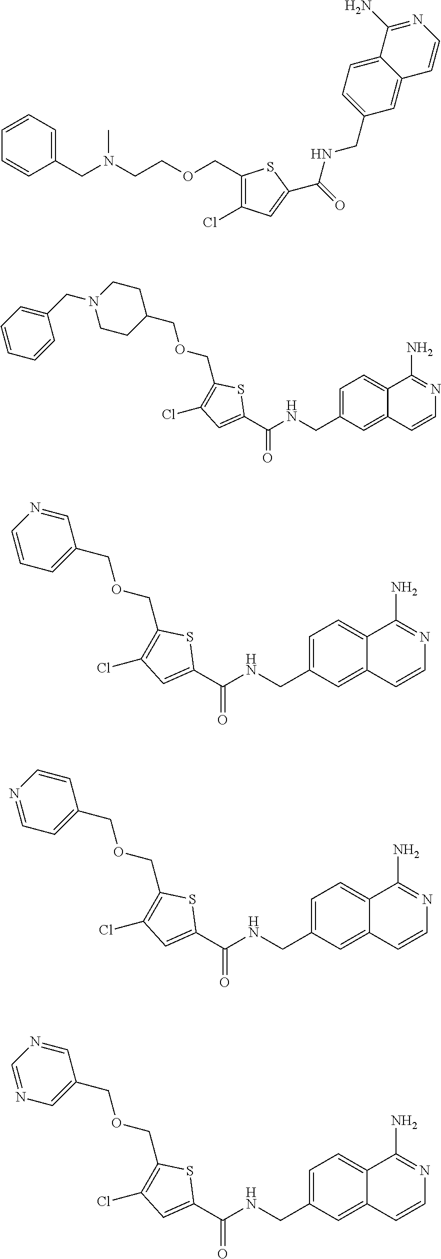 [SMILES: CN(CCOCc1sc(C(=O)NCc2ccc3c(N)nccc3c2)cc1Cl)Cc1ccccc1 ; Nc1nccc2cc(CNC(=O)c3cc(Cl)c(COCC4CCN(Cc5ccccc5)CC4)s3)ccc12 ; Nc1nccc2cc(CNC(=O)c3cc(Cl)c(COCc4cccnc4)s3)ccc12 ; Nc1nccc2cc(CNC(=O)c3cc(Cl)c(COCc4ccncc4)s3)ccc12 ; Nc1nccc2cc(CNC(=O)c3cc(Cl)c(COCc4cncnc4)s3)ccc12] 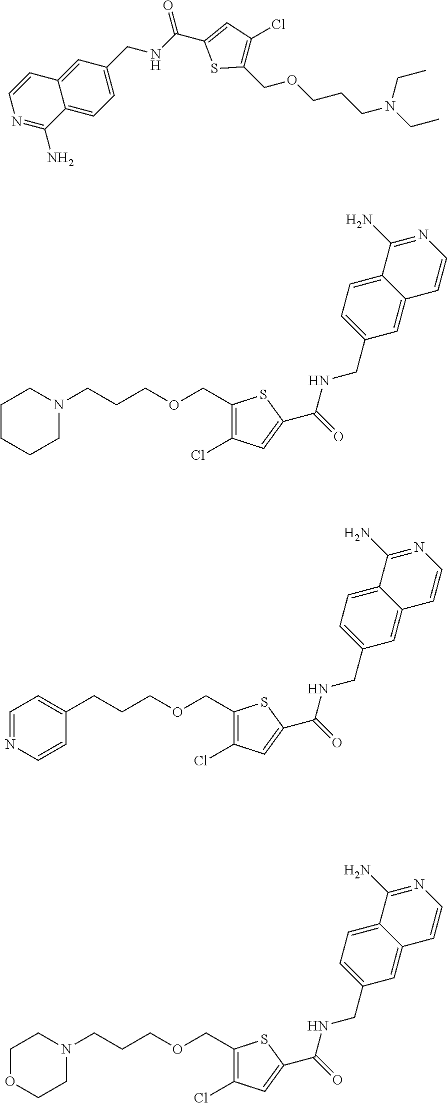 [SMILES: CCN(CC)CCCOCc1sc(C(=O)NCc2ccc3c(N)nccc3c2)cc1Cl ; Nc1nccc2cc(CNC(=O)c3cc(Cl)c(COCCCN4CCCCC4)s3)ccc12 ; Nc1nccc2cc(CNC(=O)c3cc(Cl)c(COCCCc4ccncc4)s3)ccc12 ; Nc1nccc2cc(CNC(=O)c3cc(Cl)c(COCCCN4CCOCC4)s3)ccc12] 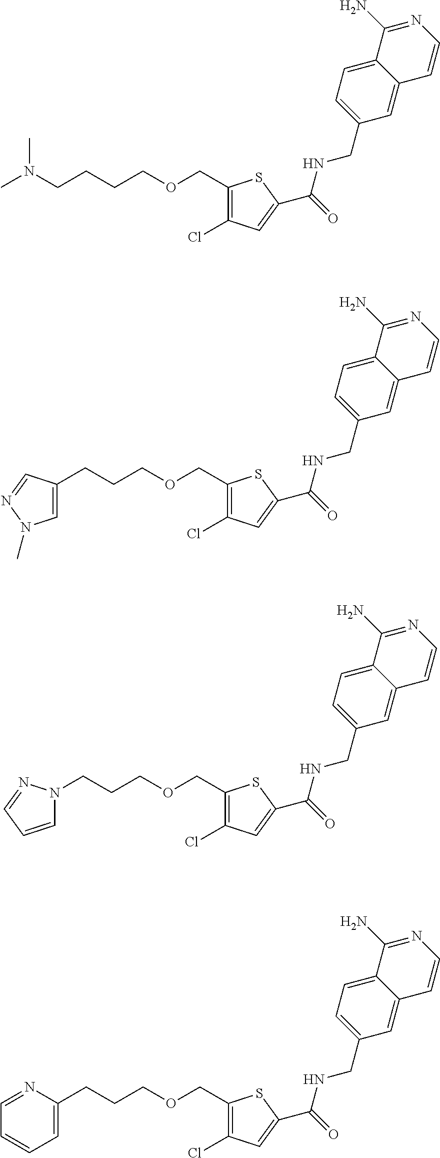 [SMILES: CN(C)CCCCOCc1sc(C(=O)NCc2ccc3c(N)nccc3c2)cc1Cl ; Cn1cc(CCCOCc2sc(C(=O)NCc3ccc4c(N)nccc4c3)cc2Cl)cn1 ; Nc1nccc2cc(CNC(=O)c3cc(Cl)c(COCCCn4cccn4)s3)ccc12 ; Nc1nccc2cc(CNC(=O)c3cc(Cl)c(COCCCc4ccccn4)s3)ccc12] 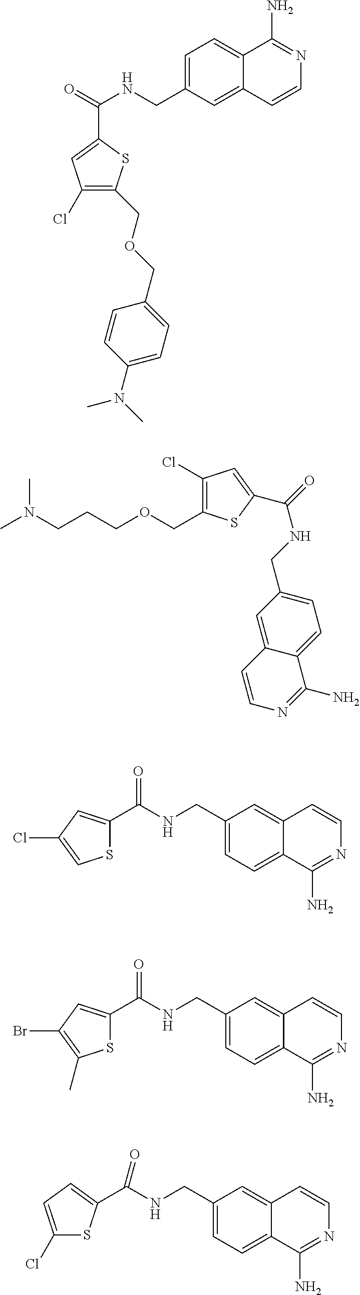 [SMILES: CN(C)c1ccc(COCc2sc(C(=O)NCc3ccc4c(N)nccc4c3)cc2Cl)cc1 ; CN(C)CCCOCc1sc(C(=O)NCc2ccc3c(N)nccc3c2)cc1Cl ; Nc1nccc2cc(CNC(=O)c3cc(Cl)cs3)ccc12 ; Cc1sc(C(=O)NCc2ccc3c(N)nccc3c2)cc1Br ; Nc1nccc2cc(CNC(=O)c3ccc(Cl)s3)ccc12] 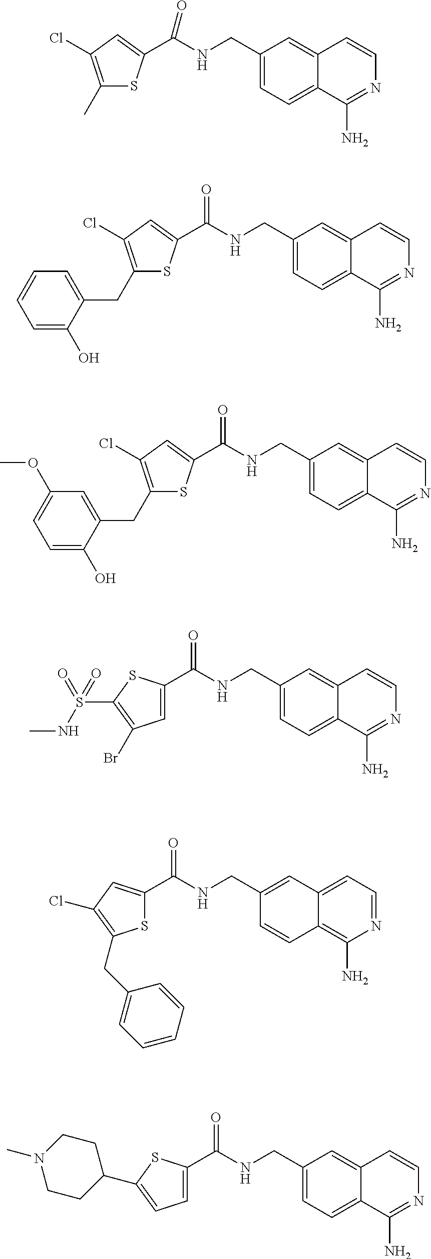 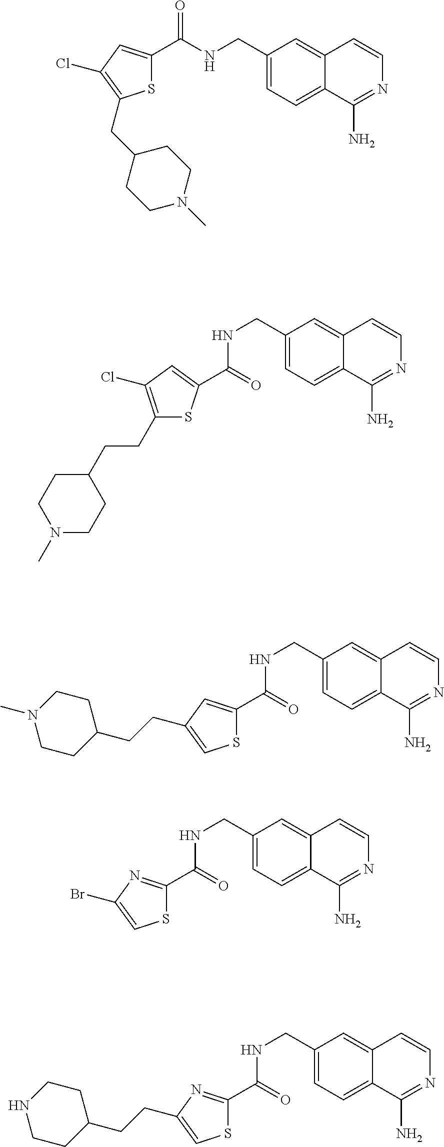 [SMILES: CN1CCC(Cc2sc(C(=O)NCc3ccc4c(N)nccc4c3)cc2Cl)CC1 ; CN1CCC(CCc2sc(C(=O)NCc3ccc4c(N)nccc4c3)cc2Cl)CC1 ; CN1CCC(CCc2csc(C(=O)NCc3ccc4c(N)nccc4c3)c2)CC1 ; Nc1nccc2cc(CNC(=O)c3nc(Br)cs3)ccc12 ; Nc1nccc2cc(CNC(=O)c3nc(CCC4CCNCC4)cs3)ccc12] 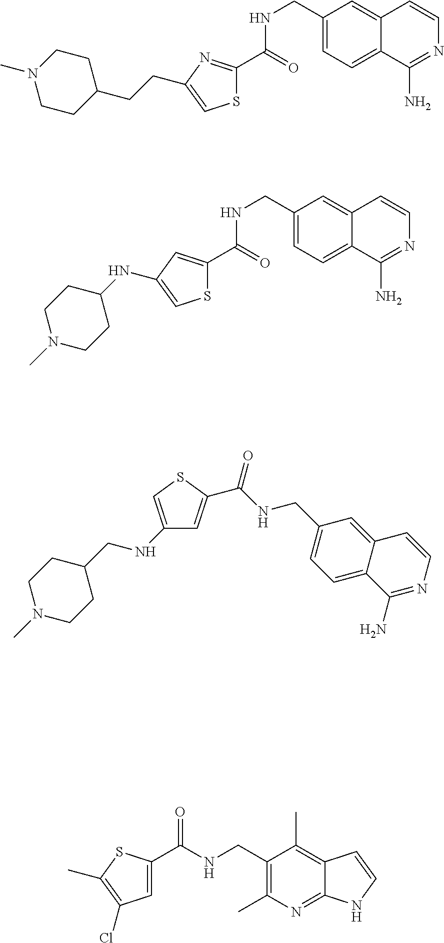 [SMILES: CN1CCC(CCc2csc(C(=O)NCc3ccc4c(N)nccc4c3)n2)CC1 ; CN1CCC(Nc2csc(C(=O)NCc3ccc4c(N)nccc4c3)c2)CC1 ; CN1CCC(CNc2csc(C(=O)NCc3ccc4c(N)nccc4c3)c2)CC1 ; Cc1nc2[nH]ccc2c(C)c1CNC(=O)c1cc(Cl)c(C)s1] 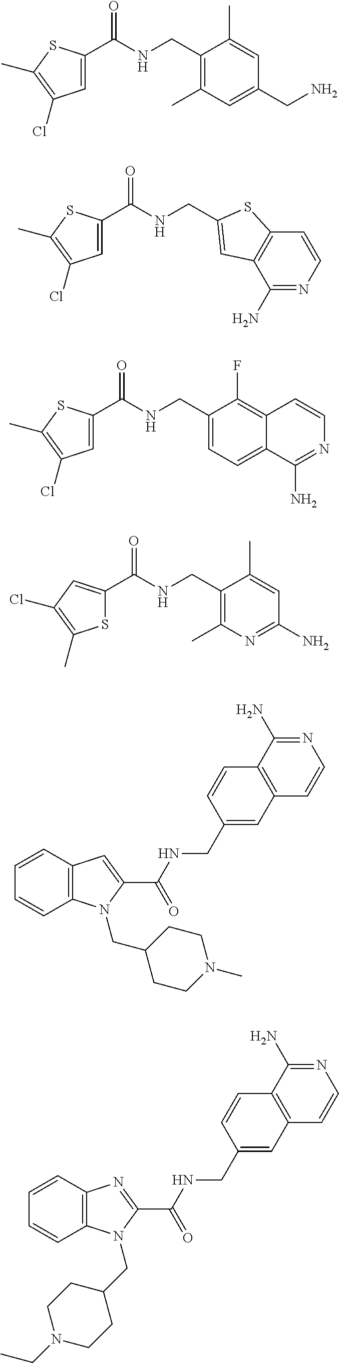 [SMILES: Cc1cc(CN)cc(C)c1CNC(=O)c1cc(Cl)c(C)s1 ; Cc1sc(C(=O)NCc2cc3c(N)nccc3s2)cc1Cl ; Cc1sc(C(=O)NCc2ccc3c(N)nccc3c2F)cc1Cl ; Cc1cc(N)nc(C)c1CNC(=O)c1cc(Cl)c(C)s1 ; CN1CCC(Cn2c(C(=O)NCc3ccc4c(N)nccc4c3)cc3ccccc32)CC1 ; CCN1CCC(Cn2c(C(=O)NCc3ccc4c(N)nccc4c3)nc3ccccc32)CC1] 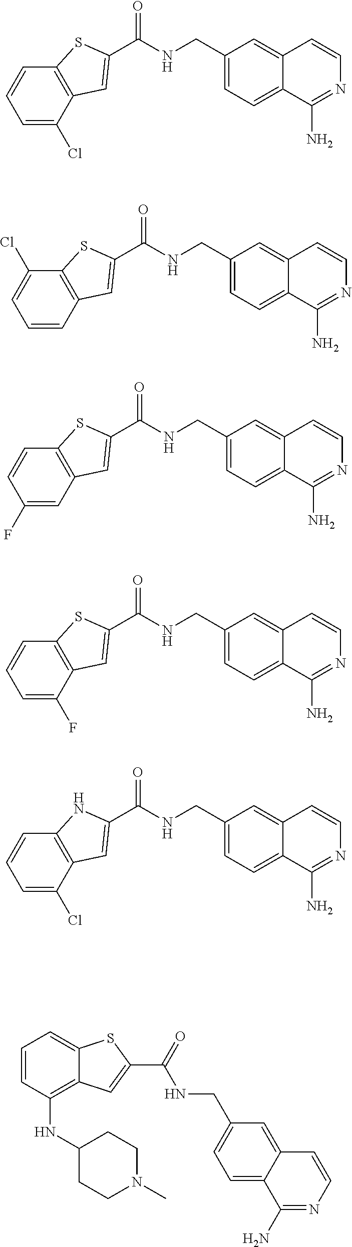 [SMILES: Nc1nccc2cc(CNC(=O)c3cc4c(Cl)cccc4s3)ccc12 ; Nc1nccc2cc(CNC(=O)c3cc4cccc(Cl)c4s3)ccc12 ; Nc1nccc2cc(CNC(=O)c3cc4cc(F)ccc4s3)ccc12 ; Nc1nccc2cc(CNC(=O)c3cc4c(F)cccc4s3)ccc12 ; Nc1nccc2cc(CNC(=O)c3cc4c(Cl)cccc4[nH]3)ccc12 ; CN1CCC(Nc2cccc3sc(C(=O)NCc4ccc5c(N)nccc5c4)cc23)CC1] 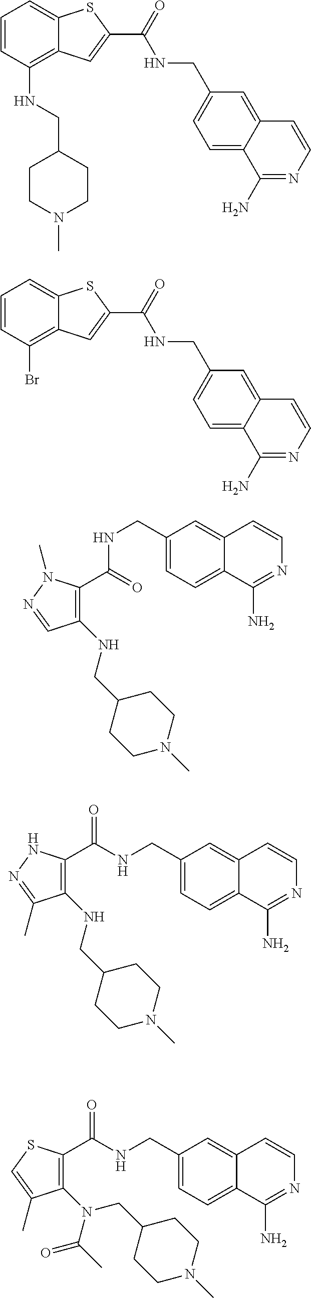 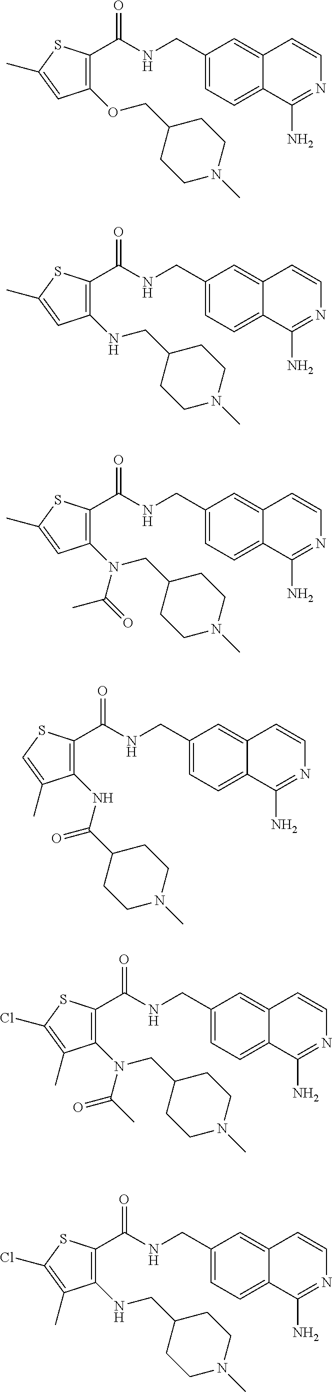 [SMILES: Cc1cc(OCC2CCN(C)CC2)c(C(=O)NCc2ccc3c(N)nccc3c2)s1 ; Cc1cc(NCC2CCN(C)CC2)c(C(=O)NCc2ccc3c(N)nccc3c2)s1 ; CC(=O)N(CC1CCN(C)CC1)c1cc(C)sc1C(=O)NCc1ccc2c(N)nccc2c1 ; Cc1csc(C(=O)NCc2ccc3c(N)nccc3c2)c1NC(=O)C1CCN(C)CC1 ; CC(=O)N(CC1CCN(C)CC1)c1c(C(=O)NCc2ccc3c(N)nccc3c2)sc(Cl)c1C ; Cc1c(Cl)sc(C(=O)NCc2ccc3c(N)nccc3c2)c1NCC1CCN(C)CC1] 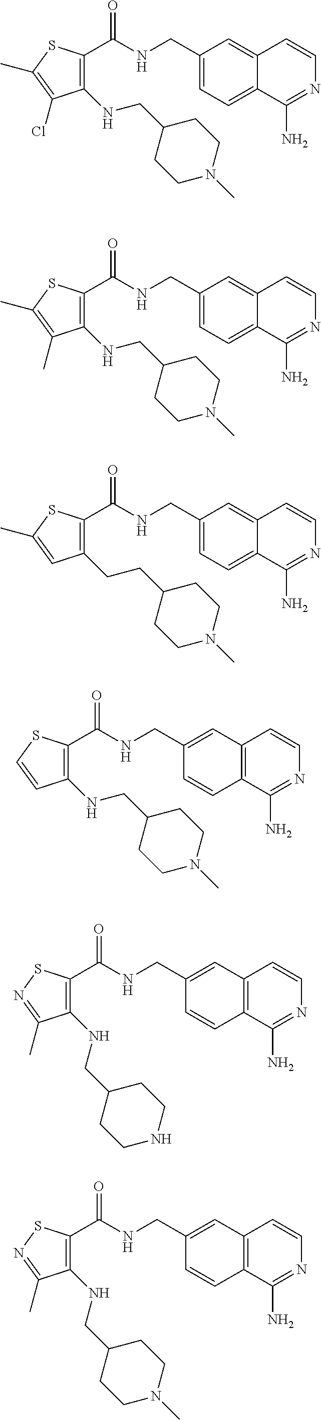 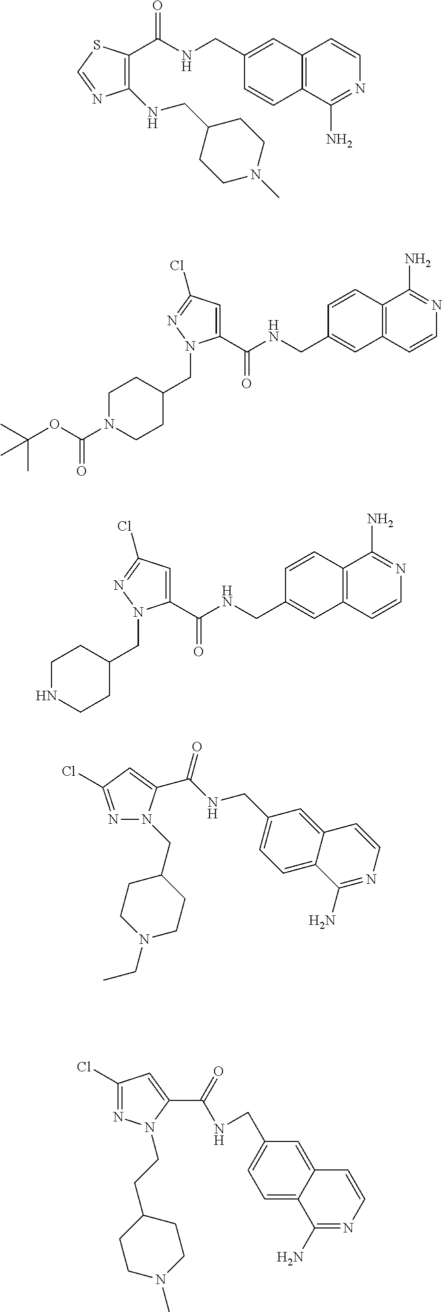 [SMILES: CN1CCC(CNc2ncsc2C(=O)NCc2ccc3c(N)nccc3c2)CC1 ; CC(C)(C)OC(=O)N1CCC(Cn2nc(Cl)cc2C(=O)NCc2ccc3c(N)nccc3c2)CC1 ; Nc1nccc2cc(CNC(=O)c3cc(Cl)nn3CC3CCNCC3)ccc12 ; CCN1CCC(Cn2nc(Cl)cc2C(=O)NCc2ccc3c(N)nccc3c2)CC1 ; CN1CCC(CCn2nc(Cl)cc2C(=O)NCc2ccc3c(N)nccc3c2)CC1] 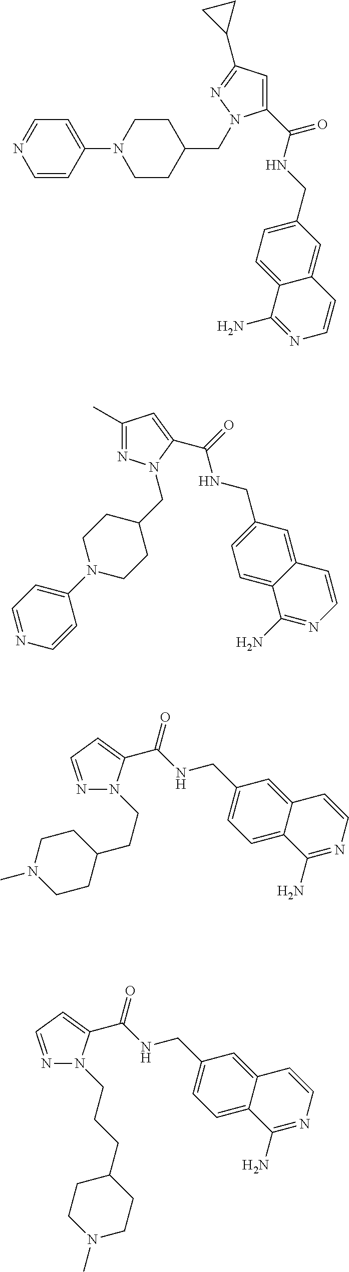 [SMILES: Nc1nccc2cc(CNC(=O)c3cc(C4CC4)nn3CC3CCN(c4ccncc4)CC3)ccc12 ; Cc1cc(C(=O)NCc2ccc3c(N)nccc3c2)n(CC2CCN(c3ccncc3)CC2)n1 ; CN1CCC(CCn2nccc2C(=O)NCc2ccc3c(N)nccc3c2)CC1 ; CN1CCC(CCCn2nccc2C(=O)NCc2ccc3c(N)nccc3c2)CC1] 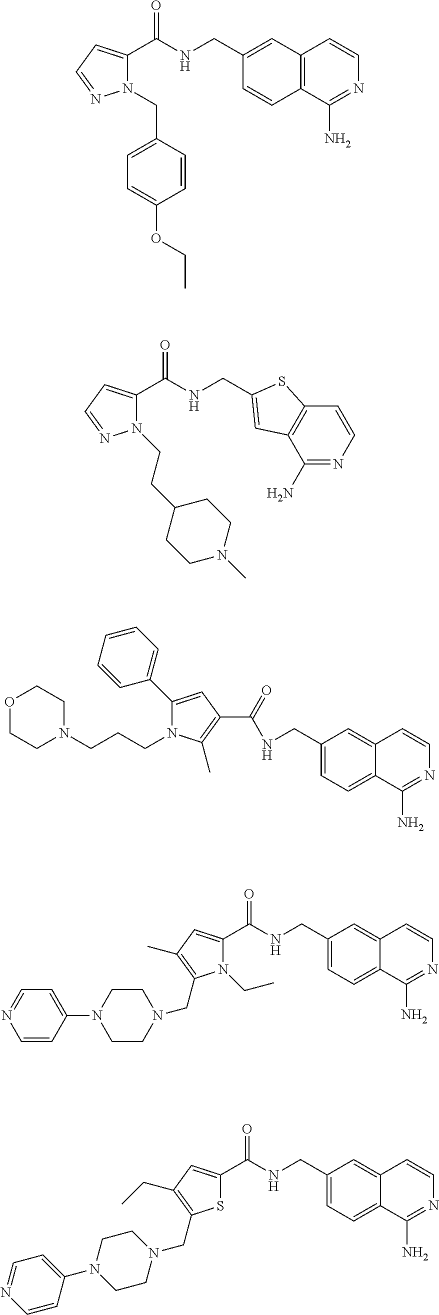 [SMILES: CCOc1ccc(Cn2nccc2C(=O)NCc2ccc3c(N)nccc3c2)cc1 ; CN1CCC(CCn2nccc2C(=O)NCc2cc3c(N)nccc3s2)CC1 ; Cc1c(C(=O)NCc2ccc3c(N)nccc3c2)cc(-c2ccccc2)n1CCCN1CCOCC1 ; CCn1c(C(=O)NCc2ccc3c(N)nccc3c2)cc(C)c1CN1CCN(c2ccncc2)CC1 ; CCc1cc(C(=O)NCc2ccc3c(N)nccc3c2)sc1CN1CCN(c2ccncc2)CC1] 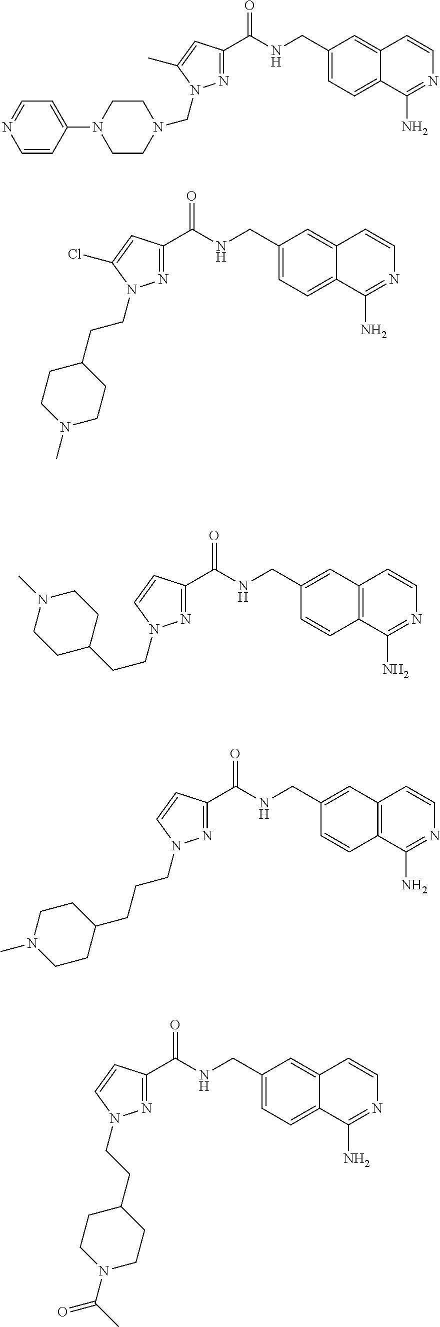 [SMILES: Cc1cc(C(=O)NCc2ccc3c(N)nccc3c2)nn1CN1CCN(c2ccncc2)CC1 ; CN1CCC(CCn2nc(C(=O)NCc3ccc4c(N)nccc4c3)cc2Cl)CC1 ; CN1CCC(CCn2ccc(C(=O)NCc3ccc4c(N)nccc4c3)n2)CC1 ; CN1CCC(CCCn2ccc(C(=O)NCc3ccc4c(N)nccc4c3)n2)CC1 ; CC(=O)N1CCC(CCn2ccc(C(=O)NCc3ccc4c(N)nccc4c3)n2)CC1] 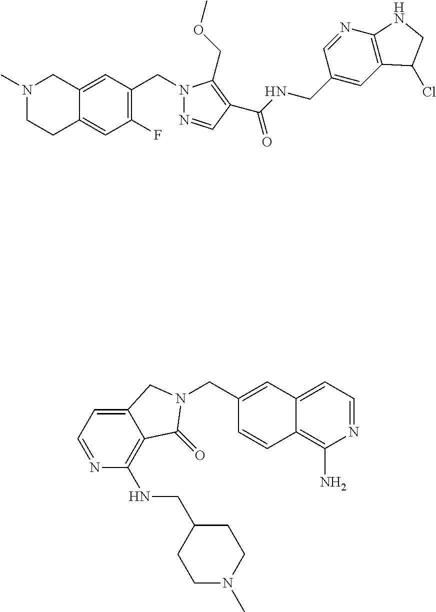 [SMILES: COCc1c(C(=O)NCc2cnc3c(c2)C(Cl)CN3)cnn1Cc1cc2c(cc1F)CCN(C)C2 ; CN1CCC(CNc2nccc3c2C(=O)N(Cc2ccc4c(N)nccc4c2)C3)CC1]
or a pharmaceutically acceptable salt and/or solvate thereof.

32. A pharmaceutical composition comprising: a compound or a pharmaceutically acceptable salt and/or solvate thereof according to claim 1, and at least one pharmaceutically acceptable excipient.

33. (canceled)

34. (canceled)

35. A method of treating a disease or condition in which Factor XIIa activity is implicated, comprising administering to a subject in need thereof a therapeutically effective amount of a compound or a pharmaceutically acceptable salt and/or solvate thereof according to claim 1.

36. (canceled)

37. The method of claim 35, wherein the disease or condition in which Factor XIIa activity is implicated is a bradykinin-mediated angioedema.

38. The method of claim 37, wherein the bradykinin-mediated angioedema is hereditary angioedema.

39. The method of claim 37, wherein the bradykinin-mediated angioedema is non hereditary.

40. The method of claim 35, wherein the disease or condition in which Factor XIIa activity is implicated is selected from vascular hyperpermeability; stroke including ischemic stroke and haemorrhagic accidents; retinal edema; diabetic retinopathy; DME; retinal vein occlusion; or AMD.

41. The method of claim 35, wherein the disease or condition in which Factor XIIa activity is implicated is a thrombotic disorder.

42. The method of claim 41, wherein the thrombotic disorder is thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget Schroetter syndrome and Budd-Chari syndrome; and atherosclerosis.

43. The method of claim 35, wherein the disease or condition in which Factor XIIa activity is implicated is selected from neuroinflammation; neuroinflammatory/neurodegenerative disorders such as MS (multiple sclerosis); other neurodegenerative diseases such as Alzheimer's disease, epilepsy and migraine, sepsis; bacterial sepsis; inflammation; vascular hyperpermeability; or anaphylaxis.

44. The method of claim 35, wherein the compound targets FXIIa.

45. A method of treating a disease or condition in which Factor XIIa activity is implicated, comprising administering to a subject in need thereof a therapeutically effective amount of the pharmaceutical composition of claim 32.

46. The compound of claim 1, wherein the stereoisomer is an enantiomer, diastereoisomer, or a racemic or scalemic mixture thereof.
